(19) United States
(12) Patent Application Publication
(10) Pub. No.: US 2012/0283411 A9
(43) Pub. Date: Nov. 8, 2012

(54) METHODS AND COMPOSITIONS FOR THE TREATMENT OF GASTROINTESTINAL DISORDERS

(75) Inventors: Mark G. Currie, Sterling, MA (US); Daniel P. Zimmer, Somerville, MA (US)

(73) Assignee: IRONWOOD PHARMACEUTICALS, INC., Cambridge, MA (US)

(21) Appl. No.: 12/306,788

(22) PCT Filed: Jun. 27, 2007
(86) PCT No.: PCT/US07/72223

§ 371 Date: Nov. 9, 2009

Related U.S. Application Data
(60) Provisional application No. 60/817,843, filed on Jun. 29, 2006.

(51) Int. Cl. C07K 14/00; C07K 7/08; C07K 7/06

(52) U.S. Cl. 530/324; 530/328; 530/327; 530/326; 530/325

(57) ABSTRACT

Compositions and related methods for treating IBS and other gastrointestinal disorders and conditions (e.g., gastrointestinal motility disorders, functional gastrointestinal disorders, gastroesophageal reflux disease (GERD), duodenogastric reflux, Crohn's disease, ulcerative colitis, inflammatory bowel disease, functional heartburn, dyspepsia (including functional dyspepsia or nonulcer dyspepsia), gastroparesis, chronic intestinal pseudo-obstruction (or colonic pseudoobstruction), disorders and conditions associated with constipation, e.g., constipation associated with use of opiate pain killers, post-surgical constipation, and constipation associated with neuropathic disorders and disorders and conditions associated with excess fluid and/or salt retention as well as other conditions and disorders are described. The compositions feature polypeptides that activate the guanylate cyclase C (GC-C) receptor.

### TECHNICAL FIELD

This invention relates to methods and compositions for treating gastrointestinal disorders, obesity, congestive heart failure, benign prostatic hyperplasia (BPH) and other disorders.

### BACKGROUND

Irritable bowel syndrome (IBS) is a common chronic disorder of the intestine that affects 20 to 60 million individuals in the US alone (Lehman Brothers, Global Healthcare-Irritable Bowel Syndrome Industry Update, September 1999). IBS is the most common disorder diagnosed by gastroenterologists (28% of patients examined) and accounts for 12% of visits to primary care physicians (Camilleri 2001 Gastroenterology 120:652-668). In the US, the economic impact of IBS is estimated at $25 billion annually, through direct costs of health care use and indirect costs of absenteeism from work (Talley 1995 Gastroenterology 109:1736-1741). Patients with IBS have three times more absenteeism from work and report a reduced quality of life. Sufferers may be unable or unwilling to attend social events, maintain employment, or travel even short distances (Drossman 1993 Dig Dis Sci 38:1569-1580). There is a tremendous unmet medical need in this population since few prescription options exist to treat IBS.

Patients with IBS suffer from abdominal pain and a disturbed bowel pattern. Three subgroups of IBS patients have been defined based on the predominant bowel habit: constipation-predominant (c-IBS), diarrhea-predominant (d-IBS) or alternating between the two (a-IBS). Estimates of individuals who suffer from c-IBS range from 20-50% of the IBS patients with 30% frequently cited. In contrast to the other two subgroups that have a similar gender ratio, c-IBS is more common in women (ratio of 3:1) (Talley et al. 1995 Am J Epidemiol 142:76-83).

The definition and diagnostic criteria for IBS have been formalized in the “Rome Criteria” (Drossman et al. 1999, Gut 45:Suppl II: 1-81), which are well accepted in clinical practice. Briefly, the criteria specify that for at least 12 weeks (consecutive or non-consecutive in the preceding 12 months of abdominal discomfort or pain at least two of the following three features must occur: (1) relieved with defecation, (2) onset associated with a change in frequency of stool, and (3) onset associated with a change in form (appearance) of stool. The Rome II criteria also state that the symptoms that cumulatively support the diagnosis of irritable bowel syndrome include: abnormal stool frequency (“abnormal” may be defined as greater than 3 bowel movements per day and less than 3 bowel movements per week), abnormal stool form (lumpy/hard or loose/watery stool), abnormal stool passage (straining, urgency, or feeling of incomplete evacuation), passage of mucus, and bloating or feeling of abdominal distension. However, the complexity of symptoms has not been explained by anatomical abnormalities or metabolic changes. This has led to the classification of IBS as a functional GI disorder, which is diagnosed on the basis of the Rome criteria and limited evaluation to exclude organic disease (Ringel et al. 2001, Annu Rev Med 52: 319-338). IBS is considered to be a “biopsychosocial” disorder resulting from a combination of three interacting mechanisms: altered bowel motility, an increased sensitivity of the intestine or colon to pain stimuli (visceral sensitivity) and psychosocial factors (Camilleri 2001, Gastroenterology 120:652-668). Recently, there has been increasing evidence for a role of inflammation in etiology of IBS. Reports indicate that subsets of IBS patients have small but significant increases in colonic inflammatory and mast cells, increased inducible nitric oxide (NO) and synthase (iNOS) and altered expression of inflammatory cytokines (reviewed by Talley 2000, Medscape Coverage of DDW week).

The guanylate cyclase-C (GC-C) receptor (reviewed by Lucas et al. 2000 Pharmacol. Rev 52:375-414 and Vaandrager et al. 2002 Molecular and Cellular Biochemistry 230:73-83) is a key regulator in mammals of intestinal function (although low levels of GC-C have been detected in other tissues). GC-C responds to the endogenous hormones, guanylin and uroguanylin, and to enteric bacterial polypeptides from the heat stable enterotoxin family (ST polypeptides). When an agonist binds to GC-C, there is an elevation of the second messenger, cyclic GMP, and an increase in chloride and bicarbonate secretion, resulting in an increase in intestinal fluid secretion. The Genbank protein GI accession number for guanylyl cyclase C homologs from multiple organisms are:

| Genbank GI | |
|---|---|
| number | organism |
| 27806993 | Cattle |
| 16555439 | Eel |
| 16555437 | Eel |
| 4521169 | Fish |
| 1850774 | Frog |
| 1495352 | Guinea pig |
| 2494861 | Guinea pig |
| 4826752 | human |
| 4505441 | human |
| 1184046 | human |
| 1230617 | mouse |
| 2708786 | mouse |
| 71894985 | mouse |
| 47523018 | Pig |
| 5930067 | rabbit |
| 6981000 | Rat |
| 40445437 | Worm |

### SUMMARY DESCRIBED HEREIN

Described herein are compositions and related methods for treating a variety of disorders, including IBS and other gastrointestinal disorders and conditions (e.g., gastrointestinal motility disorders, inflammatory bowel disease (IBD), chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, irritable bowel syndrome, post-operative ileus, ulcerative colitis, chronic constipation, and disorders and conditions associated with constipation (e.g. constipation associated with use of opiate pain killers, post-surgical constipation, and constipation associated with neuropathic disorders as well as other conditions and disorders are described herein.

The therapeutic compositions and methods described herein employ polypeptides that include at least a portion of the pro sequence of guanylin or uroguanylin or variants of such polypeptides. Without being bound by any particular theory, polypeptides that include portions of the pro sequence may bind to and activate the GC-C receptor and/or another target.

Also described herein are compositions and related methods for treating obesity, congestive heart failure (including congestive heart failure at any of stages I-IV according to New York Heart Association (NYHA) Functional Classification) and benign prostatic hyperplasia (BPH).

Without being bound by any particular theory, in the case of IBS and other gastrointestinal disorders the polypeptides are useful, in part, because they can increase gastrointestinal motility.

Without being bound by any particular theory, in the case of IBS and other gastrointestinal disorders the polypeptides are useful, in part, because they can decrease inflammation.

Without being bound by any particular theory, in the case of IBS and other gastrointestinal disorders the polypeptides are also useful because they may decrease gastrointestinal pain, visceral pain, chronic visceral hypersensitivity, or hypersensitivity to colorectal distension.

Without being bound by any particular theory, in the case of salt retention, fluid retention disorders and combinations thereof the polypeptides are also useful because they may elicit one or more of diuresis, naturesis and/or kaliuresis. Thus the peptides described herein may be diuretics.

Described are pharmaceutical compositions comprising a polypeptide described herein as well as combination compositions comprising a polypeptide described herein and one or more additional therapeutic agents including, without limitation, the agents described herein. The other agents can be administered with the polypeptides described herein (simultaneously or sequentially). They can also be linked to a polypeptide described herein to create therapeutic conjugates.

Described herein are various useful polypeptides that include all or a portion of the sequence of the pro sequence of human guanylin. Thus, certain useful polypeptides include all or a portion of the sequence:

VTVQDGNFSFSLESVKKLKDLQEPQEPRVGKLRNFAPIPGEPVVPILCSNPNFPEELKPLC KEPNAQEILQRLEEIAED (SEQ ID NO:X′; human guanylin pro sequence). Other useful polypeptides include all or portion of the pro sequence of human guanylin together with all or a portion of the sequence of mature human guanylin. Thus, certain useful polypeptides include all or a portion of the sequence:

(SEQ ID NO: X; human proguanylin; mature guanylin portion underlined).
VTVQDGNFSFSLESVKKLKDLQEPQEPRVGKLRNFAPIPGEPVVPILCSNPNFPEELKPLC
KEPNAQEILQRLEEIAEDPGTCEICAYAACTGC

Described herein are various useful polypeptides that include all or a portion of the sequence of the pro sequence of human uroguanylin. Thus, certain useful polypeptides include all or a portion of the sequence:

VYIQYQGFRVQLESMKKLSDLEAQWAPSPRLQAQSLLPAVCHHPALPQDLQPVCASQE ASSIFKTLRTIA (SEQ ID NO:ZZ′-human uroguanylin prosequence). Other useful polypeptides include all or portion of the pro sequence of human uroguanylin together with all or a portion of the sequence of mature human uroguanylin. Thus, certain useful polypeptides include all or a portion of the sequence:

(SEQ ID NO: ZZ-human uroguanylin; mature
uroguanylin portion underlined).
VYIQYQGFRVQLESMKKLSDLEAQWAPSPRLQAQSLLPAVCHHPALPQDL
QPVCASQEASSIFKTLRTIANDDCELCVNVACTGCL

Other useful polypeptides include all or a portion of a polypeptide, SEQ ID NO:X1, that is related to human proguanylin. Thus, useful polypeptides include a polypeptide comprising (or consisting of or consisting essentially of) at least 10 contiguous amino acid of a polypeptide having the sequence: X₁ X₂ X₃ X₄ X₅ X₆ X₇ X₈ X₉ X₁₀ X₁₁ L E X₁₄ V K X₁₇ L X₁₉ X₂₀ L X₂₂ X₂₃ X₂₄ X₂₅ X₂₆ X₂₇ X₂₈ X₂₉ X₃₀ X₃₁ X₃₂ X₃₃ X₃₄ X₃₅ X₃₆ X₃₇ X₃₈ X₃₉ X₄₀ X₄₁ X₄₂ X₄₃ X₄₄ X₄₅ X₄₆ X₄₇ X₄₈ X₄₉ X₅₀ C X₅₂ X₅₃ X₅₄ X₅₅ X₅₆ X₅₇ P X₅₉ X₆₀ X₆₁ X₆₂ P X₆₄ C X₆₆ X₆₇ X₆₈ X₆₉ X₇₀ X₇₁ X₇₂ X₇₃ X₇₄ X₇₅ R L X₇₈ X₇₉ X₈₀ X₈₁ X₈₂ X₈₃ P G T C E I C A Y A A C T G C X₉₉ (SEQ ID NO:X1-guanylin consensus 1)

wherein:

### X₁ is V or S;

### X₂ is T, L, I, Y or E;

### X₃ is V or F;

### X₄ is Q or K;

### X₅ is D or E;

### X₆ is G or N;

### X₇ is D, N, E or G;

### X₈ is F or L;

### X₉ is S, T or K;

### X₁₀ is F or Y;

### X₁₁ is S or P;

### X₁₄ is S or A;

### X₁₇ is K, Q or R;

### X₁₉ is K or H;

### X₂₀ is D, E, A, H or G;

### X₂₂ is Q, R, G, M, A;

### X₂₃ is E, Q or D;

### X₂₄ is A, S, E, V, L or P;

### X₂₅ is Q, N, P, G or S;

### X₂₆ is E, K, M or V;

X₂₇ is G, L or is missing;
X₂₈ is Q, S, R, A or is missing;
X₂₉ is E, K, S, A or is missing;

### X₃₀ is P, V, M or A;

X₃₁ is R, Q, T, I, A or is missing;

### X₃₂ is L, V, I, G, N or S;

### X₃₃ is P, G, R, V, M, A, P;

### X₃₄ is S, R or K;

### X₃₅ is H, L, I, N or K;

X₃₆ is R, K or is missing;
X₃₇ is N, K or is missing;
X₃₈ is F or is missing;
X₃₉ is A or is missing;
X₄₀ is P, L or is missing;
X₄₁ is I, R or is missing;
X₄₂ is L, P, F, V, R or is missing;
X₄₃ is G, V, D, P, L, A or is missing;

### X₄₄ is G, E, K, A, Q, R or S;

### X₄₅ is P, S, H or K;

### X₄₆ is V, I, P, A or Q;

### X₄₇ is A, V, I, A, L, G or T;

X₄₈ is P, A, S, Y or is missing;

### X₄₉ is I, Q, V, N, G, E, H, S or F;

### X₅₀ is L, A or P;

### X₅₂ is S, N, A, Q or G;

X₅₃ is S or missing;

### X₅₄ is H, N, D, S, L, F, or Q;

### X₅₅ is P, S, L or K;

### X₅₆ is A, K, N, T, G, or Q;

### X₅₇ is F or L;

### X₅₉ is E, K or Q;

### X₆₀ is E, A or D;

### X₆₁ is L or F;

### X₆₂ is K, R, Q or L;

### X₆₄ is L, I or V;

### X₆₆ is K, E, Q, T or R;

### X₆₇ is E, K, R or Q;

### X₆₈ is P, S, E or R;

### X₆₉ is N, D or G;

### X₇₀ is A or S;

### X₇₁ is E, Q, P, A or S;

### X₇₂ is E, D, Q, M or A;

### X₇₃ is I, A, or S;

### X₇₄ is L, F or V;

### X₇₅ is Q, E, D, N, G or A;

### X₇₈ is E, A, G or C;

### X₇₉ is E, A, V, S, L or M;

### X₈₀ is I or V;

### X₈₁ is A or P;

### X₈₂ is E, Q, A or S;

### X₈₃ is D or E; and

X₉₉ is F or is missing.

Other useful polypeptides include all or a portion of a polypeptide, SEQ ID NO:X2, that, like SEQ ID NO:X1, is related to human proguanylin. Thus, useful polypeptides include a polypeptide comprising (or consisting of or consisting essentially of) at least 10 contiguous amino acid of a polypeptide having the sequence:

(SEQ ID NO: X2-guanylin consensus 2),
X₁ X₂ X₃ X₄ X₅ X₆ X₇ X₈ X₉ X₁₀ X₁₁ L E X₁₄ V K X₁₇ L X₁₉ X₂₀ L X₂₂ X₂₃ X₂₄ X₂₅ X₂₆ X₂₇ X₂₈ X₂₉
X₃₀ X₃₁ X₃₂ X₃₃ X₃₄ X₃₅ X₃₆ X₃₇ X₃₈ X₃₉ X₄₀ X₄₁ X₄₂ X₄₃ X₄₄ X₄₅ X₄₆ X₄₇ X₄₈ X₄₉ X₅₀ C X₅₂ X₅₃ X₅₄
X₅₅ X₅₆ X₅₇ P X₅₉ X₆₀ X₆₁ X₆₂ P X₆₄ C X₆₆ X₆₇ X₆₈ X₆₉ X₇₀ X₇₁ X₇₂ X₇₃ X₇₄ X₇₅ R L X₇₈ X₇₉ X₈₀ X₈₁
X₈₂ X₈₃ P X₈₅ X₈₆ C E I C A X₉₂ A A C X₉₆ G C X₉₉

wherein:

### X₁ is V or S;

### X₂ is T, L, I, Y or E;

### X₃ is V or F;

### X₄ is Q or K;

### X₅ is D or E;

### X₆ is G or N;

### X₇ is D, N, E or G;

### X₈ is F or L;

### X₉ is S, T or K;

### X₁₀ is F or Y;

### X₁₁ is S or P;

### X₁₄ is S or A;

### X₁₇ is K, Q or R;

### X₁₉ is K or H;

### X₂₀ is D, E, A, H or G;

### X₂₂ is Q, R, G, M, A;

### X₂₃ is E, Q or D;

### X₂₄ is A, S, E, V, L or P;

### X₂₅ is Q, N, P, G or S;

### X₂₆ is E, K, M or V;

X₂₇ is G, L or is missing;
X₂₈ is Q, S, R, A or is missing;
X₂₉ is E, K, S, A or is missing;

### X₃₀ is P, V, M or A;

X₃₁ is R, Q, T, I, A or is missing;

### X₃₂ is L, V, I, G, N or S;

### X₃₃ is P, G, R, V, M, A, P;

### X₃₄ is S, R or K;

### X₃₅ is H, L, I, N or K;

X₃₆ is R, K or is missing;
X₃₇ is N, K or is missing;
X₃₈ is F or is missing;
X₃₉ is A or is missing;
X₄₀ is P, L or is missing;
X₄₁ is I, R or is missing;
X₄₂ is L, P, F, V, R or is missing;
X₄₃ is G, V, D, P, L, A or is missing;

### X₄₄ is G, E, K, A, Q, R or S;

### X₄₅ is P, S, H or K;

### X₄₆ is V, I, P, A or Q;

### X₄₇ is A, V, I, A, L, G or T;

X₄₈ is P, A, S, Y or is missing;

### X₄₉ is I, Q, V, N, G, E, H, S or F;

### X₅₀ is L, A or P;

### X₅₂ is S, N, A, Q or G;

X₅₃ is S or missing;

### X₅₄ is H, N, D, S, L, F, or Q;

### X₅₅ is P, S, L or K;

### X₅₆ is A, K, N, T, G, or Q;

### X₅₇ is F or L;

### X₅₉ is E, K or Q;

### X₆₀ is E, A or D;

### X₆₁ is L or F;

### X₆₂ is K, R, Q or L;

### X₆₄ is L, I, or V;

### X₆₆ is K, E, Q, T or R;

### X₆₇ is E, K, R or Q;

### X₆₈ is P, S, E or R;

### X₆₉ is N, D or G;

### X₇₀ is A or S;

### X₇₁ is E, Q, P, A or S;

### X₇₂ is E, D, Q, M or A;

### X₇₃ is I, A, or S;

### X₇₄ is L, F or V;

### X₇₅ is Q, E, D, N, G or A;

### X₇₈ is E, A, G or C;

### X₇₉ is E, A, V, S, L or M;

### X₈₀ is I or V;

### X₈₁ is A or P;

### X₈₂ is E, Q, A or S;

### X₈₃ is D or E;

### X₈₅ is G, S, R, or N;

### X₈₆ is S or T;

### X₉₂ is Y or F;

### X₉₆ is T or A; and

X₉₉ is F or is missing.

Other useful polypeptide include all or a portion of a polypeptide, SEQ ID NO:ZZ1, that is related to human prouroguanylin. Thus, useful polypeptides include a polypeptide comprising (or consisting of or consisting essentially of) at least 10 contiguous amino acid of a polypeptide having the sequence: V X₂ I X₄ Y X₆ G X₈ X₉ V X₁₁ L X₁₃ S X₁₅ K X₁₇ L X₁₉ X₂₀ L X₂₂ X₂₃ X₂₄ X₂₅ X₂₆ X₂₇ X₂₈ X₂₉ X₃₀ X₃₁ X₃₂ X₃₃ X₃₄ X₃₅ X₃₆ X₃₇ X₃₈ X₃₉ X₄₀ X₄₁ C X₄₃ X₄₄ X₄₅ A L P X₄₉ D L X₅₂ P X₅₄ C X₅₆ X₅₇ X₅₈ X₅₉ X₆₀ X₆₁ X₆₂ X₆₃ X₆₄ X₆₅ X₆₆ L R X₆₉ X₇₀ X₇₁N D D C E L C V N V A (SEQ ID NO:ZZ1-uroguanylin consensus 1), wherein:

### X₂ is Y or D;

### X₄ is Q or K;

### X₆ is Q, H or E;

### X₈ is F or Y;

### X₉ is R or Q;

### X₁₁ is Q or K;

### X₁₃ is E, K or D;

### X₁₅ is M or V;

### X₁₇ is K or Q;

### X₁₉ is S, N, K or D;

### X₂₀ is D, E or A;

### X₂₂ is E, V or L;

### X₂₃ is A, E or G;

### X₂₄ is Q or K;

### X₂₅ is W, Q, E or P;

### X₂₆ is A, M, V or R;

### X₂₇ is P or S;

### X₂₈ is S, N, D or F;

### X₂₉ is P or R;

### X₃₀ is R, Q, G or H;

### X₃₁ is L, P, Q or R;

### X₃₂ is Q, R or M;

### X₃₃ is A, K, R, D or G;

### X₃₄ is Q, S or T;

### X₃₅ is S, G, D or Q;

X₃₆ is L, R or is missing;

### X₃₇ is L, P or D;

### X₃₈ is L, Q or P;

### X₃₉ is P or S;

### X₄₀ is A, S, D or V;

### X₄₁ is V or L;

### X₄₃ is H, Y or S;

### X₄₄ is H, N or D;

### X₄₅ is P or S;

### X₄₉ is Q, L, P or S;

### X₅₂ is Q or R;

### X₅₄ is V or I;

### X₅₆ is A, Q, T or E;

### X₅₇ is S or N;

### X₅₈ is Q, E, K or S;

### X₅₉ is E, D or Q;

### X₆₀ is A or V;

### X₆₁ is S or A;

### X₆₂ is S or N;

### X₆₃ is I or T;

### X₆₄ is F or L;

### X₆₅ is K, Q or L;

### X₆₆ is T or A;

### X₆₉ is T or S;

### X₇₀ is I or M; and

### X₇₁ is A, S or D.

Other useful polypeptides include all or a portion of a polypeptide, SEQ ID NO:ZZ2, that, like SEQ ID NO:ZZ1, is related to human prouroguanylin. Thus, useful polypeptides include a polypeptide comprising (or consisting of or consisting essentially of) at least 10 contiguous amino acid of a polypeptide having the sequence

V X₂, X₄ Y X₆ G X₈ X₉ V X₁₁ L X₁₃ S X₁₅ K X₁₇ L X₁₉ X₂₀ L X₂₂ X₂₃ X₂₄ X₂₅ X₂₆ X₂₇ X₂₈ X₂₉ X₃₀ X₃₁ X₃₂ X₃₃ X₃₄ X₃₅ X₃₆ X₃₇ X₃₈ X₃₉ X₄₀ X₄₁ C X₄₃ X₄₄ X₄₅ A L P X₄₉ D L X₅₂ P X₅₄ C X₅₆ X₅₇ X₅₈ X₅₉ X₆₀ X₆₁ X₆₂ X₆₃ X₆₄ X₆₅ X₆₆ L R X₆₉ X₇₀ X₇₁ X₇₂ D X₇₄ C E L C X₇₉ N V A (SEQ ID NO:ZZ2-uroguanylin consensus 2), wherein:

### X₂ is Y or D;

### X₄ is Q or K;

### X₆ is Q, H or E;

### X₈ is F or Y;

### X₉ is R or Q;

### X₁₁ is Q or K;

### X₁₃ is E, K or D;

### X₁₅ is M or V;

### X₁₇ is K or Q;

### X₁₉ is S, N, K or D;

### X₂₀ is D, E or A;

### X₂₂ is E, V or L;

### X₂₃ is A, E or G;

### X₂₄ is Q or K;

### X₂₅ is W, Q, E or P;

### X₂₆ is A, M, V or R;

### X₂₇ is P or S;

### X₂₈ is S, N, D or F;

### X₂₉ is P or R;

### X₃₀ is R, Q, G or H;

### X₃₁ is L, P, Q or R;

### X₃₂ is Q, R or M;

### X₃₃ is A, K, R, D or G;

### X₃₄ is Q, S or T;

### X₃₅ is S, G, D or Q;

X₃₆ is L, R or is missing;

### X₃₇ is L, P or D;

### X₃₈ is L, Q or P;

### X₃₉ is P or S;

### X₄₀ is A, S, D or V;

### X₄₁ is V or L;

### X₄₃ is H, Y or S;

### X₄₄ is H, N or D;

### X₄₅ is P or S;

### X₄₉ is Q, L, P or S;

### X₅₂ is Q or R;

### X₅₄ is V or I;

### X₅₆ is A, Q, T or E;

### X₅₇ is S or N;

### X₅₈ is Q, E, K or S;

### X₅₉ is E, D or Q;

### X₆₀ is A or V;

### X₆₁ is S or A;

### X₆₂ is S or N;

### X₆₃ is I or T;

### X₆₄ is F or L;

### X₆₅ is K, Q or L;

### X₆₆ is T or A;

### X₆₉ is T or S;

### X₇₀ is I or M;

### X₇₁ is A, S or D;

### X₇₂ is N, T, G or Q;

### X₇₄ is D or E; and

### X₇₉ is V or I.

Thus, described herein are 1) purified polypeptides comprising (or consisting of or consisting essentially of) at least 10 contiguous amino acids of SEQ ID NO:X′ and 2) purified polypeptides consisting of a polypeptide fragment of SEQ ID NO:X′. comprising (or consisting of or consisting essentially of) at least 10 contiguous amino acids of SEQ ID NO:X′ VTVQDGNFSFSLESVKKLKDLQEPQEPRVGKLRNFAPIPGEPVVPILCSNPNFPEELKPLC KEPNAQEILQRLEEIAED (SEQ ID NO:X′; human guanylin prosequence). In various embodiments the purified polypeptide comprises at least 15 amino acids, at least 20 amino acids, at least 25 amino acids, at least 30 amino acids, at least 40 amino acids, at least 45 amino acids, at least 50 amino acids, at least 55 amino acids, at least 60 amino acids, at least 65 amino acids, at least 70 amino acids, at least 75 amino acids, or at least 80 amino acids. In various embodiments the purified polypeptide comprises fewer than 80, 75, 70 65, 60, 55, 50, 45, 40, 35, 30, 25 or 20 contiguous amino acids of SEQ ID NO:X′, and has the ability to bind and/or activate the GC-C receptor and/or elicit diuresis when administered to a subject.

Thus, described herein are 1) purified polypeptides comprising (or consisting of or consisting essentially of) at least 10 contiguous amino acids of SEQ ID NO:X and 2) purified polypeptides consisting of a polypeptide fragment of SEQ ID NO:X comprising (or consisting of or consisting essentially of) at least 10 contiguous amino acids of SEQ ID NO:X

VTVQDGNFSFSLESVKKLKDLQEPQEPRVGKLRNFAPIPGEPVVPILCSNPNFPEELKPLC KEPNAQEILQRLEEIAEDPGTCEICAYAACTGC (SEQ ID NO:X; human proguanylin). In various embodiments: the purified polypeptide comprises at least 15 amino acids, at least 20 amino acids, at least 25 amino acids, at least 30 amino acids, at least 40 amino acids, at least 45 amino acids, at least 50 amino acids, at least 55 amino acids, at least 60 amino acids, at least 65 amino acids, at least 70 amino acids, at least 75 amino acids, or at least 80 amino acids. In various embodiments the purified polypeptide comprises fewer than 80, 75, 70 65, 60, 55, 50, 45, 40, 35, 30, 25 or 20 contiguous amino acids of SEQ ID NO:X and has the ability to bind and/or activate the GC-C receptor and/or elicit diuresis when administered to a subject.

Compositions, including pharmaceutical compositions, can include at least one such polypeptide or can include at least two (three, four or more) such polypeptides which are different. In the compositions containing two or more such polypeptides the polypeptides can be separate or they can be covalently direct linked, e.g, by a peptide bond or a linker or they can be indirectly linked. For example, two such polypeptide sequences can be contained within a larger polypeptide and the two polypeptide sequences can be separated by other polypeptide sequences.

Among the examples of a purified polypeptide comprising at least 10 contiguous amino acids of SEQ ID NO:X and the examples of a purified polypeptide consisting of a polypeptide fragment of SEQ ID NO:X comprising at least 10 contiguous amino acids of SEQ ID NO:X are:
a) a polypeptide comprising amino acids 1-16 of SEQ ID NO:X;
b) a polypeptide comprising amino acids 1-47 of SEQ ID NO:X;
c) a polypeptide comprising amino acids 1-61 of SEQ ID NO:X;
d) a polypeptide comprising amino acids 1-72 of SEQ ID NO:X;
e) a polypeptide comprising amino acids 17-47 of SEQ ID NO:X;
f) a polypeptide comprising amino acids 17-61 of SEQ ID NO:X;
g) a polypeptide comprising amino acids 17-72 of SEQ ID NO:X;
h) a polypeptide comprising amino acids 17-94 of SEQ ID NO:X;
i) a polypeptide comprising amino acids 48-61 of SEQ ID NO:X;
j) a polypeptide comprising amino acids 48-72 of SEQ ID NO:X;
k) a polypeptide comprising amino acids 48-94 of SEQ ID NO:X;
l) a polypeptide comprising amino acids 62-72 of SEQ ID NO:X;
m) a polypeptide comprising amino acids 62-94 of SEQ ID NO:X;
n) a polypeptide comprising amino acids 73-94 of SEQ ID NO:X;
o) a polypeptide comprising amino acids 1-23 of SEQ ID NO:X;
p) a polypeptide comprising amino acids 48-66 of SEQ ID NO:X;
q) a polypeptide comprising amino acids 48-71 of SEQ ID NO:X;
r) a polypeptide comprising amino acids 48-76 of SEQ ID NO:X;
s) a polypeptide comprising amino acids 43-61 of SEQ ID NO:X;
t) a polypeptide comprising amino acids 38-61 of SEQ ID NO:X;
u) a polypeptide comprising amino acids 33-61 of SEQ ID NO:X;
v) a polypeptide comprising amino acids 43-66 of SEQ ID NO:X;
w) a polypeptide comprising amino acids 38-66 of SEQ ID NO:X;
x) a polypeptide comprising amino acids 33-66 of SEQ ID NO:X;
y) a polypeptide comprising amino acids 43-71 of SEQ ID NO:X;
z) a polypeptide comprising amino acids 38-71 of SEQ ID NO:X;
aa) a polypeptide comprising amino acids 33-71 of SEQ ID NO:X;
ab) a polypeptide comprising amino acids 43-76 of SEQ ID NO:X;
ac) a polypeptide comprising amino acids 38-76 of SEQ ID NO:X; and
ad) a polypeptide comprising amino acids 33-76 of SEQ ID NO:X.

The contemplated purified polypeptides include any subset of the aforementioned polypeptides as well as each one of the forgoing polypeptides.

Among the examples of a purified polypeptide comprising at least 10 contiguous amino acids of SEQ ID NO:X and the examples of a purified polypeptide consisting of a polypeptide fragment of SEQ ID NO:X comprising at least 10 contiguous amino acids of SEQ ID NO:X are:
a) a polypeptide consisting of amino acids 1-16 of SEQ ID NO:X;
b) a polypeptide consisting of amino acids 1-47 of SEQ ID NO:X;
c) a polypeptide consisting of amino acids 1-61 of SEQ ID NO:X;
d) a polypeptide consisting of amino acids 1-72 of SEQ ID NO:X;
e) a polypeptide consisting of amino acids 17-47 of SEQ ID NO:X;
f) a polypeptide consisting of amino acids 17-61 of SEQ ID NO:X;
g) a polypeptide consisting of amino acids 17-72 of SEQ ID NO:X;
h) a polypeptide consisting of amino acids 17-94 of SEQ ID NO:X;
i) a polypeptide consisting of amino acids 48-61 of SEQ ID NO:X;
j) a polypeptide consisting of amino acids 48-72 of SEQ ID NO:X;
k) a polypeptide consisting of amino acids 48-94 of SEQ ID NO:X;
l) a polypeptide consisting of amino acids 62-72 of SEQ ID NO:X;
m) a polypeptide consisting of amino acids 62-94 of SEQ ID NO:X;
n) a polypeptide consisting of amino acids 73-94 of SEQ ID NO:X;
o) a polypeptide consisting of amino acids 1-23 of SEQ ID NO:X;
p) a polypeptide consisting of amino acids 48-66 of SEQ ID NO:X;
q) a polypeptide consisting of amino acids 48-71 of SEQ ID NO:X;
r) a polypeptide consisting of amino acids 48-76 of SEQ ID NO:X;
s) a polypeptide consisting of amino acids 43-61 of SEQ ID NO:X;
t) a polypeptide consisting of amino acids 38-61 of SEQ ID NO:X;
u) a polypeptide consisting of amino acids 33-61 of SEQ ID NO:X;
v) a polypeptide consisting of amino acids 43-66 of SEQ ID NO:X;
w) a polypeptide consisting of amino acids 38-66 of SEQ ID NO:X;
x) a polypeptide consisting of amino acids 33-66 of SEQ ID NO:X;
y) a polypeptide consisting of amino acids 43-71 of SEQ ID NO:X;
z) a polypeptide consisting of amino acids 38-71 of SEQ ID NO:X;
aa) a polypeptide consisting of amino acids 33-71 of SEQ ID NO:X;
ab) a polypeptide consisting of amino acids 43-76 of SEQ ID NO:X;
ac) a polypeptide consisting of amino acids 38-76 of SEQ ID NO:X; and
ad) a polypeptide consisting of amino acids 33-76 of SEQ ID NO:X.

The contemplated purified polypeptides include any subset of the aforementioned polypeptides as well as each one of the forgoing polypeptides.

Among the examples of a purified polypeptide comprising at least 10 contiguous amino acids of SEQ ID NO:X and the examples of a purified polypeptide consisting of a polypeptide fragment of SEQ ID NO:X comprising at least 10 contiguous amino acids of SEQ ID NO:X are:
a) a polypeptide consisting essentially of amino acids 1-16 of SEQ ID NO:X;
b) a polypeptide consisting essentially of amino acids 1-47 of SEQ ID NO:X;
c) a polypeptide consisting essentially of amino acids 1-61 of SEQ ID NO:X;
d) a polypeptide consisting essentially of amino acids 1-72 of SEQ ID NO:X;
e) a polypeptide consisting essentially of amino acids 17-47 of SEQ ID NO:X;
f) a polypeptide consisting essentially of amino acids 17-61 of SEQ ID NO:X;
g) a polypeptide consisting essentially of amino acids 17-72 of SEQ ID NO:X;
h) a polypeptide consisting essentially of amino acids 17-94 of SEQ ID NO:X;
i) a polypeptide consisting essentially of amino acids 48-61 of SEQ ID NO:X;
j) a polypeptide consisting essentially of amino acids 48-72 of SEQ ID NO:X;
k) a polypeptide consisting essentially of amino acids 48-94 of SEQ ID NO:X;
l) a polypeptide consisting essentially of amino acids 62-72 of SEQ ID NO:X;
m) a polypeptide consisting essentially of amino acids 62-94 of SEQ ID NO:X;
n) a polypeptide consisting essentially of amino acids 73-94 of SEQ ID NO:X;
o) a polypeptide consisting essentially of amino acids 1-23 of SEQ ID NO:X;
p) a polypeptide consisting essentially of amino acids 48-66 of SEQ ID NO:X;
q) a polypeptide consisting essentially of amino acids 48-71 of SEQ ID NO:X;
r) a polypeptide consisting essentially of amino acids 48-76 of SEQ ID NO:X;
s) a polypeptide consisting essentially of amino acids 43-61 of SEQ ID NO:X;
t) a polypeptide consisting essentially of amino acids 38-61 of SEQ ID NO:X;
u) a polypeptide consisting essentially of amino acids 33-61 of SEQ ID NO:X;
v) a polypeptide consisting essentially of amino acids 43-66 of SEQ ID NO:X;
w) a polypeptide consisting essentially of amino acids 38-66 of SEQ ID NO:X;
x) a polypeptide consisting essentially of amino acids 33-66 of SEQ ID NO:X;
y) a polypeptide consisting essentially of amino acids 43-71 of SEQ ID NO:X;
z) a polypeptide consisting essentially of amino acids 38-71 of SEQ ID NO:X;
aa) a polypeptide consisting essentially of amino acids 33-71 of SEQ ID NO:X;
ab) a polypeptide consisting essentially of amino acids 43-76 of SEQ ID NO:X;
ac) a polypeptide consisting essentially of amino acids 38-76 of SEQ ID NO:X; and
ad) a polypeptide consisting essentially of amino acids 33-76 of SEQ ID NO:X.

The contemplated purified polypeptides include any subset of the aforementioned polypeptides as well as each one of the forgoing polypeptides.

For SEQ ID NO:X useful compositions, including pharmaceutical compositions, that include, for example, more than one of polypeptides (a)-(ad) (or polypeptides comprising, consisting of or consisting essentially of one or more of (a)-(ad) or a consisting of a polypeptide fragment of SEQ ID NO:X comprising one or more of (a)-(ad)) include combinations 1-38 below:

| | |
|---|---|
| 1 | (a), (e) |
| 2 | (a), (e), (i) |
| 3 | (a), (e), (i), (l) |
| 4 | (a), (e), (i), (l), (n) |
| 5 | (a), (e), (i), (m) |
| 6 | (a), (e), (k) |
| 7 | (a), (f) |
| 8 | (a), (f), (l) |
| 9 | (a), (f), (l), (n) |
| 10 | (a), (f), (m) |
| 11 | (a), (g) |
| 12 | (a), (g), (n) |
| 13 | (a), (h) |
| 14 | (b), (i) |
| 15 | (b), (i), (l) |
| 16 | (b), (i), (l), (n) |
| 17 | (b), (i), (m) |
| 18 | (b), (j) |
| 19 | (b), (j), (n) |
| 20 | (b), (k) |
| 21 | (c), (l) |
| 22 | (c), (l), (n) |
| 23 | (d), (n) |
| 24 | (e), (i) |
| 25 | (e), (i), (l) |
| 26 | (e), (i), (l), (n) |
| 27 | (e), (i), (m) |
| 28 | (e), (j) |
| 29 | (e), (j), (n) |
| 30 | (e), (k) |
| 31 | (f), (l) |
| 32 | (f), (l), (n) |
| 33 | (f), (m) |
| 34 | (g), (n) |
| 35 | (i), (l), (n) |
| 36 | (i), (m) |
| 37 | (j), (n) |
| 38 | (l), (n) |

For SEQ ID NO:X, useful pharmaceutical compositions include: a pharmaceutical composition comprising at least two (three, four or more) of polypeptides (a)-(ad) and a pharmaceutically acceptable carrier. In various embodiments the pharmaceutical composition comprises:
polypeptide (a) and at least one of: polypeptides (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (b) and at least one of polypeptides (c), (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (c) and at least one of polypeptides (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (d) and at least one of polypeptides (e), (f), (g), (h), (i), (j), (k), (l), (m), (n), (o), (p), (q), (r), (s), (t), (u), (v), (w), (x), (y), (z), (aa), (ab), (ac), (ad);
polypeptide (e) and at least one of polypeptides (f), (g), (h), (i), (j), (k), (l), (m), (n), (o), (p), (q), (r), (s), (t), (u), (v), (w), (x), (y), (z), (aa), (ab), (ac), (ad);
polypeptide (f) and at least one of polypeptides (g), (h), (i), (j), (k), (l), (m), (n), (o), (p), (q), (r), (s), (t), (u), (v), (w), (x), (y), (z), (aa), (ab), (ac), (ad);
polypeptide (g) and at least one of polypeptides (h), (i), (j), (k), (l), (m), (n), (o), (p), (q), (r), (s), (t), (u), (v), (w), (x), (y), (z), (aa), (ab), (ac), (ad);
polypeptide (h) and at least one of polypeptides (i), (j), (k), (l), (m), (n), (o), (p), (q), (r), (s), (t), (u), (v), (w), (x), (y), (z), (aa), (ab), (ac), (ad);
polypeptide (i) and at least one of polypeptides (j), (k), (l), (m), (n), (o), (p), (q), (r), (s), (t), (u), (v), (w), (x), (y), (z), (aa), (ab), (ac), (ad);
polypeptide (j) and at least one of polypeptides (k), (l), (m), (n), (o), (p), (q), (r), (s), (t), (u), (v), (w), (x), (y), (z), (aa), (ab), (ac), (ad);
polypeptide (k) and at least one of polypeptides (l), (m), (n), (o), (p), (q), (r), (s), (t), (u), (v), (w), (x), (y), (z), (aa), (ab), (ac), (ad);
polypeptide (l) and at least one of polypeptides (m), (n), (o), (p), (q), (r), (s), (t), (u), (v), (w), (x), (y), (z), (aa), (ab), (ac), (ad);
polypeptide (m) and at least one of polypeptides (n), (o), (p), (q), (r), (s), (t), (u), (v), (w), (x), (y), (z), (aa), (ab), (ac), (ad);
polypeptide (n) and at least one of polypeptides (o), (p), (q), (r), (s), (t), (u), (v), (w), (x), (y), (z), (aa), (ab), (ac), (ad);
polypeptide (o) and at least one of polypeptides (p), (q), (r), (s), (t), (u), (v), (w), (x), (y), (z), (aa), (ab), (ac), (ad);
polypeptide (p) and at least one of polypeptides (q), (r), (s), (t), (u), (v), (w), (x), (y), (z), (aa), (ab), (ac), (ad);
polypeptide (q) and at least one of polypeptides (r), (s), (t), (u), (v), (w), (x), (y), (z), (aa), (ab), (ac), (ad);
polypeptide (r) and at least one of polypeptides (s), (t), (u), (v), (w), (x), (y), (z), (aa), (ab), (ac), (ad);
polypeptide (s) and at least one of polypeptides (t), (u), (v), (w), (x), (y), (z), (aa), (ab), (ac), (ad);
polypeptide (t) and at least one of polypeptides (u), (v), (w), (x), (y), (z), (aa), (ab), (ac), (ad);
polypeptide (u) and at least one of polypeptides (v), (w), (x), (y), (z), (aa), (ab), (ac), (ad);
polypeptide (v) and at least one of polypeptides (w), (x), (y), (z), (aa), (ab), (ac), (ad);
polypeptide (w) and at least one of polypeptides (x), (y), (z), (aa), (ab), (ac), (ad);
polypeptide (x) and at least one of polypeptides (y), (z), (aa), (ab), (ac), (ad);
polypeptide (y) and at least one of polypeptides (z), (aa), (ab), (ac), (ad);
polypeptide (z) and at least one of polypeptides (aa), (ab), (ac), (ad);
polypeptide (aa) and at least one of polypeptides (ab), (ac), (ad);
polypeptide (ab) and at least one of polypeptides (ac), (ad); and
polypeptide (ac) and at least polypeptide (ad).

Also described is: 1) a purified polypeptide comprising (or consisting of or consisting essentially of) at least 10 contiguous amino acids of SEQ ID NO:X1 and 2) a purified polypeptide consisting of a polypeptide fragment of SEQ ID NO:X1 comprising (or consisting of or consisting essentially of) at least 10 contiguous amino acids of SEQ ID NO:X1:

(SEQ ID NO: X1-guanylin consensus 1)
X₁ X₂ X₃ X₄ X₅ X₆ X₇ X₈ X₉ X₁₀ X₁₁ L E X₁₄ V K X₁₇ L X₁₉ X₂₀ L X₂₂ X₂₃ X₂₄ X₂₅ X₂₆ X₂₇ X₂₈ X₂₉
X₃₀ X₃₁ X₃₂ X₃₃ X₃₄ X₃₅ X₃₆ X₃₇ X₃₈ X₃₉ X₄₀ X₄₁ X₄₂ X₄₃ X₄₄ X₄₅ X₄₆ X₄₇ X₄₈ X₄₉ X₅₀ C X₅₂ X₅₃ X₅₄
X₅₅ X₅₆ X₅₇ P X₅₉ X₆₀ X₆₁ X₆₂ P X₆₄ C X₆₆ X₆₇ X₆₈ X₆₉ X₇₀ X₇₁ X₇₂ X₇₃ X₇₄ X₇₅ R L X₇₈ X₇₉ X₈₀ X₈₁
X₈₂ X₈₃ P G T C E I C A Y A A C T G C X₉₉

wherein:

### X₁ is V or S;

### X₂ is T, L, I, Y or E;

### X₃ is V or F;

### X₄ is Q or K;

### X₅ is D or E;

### X₆ is G or N;

### X₇ is D, N, E or G;

### X₈ is F or L;

### X₉ is S, T or K;

### X₁₀ is F or Y;

### X₁₁ is S or P;

### X₁₄ is S or A;

### X₁₇ is K, Q or R;

### X₁₉ is K or H;

### X₂₀ is D, E, A, H or G;

### X₂₂ is Q, R, G, M, A;

### X₂₃ is E, Q or D;

### X₂₄ is A, S, E, V, L or P;

### X₂₅ is Q, N, P, G or S;

### X₂₆ is E, K, M or V;

X₂₇ is G, L or is missing;
X₂₈ is Q, S, R, A or is missing;
X₂₉ is E, K, S, A or is missing;

### X₃₀ is P, V, M or A;

X₃₁ is R, Q, T, I, A or is missing;

### X₃₂ is L, V, I, G, N or S;

### X₃₃ is P, G, R, V, M, A, P;

### X₃₄ is S, R or K;

### X₃₅ is H, L, I, N or K;

X₃₆ is R, K or is missing;
X₃₇ is N, K or is missing;
X₃₈ is F or is missing;
X₃₉ is A or is missing;
X₄₀ is P, L or is missing;
X₄₁ is I, R or is missing;
X₄₂ is L, P, F, V, R or is missing;
X₄₃ is G, V, D, P, L, A or is missing;

### X₄₄ is G, E, K, A, Q, R or S;

### X₄₅ is P, S, H or K;

### X₄₆ is V, I, P, A or Q;

### X₄₇ is A, V, I, A, L, G or T;

X₄₈ is P, A, S, Y or is missing;

### X₄₉ is I, Q, V, N, G, E, H, S or F;

### X₅₀ is L, A or P;

### X₅₂ is S, N, A, Q or G;

X₅₃ is S or missing;

### X₅₄ is H, N, D, S, L, F, or Q;

### X₅₅ is P, S, L or K;

### X₅₆ is A, K, N, T, G, or Q;

### X₅₇ is F or L;

### X₅₉ is E, K or Q;

### X₆₀ is E, A or D;

### X₆₁ is L or F;

### X₆₂ is K, R, Q or L;

### X₆₄ is L, I or V;

### X₆₆ is K, E, Q, T or R;

### X₆₇ is E, K, R or Q;

### X₆₈ is P, S, E or R;

### X₆₉ is N, D or G;

### X₇₀ is A or S;

### X₇₁ is E, Q, P, A or S;

### X₇₂ is E, D, Q, M or A;

### X₇₃ is I, A, or S;

### X₇₄ is L, F or V;

### X₇₅ is Q, E, D, N, G or A;

### X₇₈ is E, A, G or C;

### X₇₉ is E, A, V, S, L or M;

### X₈₀ is I or V;

### X₈₁ is A or P;

### X₈₂ is E, Q, A or S;

### X₈₃ is D or E; and

X₉₉ is F or is missing.

In various embodiments: the purified polypeptide comprises at least 15 amino acids, at least 20 amino acids, at least 25 amino acids, at least 30 amino acids, at least 40 amino acids, at least 45 amino acids, at least 50 amino acids, at least 55 amino acids, at least 60 amino acids, at least 65 amino acids, at least 70 amino acids, at least 75 amino acids, or at least 80 amino acids. In various embodiments the purified polypeptide comprises fewer than 80, 75, 70 65, 60, 55, 50, 45, 40, 35, 30, 25 or 20 contiguous amino acids of SEQ ID NO:X1 and has the ability to bind and/or activate the GC-C receptor and/or elicit diuresis when administered to a subject.

Compositions, including pharmaceutical compositions, can include at least one such polypeptide or can include at least two (three, four or more) such polypeptides which are different. In the compositions containing two or more such polypeptides the polypeptides can be separate or they can be covalently direct linked, e.g, by a peptide bond or a linker or they can be indirectly linked. For example, two such polypeptide sequences can be contained within a larger polypeptide and the two polypeptide sequences can be separated by other polypeptide sequences.

In certain embodiments of the purified polypeptide comprising at least 10 contiguous amino acids of SEQ ID NO:X1 and in certain embodiments of the polypeptide consisting of a polypeptide fragment of SEQ ID NO:X1 comprising at least 10 contiguous amino acids of SEQ ID NO:X1:

### X₁ is V;

### X₂ is T or L;

### X₃ is V or F;

### X₄ is Q or K;

### X₅ is D or E;

### X₆ is G or N;

### X₇ is D or N;

### X₈ is F or L;

### X₉ is S;

### X₁₀ is F or Y;

### X₁₁ is S or P;

### X₁₄ is S;

### X₁₇ is K;

### X₁₉ is K;

### X₂₀ is D or E;

### X₂₂ is Q or R;

### X₂₃ is E;

### X₂₄ is V, L or P;

### X₂₅ is Q or P;

### X₂₆ is E or K;

X₂₇ is missing;
X₂₈ is missing;
X₂₉ is missing;

### X₃₀ is P or V;

### X₃₁ is R

### X₃₂ is L or V;

### X₃₃ is P or G;

### X₃₄ is S, R or K;

### X₃₅ is H or L;

X₃₆ is R, K or is missing;
X₃₇ is N, K or is missing;
X₃₈ is F or is missing;
X₃₉ is A or is missing;
X₄₀ is P or is missing;
X₄₁ is I, R or is missing;

### X₄₂ is L or P;

### X₄₃ is G or L;

### X₄₄ is G, E or K,

### X₄₅ is P or S;

### X₄₆ is V or A;

### X₄₇ is A or V;

X₄₈ is P or is missing;

### X₄₉ is I or Q;

### X₅₀ is L;

### X₅₂ is S;

X₅₃ is missing;

### X₅₄ is H, N, or D;

### X₅₅ is P or S;

### X₅₆ is A, K or N;

### X₅₇ is F or L;

### X₅₉ is E;

### X₆₀ is E or A;

### X₆₁ is L;

### X₆₂ is K or R;

### X₆₄ is L, I or V;

### X₆₆ is K, E, Q, T or R;

### X₆₇ is E or K;

### X₆₈ is P;

### X₆₉ is N;

### X₇₀ is A;

### X₇₁ is E or Q;

### X₇₂ is E;

### X₇₃ is I;

### X₇₄ is L;

### X₇₅ is Q or E;

### X₇₈ is E or A;

### X₇₉ is E or A;

### X₈₀ is I;

### X₈₁ is A;

### X₈₂ is E or Q;

### X₈₃ is D; and

X₉₉ is missing.

Also described is 1) a purified polypeptide comprising (or consisting of or consisting essentially of) at least 10 contiguous amino acids of SEQ ID NO:X2 and 2) a purified polypeptide consisting of a polypeptide fragment of SEQ ID NO:X2 comprising (or consisting of or consisting essentially of) at least 10 contiguous amino acids of SEQ ID NO:X2:

(SEQ ID NO: X2-guanylin consensus 2),
X₁ X₂ X₃ X₄ X₅ X₆ X₇ X₈ X₉ X₁₀ X₁₁ L E X₁₄ V K X₁₇ L X₁₉ X₂₀ L X₂₂ X₂₃ X₂₄ X₂₅ X₂₆ X₂₇ X₂₈ X₂₉
X₃₀ X₃₁ X₃₂ X₃₃ X₃₄ X₃₅ X₃₆ X₃₇ X₃₈ X₃₉ X₄₀ X₄₁ X₄₂ X₄₃ X₄₄ X₄₅ X₄₆ X₄₇ X₄₈ X₄₉ X₅₀ C X₅₂ X₅₃ X₅₄
X₅₅ X₅₆ X₅₇ P X₅₉ X₆₀ X₆₁ X₆₂ P X₆₄ C X₆₆ X₆₇ X₆₈ X₆₉ X₇₀ X₇₁ X₇₂ X₇₃ X₇₄ X₇₅ R L X₇₈ X₇₉ X₈₀ X₈₁
X₈₂ X₈₃ P X₈₅ X₈₆ C E I C A X₉₂ A A C X₉₆ G C X₉₉

wherein:

### X₁ is V or S;

### X₂ is T, L, I, Y or E;

### X₃ is V or F;

### X₄ is Q or K;

### X₅ is D or E;

### X₆ is G or N;

### X₇ is D, N, E or G;

### X₈ is F or L;

### X₉ is S, T or K;

### X₁₀ is F or Y;

### X₁₁ is S or P;

### X₁₄ is S or A;

### X₁₇ is K, Q or R;

### X₁₉ is K or H;

### X₂₀ is D, E, A, H or G;

### X₂₂ is Q, R, G, M, A;

### X₂₃ is E, Q or D;

### X₂₄ is A, S, E, V, L or P;

### X₂₅ is Q, N, P, G or S;

### X₂₆ is E, K, M or V;

X₂₇ is G, L or is missing;
X₂₈ is Q, S, R, A or is missing;
X₂₉ is E, K, S, A or is missing;

### X₃₀ is P, V, M or A;

X₃₁ is R, Q, T, I, A or is missing;

### X₃₂ is L, V, I, G, N or S;

### X₃₃ is P, G, R, V, M, A, P;

### X₃₄ is S, R or K;

### X₃₅ is H, L, I, N or K;

X₃₆ is R, K or is missing;
X₃₇ is N, K or is missing;
X₃₈ is F or is missing;
X₃₉ is A or is missing;
X₄₀ is P, L or is missing;
X₄₁ is I, R or is missing;
X₄₂ is L, P, F, V, R or is missing;
X₄₃ is G, V, D, P, L, A or is missing;

### X₄₄ is G, E, K, A, Q, R or S;

### X₄₅ is P, S, H or K;

### X₄₆ is V, I, P, A or Q;

### X₄₇ is A, V, I, A, L, G or T;

X₄₈ is P, A, S, Y or is missing;

### X₄₉ is I, Q, V, N, G, E, H, S or F;

### X₅₀ is L, A or P;

### X₅₂ is S, N, A, Q or G;

X₅₃ is S or missing;

### X₅₄ is H, N, D, S, L, F, or Q;

### X₅₅ is P, S, L or K;

### X₅₆ is A, K, N, T, G, or Q;

### X₅₇ is F or L;

### X₅₉ is E, K or Q;

### X₆₀ is E, A or D;

### X₆₁ is L or F;

### X₆₂ is K, R, Q or L;

### X₆₄ is L, I or V;

### X₆₆ is K, E, Q, T or R;

### X₆₇ is E, K, R or Q;

### X₆₈ is P, S, E or R;

### X₆₉ is N, D or G;

### X₇₀ is A or S;

### X₇₁ is E, Q, P, A or S;

### X₇₂ is E, D, Q, M or A;

### X₇₃ is I, A, or S;

### X₇₄ is L, F or V;

### X₇₅ is Q, E, D, N, G or A;

### X₇₈ is E, A, G or C;

### X₇₉ is E, A, V, S, L or M;

### X₈₀ is I or V;

### X₈₁ is A or P;

### X₈₂ is E, Q, A or S;

### X₈₃ is D or E;

### X₈₅ is G, S, R, or N;

### X₈₆ is S or T;

### X₉₂ is Y or F;

### X₉₆ is T or A; and

X₉₉ is F or is missing.

In various embodiments: the purified polypeptide comprises at least 15 amino acids, at least 20 amino acids, at least 25 amino acids, at least 30 amino acids, at least 40 amino acids, at least 45 amino acids, at least 50 amino acids, at least 55 amino acids, at least 60 amino acids, at least 65 amino acids, at least 70 amino acids, at least 75 amino acids, or at least 80 amino acids. In various embodiments the purified polypeptide comprises fewer than 80, 75, 70 65, 60, 55, 50, 45, 40, 35, 30, 25 or 20 contiguous amino acids of SEQ ID NO:X2 and has the ability to bind and/or activate the GC-C receptor and/or elicit diuresis when administered to a subject.

Compositions, including pharmaceutical compositions, can include at least one such polypeptide or can include at least two (three, four or more) such polypeptides which are different. In the compositions containing two or more such polypeptides the polypeptides can be separate or they can be covalently direct linked, e.g, by a peptide bond or a linker or they can be indirectly linked. For example, two such polypeptide sequences can be contained within a larger polypeptide and the two polypeptide sequences can be separated by other polypeptide sequences.

In certain embodiments of the purified polypeptide comprising at least 10 contiguous amino acids of SEQ ID NO:X2 and in certain embodiments of the polypeptide consisting of a polypeptide fragment of SEQ ID NO:X2 comprising at least 10 contiguous amino acids of SEQ ID NO:X2:

### X₁ is V;

### X₂ is T or L;

### X₃ is V or F;

### X₄ is Q or K;

### X₅ is D or E;

### X₆ is G or N;

### X₇ is D or N;

### X₈ is F or L;

### X₉ is S;

### X₁₀ is F or Y;

### X₁₁ is S or P;

### X₁₄ is S;

### X₁₇ is K;

### X₁₉ is K;

### X₂₀ is D or E;

### X₂₂ is Q or R;

### X₂₃ is E;

### X₂₄ is V, L or P;

### X₂₅ is Q or P;

### X₂₆ is E or K;

X₂₇ is missing;
X₂₈ is missing;
X₂₉ is missing;

### X₃₀ is P or V;

### X₃₁ is R

### X₃₂ is L or V;

### X₃₃ is P or G;

### X₃₄ is S, R or K;

### X₃₅ is H or L;

X₃₆ is R, K or is missing;
X₃₇ is N, K or is missing;
X₃₈ is F or is missing;
X₃₉ is A or is missing;
X₄₀ is P or is missing;
X₄₁ is I, R or is missing;

### X₄₂ is L or P;

### X₄₃ is G or L;

### X₄₄ is G, E or K,

### X₄₅ is P or S;

### X₄₆ is V or A;

### X₄₇ is A or V;

X₄₈ is P or is missing;

### X₄₉ is I or Q;

### X₅₀ is L;

### X₅₂ is S;

X₅₃ is missing;

### X₅₄ is H, N, or D;

### X₅₅ is P or S;

### X₅₆ is A, K or N;

### X₅₇ is F or L;

### X₅₉ is E;

### X₆₀ is E or A;

### X₆₁ is L;

### X₆₂ is K or R;

### X₆₄ is L, I or V;

### X₆₆ is K, E, Q, T or R;

### X₆₇ is E or K;

### X₆₈ is P;

### X₆₉ is N;

### X₇₀ is A;

### X₇₁ is E or Q;

### X₇₂ is E;

### X₇₃ is I;

### X₇₄ is L;

### X₇₅ is Q or E;

### X₇₈ is E or A;

### X₇₉ is E or A;

### X₈₀ is I;

### X₈₁ is A;

### X₈₂ is E or Q;

### X₈₃ is D;

### X₈₅ is G, or S;

### X₈₆ is T;

### X₉₂ is Y;

### X₉₆ is T; and

X₉₉ is missing.

Among the examples of a purified polypeptide comprising at least 10 contiguous amino acids of SEQ ID NO:X1 or X2 and the examples of a purified polypeptide consisting of a polypeptide fragment of SEQ ID NO:X1 or X2 comprising at least 10 contiguous amino acids of SEQ ID NO:X1 or X2 are:
a) a polypeptide comprising amino acids 1-16 of SEQ ID NO:X1 OR SEQ ID NO:X2;
b) a polypeptide comprising amino acids 1-50 of SEQ ID NO:X1 OR SEQ ID NO:X2;
c) a polypeptide comprising amino acids 1-65 of SEQ ID NO:X1 OR SEQ ID NO:X2;
d) a polypeptide comprising amino acids 1-76 of SEQ ID NO:X1 OR SEQ ID NO:X2;
e) a polypeptide comprising amino acids 17-50 of SEQ ID NO:X1 OR SEQ ID NO:X2;
f) a polypeptide comprising amino acids 17-65 of SEQ ID NO:X1 OR SEQ ID NO:X2;
g) a polypeptide comprising amino acids 17-76 of SEQ ID NO:X1 OR SEQ ID NO:X2;
h) a polypeptide comprising amino acids 17-99 of SEQ ID NO:X1 OR SEQ ID NO:X2.
i) a polypeptide comprising amino acids 51-65 of SEQ ID NO:X1 OR SEQ ID NO:X2;
j) a polypeptide comprising amino acids 51-76 of SEQ ID NO:X1 OR SEQ ID NO:X2;
k) a polypeptide comprising amino acids 51-99 of SEQ ID NO:X1 OR SEQ ID NO:X2.
l) a polypeptide comprising amino acids 66-76 of SEQ ID NO:X1 OR SEQ ID NO:X2;
m) a polypeptide comprising amino acids 66-99 of SEQ ID NO:X1 OR SEQ ID NO:X2.
n) a polypeptide comprising amino acids 77-99 of SEQ ID NO:X1 OR SEQ ID NO:X2;
o) a polypeptide comprising amino acids 1-23 of SEQ ID NO:X1 OR SEQ ID NO:X2;
p) a polypeptide comprising amino acids 51-70 of SEQ ID NO:X1 OR SEQ ID NO:X2;
q) a polypeptide comprising amino acids 51-75 of SEQ ID NO:X1 OR SEQ ID NO:X2;
r) a polypeptide comprising amino acids 51-80 of SEQ ID NO:X1 OR SEQ ID NO:X2;
s) a polypeptide comprising amino acids 46-65 of SEQ ID NO:X1 OR SEQ ID NO:X2;
t) a polypeptide comprising amino acids 41-65 of SEQ ID NO:X1 OR SEQ ID NO:X2;
u) a polypeptide comprising amino acids 36-65 of SEQ ID NO:X1 OR SEQ ID NO:X2;
v) a polypeptide comprising amino acids 46-70 of SEQ ID NO:X1 OR SEQ ID NO:X2;
w) a polypeptide comprising amino acids 41-70 of SEQ ID NO:X1 OR SEQ ID NO:X2;
x) a polypeptide comprising amino acids 36-70 of SEQ ID NO:X1 OR SEQ ID NO:X2;
y) a polypeptide comprising amino acids 46-75 of SEQ ID NO:X1 OR SEQ ID NO:X2;
z) a polypeptide comprising amino acids 41-75 of SEQ ID NO:X1 OR SEQ ID NO:X2;
aa) a polypeptide comprising amino acids 36-75 of SEQ ID NO:X1 OR SEQ ID NO:X2;
ab) a polypeptide comprising amino acids 46-80 of SEQ ID NO:X1 OR SEQ ID NO:X2;
ac) a polypeptide comprising amino acids 41-80 of SEQ ID NO:X1 OR SEQ ID NO:X2; and
ad) a polypeptide comprising amino acids 36-80 of SEQ ID NO:X1 OR SEQ ID NO:X2.

The contemplated purified polypeptides include any subset of the aforementioned polypeptides as well as each one of the forgoing polypeptides.

Among the examples of a purified polypeptide comprising at least 10 contiguous amino acids of SEQ ID NO:X1 or X2 and the examples of a purified polypeptide consisting of a polypeptide fragment of SEQ ID NO:X1 or X2 comprising at least 10 contiguous amino acids of SEQ ID NOX1 or X2 are:
a) a polypeptide consisting of amino acids 1-16 of SEQ ID NO:X1 OR SEQ ID NO:X2;
b) a polypeptide consisting of amino acids 1-50 of SEQ ID NO:X1 OR SEQ ID NO:X2;
c) a polypeptide consisting of amino acids 1-65 of SEQ ID NO:X1 OR SEQ ID NO:X2;
d) a polypeptide consisting of amino acids 1-76 of SEQ ID NO:X1 OR SEQ ID NO:X2;
e) a polypeptide consisting of amino acids 17-50 of SEQ ID NO:X1 OR SEQ ID NO:X2;
f) a polypeptide consisting of amino acids 17-65 of SEQ ID NO:X1 OR SEQ ID NO:X2;
g) a polypeptide consisting of amino acids 17-76 of SEQ ID NO:X1 OR SEQ ID NO:X2;
h) a polypeptide consisting of amino acids 17-99 of SEQ ID NO:X1 OR SEQ ID NO:X2.
i) a polypeptide consisting of amino acids 51-65 of SEQ ID NO:X1 OR SEQ ID NO:X2;
j) a polypeptide consisting of amino acids 51-76 of SEQ ID NO:X1 OR SEQ ID NO:X2;
k) a polypeptide consisting of amino acids 51-99 of SEQ ID NO:X1 OR SEQ ID NO:X2.
l) a polypeptide consisting of amino acids 66-76 of SEQ ID NO:X1 OR SEQ ID NO:X2;
m) a polypeptide consisting of amino acids 66-99 of SEQ ID NO:X1 OR SEQ ID NO:X2.
n) a polypeptide consisting of amino acids 77-99 of SEQ ID NO:X1 OR SEQ ID NO:X2;
o) a polypeptide consisting of amino acids 1-23 of SEQ ID NO:X1 OR SEQ ID NO:X2;
p) a polypeptide consisting of amino acids 51-70 of SEQ ID NO:X1 OR SEQ ID NO:X2;
q) a polypeptide consisting of amino acids 51-75 of SEQ ID NO:X1 OR SEQ ID NO:X2;
r) a polypeptide consisting of amino acids 51-80 of SEQ ID NO:X1 OR SEQ ID NO:X2;
s) a polypeptide consisting of amino acids 46-65 of SEQ ID NO:X1 OR SEQ ID NO:X2;
t) a polypeptide consisting of amino acids 41-65 of SEQ ID NO:X1 OR SEQ ID NO:X2;
u) a polypeptide consisting of amino acids 36-65 of SEQ ID NO:X1 OR SEQ ID NO:X2;
v) a polypeptide consisting of amino acids 46-70 of SEQ ID NO:X1 OR SEQ ID NO:X2;
w) a polypeptide consisting of amino acids 41-70 of SEQ ID NO:X1 OR SEQ ID NO:X2;
x) a polypeptide consisting of amino acids 36-70 of SEQ ID NO:X1 OR SEQ ID NO:X2;
y) a polypeptide consisting of amino acids 46-75 of SEQ ID NO:X1 OR SEQ ID NO:X2;
z) a polypeptide consisting of amino acids 41-75 of SEQ ID NO:X1 OR SEQ ID NO:X2;
aa) a polypeptide consisting of amino acids 36-75 of SEQ ID NO:X1 OR SEQ ID NO:X2;
ab) a polypeptide consisting of amino acids 46-80 of SEQ ID NO:X1 OR SEQ ID NO:X2;
ac) a polypeptide consisting of amino acids 41-80 of SEQ ID NO:X1 OR SEQ ID NO:X2; and
ad) a polypeptide consisting of amino acids 36-80 of SEQ ID NO:X1 OR SEQ ID NO:X2.

The contemplated purified polypeptides include any subset of the aforementioned polypeptides as well as each one of the forgoing polypeptides.

Among the examples of a purified polypeptide comprising at least 10 contiguous amino acids of SEQ ID NO:X1 or X₂ and the examples of a purified polypeptide consisting of a polypeptide fragment of SEQ ID NO:X1 or X2 comprising at least 10 contiguous amino acids of SEQ ID NOX1 or X2 are:
a) a polypeptide consisting essentially of amino acids 1-16 of SEQ ID NO:X1 OR SEQ ID NO:X2;
b) a polypeptide consisting essentially of amino acids 1-50 of SEQ ID NO:X1 OR SEQ ID NO:X2;
c) a polypeptide consisting essentially of amino acids 1-65 of SEQ ID NO:X1 OR SEQ ID NO:X2;
d) a polypeptide consisting essentially of amino acids 1-76 of SEQ ID NO:X1 OR SEQ ID NO:X2;
e) a polypeptide consisting essentially of amino acids 17-50 of SEQ ID NO:X1 OR SEQ ID NO:X2;
f) a polypeptide consisting essentially of amino acids 17-65 of SEQ ID NO:X1 OR SEQ ID NO:X2;
g) a polypeptide consisting essentially of amino acids 17-76 of SEQ ID NO:X1 OR SEQ ID NO:X2;
h) a polypeptide consisting essentially of amino acids 17-99 of SEQ ID NO:X1 OR SEQ ID NO:X2.
i) a polypeptide consisting essentially of amino acids 51-65 of SEQ ID NO:X1 OR SEQ ID NO:X2;
j) a polypeptide consisting essentially of amino acids 51-76 of SEQ ID NO:X1 OR SEQ ID NO:X2;
k) a polypeptide consisting essentially of amino acids 51-99 of SEQ ID NO:X1 OR SEQ ID NO:X2.
l) a polypeptide consisting essentially of amino acids 66-76 of SEQ ID NO:X1 OR SEQ ID NO:X2;
m) a polypeptide consisting essentially of amino acids 66-99 of SEQ ID NO:X1 OR SEQ ID NO:X2.
n) a polypeptide consisting essentially of amino acids 77-99 of SEQ ID NO:X1 OR SEQ ID NO:X2;
o) a polypeptide consisting essentially of amino acids 1-23 of SEQ ID NO:X1 OR SEQ ID NO:X2;
p) a polypeptide consisting essentially of amino acids 51-70 of SEQ ID NO:X1 OR SEQ ID NO:X2;
q) a polypeptide consisting essentially of amino acids 51-75 of SEQ ID NO:X1 OR SEQ ID NO:X2;
r) a polypeptide consisting essentially of amino acids 51-80 of SEQ ID NO:X1 OR SEQ ID NO:X2;
s) a polypeptide consisting essentially of amino acids 46-65 of SEQ ID NO:X1 OR SEQ ID NO:X2;
t) a polypeptide consisting essentially of amino acids 41-65 of SEQ ID NO:X1 OR SEQ ID NO:X2;
u) a polypeptide consisting essentially of amino acids 36-65 of SEQ ID NO:X1 OR SEQ ID NO:X2;
v) a polypeptide consisting essentially of amino acids 46-70 of SEQ ID NO:X1 OR SEQ ID NO:X2;
w) a polypeptide consisting essentially of amino acids 41-70 of SEQ ID NO:X1 OR SEQ ID NO:X2;
x) a polypeptide consisting essentially of amino acids 36-70 of SEQ ID NO:X1 OR SEQ ID NO:X2;
y) a polypeptide consisting essentially of amino acids 46-75 of SEQ ID NO:X1 OR SEQ ID NO:X2;
z) a polypeptide consisting essentially of amino acids 41-75 of SEQ ID NO:X1 OR SEQ ID NO:X2;
aa) a polypeptide consisting essentially of amino acids 36-75 of SEQ ID NO:X1 OR SEQ ID NO:X2;
ab) a polypeptide consisting essentially of amino acids 46-80 of SEQ ID NO:X1 OR SEQ ID NO:X2;
ac) a polypeptide consisting essentially of amino acids 41-80 of SEQ ID NO:X1 OR SEQ ID NO:X2; and
ad) a polypeptide consisting essentially of amino acids 36-80 of SEQ ID NO:X1 OR SEQ ID NO:X2.

The contemplated purified polypeptides include any subset of the aforementioned polypeptides as well as each one of the forgoing polypeptides.

For SEQ ID NO:X1 and SEQ ID NO:X2 useful compositions, including pharmaceutical compositions, that include, for example, more than one of polypeptides (a)-(ad) (or polypeptides comprising, consisting of or consisting essentially of one or more of (a)-(ad) or a consisting of a polypeptide fragment of SEQ ID NO:X1 or X2 comprising one or more of (a)-(ad)) include combinations 1-38 below:

| | |
|---|---|
| 1 | (a), (e) |
| 2 | (a), (e), (i) |
| 3 | (a), (e), (i), (l) |
| 4 | (a), (e), (i), (l), (n) |
| 5 | (a), (e), (i), (m) |
| 6 | (a), (e), (k) |
| 7 | (a), (f) |
| 8 | (a), (f), (l) |
| 9 | (a), (f), (l), (n) |
| 10 | (a), (f), (m) |
| 11 | (a), (g) |
| 12 | (a), (g), (n) |
| 13 | (a), (h) |
| 14 | (b), (i) |
| 15 | (b), (i), (l) |
| 16 | (b), (i), (l), (n) |
| 17 | (b), (i), (m) |
| 18 | (b), (j) |
| 19 | (b), (j), (n) |
| 20 | (b), (k) |
| 21 | (c), (l) |
| 22 | (c), (l), (n) |
| 23 | (d), (n) |
| 24 | (e), (i) |
| 25 | (e), (i), (l) |
| 26 | (e), (i), (l), (n) |
| 27 | (e), (i), (m) |
| 28 | (e), (j) |
| 29 | (e), (j), (n) |
| 30 | (e), (k) |
| 31 | (f), (l) |
| 32 | (f), (l), (n) |
| 33 | (f), (m) |
| 34 | (g), (n) |
| 35 | (i), (l), (n) |
| 36 | (i), (m) |
| 37 | (j), (n) |
| 38 | (l), (n) |

For SEQ ID NO:X1 and SEQ ID NO:X2, useful pharmaceutical compositions include: a pharmaceutical composition comprising at least two (three, four or more) of polypeptides (a)-(ad) and a pharmaceutically acceptable carrier. In various embodiments the pharmaceutical composition comprises:
polypeptide (a) and at least one of: polypeptides (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (b) and at least one of polypeptides (c), (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (c) and at least one of polypeptides (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (d) and at least one of polypeptides (e), (f), (g), (h), (i), (j), (k), (l), (m), (n), (o), (p), (q), (r), (s), (t), (u), (v), (w), (x), (y), (z), (aa), (ab), (ac), (ad);
polypeptide (e) and at least one of polypeptides (f), (g), (h), (i), (j), (k), (l), (m), (n), (o), (p), (q), (r), (s), (t), (u), (v), (w), (x), (y), (z), (aa), (ab), (ac), (ad);
polypeptide (f) and at least one of polypeptides (g), (h), (i), (j), (k), (l), (m), (n), (o), (p), (q), (r), (s), (t), (u), (v), (w), (x), (y), (z), (aa), (ab), (ac), (ad);
polypeptide (g) and at least one of polypeptides (h), (i), (j), (k), (l), (m), (n), (o), (p), (q), (r), (s), (t), (u), (v), (w), (x), (y), (z), (aa), (ab), (ac), (ad);
polypeptide (h) and at least one of polypeptides (i), (j), (k), (l), (m), (n), (o), (p), (q), (r), (s), (t), (u), (v), (w), (x), (y), (z), (aa), (ab), (ac), (ad);
polypeptide (i) and at least one of polypeptides (j), (k), (l), (m), (n), (o), (p), (q), (r), (s), (t), (u), (v), (w), (x), (y), (z), (aa), (ab), (ac), (ad);
polypeptide (j) and at least one of polypeptides (k), (l), (m), (n), (o), (p), (q), (r), (s), (t), (u), (v), (w), (x), (y), (z), (aa), (ab), (ac), (ad);
polypeptide (k) and at least one of polypeptides (l), (m), (n), (o), (p), (q), (r), (s), (t), (u), (v), (w), (x), (y), (z), (aa), (ab), (ac), (ad);
polypeptide (l) and at least one of polypeptides (m), (n), (o), (p), (q), (r), (s), (t), (u), (v), (w), (x), (y), (z), (aa), (ab), (ac), (ad);
polypeptide (m) and at least one of polypeptides (n), (o), (p), (q), (r), (s), (t), (u), (v), (w), (x), (y), (z), (aa), (ab), (ac), (ad);
polypeptide (n) and at least one of polypeptides (o), (p), (q), (r), (s), (t), (u), (v), (w), (x), (y), (z), (aa), (ab), (ac), (ad);
polypeptide (o) and at least one of polypeptides (p), (q), (r), (s), (t), (u), (v), (w), (x), (y), (z), (aa), (ab), (ac), (ad);
polypeptide (p) and at least one of polypeptides (q), (r), (s), (t), (u), (v), (w), (x), (y), (z), (aa), (ab), (ac), (ad);
polypeptide (q) and at least one of polypeptides (r), (s), (t), (u), (v), (w), (x), (y), (z), (aa), (ab), (ac), (ad);
polypeptide (r) and at least one of polypeptides (s), (t), (u), (v), (w), (x), (y), (z), (aa), (ab), (ac), (ad);
polypeptide (s) and at least one of polypeptides (t), (u), (v), (w), (x), (y), (z), (aa), (ab), (ac), (ad);
polypeptide (t) and at least one of polypeptides (u), (v), (w), (x), (y), (z), (aa), (ab), (ac), (ad);
polypeptide (u) and at least one of polypeptides (v), (w), (x), (y), (z), (aa), (ab), (ac), (ad);
polypeptide (v) and at least one of polypeptides (w), (x), (y), (z), (aa), (ab), (ac), (ad);
polypeptide (w) and at least one of polypeptides (x), (y), (z), (aa), (ab), (ac), (ad);
polypeptide (x) and at least one of polypeptides (y), (z), (aa), (ab), (ac), (ad);
polypeptide (y) and at least one of polypeptides (z), (aa), (ab), (ac), (ad);
polypeptide (z) and at least one of polypeptides (aa), (ab), (ac), (ad);
polypeptide (aa) and at least one of polypeptides (ab), (ac), (ad);
polypeptide (ab) and at least one of polypeptides (ac), (ad); and
polypeptide (ac) and at least polypeptide (ad).

Described herein are purified polypeptides comprising (or consisting of or consisting essentially of) at least 10 contiguous amino acids of SEQ ID NO:ZZ′ and purified polypeptides consisting of a polypeptide fragment of SEQ ID NO:ZZ′ comprising (or consisting of or consisting essentially of) at least 10 contiguous amino acids of SEQ ID NO:ZZ′ VYIQYQGFRVQLESMKKLSDLEAQWAPSPRLQAQSLLPAVCHHPALPQDLQPVCASQE ASSIFKTLRTIA (SEQ ID NO:ZZ′; human uroguanylin prosequence).

In various embodiments: the purified polypeptide comprises at least 15 amino acids, at least 20 amino acids, at least 25 amino acids, at least 30 amino acids, at least 40 amino acids, at least 45 amino acids, at least 50 amino acids, at least 55 amino acids, at least 60 amino acids, at least 65 amino acids, at least 70 amino acids, at least 75 amino acids, or at least 80 amino acids. In various embodiments the purified polypeptide comprises fewer than 80, 75, 70 65, 60, 55, 50, 45, 40, 35, 30, 25 or 20 contiguous amino acids of SEQ ID NO:ZZ′ and has the ability to bind and/or activate the GC-C receptor and/or elicit diuresis when administered to a subject.

Compositions, including pharmaceutical compositions, can include at least one such polypeptide or can include at least two (three, four or more) such polypeptides which are different. In the compositions containing two or more such polypeptides the polypeptides can be separate or they can be covalently direct linked, e.g, by a peptide bond or a linker or they can be indirectly linked. For example, two such polypeptide sequences can be contained within a larger polypeptide and the two polypeptide sequences can be separated by other polypeptide sequences.

Described herein are purified polypeptides comprising (or consisting of or consisting essentially of) at least 10 contiguous amino acids of SEQ ID NO:ZZ and purified polypeptides consisting of a polypeptide fragment of SEQ ID NO:ZZ comprising (or consisting of or consisting essentially of) at least 10 contiguous amino acids of SEQ ID NO:ZZ VYIQYQGFRVQLESMKKLSDLEAQWAPSPRLQAQSLLPAVCHHPALPQDLQPVCASQE ASSIFKTLRTIANDDCELCVNVACTGCL (SEQ ID NO:ZZ; human prouroguanylin).

In various embodiments: the purified polypeptide comprises at least 15 amino acids, at least 20 amino acids, at least 25 amino acids, at least 30 amino acids, at least 40 amino acids, at least 45 amino acids, at least 50 amino acids, at least 55 amino acids, at least 60 amino acids, at least 65 amino acids, at least 70 amino acids, at least 75 amino acids, or at least 80 amino acids. In various embodiments the purified polypeptide comprises fewer than 80, 75, 70 65, 60, 55, 50, 45, 40, 35, 30, 25 or 20 contiguous amino acids of SEQ ID NO:ZZ and has the ability to bind and/or activate the GC-C receptor and/or elicit diuresis when administered to a subject.

Compositions, including pharmaceutical compositions, can include at least one such polypeptide or can include at least two (three, four or more) such polypeptides which are different. In the compositions containing two or more such polypeptides the polypeptides can be separate or they can be covalently direct linked, e.g, by a peptide bond or a linker or they can be indirectly linked. For example, two such polypeptide sequences can be contained within a larger polypeptide and the two polypeptide sequences can be separated by other polypeptide sequences.

Among the examples of a purified polypeptide comprising at least 10 contiguous amino acids of SEQ ID NO:ZZ and the examples of a polypeptide consisting of a polypeptide fragment of SEQ ID NO:ZZ comprising at least 10 contiguous amino acids of SEQ ID NO:ZZ are:
a) a polypeptide comprising amino acids 1-16 of SEQ ID NO:ZZ;
b) a polypeptide comprising amino acids 1-40 of SEQ ID NO:ZZ;
c) a polypeptide comprising amino acids 1-54 of SEQ ID NO:ZZ;
d) a polypeptide comprising amino acids 1-66 of SEQ ID NO:ZZ;
e) a polypeptide comprising amino acids 17-40 of SEQ ID NO:ZZ;
f) a polypeptide comprising amino acids 17-54 of SEQ ID NO:ZZ;
g) a polypeptide comprising amino acids 17-66 of SEQ ID NO:ZZ;
h) a polypeptide comprising amino acids 17-86 of SEQ ID NO:ZZ;
i) a polypeptide comprising amino acids 41-54 of SEQ ID NO:ZZ;
j) a polypeptide comprising amino acids 41-66 of SEQ ID NO:ZZ;
k) a polypeptide comprising amino acids 41-86 of SEQ ID NO:ZZ;
l) a polypeptide comprising amino acids 55-66 of SEQ ID NO:ZZ;
m) a polypeptide comprising amino acids 55-86 of SEQ ID NO:ZZ;
n) a polypeptide comprising amino acids 67-86 of SEQ ID NO:ZZ;
o) a polypeptide comprising amino acids 1-21 of SEQ ID NO:ZZ;
p) a polypeptide comprising amino acids 41-59 of SEQ ID NO:ZZ;
q) a polypeptide comprising amino acids 41-64 of SEQ ID NO:ZZ;
r) a polypeptide comprising amino acids 41-69 of SEQ ID NO:ZZ;
s) a polypeptide comprising amino acids 36-54 of SEQ ID NO:ZZ;
t) a polypeptide comprising amino acids 31-54 of SEQ ID NO:ZZ;
u) a polypeptide comprising amino acids 26-54 of SEQ ID NO:ZZ;
v) a polypeptide comprising amino acids 36-59 of SEQ ID NO:ZZ;
w) a polypeptide comprising amino acids 31-59 of SEQ ID NO:ZZ;
x) a polypeptide comprising amino acids 26-59 of SEQ ID NO:ZZ;
y) a polypeptide comprising amino acids 36-64 of SEQ ID NO:ZZ;
z) a polypeptide comprising amino acids 31-64 of SEQ ID NO:ZZ;
aa) a polypeptide comprising amino acids 26-64 of SEQ ID NO:ZZ;
ab) a polypeptide comprising amino acids 36-69 of SEQ ID NO:ZZ;
ac) a polypeptide comprising amino acids 31-69 of SEQ ID NO:ZZ; and
ad) a polypeptide comprising amino acids 26-69 of SEQ ID NO:ZZ.

The contemplated purified polypeptides include any subset of the aforementioned polypeptides as well as each one of the forgoing polypeptides.

Among the examples of a purified polypeptide comprising at least 10 contiguous amino acids of SEQ ID NO:ZZ and the examples of a polypeptide consisting of a polypeptide fragment of SEQ ID NO:ZZ comprising at least 10 contiguous amino acids of SEQ ID NO:ZZ are:
a) a polypeptide consisting of amino acids 1-16 of SEQ ID NO:ZZ;
b) a polypeptide consisting of amino acids 1-40 of SEQ ID NO:ZZ;
c) a polypeptide consisting of amino acids 1-54 of SEQ ID NO:ZZ;
d) a polypeptide consisting of amino acids 1-66 of SEQ ID NO:ZZ;
e) a polypeptide consisting of amino acids 17-40 of SEQ ID NO:ZZ;
f) a polypeptide consisting of amino acids 17-54 of SEQ ID NO:ZZ;
g) a polypeptide consisting of amino acids 17-66 of SEQ ID NO:ZZ;
h) a polypeptide consisting of amino acids 17-86 of SEQ ID NO:ZZ;
i) a polypeptide consisting of amino acids 41-54 of SEQ ID NO:ZZ;
j) a polypeptide consisting of amino acids 41-66 of SEQ ID NO:ZZ;
k) a polypeptide consisting of amino acids 41-86 of SEQ ID NO:ZZ;
l) a polypeptide consisting of amino acids 55-66 of SEQ ID NO:ZZ;
m) a polypeptide consisting of amino acids 55-86 of SEQ ID NO:ZZ;
n) a polypeptide consisting of amino acids 67-86 of SEQ ID NO:ZZ;
o) a polypeptide consisting of amino acids 1-21 of SEQ ID NO:ZZ;
p) a polypeptide consisting of amino acids 41-59 of SEQ ID NO:ZZ;
q) a polypeptide consisting of amino acids 41-64 of SEQ ID NO:ZZ;
r) a polypeptide consisting of amino acids 41-69 of SEQ ID NO:ZZ;
s) a polypeptide consisting of amino acids 36-54 of SEQ ID NO:ZZ;
t) a polypeptide consisting of amino acids 31-54 of SEQ ID NO:ZZ;
u) a polypeptide consisting of amino acids 26-54 of SEQ ID NO:ZZ;
v) a polypeptide consisting of amino acids 36-59 of SEQ ID NO:ZZ;
w) a polypeptide consisting of amino acids 31-59 of SEQ ID NO:ZZ;
x) a polypeptide consisting of amino acids 26-59 of SEQ ID NO:ZZ;
y) a polypeptide consisting of amino acids 36-64 of SEQ ID NO:ZZ;
z) a polypeptide consisting of amino acids 31-64 of SEQ ID NO:ZZ;
aa) a polypeptide consisting of amino acids 26-64 of SEQ ID NO:ZZ;
ab) a polypeptide consisting of amino acids 36-69 of SEQ ID NO:ZZ;
ac) a polypeptide consisting of amino acids 31-69 of SEQ ID NO:ZZ; and
ad) a polypeptide consisting of amino acids 26-69 of SEQ ID NO:ZZ.

The contemplated purified polypeptides include any subset of the aforementioned polypeptides as well as each one of the forgoing polypeptides.

Among the examples of a purified polypeptide comprising at least 10 contiguous amino acids of SEQ ID NO:ZZ and the examples of a polypeptide consisting of a polypeptide fragment of SEQ ID NO:ZZ comprising at least 10 contiguous amino acids of SEQ ID NO:ZZ are:
a) a polypeptide consisting essentially of amino acids 1-16 of SEQ ID NO:ZZ;
b) a polypeptide consisting essentially of amino acids 1-40 of SEQ ID NO:ZZ;
c) a polypeptide consisting of amino acids 1-54 of SEQ ID NO:ZZ;
d) a polypeptide consisting of amino acids 1-66 of SEQ ID NO:ZZ;
e) a polypeptide consisting of amino acids 17-40 of SEQ ID NO:ZZ;
f) a polypeptide consisting of amino acids 17-54 of SEQ ID NO:ZZ;
g) a polypeptide consisting of amino acids 17-66 of SEQ ID NO:ZZ;
h) a polypeptide consisting of amino acids 17-86 of SEQ ID NO:ZZ;
i) a polypeptide consisting of amino acids 41-54 of SEQ ID NO:ZZ;
j) a polypeptide consisting of amino acids 41-66 of SEQ ID NO:ZZ;
k) a polypeptide consisting of amino acids 41-86 of SEQ ID NO:ZZ;
l) a polypeptide consisting of amino acids 55-66 of SEQ ID NO:ZZ;
m) a polypeptide consisting of amino acids 55-86 of SEQ ID NO:ZZ;
n) a polypeptide consisting of amino acids 67-86 of SEQ ID NO:ZZ;
o) a polypeptide consisting of amino acids 1-21 of SEQ ID NO:ZZ;
p) a polypeptide consisting of amino acids 41-59 of SEQ ID NO:ZZ;
q) a polypeptide consisting of amino acids 41-64 of SEQ ID NO:ZZ;
r) a polypeptide consisting of amino acids 41-69 of SEQ ID NO:ZZ;
s) a polypeptide consisting of amino acids 36-54 of SEQ ID NO:ZZ;
t) a polypeptide consisting of amino acids 31-54 of SEQ ID NO:ZZ;
u) a polypeptide consisting of amino acids 26-54 of SEQ ID NO:ZZ;
v) a polypeptide consisting of amino acids 36-59 of SEQ ID NO:ZZ;
w) a polypeptide consisting of amino acids 31-59 of SEQ ID NO:ZZ;
x) a polypeptide consisting of amino acids 26-59 of SEQ ID NO:ZZ;
y) a polypeptide consisting of amino acids 36-64 of SEQ ID NO:ZZ;
z) a polypeptide consisting of amino acids 31-64 of SEQ ID NO:ZZ;
aa) a polypeptide consisting of amino acids 26-64 of SEQ ID NO:ZZ;
ab) a polypeptide consisting of amino acids 36-69 of SEQ ID NO:ZZ;
ac) a polypeptide consisting of amino acids 31-69 of SEQ ID NO:ZZ; and
ad) a polypeptide consisting of amino acids 26-69 of SEQ ID NO:ZZ.

The contemplated purified polypeptides include any subset of the aforementioned polypeptides as well as each one of the forgoing polypeptides.

For SEQ ID NO:ZZ useful compositions, including pharmaceutical compositions, that include, for example, more than one of polypeptides (a)-(ad) (or polypeptides comprising, consisting of or consisting essentially of one or more of (a)-(ad) or a consisting of a polypeptide fragment of SEQ ID NO:ZZ comprising one or more of (a)-(ad)) include combinations 1-38 below:

| | |
|---|---|
| 1 | (a), (e) |
| 2 | (a), (e), (i) |
| 3 | (a), (e), (i), (l) |
| 4 | (a), (e), (i), (l), (n) |
| 5 | (a), (e), (i), (m) |
| 6 | (a), (e), (k) |
| 7 | (a), (f) |
| 8 | (a), (f), (l) |
| 9 | (a), (f), (l), (n) |
| 10 | (a), (f), (m) |
| 11 | (a), (g) |
| 12 | (a), (g), (n) |
| 13 | (a), (h) |
| 14 | (b), (i) |
| 15 | (b), (i), (l) |
| 16 | (b), (i), (l), (n) |
| 17 | (b), (i), (m) |
| 18 | (b), (j) |
| 19 | (b), (j), (n) |
| 20 | (b), (k) |
| 21 | (c), (l) |
| 22 | (c), (l), (n) |
| 23 | (d), (n) |
| 24 | (e), (i) |
| 25 | (e), (i), (l) |
| 26 | (e), (i), (l), (n) |
| 27 | (e), (i), (m) |
| 28 | (e), (j) |
| 29 | (e), (j), (n) |
| 30 | (e), (k) |
| 31 | (f), (l) |
| 32 | (f), (l), (n) |
| 33 | (f), (m) |
| 34 | (g), (n) |
| 35 | (i), (l), (n) |
| 36 | (i), (m) |
| 37 | (j), (n) |
| 38 | (l), (n) |

For SEQ ID NO:ZZ, useful pharmaceutical compositions include: a pharmaceutical composition comprising at least two (three, four or more) of polypeptides (a)-(ad) and a pharmaceutically acceptable carrier. In various embodiments the pharmaceutical composition comprises:
polypeptide (a) and at least one of: polypeptides (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (b) and at least one of polypeptides (c), (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (c) and at least one of polypeptides (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (d) and at least one of polypeptides (e), (f), (g), (h), (i), (j), (k), (l), (m), (n), (o), (p), (q), (r), (s), (t), (u), (v), (w), (x), (y), (z), (aa), (ab), (ac), (ad);
polypeptide (e) and at least one of polypeptides (f), (g), (h), (i), (j), (k), (l), (m), (n), (o), (p), (q), (r), (s), (t), (u), (v), (w), (x), (y), (z), (aa), (ab), (ac), (ad);
polypeptide (f) and at least one of polypeptides (g), (h), (i), (j), (k), (l), (m), (n), (o), (p), (q), (r), (s), (t), (u), (v), (w), (x), (y), (z), (aa), (ab), (ac), (ad);
polypeptide (g) and at least one of polypeptides (h), (i), (j), (k), (l), (m), (n), (o), (p), (q), (r), (s), (t), (u), (v), (w), (x), (y), (z), (aa), (ab), (ac), (ad);
polypeptide (h) and at least one of polypeptides (i), (j), (k), (l), (m), (n), (o), (p), (q), (r), (s), (t), (u), (v), (w), (x), (y), (z), (aa), (ab), (ac), (ad);
polypeptide (i) and at least one of polypeptides (j), (k), (l), (m), (n), (o), (p), (q), (r), (s), (t), (u), (v), (w), (x), (y), (z), (aa), (ab), (ac), (ad);
polypeptide (j) and at least one of polypeptides (k), (l), (m), (n), (o), (p), (q), (r), (s), (t), (u), (v), (w), (x), (y), (z), (aa), (ab), (ac), (ad);
polypeptide (k) and at least one of polypeptides (l), (m), (n), (o), (p), (q), (r), (s), (t), (u), (v), (w), (x), (y), (z), (aa), (ab), (ac), (ad);
polypeptide (l) and at least one of polypeptides (m), (n), (o), (p), (q), (r), (s), (t), (u), (v), (w), (x), (y), (z), (aa), (ab), (ac), (ad);
polypeptide (m) and at least one of polypeptides (n), (o), (p), (q), (r), (s), (t), (u), (v), (w), (x), (y), (z), (aa), (ab), (ac), (ad);
polypeptide (n) and at least one of polypeptides (o), (p), (q), (r), (s), (t), (u), (v), (w), (x), (y), (z), (aa), (ab), (ac), (ad);
polypeptide (o) and at least one of polypeptides (p), (q), (r), (s), (t), (u), (v), (w), (x), (y), (z), (aa), (ab), (ac), (ad);
polypeptide (p) and at least one of polypeptides (q), (r), (s), (t), (u), (v), (w), (x), (y), (z), (aa), (ab), (ac), (ad);
polypeptide (q) and at least one of polypeptides (r), (s), (t), (u), (v), (w), (x), (y), (z), (aa), (ab), (ac), (ad);
polypeptide (r) and at least one of polypeptides (s), (t), (u), (v), (w), (x), (y), (z), (aa), (ab), (ac), (ad);
polypeptide (s) and at least one of polypeptides (t), (u), (v), (w), (x), (y), (z), (aa), (ab), (ac), (ad);
polypeptide (t) and at least one of polypeptides (u), (v), (w), (x), (y), (z), (aa), (ab), (ac), (ad);
polypeptide (u) and at least one of polypeptides (v), (w), (x), (y), (z), (aa), (ab), (ac), (ad);
polypeptide (v) and at least one of polypeptides (w), (x), (y), (z), (aa), (ab), (ac), (ad);
polypeptide (w) and at least one of polypeptides (x), (y), (z), (aa), (ab), (ac), (ad);
polypeptide (x) and at least one of polypeptides (y), (z), (aa), (ab), (ac), (ad);
polypeptide (y) and at least one of polypeptides (z), (aa), (ab), (ac), (ad);
polypeptide (z) and at least one of polypeptides (aa), (ab), (ac), (ad);
polypeptide (aa) and at least one of polypeptides (ab), (ac), (ad);
polypeptide (ab) and at least one of polypeptides (ac), (ad); and
polypeptide (ac) and at least polypeptide (ad).

Also described is: 1) a purified polypeptide comprising (or consisting of or consisting essentially of) at least 10 contiguous amino acids of SEQ ID NO:ZZ1; and 2) a purified polypeptide consisting of a polypeptide fragment of SEQ ID NO:ZZ1 comprising (or consisting of or consisting essentially of) at least 10 contiguous amino acids of SEQ ID NO:ZZ1 V X₂, X₄ Y X₆ G X₈ X₉ V X₁₁ L X₁₃ S X₁₅ K X₁₇ L X₁₉ X₂₀ L X₂₂ X₂₃ X₂₄ X₂₅ X₂₆ X₂₇ X₂₈ X₂₉ X₃₀ X₃₁ X₃₂ X₃₃ X₃₄ X₃₅ X₃₆ X₃₇ X₃₈ X₃₉ X₄₀ X₄₁ C X₄₃ X₄₄ X₄₅ A L P X₄₉ D L X₅₂ P X₅₄ C X₅₆ X₅₇ X₅₈ X₅₉ X₆₀ X₆₁ X₆₂ X₆₃ X₆₄ X₆₅ X₆₆ L R X₆₉ X₇₀ X₇₁ N D D C E L C V N V A (SEQ ID NO:ZZ1-uroguanylin consensus 1), wherein:

### X₂ is Y or D;

### X₄ is Q or K;

### X₆ is Q, H or E;

### X₈ is F or Y;

### X₉ is R or Q;

### X₁₁ is Q or K;

### X₁₃ is E, K or D;

### X₁₅ is M or V;

### X₁₇ is K or Q;

### X₁₉ is S, N, K or D;

### X₂₀ is D, E or A;

### X₂₂ is E, V or L;

### X₂₃ is A, E or G;

### X₂₄ is Q or K;

### X₂₅ is W, Q, E or P;

### X₂₆ is A, M, V or R;

### X₂₇ is P or S;

### X₂₈ is S, N, D or F;

### X₂₉ is P or R;

### X₃₀ is R, Q, G or H;

### X₃₁ is L, P, Q or R;

### X₃₂ is Q, R or M;

### X₃₃ is A, K, R, D or G;

### X₃₄ is Q, S or T;

### X₃₅ is S, G, D or Q;

X₃₆ is L, R or is missing;

### X₃₇ is L, P or D;

### X₃₈ is L, Q or P;

### X₃₉ is P or S;

### X₄₀ is A, S, D or V;

### X₄₁ is V or L;

### X₄₃ is H, Y or S;

### X₄₄ is H, N or D;

### X₄₅ is P or S;

### X₄₉ is Q, L, P or S;

### X₅₂ is Q or R;

### X₅₄ is V or I;

### X₅₆ is A, Q, T or E;

### X₅₇ is S or N;

### X₅₈ is Q, E, K or S;

### X₅₉ is E, D or Q;

### X₆₀ is A or V;

### X₆₁ is S or A;

### X₆₂ is S or N;

### X₆₃ is I or T;

### X₆₄ is F or L;

### X₆₅ is K, Q or L;

### X₆₆ is T or A;

### X₆₉ is T or S;

### X₇₀ is I or M; and

### X₇₁ is A, S or D.

In various embodiments: the purified polypeptide comprises at least 15 amino acids, at least 20 amino acids, at least 25 amino acids, at least 30 amino acids, at least 40 amino acids, at least 45 amino acids, at least 50 amino acids, at least 55 amino acids, at least 60 amino acids, at least 65 amino acids, at least 70 amino acids, at least 75 amino acids, or at least 80 amino acids. In various embodiments the purified polypeptide comprises fewer than 80, 75, 70 65, 60, 55, 50, 45, 40, 35, 30, 25 or 20 contiguous amino acids of SEQ ID NO:ZZ1 and has the ability to bind and/or activate the GC-C receptor and/or elicit diuresis when administered to a subject.

Compositions, including pharmaceutical compositions, can include at least one such polypeptide or can include at least two (three, four or more) such polypeptides which are different. In the compositions containing two or more such polypeptides the polypeptides can be separate or they can be covalently direct linked, e.g, by a peptide bond or a linker or they can be indirectly linked. For example, two such polypeptide sequences can be contained within a larger polypeptide and the two polypeptide sequences can be separated by other polypeptide sequences.

In certain embodiments of the purified polypeptide comprising at least 10 contiguous amino acids of SEQ ID NO:ZZ1 and in certain embodiments of the polypeptide consisting of a polypeptide fragment of SEQ ID NO:ZZ1 comprising at least 10 contiguous amino acids of SEQ ID NO:ZZ1:

### X₂ is Y;

### X₄ is Q or K;

### X₆ is Q, or H;

### X₈ is F;

### X₉ is R or Q;

### X₁₁ is Q;

### X₁₃ is E;

### X₁₅ is M or V;

### X₁₇ is K;

### X₁₉ is S;

### X₂₀ is D;

### X₂₂ is E;

### X₂₃ is A or E;

### X₂₄ is Q or K;

### X₂₅ is W;

### X₂₆ is A or M;

### X₂₇ is P or S;

### X₂₈ is S;

### X₂₉ is P;

### X₃₀ is R or Q;

### X₃₁ is L;

### X₃₂ is Q or R;

### X₃₃ is A or K;

### X₃₄ is Q or S;

### X₃₅ is S or G;

X₃₆ is missing;

### X₃₇ is L or P;

### X₃₈ is L or Q;

### X₃₉ is P;

### X₄₀ is A;

### X₄₁ is V;

### X₄₃ is H;

### X₄₄ is H or N;

### X₄₅ is P;

### X₄₉ is Q or L;

### X₅₂ is Q;

### X₅₄ is V or I;

### X₅₆ is A;

### X₅₇ is S;

### X₅₈ is Q;

### X₅₉ is E;

### X₆₀ is A;

### X₆₁ is S or A;

### X₆₂ is S;

### X₆₃ is I or T;

### X₆₄ is F;

### X₆₅ is K;

### X₆₆ is T or A;

### X₆₉ is T;

### X₇₀ is I; and

### X₇₁ is A.

Also described is: 1) a purified polypeptide comprising (or consisting of or consisting essentially of) at least 10 contiguous amino acids of SEQ ID NO:ZZ2 and 2) a purified polypeptide consisting of a polypeptide fragment of SEQ ID NO:ZZ2 comprising (or consisting of or consisting essentially of) at least 10 contiguous amino acids of SEQ ID NO:ZZ2 V X₂, X₄ Y X₆ G X₈ X₉ V X₁₁ L X₁₃ S X₁₅ K X₁₇ L X₁₉ X₂₀ L X₂₂ X₂₃ X₂₄ X₂₅ X₂₆ X₂₇ X₂₈ X₂₉ X₃₀ X₃₁ X₃₂ X₃₃ X₃₄ X₃₅ X₃₆ X₃₇ X₃₈ X₃₉ X₄₀ X₄₁ C X₄₃ X₄₄ X₄₅ A L P X₄₉ D L X₅₂ P X₅₄ C X₅₆ X₅₇ X₅₈ X₅₉ X₆₀ X₆₁ X₆₂ X₆₃ X₆₄ X₆₅ X₆₆ I J R X₆₉ X₇₀ X₇₁ X₇₂ D X₇₄ C E L C X₇₉ N V A (SEQ ID NO:ZZ2-uroguanylin consensus 2), wherein:

### X₂ is Y or D;

### X₄ is Q or K;

### X₆ is Q, H or E;

### X₈ is F or Y;

### X₉ is R or Q;

### X₁₁ is Q or K;

### X₁₃ is E, K or D;

### X₁₅ is M or V;

### X₁₇ is K or Q;

### X₁₉ is S, N, K or D;

### X₂₀ is D, E or A;

### X₂₂ is E, V or L;

### X₂₃ is A, E or G;

### X₂₄ is Q or K;

### X₂₅ is W, Q, E or P;

### X₂₆ is A, M, V or R;

### X₂₇ is P or S;

### X₂₈ is S, N, D or F;

### X₂₉ is P or R;

### X₃₀ is R, Q, G or H;

### X₃₁ is L, P, Q or R;

### X₃₂ is Q, R or M;

### X₃₃ is A, K, R, D or G;

### X₃₄ is Q, S or T;

### X₃₅ is S, G, D or Q;

X₃₆ is L, R or is missing;

### X₃₇ is L, P or D;

### X₃₈ is L, Q or P;

### X₃₉ is P or S;

### X₄₀ is A, S, D or V;

### X₄₁ is V or L;

### X₄₃ is H, Y or S;

### X₄₄ is H, N or D;

### X₄₅ is P or S;

### X₄₉ is Q, L, P or S;

### X₅₂ is Q or R;

### X₅₄ is V or I;

### X₅₆ is A, Q, T or E;

### X₅₇ is S or N;

### X₅₈ is Q, E, K or S;

### X₅₉ is E, D or Q;

### X₆₀ is A or V;

### X₆₁ is S or A;

### X₆₂ is S or N;

### X₆₃ is I or T;

### X₆₄ is F or L;

### X₆₅ is K, Q or L;

### X₆₆ is T or A;

### X₆₉ is T or S;

### X₇₀ is I or M;

### X₇₁ is A, S or D;

### X₇₂ is N, T, G or Q;

### X₇₄ is D or E; and

### X₇₉ is V or I.

In various embodiments: the purified polypeptide comprises at least 15 amino acids, at least 20 amino acids, at least 25 amino acids, at least 30 amino acids, at least 40 amino acids, at least 45 amino acids, at least 50 amino acids, at least 55 amino acids, at least 60 amino acids, at least 65 amino acids, at least 70 amino acids, at least 75 amino acids, or at least 80 amino acids. In various embodiments the purified polypeptide comprises fewer than 80, 75, 70 65, 60, 55, 50, 45, 40, 35, 30, 25 or 20 contiguous amino acids of SEQ ID NO:ZZ2 and has the ability to bind and/or activate the GC-C receptor and/or elicit diuresis when administered to a subject.

Compositions, including pharmaceutical compositions, can include at least one such polypeptide or can include at least two (three, four or more) such polypeptides which are different. In the compositions containing two or more such polypeptides the polypeptides can be separate or they can be covalently direct linked, e.g, by a peptide bond or a linker or they can be indirectly linked. For example, two such polypeptide sequences can be contained within a larger polypeptide and the two polypeptide sequences can be separated by other polypeptide sequences.

In certain embodiments of the purified polypeptide comprising at least 10 contiguous amino acids of SEQ ID NO:ZZ2 and in certain embodiments of the polypeptide consisting of a polypeptide fragment of SEQ ID NO:ZZ2 comprising at least 10 contiguous amino acids of SEQ ID NO:ZZ2:

### X₂ is Y;

### X₄ is Q or K;

### X₆ is Q, or H;

### X₈ is F;

### X₉ is R or Q;

### X₁₁ is Q;

### X₁₃ is E;

### X₁₅ is M or V;

### X₁₇ is K;

### X₁₉ is S;

### X₂₀ is D;

### X₂₂ is E;

### X₂₃ is A or E;

### X₂₄ is Q or K;

### X₂₅ is W;

### X₂₆ is A or M;

### X₂₇ is P or S;

### X₂₈ is S;

### X₂₉ is P;

### X₃₀ is R or Q;

### X₃₁ is L;

### X₃₂ is Q or R;

### X₃₃ is A or K;

### X₃₄ is Q or S;

### X₃₅ is S or G;

X₃₆ is missing;

### X₃₇ is L or P;

### X₃₈ is L or Q;

### X₃₉ is P;

### X₄₀ is A;

### X₄₁ is V;

### X₄₃ is H;

### X₄₄ is H or N;

### X₄₅ is P;

### X₄₉ is Q or L;

### X₅₂ is Q;

### X₅₄ is V or I;

### X₅₆ is A;

### X₅₇ is S;

### X₅₈ is Q;

### X₅₉ is E;

### X₆₀ is A;

### X₆₁ is S or A;

### X₆₂ is S;

### X₆₃ is I or T;

### X₆₄ is F;

### X₆₅ is K;

### X₆₆ is T or A;

### X₆₉ is T;

### X₇₀ is I;

### X₇₁ is A;

### X₇₂ is N or T;

### X₇₄ is D or E; and

### X₇₉ is V or I.

Among the examples of a purified polypeptide comprising at least 10 contiguous amino acids of SEQ ID NO:ZZ1 or ZZ2 and the examples of a purified polypeptide consisting of a polypeptide fragment of SEQ ID NO:ZZ1 or ZZ2 comprising at least 10 contiguous amino acids of SEQ ID NO:ZZ1 or ZZ2 are:
a) a polypeptide consisting of amino acids 1-16 of SEQ ID NO:ZZ1 or SEQ ID NO:ZZ2;
b) a polypeptide consisting of amino acids 1-41 of SEQ ID NO:ZZ1 or SEQ ID NO:ZZ2;
c) a polypeptide consisting of amino acids 1-55 of SEQ ID NO:ZZ1 or SEQ ID NO:ZZ2;
d) a polypeptide consisting of amino acids 1-67 of SEQ ID NO:ZZ1 or SEQ ID NO:ZZ2;
e) a polypeptide consisting of amino acids 17-41 of SEQ ID NO:ZZ1 or SEQ ID NO:ZZ2;
f) a polypeptide consisting of amino acids 17-55 of SEQ ID NO:ZZ1 or SEQ ID NO:ZZ2;
g) a polypeptide consisting of amino acids 17-67 of SEQ ID NO:ZZ1 or SEQ ID NO:ZZ2;
h) a polypeptide consisting of amino acids 17-82 of SEQ ID NO:ZZ1 or SEQ ID NO:ZZ2;
i) a polypeptide consisting of amino acids 42-55 of SEQ ID NO:ZZ1 or SEQ ID NO:ZZ2;
j) a polypeptide consisting of amino acids 42-67 of SEQ ID NO:ZZ1 or SEQ ID NO:ZZ2;
k) a polypeptide consisting of amino acids 42-82 of SEQ ID NO:ZZ1 or SEQ ID NO:ZZ2;
l) a polypeptide consisting of amino acids 56-67 of SEQ ID NO:ZZ1 or SEQ ID NO:ZZ2;
m) a polypeptide consisting of amino acids 56-82 of SEQ ID NO:ZZ1 or SEQ ID NO:ZZ2;
n) a polypeptide consisting of amino acids 68-82 of SEQ ID NO:ZZ1 or SEQ ID NO:ZZ2;
o) a polypeptide consisting of amino acids 1-21 of SEQ ID NO:ZZ1 or SEQ ID NO:ZZ2;
p) a polypeptide consisting of amino acids 41-60 of SEQ ID NO:ZZ1 or SEQ ID NO:ZZ2;
q) a polypeptide consisting of amino acids 41-65 of SEQ ID NO:ZZ1 or SEQ ID NO:ZZ2;
r) a polypeptide consisting of amino acids 41-70 of SEQ ID NO:ZZ1 or SEQ ID NO:ZZ2;
s) a polypeptide consisting of amino acids 37-55 of SEQ ID NO:ZZ1 or SEQ ID NO:ZZ2;
t) a polypeptide consisting of amino acids 32-55 of SEQ ID NO:ZZ1 or SEQ ID NO:ZZ2;
u) a polypeptide consisting of amino acids 27-55 of SEQ ID NO:ZZ1 or SEQ ID NO:ZZ2;
v) a polypeptide consisting of amino acids 37-60 of SEQ ID NO:ZZ1 or SEQ ID NO:ZZ2;
w) a polypeptide consisting of amino acids 32-60 of SEQ ID NO:ZZ1 or SEQ ID NO:ZZ2;
x) a polypeptide consisting of amino acids 27-60 of SEQ ID NO:ZZ1 or SEQ ID NO:ZZ2;
y) a polypeptide consisting of amino acids 37-65 of SEQ ID NO:ZZ1 or SEQ ID NO:ZZ2;
z) a polypeptide consisting of amino acids 32-65 of SEQ ID NO:ZZ1 or SEQ ID NO:ZZ2;
aa) a polypeptide comprising amino acids 27-65 of SEQ ID NO:ZZ1 or SEQ ID NO:ZZ2;
ab) a polypeptide comprising amino acids 37-70 of SEQ ID NO:ZZ1 or SEQ ID NO:ZZ2;
ac) a polypeptide comprising amino acids 32-70 of SEQ ID NO:ZZ1 or SEQ ID NO:ZZ2; and
ad) a polypeptide comprising amino acids 27-70 of SEQ ID NO:ZZ1 or SEQ ID NO:ZZ2.

Among the examples of a purified polypeptide comprising at least 10 contiguous amino acids of SEQ ID NO:ZZ1 or ZZ2 and the examples of a polypeptide consisting of a polypeptide fragment of SEQ ID NO:ZZ1 or ZZ2 comprising at least 10 contiguous amino acids of SEQ ID NO:ZZ1 or ZZ2 are:
a) a polypeptide consisting of amino acids 1-16 of SEQ ID NO:ZZ1 or SEQ ID NO:ZZ2;
b) a polypeptide consisting of amino acids 1-41 of SEQ ID NO:ZZ1 or SEQ ID NO:ZZ2;
c) a polypeptide consisting of amino acids 1-55 of SEQ ID NO:ZZ1 or SEQ ID NO:ZZ2;
d) a polypeptide consisting of amino acids 1-67 of SEQ ID NO:ZZ1 or SEQ ID NO:ZZ2;
e) a polypeptide consisting of amino acids 17-41 of SEQ ID NO:ZZ1 or SEQ ID NO:ZZ2;
f) a polypeptide consisting of amino acids 17-55 of SEQ ID NO:ZZ1 or SEQ ID NO:ZZ2;
g) a polypeptide consisting of amino acids 17-67 of SEQ ID NO:ZZ1 or SEQ ID NO:ZZ2;
h) a polypeptide consisting of amino acids 17-82 of SEQ ID NO:ZZ1 or SEQ ID NO:ZZ2.
i) a polypeptide consisting of amino acids 42-55 of SEQ ID NO:ZZ1 or SEQ ID NO:ZZ2;
j) a polypeptide consisting of amino acids 42-67 of SEQ ID NO:ZZ1 or SEQ ID NO:ZZ2;
k) a polypeptide consisting of amino acids 42-82 of SEQ ID NO:ZZ1 or SEQ ID NO:ZZ2.
l) a polypeptide consisting of amino acids 56-67 of SEQ ID NO:ZZ1 or SEQ ID NO:ZZ2;
m) a polypeptide consisting of amino acids 56-82 of SEQ ID NO:ZZ1 or SEQ ID NO:ZZ2.
n) a polypeptide consisting of amino acids 68-82 of SEQ ID NO:ZZ1 or SEQ ID NO:ZZ2;
o) a polypeptide consisting of amino acids 1-21 of SEQ ID NO:ZZ1 or SEQ ID NO:ZZ2;
p) a polypeptide consisting of amino acids 41-60 of SEQ ID NO:ZZ1 or SEQ ID NO:ZZ2;
q) a polypeptide consisting of amino acids 41-65 of SEQ ID NO:ZZ1 or SEQ ID NO:ZZ2;
r) a polypeptide consisting of amino acids 41-70 of SEQ ID NO:ZZ1 or SEQ ID NO:ZZ2;
s) a polypeptide consisting of amino acids 37-55 of SEQ ID NO:ZZ1 or SEQ ID NO:ZZ2;
t) a polypeptide consisting of amino acids 32-55 of SEQ ID NO:ZZ1 or SEQ ID NO:ZZ2;
u) a polypeptide consisting of amino acids 27-55 of SEQ ID NO:ZZ1 or SEQ ID NO:ZZ2;
v) a polypeptide consisting of amino acids 37-60 of SEQ ID NO:ZZ1 or SEQ ID NO:ZZ2;
w) a polypeptide consisting of amino acids 32-60 of SEQ ID NO:ZZ1 or SEQ ID NO:ZZ2;
x) a polypeptide consisting of amino acids 27-60 of SEQ ID NO:ZZ1 or SEQ ID NO:ZZ2;
y) a polypeptide consisting of amino acids 37-65 of SEQ ID NO:ZZ1 or SEQ ID NO:ZZ2;
z) a polypeptide consisting of amino acids 32-65 of SEQ ID NO:ZZ1 or SEQ ID NO:ZZ2;
aa) a polypeptide consisting of amino acids 27-65 of SEQ ID NO:ZZ1 or SEQ ID NO:ZZ2;
ab) a polypeptide consisting of amino acids 37-70 of SEQ ID NO:ZZ1 or SEQ ID NO:ZZ2;
ac) a polypeptide consisting of amino acids 32-70 of SEQ ID NO:ZZ1 or SEQ ID NO:ZZ2; and
ad) a polypeptide consisting of amino acids 27-70 of SEQ ID NO:ZZ1 or SEQ ID NO:ZZ2.

Among the examples of a purified polypeptide comprising at least 10 contiguous amino acids of SEQ ID NO:ZZ1 or ZZ2 and the examples of a polypeptide consisting of a polypeptide fragment of SEQ ID NO:ZZ1 or ZZ2 comprising at least 10 contiguous amino acids of SEQ ID NO:ZZ1 or ZZ2 are:
a) a polypeptide consisting essentially of amino acids 1-16 of SEQ ID NO:ZZ1 or SEQ ID NO:ZZ2;
b) a polypeptide consisting essentially of amino acids 1-41 of SEQ ID NO:ZZ1 or SEQ ID NO:ZZ2;
c) a polypeptide consisting essentially of amino acids 1-55 of SEQ ID NO:ZZ1 or SEQ
ID NO:ZZ2;
d) a polypeptide consisting essentially of amino acids 1-67 of SEQ ID NO:ZZ1 or SEQ ID NO:ZZ2;
e) a polypeptide consisting essentially of amino acids 17-41 of SEQ ID NO:ZZ1 or SEQ ID NO:ZZ2;
f) a polypeptide consisting essentially of amino acids 17-55 of SEQ ID NO:ZZ1 or SEQ ID NO:ZZ2;
g) a polypeptide consisting essentially of amino acids 17-67 of SEQ ID NO:ZZ1 or SEQ ID NO:ZZ2;
h) a polypeptide consisting essentially of amino acids 17-82 of SEQ ID NO:ZZ1 or SEQ ID NO:ZZ2.
i) a polypeptide consisting essentially of amino acids 42-55 of SEQ ID NO:ZZ1 or SEQ ID NO:ZZ2;
j) a polypeptide consisting essentially of amino acids 42-67 of SEQ ID NO:ZZ1 or SEQ ID NO:ZZ2;
k) a polypeptide consisting essentially of amino acids 42-82 of SEQ ID NO:ZZ1 or SEQ ID NO:ZZ2.
l) a polypeptide consisting essentially of amino acids 56-67 of SEQ ID NO:ZZ1 or SEQ ID NO:ZZ2;
m) a polypeptide consisting essentially of amino acids 56-82 of SEQ ID NO:ZZ1 or SEQ ID NO:ZZ2.
n) a polypeptide consisting essentially of amino acids 68-82 of SEQ ID NO:ZZ1 or SEQ ID NO:ZZ2;
o) a polypeptide consisting essentially of amino acids 1-21 of SEQ ID NO:ZZ1 or SEQ ID NO:ZZ2;
p) a polypeptide consisting essentially of amino acids 41-60 of SEQ ID NO:ZZ1 or SEQ ID NO:ZZ2;
q) a polypeptide consisting essentially of amino acids 41-65 of SEQ ID NO:ZZ1 or SEQ ID NO:ZZ2;
r) a polypeptide consisting essentially of amino acids 41-70 of SEQ ID NO:ZZ1 or SEQ ID NO:ZZ2;
s) a polypeptide consisting essentially of amino acids 37-55 of SEQ ID NO:ZZ1 or SEQ ID NO:ZZ2;
t) a polypeptide consisting essentially of amino acids 32-55 of SEQ ID NO:ZZ1 or SEQ ID NO:ZZ2;
u) a polypeptide consisting essentially of amino acids 27-55 of SEQ ID NO:ZZ1 or SEQ ID NO:ZZ2;
v) a polypeptide consisting essentially of amino acids 37-60 of SEQ ID NO:ZZ1 or SEQ ID NO:ZZ2;
w) a polypeptide consisting essentially of amino acids 32-60 of SEQ ID NO:ZZ1 or SEQ ID NO:ZZ2;
x) a polypeptide consisting essentially of amino acids 27-60 of SEQ ID NO:ZZ1 or SEQ ID NO:ZZ2;
y) a polypeptide consisting essentially of amino acids 37-65 of SEQ ID NO:ZZ1 or SEQ ID NO:ZZ2;
z) a polypeptide consisting essentially of amino acids 32-65 of SEQ ID NO:ZZ1 or SEQ ID NO:ZZ2;
aa) a polypeptide consisting essentially of amino acids 27-65 of SEQ ID NO:ZZ1 or SEQ ID NO:ZZ2;
ab) a polypeptide consisting essentially of amino acids 37-70 of SEQ ID NO:ZZ1 or SEQ ID NO:ZZ2;
ac) a polypeptide consisting essentially of amino acids 32-70 of SEQ ID NO:ZZ1 or SEQ ID NO:ZZ2; and
ad) a polypeptide consisting essentially of amino acids 27-70 of SEQ ID NO:ZZ1 or SEQ ID NO:ZZ2.

The contemplated purified polypeptides include any subset of the aforementioned polypeptides as well as each one of the forgoing polypeptides.

For SEQ ID NO:ZZ1 and SEQ ID NO:ZZ2 useful compositions, including useful pharmaceutical compositions, that include, for example, more than one of polypeptides (a)-(ad) (or polypeptides comprising, consisting of or consisting essentially of one or more of (a)-(ad) or a consisting of a polypeptide fragment of SEQ ID NO:ZZ1 or ZZ2) comprising one or more of (a)-(ad)) include combinations 1-38 below:

| | |
|---|---|
| 1 | (a), (e) |
| 2 | (a), (e), (i) |
| 3 | (a), (e), (i), (l) |
| 4 | (a), (e), (i), (l), (n) |
| 5 | (a), (e), (i), (m) |
| 6 | (a), (e), (k) |
| 7 | (a), (f) |
| 8 | (a), (f), (l) |
| 9 | (a), (f), (l), (n) |
| 10 | (a), (f), (m) |
| 11 | (a), (g) |
| 12 | (a), (g), (n) |
| 13 | (a), (h) |
| 14 | (b), (i) |
| 15 | (b), (i), (l) |
| 16 | (b), (i), (l), (n) |
| 17 | (b), (i), (m) |
| 18 | (b), (j) |
| 19 | (b), (j), (n) |
| 20 | (b), (k) |
| 21 | (c), (l) |
| 22 | (c), (l), (n) |
| 23 | (d), (n) |
| 24 | (e), (i) |
| 25 | (e), (i), (l) |
| 26 | (e), (i), (l), (n) |
| 27 | (e), (i), (m) |
| 28 | (e), (j) |
| 29 | (e), (j), (n) |
| 30 | (e), (k) |
| 31 | (f), (l) |
| 32 | (f), (l), (n) |
| 33 | (f), (m) |
| 34 | (g), (n) |
| 35 | (i), (l), (n) |
| 36 | (i), (m) |
| 37 | (j), (n) |
| 38 | (l), (n) |

For SEQ ID NO:ZZ1 and SEQ ID NO:ZZ2, useful pharmaceutical compositions include: a pharmaceutical composition comprising at least two (three, four or more) of polypeptides (a)-(ad) and a pharmaceutically acceptable carrier. In various embodiments the pharmaceutical composition comprises:
polypeptide (a) and at least one of: polypeptides (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (b) and at least one of polypeptides (c), (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (c) and at least one of polypeptides (d), (e), (f), (g), (h), (i), (j), (k), (l), (m), (n), (o), (p), (q), (r), (s), (t), (u), (v), (w), (x), (y), (z), (aa), (ab), (ac), (ad);
polypeptide (d) and at least one of polypeptides (e), (f), (g), (h), (i), (j), (k), (l), (m), (n), (o), (p), (q), (r), (s), (t), (u), (v), (w), (x), (y), (z), (aa), (ab), (ac), (ad);
polypeptide (e) and at least one of polypeptides (f), (g), (h), (i), (j), (k), (l), (m), (n), (o), (p), (q), (r), (s), (t), (u), (v), (w), (x), (y), (z), (aa), (ab), (ac), (ad);
polypeptide (f) and at least one of polypeptides (g), (h), (i), (j), (k), (l), (m), (n), (o), (p), (q), (r), (s), (t), (u), (v), (w), (x), (y), (z), (aa), (ab), (ac), (ad);
polypeptide (g) and at least one of polypeptides (h), (i), (j), (k), (l), (m), (n), (o), (p), (q), (r), (s), (t), (u), (v), (w), (x), (y), (z), (aa), (ab), (ac), (ad);
polypeptide (h) and at least one of polypeptides (i), (j), (k), (l), (m), (n), (o), (p), (q), (r), (s), (t), (u), (v), (w), (x), (y), (z), (aa), (ab), (ac), (ad);
polypeptide (i) and at least one of polypeptides (j), (k), (l), (m), (n), (o), (p), (q), (r), (s), (t), (u), (v), (w), (x), (y), (z), (aa), (ab), (ac), (ad);
polypeptide (j) and at least one of polypeptides (k), (l), (m), (n), (o), (p), (q), (r), (s), (t), (u), (v), (w), (x), (y), (z), (aa), (ab), (ac), (ad);
polypeptide (k) and at least one of polypeptides (l), (m), (n), (o), (p), (q), (r), (s), (t), (u), (v), (w), (x), (y), (z), (aa), (ab), (ac), (ad);
polypeptide (l) and at least one of polypeptides (m), (n), (o), (p), (q), (r), (s), (t), (u), (v), (w), (x), (y), (z), (aa), (ab), (ac), (ad);
polypeptide (m) and at least one of polypeptides (n), (o), (p), (q), (r), (s), (t), (u), (v), (w), (x), (y), (z), (aa), (ab), (ac), (ad);
polypeptide (n) and at least one of polypeptides (o), (p), (q), (r), (s), (t), (u), (v), (w), (x), (y), (z), (aa), (ab), (ac), (ad);
polypeptide (o) and at least one of polypeptides (p), (q), (r), (s), (t), (u), (v), (w), (x), (y), (z), (aa), (ab), (ac), (ad);
polypeptide (p) and at least one of polypeptides (q), (r), (s), (t), (u), (v), (w), (x), (y), (z), (aa), (ab), (ac), (ad);
polypeptide (q) and at least one of polypeptides (r), (s), (t), (u), (v), (w), (x), (y), (z), (aa), (ab), (ac), (ad);
polypeptide (r) and at least one of polypeptides (s), (t), (u), (v), (w), (x), (y), (z), (aa), (ab), (ac), (ad);
polypeptide (s) and at least one of polypeptides (t), (u), (v), (w), (x), (y), (z), (aa), (ab), (ac), (ad);
polypeptide (t) and at least one of polypeptides (u), (v), (w), (x), (y), (z), (aa), (ab), (ac), (ad);
polypeptide (u) and at least one of polypeptides (v), (w), (x), (y), (z), (aa), (ab), (ac), (ad);
polypeptide (v) and at least one of polypeptides (w), (x), (y), (z), (aa), (ab), (ac), (ad);
polypeptide (w) and at least one of polypeptides (x), (y), (z), (aa), (ab), (ac), (ad);
polypeptide (x) and at least one of polypeptides (y), (z), (aa), (ab), (ac), (ad);
polypeptide (y) and at least one of polypeptides (z), (aa), (ab), (ac), (ad);
polypeptide (z) and at least one of polypeptides (aa), (ab), (ac), (ad);
polypeptide (aa) and at least one of polypeptides (ab), (ac), (ad);
polypeptide (ab) and at least one of polypeptides (ac), (ad); and
polypeptide (ac) and at least polypeptide (ad).

Useful polypeptides include those comprising, consisting of, or consisting essentially of: amino acids 1-23 of SEQ ID NO:X; amino acids 48-66 of SEQ ID NO:X; amino acids 48-71 of SEQ ID NO:X; amino acids 48-76 of SEQ ID NO:X; amino acids 43-61 of SEQ ID NO:X; amino acids 38-61 of SEQ ID NO:X; amino acids 33-61 of SEQ ID NO:X; amino acids 43-66 of SEQ ID NO:X; amino acids 38-66 of SEQ ID NO:X; amino acids 33-66 of SEQ ID NO:X; amino acids 43-71 of SEQ ID NO:X; amino acids 38-71 of SEQ ID NO:X; amino acids 33-71 of SEQ ID NO:X; amino acids 43-76 of SEQ ID NO:X; amino acids 38-76 of SEQ ID NO:X; amino acids 33-76 of SEQ ID NO:X; amino acids 1-23 of SEQ ID NO:X1; amino acids 51-70 of SEQ ID NO:X1; amino acids 51-75 of SEQ ID NO:X1; amino acids 51-80 of SEQ ID NO:X1; amino acids 46-65 of SEQ ID NO:X1; amino acids 41-65 of SEQ ID NO:X1; amino acids 36-65 of SEQ ID NO:X1; amino acids 46-70 of SEQ ID NO:X1; amino acids 41-70 of SEQ ID NO:X1; amino acids 36-70 of SEQ ID NO:X1; amino acids 46-75 of SEQ ID NO:X1; amino acids 41-75 of SEQ ID NO:X1; amino acids 36-75 of SEQ ID NO:X1; amino acids 46-80 of SEQ ID NO:X1; amino acids 41-80 of SEQ ID NO:X1; amino acids 36-80 of SEQ ID NO:X1; amino acids 1-23 of SEQ ID NO: X2; amino acids 51-70 of SEQ ID NO: X2; amino acids 51-75 of SEQ ID NO: X2; amino acids 51-80 of SEQ ID NO: X2; amino acids 46-65 of SEQ ID NO: X2; amino acids 41-65 of SEQ ID NO: X2; amino acids 36-65 of SEQ ID NO: X2; amino acids 46-70 of SEQ ID NO: X2; amino acids 41-70 of SEQ ID NO: X2; amino acids 36-70 of SEQ ID NO: X2; amino acids 46-75 of SEQ ID NO: X2; amino acids 41-75 of SEQ ID NO: X2; amino acids 36-75 of SEQ ID NO: X2; amino acids 46-80 of SEQ ID NO: X2; amino acids 41-80 of SEQ ID NO: X2; and amino acids 36-80 of SEQ ID NO: X2. The contemplated compositions include any subset of the aforementioned polypeptides as well as each one of the forgoing polypeptides.

Useful polypeptides include those comprising, consisting of, or consisting essentially of: amino acids 1-21 of SEQ ID NO:ZZ; amino acids 41-59 of SEQ ID NO:ZZ; amino acids 41-64 of SEQ ID NO:ZZ; amino acids 41-69 of SEQ ID NO:ZZ; amino acids 36-54 of SEQ ID NO:ZZ; amino acids 31-54 of SEQ ID NO:ZZ; amino acids 26-54 of SEQ ID NO:ZZ; amino acids 36-59 of SEQ ID NO:ZZ; amino acids 31-59 of SEQ ID NO:ZZ; amino acids 26-59 of SEQ ID NO:ZZ; amino acids 36-64 of SEQ ID NO:ZZ; amino acids 31-64 of SEQ ID NO:ZZ; amino acids 26-64 of SEQ ID NO:ZZ; amino acids 36-69 of SEQ ID NO:ZZ; amino acids 31-69 of SEQ ID NO:ZZ; amino acids 26-69 of SEQ ID NO:ZZ; amino acids 1-21 of SEQ ID NO:ZZ1; amino acids 41-60 of SEQ ID NO:ZZ1; amino acids 41-65 of SEQ ID NO:ZZ1; amino acids 41-70 of SEQ ID NO:ZZ1; amino acids 37-55 of SEQ ID NO:ZZ1; amino acids 32-55 of SEQ ID NO:ZZ1; amino acids 27-55 of SEQ ID NO:ZZ1; amino acids 37-60 of SEQ ID NO:ZZ1; amino acids 32-60 of SEQ ID NO:ZZ1; amino acids 27-60 of SEQ ID NO:ZZ1; amino acids 37-65 of SEQ ID NO:ZZ1; amino acids 32-65 of SEQ ID NO:ZZ1; amino acids 27-65 of SEQ ID NO:ZZ1; amino acids 37-70 of SEQ ID NO:ZZ1; amino acids 32-70 of SEQ ID NO:ZZ1; amino acids 27-70 of SEQ ID NO:ZZ1; amino acids 1-21 of SEQ ID NO: ZZ2; amino acids 41-60 of SEQ ID NO: ZZ2; amino acids 41-65 of SEQ ID NO: ZZ2; amino acids 41-70 of SEQ ID NO: ZZ2; amino acids 37-55 of SEQ ID NO: ZZ2; amino acids 32-55 of SEQ ID NO: ZZ2; amino acids 27-55 of SEQ ID NO: ZZ2; amino acids 37-60 of SEQ ID NO: ZZ2; amino acids 32-60 of SEQ ID NO: ZZ2; amino acids 27-60 of SEQ ID NO: ZZ2; amino acids 37-65 of SEQ ID NO: ZZ2; amino acids 32-65 of SEQ ID NO: ZZ2; amino acids 27-65 of SEQ ID NO: ZZ2; amino acids 37-70 of SEQ ID NO: ZZ2; amino acids 32-70 of SEQ ID NO: ZZ2; and amino acids 27-70 of SEQ ID NO: ZZ2. The contemplated compositions include any subset of the aforementioned polypeptides as well as each one of the forgoing polypeptides.

In various embodiments of the aforementioned purified polypeptides: the polypeptide binds to the GC-C receptor; the polypeptide increases cGMP levels when administered to a subject; the polypeptide increases cGMP levels in the T84 assay; the polypeptide increases intestinal transit when administered to a subject; the polypeptide decreases intestinal transit when administered to a subject; the polypeptide decreases stool firmness when administered to a subject; the polypeptide increases stool frequency when administered to a subject; the polypeptide decreases visceral pain when administered to a subject; the polypeptide modulates activity of the GC-C receptor; the polypeptide increases the activity of the GC-C receptor and the polypeptide decreases the activity of the GC-C receptor, the polypeptide increases urine output in a rodent diuresis assay, the polypeptide elicits diuresis when administered to a subject, the polypeptide elicits naturesis when administered to a subject, the polypeptide elicits kaluresis when administered to a subject

Also described is a method of treating a disorder associated with reduced gastrointestinal transit rate or reduced gastrointestinal motility comprising administering a pharmaceutical composition comprising an aforementioned polypeptide to a patient in need thereof.

Also described is a method of treating a gastrointestinal hypomotility disorder comprising administering a pharmaceutical composition comprising an aforementioned polypeptide to a patient in need thereof. In various embodiments: the disorder is selected from the group consisting of constipation, constipation dominant irritable bowel syndrome and pelvic floor dyssynergia.

Also described is a method of treating a non-inflammatory gastrointestinal disorder comprising administering a pharmaceutical composition comprising an aforementioned polypeptide to a patient in need thereof.

Also described is a method of treating a gastrointestinal disorder other than Crohn's disease and ulcerative colitis comprising administering a pharmaceutical composition comprising an aforementioned polypeptide to a patient in need thereof.

Also described is a method of treating a gastrointestinal disorder comprising administering a pharmaceutical composition comprising an aforementioned polypeptide to a patient in need thereof. In various embodiments the gastrointestinal disorder is: a gastrointestinal motility disorder, irritable bowel syndrome, a functional gastrointestinal disorder, gastroesophageal reflux disease, duodenogastric reflux, functional heartburn, dyspepsia, functional dyspepsia, nonulcer dyspepsia, gastroparesis, chronic intestinal pseudo-obstruction, or colonic pseudo-obstruction.

Also described is a method of treating obesity comprising administering a pharmaceutical composition comprising an aforementioned polypeptide to a patient in need thereof.

Also described is a method of treating congestive heart failure comprising administering a pharmaceutical composition of any comprising an aforementioned polypeptide to a patient in need thereof. In various embodiments the congestive heart failure is categorized as Class II congestive heart failure; the congestive heart failure is categorized as Class III congestive heart failure; and the congestive heart failure is categorized as Class IV congestive heart failure. The New York Heart Association (NYHA) functional classification system relates congestive heart failure symptoms to everyday activities and the patient's quality of life. The NYHA defines the classes of patient symptoms relating to congestive heart failure as: Class II-slight limitation of physical activity, comfortable at rest, but ordinary physical activity results in fatigue, palpitation, or dyspnea; Class III—marked limitation of physical activity, comfortable at rest, but less than ordinary activity causes fatigue, palpitation, or dyspnea and Class IV—unable to carry out any physical activity without discomfort, symptoms of cardiac insufficiency at rest, if any physical activity is undertaken, discomfort is increased. Heart failure treatment using the polypeptides and methods described herein can also be classified according to the ACC/AHA guidelines (Stage A: At risk for developing heart failure without evidence of cardiac dysfunction; Stage B: Evidence of cardiac dysfunction without symptoms; Stage C: Evidence of cardiac dysfunction with symptoms; and Stage D: Symptoms of heart failure despite maximal therapy).

Also described is a method of treating benign prostatic hyperplasia comprising administering a pharmaceutical composition comprising an aforementioned polypeptide to a patient in need thereof.

Also described is a method of treating constipation comprising administering the pharmaceutical composition comprising an aforementioned polypeptide to a patient in need thereof.

Also described is a method of treating a gastrointestinal motility disorder in a patient, the method comprising administering a pharmaceutical composition comprising an aforementioned polypeptide to a patient in need thereof.

Also described is a method of treating gastrointestinal pain or visceral pain in a patient, the method comprising administering a pharmaceutical composition comprising an aforementioned polypeptide to a patient in need thereof.

Also described is a method for increasing the activity of an intestinal guanylate cyclase (GC-C) receptor in a patient, the method comprising administering a pharmaceutical composition comprising an aforementioned polypeptide to a patient in need thereof.

Also described is a method of treating a disorder characterized by salt retention, fluid retention, and combinations thereof, in a patient in need thereof, the method comprising administering a pharmaceutical composition comprising an aforementioned polypeptide to a patient in need thereof.

Unless otherwise specified, the term “agonist” used herein refers to an agonist of the GC-C receptor. The polypeptide can contain up to four cysteines that form one or two disulfide bonds or do not form a disulfide bond. In certain embodiments the disulfide bonds are replaced by other covalent cross-links and in some cases the cysteines are substituted by other residues to provide for alternative covalent cross-links. The polypeptides may also include at least one trypsin or chymotrypsin cleavage site and/or a carboxy-terminal analgesic polypeptide or small molecule, e.g., AspPhe or some other analgesic polypeptide. When present within the polypeptide, the analgesic polypeptide or small molecule may be preceded by a chymotrypsin or trypsin cleavage site that allows release of the analgesic polypeptide or small molecule. The polypeptides and methods are also useful for treating pain and inflammation associated with various disorders, including gastrointestinal disorders. Certain polypeptides include a functional chymotrypsin or trypsin cleavage site located so as to allow inactivation of the polypeptide upon cleavage. Certain polypeptides having a functional cleavage site undergo cleavage and gradual inactivation in the digestive tract, and this is desirable in some circumstances. In certain polypeptides, a functional chymotrypsin site is altered, increasing the stability of the polypeptide in vivo (e.g., guanylin).

Among the methods described herein are: a method for increasing intestinal motility comprising administering a GC-C receptor agonist, e.g., a polypeptide described herein, to a patient in need thereof; a method for treating a disorder associated with reduced gastrointestinal transit rates or reduced gastrointestinal motility comprising administering a GC-C receptor agonist, e.g., a polypeptide described herein, to a patient in need thereof; a method for treating a gastrointestinal hypomotility disorder comprising administering a GC-C receptor agonist, e.g., a polypeptide described herein, to a patient in need thereof; a method treating a non-inflammatory gastrointestinal disorder comprising administering a GC-C receptor agonist, e.g., a polypeptide described herein, to a patient in need thereof.; a method treating a gastrointestinal disorder other than Crohn's disease and ulcerative colitis comprising administering a GC-C receptor agonist, e.g., a polypeptide described herein, to a patient in need thereof.

The disorders which can be treated by administering a GC-C receptor agonist, e.g., a polypeptide described herein, include constipation, constipation dominant irritable bowel syndrome and pelvic floor dyssynergia.

Also described are methods for treating other disorders such as congestive heart failure and benign prostatic hyperplasia by administering a polypeptide or small molecule (parenterally or orally) that acts as an agonist of the GC-C receptor. Such agents can be used in combination with natriuretic polypeptides (e.g., atrial natriuretic polypeptide, brain natriuretic polypeptide or C-type natriuretic polypeptide), a diuretic, or an inhibitor of angiotensin converting enzyme.

Also described are methods and compositions for increasing intestinal motility. Intestinal motility involves spontaneous coordinated distentions and contractions of the stomach, intestines, colon and rectum to move food through the gastrointestinal tract during the digestive process.

In certain embodiments the patient has been diagnosed as suffering from IBS according to the Rome criteria. In certain embodiments the patient is female.

The polypeptide can contain additional carboxy terminal or amino terminal amino acids or both. For example, the polypeptide can include an amino terminal sequence that facilitates a recombinant production of the polypeptide and is cleaved prior to administration of the polypeptide to a patient. The polypeptide can also include other amino terminal or carboxy terminal amino acids. In some cases the additional amino acids protect the polypeptide, stabilize the polypeptide or alter the activity of the polypeptide. In some cases some or all of these additional amino acids are removed prior to administration of the polypeptide to a patient. The polypeptide can include 1, 2, 3, 4, 5, 10, 15, 20, 25, 30, 40, 50, 60, 70 80, 90, 100 or more amino acids at its amino terminus or carboxy terminus or both. The number of flanking amino acids need not be the same. For example, there can be 10 additional amino acids at the amino terminus of the polypeptide and none at the carboxy terminus.

The polypeptides described herein can be administered together with: mature human guanylin or a polypeptide comprising the amino acid sequence of mature human guanylin, mature human uroguanylin or a polypeptide comprising the amino acid sequence of mature human uroguanylin, or certain other polypeptides that act as GC-C receptor agonists.

Useful variants of PGTCEICAYAACTGC (human guanylin) (SEQ ID NO:) that can be combined with the polypeptide described herein include:

| | |
|---|---|
| PGTCEICATAACTGC | (SEQ ID NO: ) |
| PGTCEICANAACTGC | (SEQ ID NO: ) |
| PGTCEICAQAACTGC | (SEQ ID NO: ) |
| PGTCEICARAACTGC | (SEQ ID NO: ) |
| PGTCEICAEAACTGC | (SEQ ID NO: ) |
| PGTCEICADAACTGC | (SEQ ID NO: ) |
| PGTCEICAGAACTGC | (SEQ ID NO: ) |
| PGTCEICAAAACTGC | (SEQ ID NO: ) |
| PGTCEICAMAACTGC | (SEQ ID NO: ) |
| PGTCEICAIAACTGC | (SEQ ID NO: ) |
| PGTCEICALAACTGC | (SEQ ID NO: ) |
| PGTCEICAVAACTGC | (SEQ ID NO: ) |
| PGTCEICAHAACTGC | (SEQ ID NO: ) |
| PGTCEGICAYAACTGC | (SEQ ID NO: ) |
| PGTCEIGCAYAACTGC | (SEQ ID NO: ) |
| PGTCEICGAYAACTGC | (SEQ ID NO: ) |
| PGTCEICAGYAACTGC | (SEQ ID NO: ) |
| PGTCEICAYGAACTGC | (SEQ ID NO: ) |
| PGTCEICAYAGACTGC | (SEQ ID NO: ) |
| PGTCEICAYAAGCTGC | (SEQ ID NO: ) |
| PGTCEICAYAACGTGC | (SEQ ID NO: ) |
| PGTCEICAYAACTGGC | (SEQ ID NO: ) |
| PGTCAEICAYAACTGC | (SEQ ID NO: ) |
| PGTCEAICAYAACTGC | (SEQ ID NO: ) |
| PGTCEIACAYAACTGC | (SEQ ID NO: ) |
| PGTCEICAAYAACTGC | (SEQ ID NO: ) |
| PGTCEICAYAAACTGC | (SEQ ID NO: ) |
| PGTCEICAYAACATGC | (SEQ ID NO: ) |
| PGTCEICAYAACTAGC | (SEQ ID NO: ) |
| PGTCEICAYAACTGAC | (SEQ ID NO: ) |
| PGTCAEICAAYAACTGC | (SEQ ID NO: ) |
| PGTCEAICAAYAACTGC | (SEQ ID NO: ) |
| PGTCEIACAAYAACTGC | (SEQ ID NO: ) |

The polypeptides described herein can be attached to one, two or more different moieties each providing the same or different functions. For example, the polypeptide can be linked to a molecule that is an analgesic and to a polypeptide that is used to treat obesity. The polypeptide and various moieties can be ordered in various ways. For example, a polypeptide described herein can have an analgesic polypeptide linked to its amino terminus and an anti-obesity polypeptide linked to its carboxy terminus. The additional moieties can be directly covalently bonded to the polypeptide or can be bonded via linkers.

The polypeptides described herein can be a cyclic polypeptide or a linear polypeptide. In addition, multiple copies of the same polypeptide can be incorporated into a single cyclic or linear polypeptide.

The polypeptides can include the amino acid sequence of a polypeptide that occurs naturally in a vertebrate (e.g., mammalian) species or in a bacterial species. In addition, the polypeptides can be partially or completely non-naturally occurring polypeptides. Also within the invention are peptidomimetics corresponding to the polypeptides described herein.

In the case of mature uroguanylin and mature guanylin, when fully folded, disulfide bonds are present between the first and third cysteines and between the second and fourth cysteines in the mature protein, e.g., in mature uroguanylin there is a disulfide bond between Cys₄ and Cys₁₂ and a disulfide bond between Cys₇ and Cys₁₅. In some embodiments, the polypeptide has only one disulfide bond, e.g., between the first and third cysteines. In certain embodiments one or more Cys can be replaced by Mpt (mercaptoproline) or Pen (penicillamine) or Dpr (diaminopropionic acid) or some other amino acid that can covalently link to another amino acid (e.g., Cys, Mpt, Pen or Dpr). In other embodiments, the polypeptide is a reduced polypeptide having no disulfide bonds.

In some embodiments, one or both members of a pair of Cys residues which normally form a disulfide bond can be replaced by homocysteine, penicillamine, 3-mercaptoproline (Kolodziej et al. 1996 Int J Pept Protein Res 48:274); β,β-dimethylcysteine (Hunt et al. 1993 Int J Pept Protein Res 42:249) or diaminopropionic acid (Smith et al. 1978 J Med Chem 21:117) to form alternative internal cross-links at the positions of the normal disulfide bonds.

In addition, one or more disulfide bonds can be replaced by alternative covalent cross-links, e.g., an amide linkage (—CH₂CH(O)NHCH₂— or —CH₂NHCH(O)CH₂—), an ester linkage, a thioester linkage, a lactam bridge, a carbamoyl linkage, a urea linkage, a thiourea linkage, a phosphonate ester linkage, an alkyl linkage (—CH₂CH₂CH₂CH₂—), an alkenyl linkage (—CH₂CH═CHCH₂—), an ether linkage (—CH₂CH₂OCH₂— or —CH₂OCH₂CH₂—), a thioether linkage (—CH²CH₂SCH₂— or —CH₂SCH₂CH₂—), an amine linkage (—CH₂CH₂NHCH₂— or —CH₂NHCH₂CH₂—) or a thioamide linkage (—CH₂CH(S)HNHCH₂— or —CH₂NHCH(S)CH₂—). For example, Ledu et al. (Proc Nat'l Acad. Sci. 100:11263-78, 2003) describe methods for preparing lactam and amide cross-links. Schafmeister et al. (J. Am. Chem. Soc. 122:5891, 2000) describe stable, hydrocarbon cross-links. Hydrocarbon cross links can be produced via metathesis (or methathesis followed by hydrogenation in the case of saturated hydrocarbons cross-links) using one or another of the Grubbs catalysts (available from Materia, Inc. and Sigma-Aldrich and described, for example, in U.S. Pat. Nos. 5,831,108 and 6,111,121). In some cases, the generation of such alternative cross-links requires replacing the Cys residues with other residues such as Lys or Glu or non-naturally occurring amino acids. In addition the lactam, amide and hydrocarbon cross-links can be used to stabilize the polypeptide even if they link amino acids at positions other than those occupied by Cys. Such cross-links can occur between two amino acids that are separated by two amino acids or between two amino acids that are separated by six amino acids (see, e.g., Schafmeister et al. (J. Am. Chem. Soc. 122:5891, 2000)).

In certain embodiments, one or more amino acids can be replaced by a non-naturally occurring amino acid or a naturally or non-naturally occurring amino acid analog. There are many amino acids beyond the standard 20 (Ala, Arg, Asn, Asp, Cys, Gln, Glu, Gly, H is, Ile, Leu, Lys, Met, Phe, Pro, Ser, Thr, Tip, Tyr, and Val). Some are naturally-occurring others are not (see, for example, Hunt, The Non-Protein Amino Acids: In Chemistry and Biochemistry of the Amino Acids, Barrett, Chapman and Hall, 1985). For example, an aromatic amino acid can be replaced by 3,4-dihydroxy-L-phenylalanine, 3-iodo-L-tyrosine, triiodothyronine, L-thyroxine, phenylglycine (Phg) or nor-tyrosine (norTyr). Phg and norTyr and other amino acids including Phe and Tyr can be substituted by, e.g., a halogen, —CH₃, —OH, —CH₂NH₃, —C(O)H, —CH₂CH₃, —CN, —CH₂CH₂CH₃, —SH, or another group. Any amino acid can be substituted by the D-form of the amino acid.

With regard to non-naturally occurring amino acids or a naturally and non-naturally occurring amino acid analogs, a number of substitutions in the polypeptide and agonists described herein are possible alone or in combination.

For example, glutamine residues can be substituted with gamma-Hydroxy-Glu or gamma-Carboxy-Glu. Tyrosine residues can be substituted with an alpha substituted amino acid such as L-alpha-methylphenylalanine or by analogues such as: 3-Amino-Tyr; Tyr(CH3); Tyr(PO₃(CH₃)₂); Tyr(SO₃H); beta-Cyclohexyl-Ala; beta-(1-Cyclopentenyl)-Ala; beta-Cyclopentyl-Ala; beta-Cyclopropyl-Ala; beta-Quinolyl-Ala; beta-(2-Thiazolyl)-Ala; beta-(Triazole-1-yl)-Ala; beta-(2-Pyridyl)-Ala; beta-(3-Pyridyl)-Ala; Amino-Phe; Fluoro-Phe; Cyclohexyl-Gly; tBu-Gly; beta-(3-benzothienyl)-Ala; beta-(2-thienyl)-Ala; 5-Methyl-Trp; and 4-Methyl-Trp. Proline residues can be substituted with homopro (L-pipecolic acid); hydroxy-Pro; 3,4-Dehydro-Pro; 4-fluoro-Pro; or alpha-methyl-Pro or an N(alpha)-C(alpha) cyclized amino acid analogues with the structure:

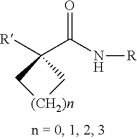

Alanine residues can be substituted with alpha-substituted or N-methylated amino acid such as alpha-amino isobutyric acid (aib), L/D-alpha-ethylalanine (L/D-isovaline), L/D-methylvaline, or L/D-alpha-methylleucine or a non-natural amino acid such as beta-fluoro-Ala. Alanine can also substituted with:

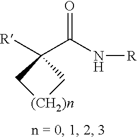

Glycine residues can be substituted with alpha-amino isobutyric acid (aib) or L/D-alpha-ethylalanine (L/D-isovaline).

Further examples of unnatural amino acids include: an unnatural analogue of tyrosine; an unnatural analogue of glutamine; an unnatural analogue of phenylalanine; an unnatural analogue of serine; an unnatural analogue of threonine; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; an amino acid that is amidated at a site that is not naturally amidated, a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid (e.g., an amino acid containing deuterium, tritium, ¹³C, ¹⁵N or ¹⁸O); a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α.-hydroxy containing acid; an amino thio acid containing amino acid; an α,α disubstituted amino acid; a β-amino acid; a cyclic amino acid other than proline; an O-methyl-L-tyrosine; an L-3-(2-naphthyl)alanine; a 3-methyl-phenylalanine; a p-acetyl-L-phenylalanine; an 0-4-allyl-L-tyrosine; a 4-propyl-L-tyrosine; a tri-O-acetyl-GlcNAcβ-serine; an L-Dopa; a fluorinated phenylalanine; an isopropyl-L-phenylalanine; a p-azido-L-phenylalanine; a p-acyl-L-phenylalanine; a p-benzoyl-L-phenylalanine; an L-phosphoserine; a phosphonoserine; a phosphonotyrosine; a p-iodo-phenylalanine; a 4-fluorophenylglycine; a p-bromophenylalanine; a p-amino-L-phenylalanine; an isopropyl-L-phenylalanine; L-3-(2-naphthyl)alanine; an amino-, isopropyl-, or O-allyl-containing phenylalanine analogue; a dopa, O-methyl-L-tyrosine; a glycosylated amino acid; a p-(propargyloxy)phenylalanine; dimethyl-Lysine; hydroxy-proline; mercaptopropionic acid; methyl-lysine; 3-nitro-tyrosine; norleucine; gyro-glutamic acid; Z (Carbobenzoxyl); ε-Acetyl-Lysine; β-alanine; aminobenzoyl derivative; aminobutyric acid (Abu); citrulline; aminohexanoic acid; aminoisobutyric acid; cyclohexylalanine; d-cyclohexylalanine; hydroxyproline; nitro-arginine; nitro-phenylalanine; nitro-tyrosine; norvaline; octahydroindole carboxylate; ornithine; penicillamine; tetrahydroisoquinoline; acetamidomethyl protected amino acids and pegylated amino acids. Further examples of unnatural amino acids and amino acid analogs can be found in U.S. 20030108885, U.S. 20030082575, US20060019347 (paragraphs 410-418) and the references cited therein. The polypeptides of the invention can include further modifications including those described in US20060019347, paragraph 589.

In some embodiments, an amino acid can be replaced by a naturally-occurring, non-essential amino acid, e.g., taurine.

Methods to manufacture polypeptides containing unnatural amino acids can be found in, for example, U.S. 20030108885, U.S. 20030082575, US20060019347, Deiters et al., J Am Chem. Soc. (2003) 125:11782-3, Chin et al., Science (2003) 301:964-7, and the references cited therein.

Peptides that include non-natural amino acids can also be prepared using the methods described in WO02086075.

The polypeptides can have one or more conventional polypeptide bonds replaced by an alternative bond. Such replacements can increase the stability of the polypeptide. For example, replacement of the polypeptide bond between a residue amino terminal to an aromatic residue (e.g. Tyr, Phe, Trp) with an alternative bond can reduce cleavage by carboxy peptidases and may increase half-life in the digestive tract. Bonds that can replace polypeptide bonds include: a retro-inverso bond (C(O)—NH instead of NH—C(O); a reduced amide bond (NH—CH2); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefine bond (CH═CH); a fluoro substituted trans-olefine bond (CF═CH); a ketomethylene bond (C(O)—CHR or CHR—C(O) wherein R is H or CH₃; and a fluoro-ketomethylene bond (C(O)—CFR or CFR—C(O) wherein R is H or F or CH₃.

The polypeptides can be modified using standard modifications. Modifications may occur at the amino (N—), carboxy (C—) terminus, internally or a combination of any of the preceeding. In one aspect described herein, there may be more than one type of modification on the polypeptide. Modifications include but are not limited to: acetylation, amidation, biotinylation, cinnamoylation, farnesylation, formylation, myristoylation, palmitoylation, phosphorylation (Ser, Tyr or Thr), stearoylation, succinylation, sulfurylation and cyclisation (via disulfide bridges or amide cyclisation), and modification by Cy3 or Cy5. The polypeptides described herein may also be modified by 2,4-dinitrophenyl (DNP), DNP-lysin, modification by 7-Amino-4-methyl-coumarin (AMC), flourescein, NBD (7-Nitrobenz-2-Oxa-1,3-Diazole), p-nitro-anilide, rhodamine B, EDANS (5-((2-aminoethyl)amino)naphthalene-1-sulfonic acid), dabcyl, dabsyl, dansyl, texas red, FMOC, and Tamra (Tetramethylrhodamine). The polypeptides described herein may also be conjugated to, for example, polyethylene glycol (PEG); alkyl groups (e.g., C1-C20 straight or branched alkyl groups); fatty acid radicals; combinations of PEG, alkyl groups and fatty acid radicals (see U.S. Pat. No. 6,309,633; Soltero et al., 2001 Innovations in Pharmaceutical Technology 106-110); BSA and KLH (Keyhole Limpet Hemocyanin). The addition of PEG and other polymers which can be used to modify polypeptides of the invention is described in US2006019347 section IX.

The polypeptides and agonists described herein can be chemically modified to increase therapeutic activity by synthetically adding sugar moieties (WO 88/02756; WO 89/09786; DE 3910667 A1, EP 0 374 089 A2; and U.S. Pat. No. 4,861,755), adding cationic anchors (EP0363589), lipid moieties (WO91/09837; U.S. Pat. No. 4,837,303) or the substituents described as compounds I, II, and III in U.S. Pat. No. 5,552,520.

Two or more polypeptides described herein can be joined by a peptide bond or by a polypeptide sequence (e.g., a sequence of 1 or more amino acids) or they can be joined by a linker. Many methods for protein cross-linking are known. Many cross-linking methods employ a thiol group and various methods can be used to introduce a thiol group, including the reduction of intrinsic disulfides, as well as the conversion of amine or carboxylic acid groups to thiol groups. In the present polypeptide it is often desirable to preserve disulfide bonds. Thus, it may be desirable to introduce a thiol group. Amines can be indirectly thiolated by reaction with succinimidyl 3-(2-pyridyldithio)propionate 4 (SPDP), followed by reduction of the 3-(2-pyridyldithio)propionyl conjugate with DTT or TCEP. Amines can be indirectly thiolated by reaction with succinimidyl acetylthioacetate 5 (SATA), followed by removal of the acetyl group. Thiols can be incorporated at carboxylic acid groups by an EDAC-mediated reaction with cystamine, followed by reduction of the disulfide. Polypeptides can also be crosslinked through amines. An amine on a first polypeptide can be thiolated and an amines on the second polypeptide can be converted to a thiol-reactive functional group such as a maleimide or iodoacetamide. Indirect crosslinking of the amines in a first polypeptide to the thiols in a second polypeptide is useful for forming a heteroconjugate. Thiol-reactive groups such as maleimides are typically introduced into the second polypeptide by modifying an amine with a heterobifunctional crosslinker containing both a succinimidyl ester and a maleimide. The maleimide-modified polypeptide is then reacted with the thiol-containing biomolecule to form a stable thioether crosslink.

The peptides described herein can be in the form or a salt, e.g., a pharmaceutically acceptable salt. A salt of a peptide can be formed by acid or base addition depending on the nature of the peptide. Suitable salts include base salts such as alkali metal salts, e.g., sodium, potassium, magnesium, and calcium salts. Other suitable base salts include substituted and unsubstituted ammonium salts (e.g., dimethyl-, diethyl- or diisopropylammonium salts, monoethanol-, diethanol- or diisopropylammonium salts, cyclohexyl- or dicyclohexylammonium salts and dibenzylethylenediammonium salts). Acid addition salts include, but are note limited to: hydrochloride, acetate and trifluoroacetate salts. Inorganic acids which can be used to form acid addition salts include, but are not limited to: sulfuric acid, nitric acid, hydrohalic acids such as hydrochloric acid or hydrobromic acid, phosphoric acids, and sulfamic acid. Organic acids which can be used to form acid addition salts include, but are not limited to: e.g. formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethane-sulfonic acid, ethanedisulfonic acid, 2-hydroxyethanesulfonic acid, benzenesulfonic acid, p-toluene-sulfonic acid, naphthalenemono- and -disulfonic acids, and laurylsulfuric acid.

The various polypeptides can be present with a counterion. Useful counterions include salts of: acetate, benzenesulfonate, benzoate, calcium edetate, camsylate, carbonate, citrate, edetate (EDTA), edisylate, embonate, esylate, fumarate, gluceptate, gluconate, glutamate, glycollylarsanilate, hexylresorcinate, iodide, bromide, chloride, hydroxynaphthoate, isethionate, lactate, lactobionate, estolate, maleate, malate, mandelate, mesylate, mucate, napsylate, nitrate, pantothenate, phosphate, salicylate, stearate, succinate, sulfate, tartarate, theoclate, acetamidobenzoate, adipate, alginate, aminosalicylate, anhydromethylenecitrate, ascorbate, aspartate, camphorate, caprate, caproate, caprylate, cinnamate, cyclamate, dichloroacetate, formate, gentisate, glucuronate, glycerophosphate, glycolate, hippurate, fluoride, malonate, napadisylate, nicotinate, oleate, orotate, oxalate, oxoglutarate, palmitate, pectinate, pectinate polymer, phenylethylbarbiturate, picrate, propionate, pidolate, sebacate, rhodanide, tosylate, and tannate.

For the treatment of gastrointestinal disorders, the polypeptide can be administered orally, by rectal suppository or parenterally.

In various embodiments of the treatment methods: the patient is suffering from a gastrointestinal disorder; the patient is suffering from a disorder selected from the group consisting of: gastrointestinal motility disorders, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, irritable bowel syndrome, post-operative ileus, ulcerative colitis, chronic constipation, and disorders and conditions associated with constipation (e.g. constipation associated with use of opiate pain killers, post-surgical constipation, and constipation associated with neuropathic disorders as well as other conditions and disorders are described herein); the patient is suffering from a gastrointestinal motility disorder, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, inflammatory bowel disease, irritable bowel syndrome, post-operative ileus, ulcerative colitis, chronic constipation, and disorders and conditions associated with constipation (e.g. constipation associated with use of opiate pain killers, post-surgical constipation, and constipation associated with neuropathic disorders as well as other conditions and disorders are described herein); and the composition is administered orally.

As noted, the polypeptides can be used to treat a patient suffering from constipation. Clinically accepted criteria that define constipation range from the frequency of bowel movements, the consistency of feces and the ease of bowel movement. One common definition of constipation is less than three bowel movements per week. Other definitions include abnormally hard stools or defecation that requires excessive straining (Schiller 2001 Aliment Pharmacol Ther 15:749-763). Constipation may be idiopathic (functional constipation or slow transit constipation) or secondary to other causes including neurologic, metabolic or endocrine disorders. These disorders include diabetes mellitus, hypothyroidism, hyperthyroidism, hypocalcaemia, Multiple sclerosis, Parkinson's disease, spinal cord lesions, Neurofibromatosis, autonomic neuropathy, Chagas disease, Hirschsprung disease and cystic fibrosis. Constipation may also be the result of surgery or due to the use of drugs such as analgesics (like opioids), antihypertensives, anticonvulsants, antidepressants, antispasmodics and antipsychotics. In various embodiments, the constipation is associated with use of a therapeutic agent; the constipation is associated with a neuropathic disorder; the constipation is post-surgical constipation; the constipation is associated with a gastrointestinal disorder; the constipation is idiopathic (functional constipation or slow transit constipation); the constipation is associated with neuropathic, metabolic or endocrine disorder (e.g., diabetes mellitus, hypothyroidism, hyperthyroidism, hypocalcaemia, Multiple Sclerosis, Parkinson's disease, spinal cord lesions, neurofibromatosis, autonomic neuropathy, Chagas disease, Hirschsprung disease or cystic fibrosis). Constipation may also be the result of surgery or due to the use of drugs such as analgesics (e.g., opioids), antihypertensives, anticonvulsants, antidepressants, antispasmodics and antipsychotics.

As noted, the polypeptides can be used to treat a patient suffering from a gastrointestinal disorder. In various embodiments, the patient is suffering from a gastrointestinal disorder; the patient is suffering from a disorder selected from the group consisting of: gastrointestinal motility disorders, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, irritable bowel syndrome, post-operative ileus, ulcerative colitis, chronic constipation, and disorders and conditions associated with constipation (e.g. constipation associated with use of opiate pain killers, post-surgical constipation, and constipation associated with neuropathic disorders as well as other conditions and disorders are described herein), obesity, congestive heart failure, or benign prostatic hyperplasia

Also featured is an isolated nucleic acid molecule comprising a nucleotide sequence encoding a polypeptide above.

As noted, the polypeptides can be used to treat a patient suffering from obesity. The polypeptide can be administered in combination with one or more agents for treatment of obesity, including, without limitation, the anti-obesity agents described herein. A polypeptide useful for treating obesity can be administered as a co-therapy with a polypeptide described herein either as a distinct molecule or as part of a fusion protein with a polypeptide described herein. Thus, for example, PYY₃₋₃₆ can be fused to the carboxy or amino terminus of a polypeptide described herein. Such a fusion protein can include a chymostrypsin or trypsin cleavage site that can permit cleavage to separate the two polypeptides.

As noted, the polypeptides can be used to treat a patient suffering from congestive heart failure. The polypeptide can be administered in combination with one or more agents for treatment of congestive heart failure, for example, a natriuretic polypeptide such as atrial natriuretic polypeptide, brain natriuretic polypeptide or C-type natriuretic polypeptide), nesiritide (Natrecor®), a diuretic, or an inhibitor of angiotensin converting enzyme.

As noted, the polypeptides can be used to treat congestive heart failure. The polypeptide can act in the kidney and adrenal gland to control natriuresis, kaliuresis, and diuresis thereby reducing the build-up of fluid associated with congestive heart failure (Lorenz et al. J Clin Invest 112:1138, 2003; Carrithers et al. Kidney Int 65:40, 2004). The polypeptide can be administered in combination with one or more agents for treatment of congestive heart failure, including but not limited to the agents useful for combitherapy described herein. For example, the polypeptide can be administered in combination with a natriuretic polypeptide such as atrial natriuretic polypeptide, brain natriuretic polypeptide or C-type natriuretic polypeptide), a diuretic, or an inhibitor of angiotensin converting enzyme.

The polypeptides can be used to treat, for example, constipation, decreased intestinal motility, slow digestion, slow stomach emptying. The polypeptides can be used to relieve one or more symptoms of IBS (bloating, pain, constipation), GERD (acid reflux into the esophagus), duodenogastric reflux, functional dyspepsia, or gastroparesis (nausea, vomiting, bloating, delayed gastric emptying) and other disorders described herein.

Clinically accepted criteria that define constipation range from the frequency of bowel movements, the consistency of feces and the ease of bowel movement. One common definition of constipation is less than three bowel movements per week. Other definitions include abnormally hard stools or defecation that requires excessive straining (Schiller 2001, Aliment Pharmacol Ther 15:749-763). Constipation may be idiopathic (functional constipation or slow transit constipation) or secondary to other causes including neurologic, metabolic or endocrine disorders. These disorders include diabetes mellitus, hypothyroidism, hyperthyroidism, hypocalcaemia, Multiple Sclerosis, Parkinson's disease, spinal cord lesions, Neurofibromatosis, autonomic neuropathy, Chagas disease, Hirschsprung's disease and cystic fibrosis. Constipation may also be the result of surgery or due to the use of drugs such as analgesics (like opioids), antihypertensives, anticonvulsants, antidepressants, antispasmodics and antipsychotics.

Also featured are isolated nucleic acid molecules comprising or consisting of a sequence encoding a polypeptide described herein. The invention also features vectors, e.g., expression vectors that include such nucleic acid molecules and can be used to express a polypeptide described herein in a cultured cell (e.g., a eukaryotic cell or a prokaryotic cell). The vector can further include one or more regulatory elements, e.g., a heterologous promoter or elements required for translation operably linked to the sequence encoding the polypeptide. In some cases the nucleic acid molecule will encode an amino acid sequence that includes the amino acid sequence of a polypeptide described herein. For example, the nucleic acid molecule can encode a preprotein or a preproprotein that can be processed to produce a polypeptide described herein. In cases where unnatural amino acids are present in the polypeptides described herein, selector codons can be utilized in the synthesis of such polypeptides similar to that described in US20060019347 (for example, paragraphs 398-408, 457-499, and 576-588) herein incorporated by reference.

A vector that includes a nucleotide sequence encoding a polypeptide described herein or a polypeptide or polypeptide comprising a polypeptide described herein may be either RNA or DNA, single- or double-stranded, prokaryotic, eukaryotic, or viral. Vectors can include transposons, viral vectors, episomes, (e.g., plasmids), chromosomes inserts, and artificial chromosomes (e.g. BACs or YACs). Suitable bacterial hosts for expression of the encode polypeptide or polypeptide include, but are not limited to, E. coli. Suitable eukaryotic hosts include yeast such as S. cerevisiae, other fungi, vertebrate cells, invertebrate cells (e.g., insect cells), plant cells, human cells, human tissue cells, and whole eukaryotic organisms. (e.g., a transgenic plant or a transgenic animal). Further, the vector nucleic acid can be used to generate a virus such as vaccinia or baculovirus (for example using the Bac-to-Bac® Baculovirus expression system (Invitrogen Life Technologies, Carlsbad, Calif.)).

As noted above the invention includes vectors and genetic constructs suitable for production of a polypeptide described herein or a polypeptide or polypeptide comprising such a polypeptide. Generally, the genetic construct also includes, in addition to the encoding nucleic acid molecule, elements that allow expression, such as a promoter and regulatory sequences. The expression vectors may contain transcriptional control sequences that control transcriptional initiation, such as promoter, enhancer, operator, and repressor sequences. A variety of transcriptional control sequences are well known to those in the art and may be functional in, but are not limited to, a bacterium, yeast, plant, or animal cell. The expression vector can also include a translation regulatory sequence (e.g., an untranslated 5′ sequence, an untranslated 3′ sequence, a poly A addition site, or an internal ribosome entry site), a splicing sequence or splicing regulatory sequence, and a transcription termination sequence. The vector can be capable of autonomous replication or it can integrate into host DNA.

The invention also includes isolated host cells harboring one of the forgoing nucleic acid molecules and methods for producing a polypeptide by culturing such a cell and recovering the polypeptide or a precursor of the polypeptide. Recovery of the polypeptide or precursor may refer to collecting the growth solution and need not involve additional steps of purification. Proteins of the present invention, however, can be purified using standard purification techniques, such as, but not limited to, affinity chromatography, thermaprecipitation, immunoaffinity chromatography, ammonium sulfate precipitation, ion exchange chromatography, filtration, electrophoresis and hydrophobic interaction chromatography.

The polypeptides can be purified. Purified polypeptides are polypeptides separated from other proteins, lipids, and nucleic acids or from the compounds from which is it synthesized. The polypeptide can constitute at least 10, 20, 50 70, 80 or 95% by dry weight of the purified preparation.

The invention features a method for preparing a polypeptide described herein by: chemically synthesizing the polypeptide and at least partially purifying the synthesized polypeptide.

The invention features a method for preparing a polypeptide described herein by: providing a host cells (e.g., a bacterial or mammalian or insect cell) harboring a nucleic acid molecule encoding the polypeptide, culturing the cell under conditions suitable for expression of the polypeptide, and at least partially purifying the polypeptide from the cell or the culture media in which the cell is cultured.

The invention features a method for treating inflammation, including inflammation of the gastrointestinal tract, e.g., inflammation associated with a gastrointestinal disorder or infection or some other disorder, the method comprising administering to a patient a pharmaceutical composition comprising a purified polypeptide described herein. In various embodiments the inflammation is associated with a gastrointestinal disorder, the inflammation is not associated with a gastrointestinal disorder.

The invention features a method for treating hypertension. The method comprises: administering to the patient a pharmaceutical composition comprising, consisting essentially of, or consisting of a polypeptide described herein and a pharmaceutically acceptable carrier. The composition can be administered in combination with another agent for treatment of hypertension, for example, a diuretic, an ACE inhibitor, an angiotensin receptor blocker, a beta-blocker, or a calcium channel blocker.

The invention features a method for treating secondary hyperglycemias in connection with pancreatic diseases (chronic pancreatitis, pancreasectomy, hemochromatosis) or endocrine diseases (acromegaly, Cushing's syndrome, pheochromocytoma or hyperthyreosis), drug-induced hyperglycemias (benzothiadiazine saluretics, diazoxide or glucocorticoids), pathologic glucose tolerance, hyperglycemias, dyslipoproteinemias, adiposity, hyperlipoproteinemias and/or hypotensions is described. The method comprises: administering to the patient a pharmaceutical composition comprising, consisting essentially of, or consisting of a polypeptide described herein and a pharmaceutically acceptable carrier.

Also described are methods for producing any of the forgoing polypeptides comprising providing a cell harboring a nucleic acid molecule encoding the polypeptide, culturing the cell under conditions in which the polypeptide is expressed, and isolating the expressed polypeptide.

Also described are methods for producing any of the forgoing polypeptides comprising chemically synthesizing the polypeptide and then purifying the synthesized polypeptide.

Also described are pharmaceutical compositions comprising the forgoing polypeptides.

Also described are nucleic acid molecules encoding any of the forgoing polypeptides, vectors (e.g., expression vectors) containing such nucleic acid molecules and host cells harboring the nucleic acid molecules or vectors.

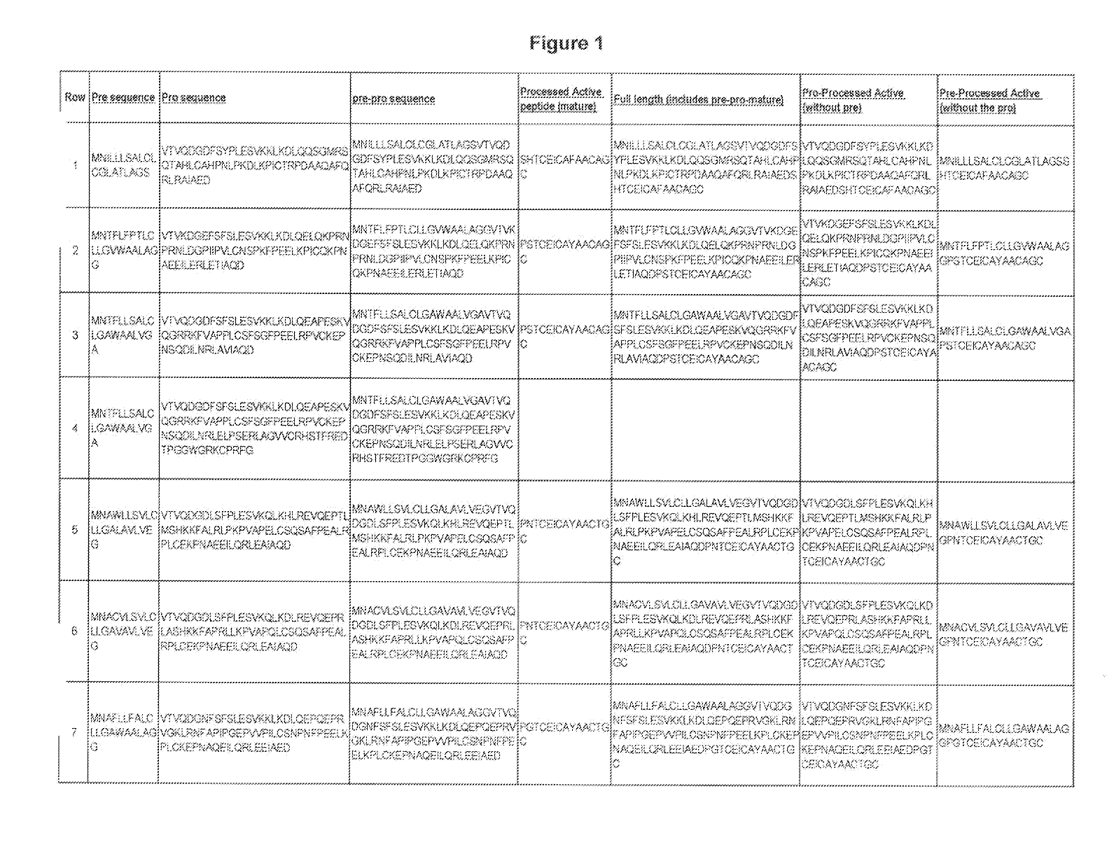

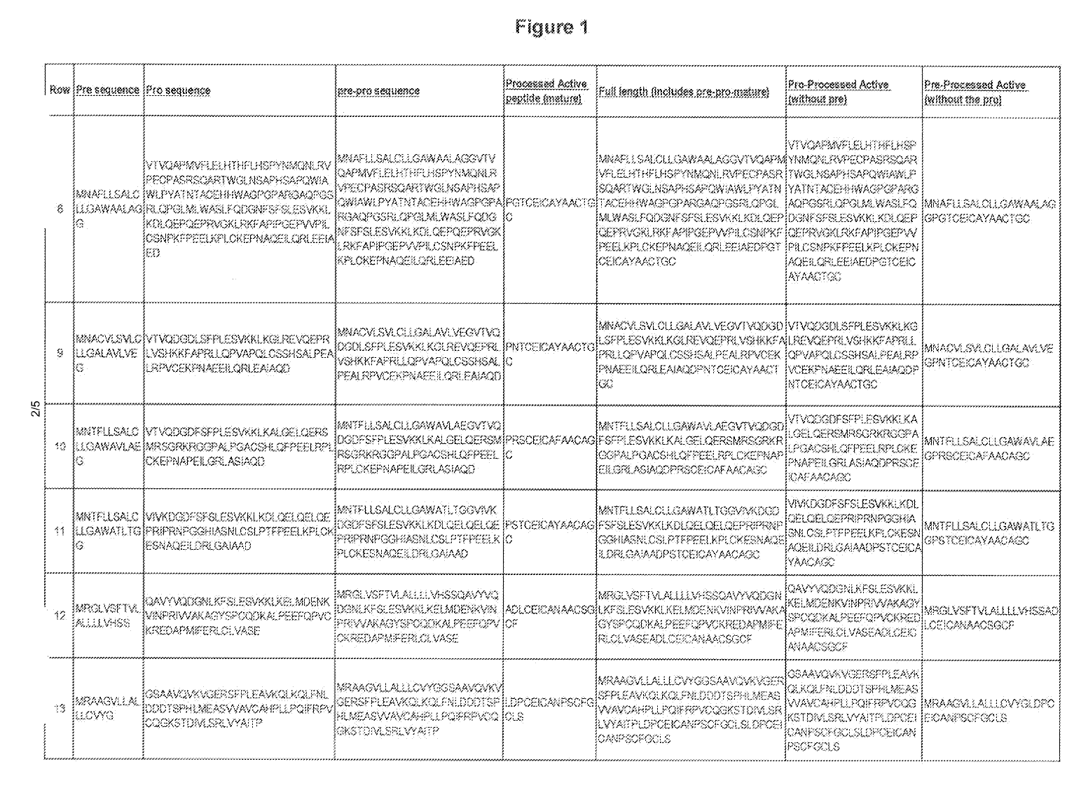

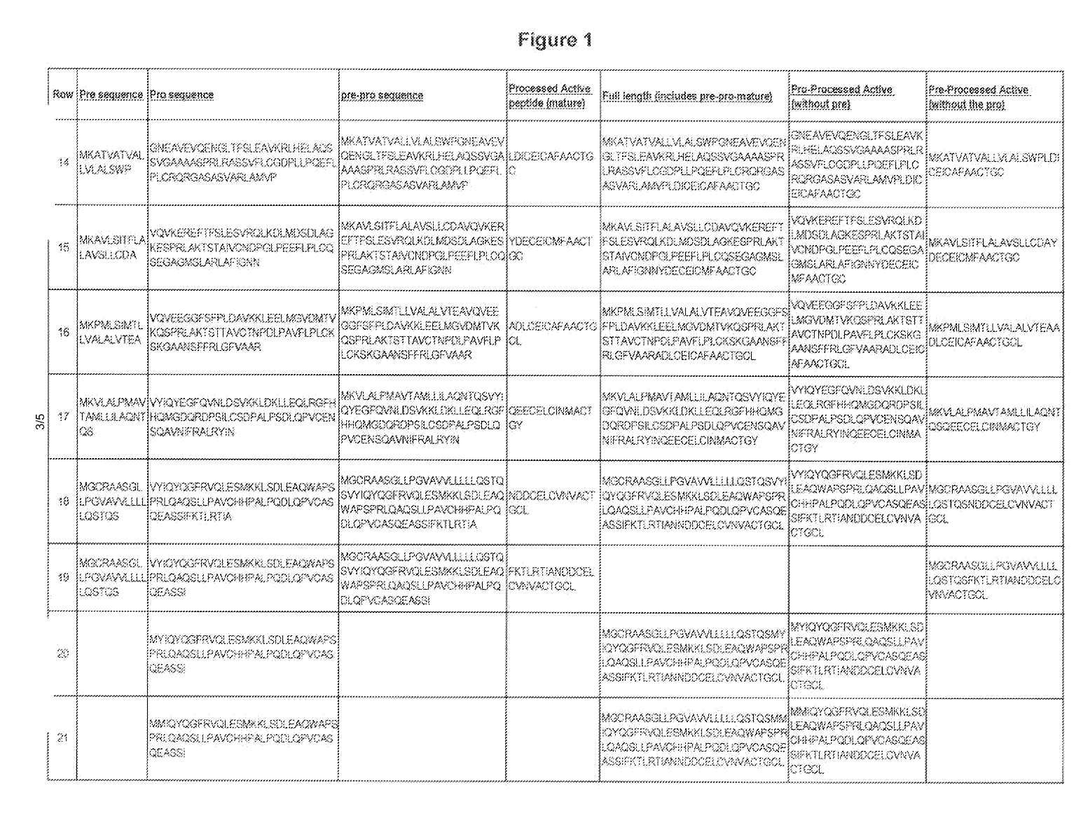

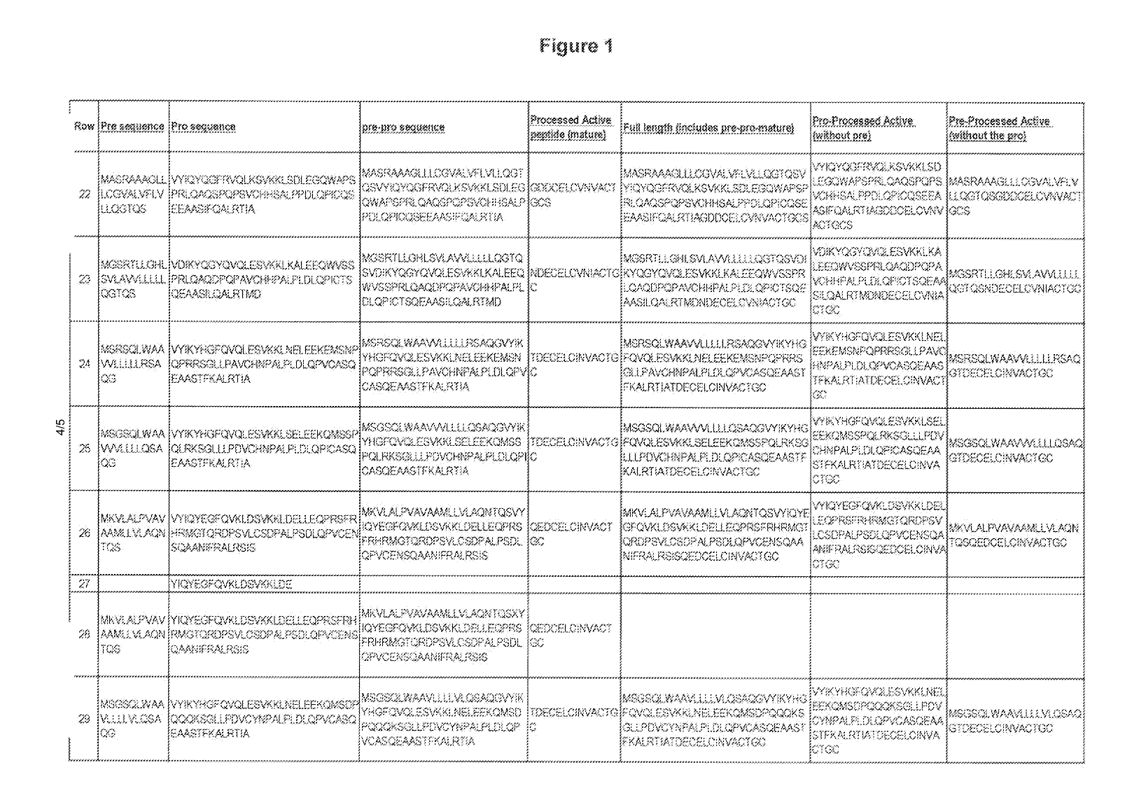

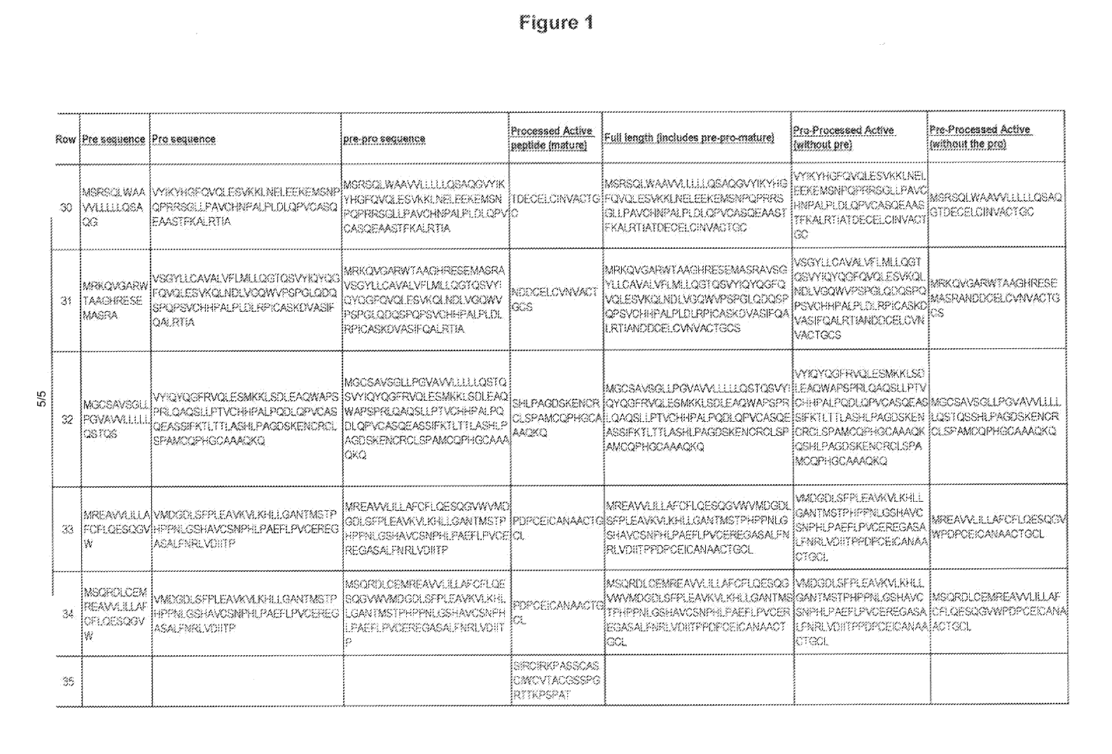

The various polypeptides described herein can include at least 10 contiguous amino acids of the pro sequence, a pre sequence or both a pre sequence and a pro sequence (a “prepro sequence”) of guanylin or uroguanylin from humans or other species. Thus, useful polypeptides can include a pre sequence, a pro sequence or a prepro sequence preceding (amino-terminal to) a GC-C receptor agonist polypeptide described herein. FIG. 1 depicts the pre sequence (SEQ ID NOs: ______-______), pro sequence (SEQ ID NOs: ______-______), prepro sequence (SEQ ID NOs: ______-______), and mature sequence for a number guanylin and uroguanylin polypeptides as well a various combinations thereof (e.g., a polypeptide consisting of a pre sequence and a mature polypeptide).

Among the useful GC-C receptor polypeptides that can modified by the addition of a polypeptide described herein are:

| | |
|---|---|
| PGTCEICASAACTGC | (SEQ ID NO: ) |
| PGTCEICATAACTGC | (SEQ ID NO: ) |
| PGTCEICANAACTGC | (SEQ ID NO: ) |
| PGTCEICAQAACTGC | (SEQ ID NO: ) |
| PGTCEICARAACTGC | (SEQ ID NO: ) |
| PGTCEICAEAACTGC | (SEQ ID NO: ) |
| PGTCEICADAACTGC | (SEQ ID NO: ) |
| PGTCEICAGAACTGC | (SEQ ID NO: ) |
| PGTCEICAAAACTGC | (SEQ ID NO: ) |
| PGTCEICAMAACTGC | (SEQ ID NO: ) |
| PGTCEICAIAACTGC | (SEQ ID NO: ) |
| PGTCEICALAACTGC | (SEQ ID NO: ) |
| PGTCEICAVAACTGC | (SEQ ID NO: ) |
| PGTCEICAHAACTGC | (SEQ ID NO: ) |
| PGTCEGICAYAACTGC | (SEQ ID NO: ) |
| PGTCEIGCAYAACTGC | (SEQ ID NO: ) |
| PGTCEICGAYAACTGC | (SEQ ID NO: ) |
| PGTCEICAGYAACTGC | (SEQ ID NO: ) |
| PGTCEICAYGAACTGC | (SEQ ID NO: ) |
| PGTCEICAYAGACTGC | (SEQ ID NO: ) |
| PGTCEICAYAAGCTGC | (SEQ ID NO: ) |
| PGTCEICAYAACGTGC | (SEQ ID NO: ) |
| PGTCEICAYAACTGGC | (SEQ ID NO: ) |
| PGTCAEICAYAACTGC | (SEQ ID NO: ) |
| PGTCEAICAYAACTGC | (SEQ ID NO: ) |
| PGTCEIACAYAACTGC | (SEQ ID NO: ) |
| PGTCEICAAYAACTGC | (SEQ ID NO: ) |
| PGTCEICAYAAACTGC | (SEQ ID NO: ) |
| PGTCEICAYAACATGC | (SEQ ID NO: ) |
| PGTCEICAYAACTAGC | (SEQ ID NO: ) |
| PGTCEICAYAACTGAC | (SEQ ID NO: ) |
| PGTCAEICAAYAACTGC | (SEQ ID NO: ) |
| PGTCEAICAAYAACTGC | (SEQ ID NO: ) |
| and | |
| PGTCEIACAAYAACTGC. | (SEQ ID NO: ) |

In some cases a polypeptide will be produced, e.g., recombinantly, with a pre sequence and/or a pro sequence. In certain cases the pre sequence and/or pro sequence is removed prior to administration of the polypeptide to a patient. In other cases the prepropolypeptide, propolypeptide or the prepolypeptide is administered to the patient. The pre sequence and/or the pro sequence may stabilize the polypeptide or an active isomer thereof, facilitate efficient folding of the polypeptide or protect the polypeptide from degradation in the patient's body. Thus, pre sequences, pro sequences and/or preprosequences that do not significantly interfere with GC-C receptor agonist activity can be beneficial. In some cases the pre sequence and/or the prosequence are removed by physiological processes after administration.

Pro sequences that include Cys residues that may form a disulfide bond. For example, many pro sequences include two Cys residues separated by 12 or 13 amino acids. These Cys residues may form a disulfide bond. These Cys residues can be replaced by homocysteine, penicillamine, 3-mercaptoproline (Kolodziej et al. 1996 Int J Pept Protein Res 48:274); β,β-dimethylcysteine (Hunt et al. 1993 Int J Pept Protein Res 42:249) or diaminopropionic acid (Smith et al. 1978 J Med Chem 21:117) to form alternative internal cross-links at the positions of the normal disulfide bonds.

### Metabolites of Asparagine

In some cases an asparagine (Asn) within a polypeptide can be metabolized to have a different structure and the GC receptor agonist containing such a metabolite of Asn may retain activity. Polypeptides where one or more Asn, e.g., one or more Asn within an embodiment of one or more of SEQ ID NO:X′, SEQ ID NO:X, SEQ ID NO:X1, SEQ ID NO:X2, SEQ ID NO:ZZ′, SEQ ID NO:ZZ, SEQ ID NO:ZZ1, and SEQ ID NO:ZZ2 described herein are replaced by a metabolite of Asn can be useful in the methods described herein and can be present in a pharmaceutical composition that optionally contains one or more additional active ingredients.

For example, one or more Asn within a polypeptide and the peptide bond carboxy terminal thereto having the structure:

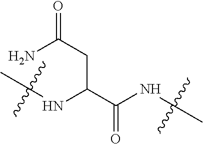

can replaced by a group having a structure selected from:

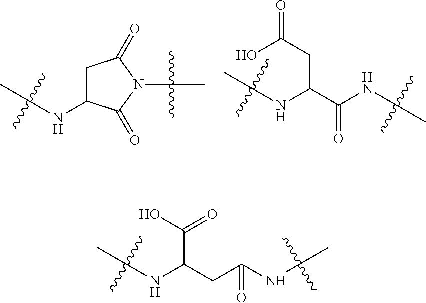

Thus, the Asn and the peptide bond carboxy terminal there to can be replaced by a cyclic imide:

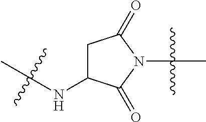

### Asp:

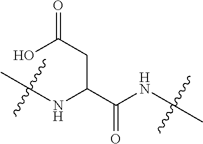

or isoAsn:

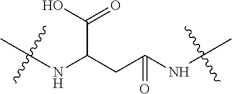

The Asp can be L-Asp or D-Asp. The isoAsn can be D-isoAsn or L-isoAsn.

Considering the asparagine only, one or more Asn having the structure:

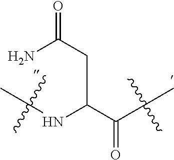

is can be optionally replaced by a group having a structure selected from (a), (b) and (c):

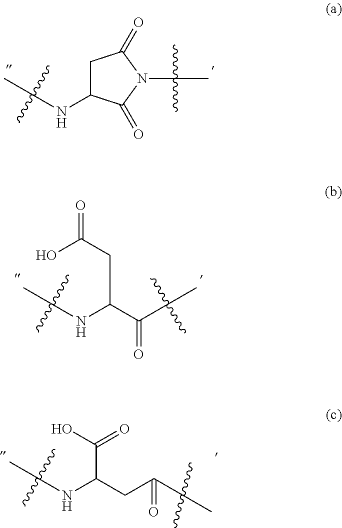

provided that an Asn at the carboxy terminus is not replaced by structure (a) or structure (c). When the Asn is at the carboxy terminus of the peptide, structure (a) cannot form. Since structure (c) is formed through structure (a), structure (c) cannot be formed when the Asn is at the carboxy terminus.

The formation of the various metabolites of Asp is depicted below.

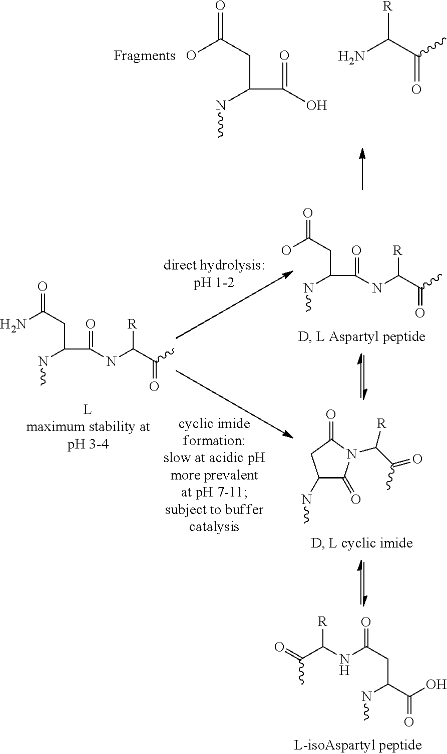

The details of one or more embodiments described herein are set forth in the accompanying description. All of the publications, patents and patent applications are hereby incorporated by reference.

### FIGURE

The FIGURE depicts the pre sequence (SEQ ID NOs: ______-______), pro sequence (SEQ ID NOs: ______-______), prepro sequence (SEQ ID NOs: ______-______), and mature sequence for a number guanylin and uroguanylin polypeptides as well a various combinations thereof (e.g., a polypeptide consisting of a pre sequence and a mature polypeptide).

### DETAILED DESCRIPTION

The polypeptides described herein bind to the guanylate cyclase (GC-C) receptor, a key regulator of fluid and electrolyte balance in the intestine and kidney. When stimulated, this receptor, which is located on the apical membrane of the intestinal epithelial surface, causes an increase in intestinal epithelial cyclic GMP (cGMP). This increase in cGMP is believed to cause a decrease in water and sodium absorption and an increase in chloride and potassium ion secretion, leading to changes in intestinal fluid and electrolyte transport and increased intestinal motility. The intestinal GC-C receptor possesses an extracellular ligand binding region, a transmembrane region, an intracellular protein kinase-like region and a cyclase catalytic domain. Proposed functions for the GC-C receptor are the fluid and electrolyte homeostasis, the regulation of epithelial cell proliferation and the induction of apoptosis (Shailhubhai 2002 Curr Opin Drug Dis Devel 5:261-268).

In addition to being expressed in gastrointestinal epithelial cells, GC-C is expressed in extra-intestinal tissues including kidney, lung, pancreas, pituitary, adrenal, developing liver, heart and male and female reproductive tissues (reviewed in Vaandrager 2002 Mol Cell Biochem 230:73-83). This suggests that the GC-C receptor agonists can be used in the treatment of disorders outside the GI tract, for example, congestive heart failure and benign prostatic hyperplasia.

Ghrelin, a polypeptide hormone secreted by the stomach, is a key regulator of appetite in humans. Ghrelin expression levels are regulated by fasting and by gastric emptying. (Kim et al., 2003, Neuroreprt 14:1317-20; Gualillo et al., 2003, FEBS Letts 552: 105-9). Thus, by increasing gastrointestinal motility, GC-C receptor agonists may also be used to regulate obesity.

In humans, the GC-C receptor is activated by guanylin (Gn) (U.S. Pat. No. 5,96,097), uroguanylin (Ugn) (U.S. Pat. No. 5,140,102) and lymphoguanylin (Forte et al. 1999 Endocrinology 140:1800-1806).

Many gastrointestinal disorders, including IBS, are associated with abdominal or visceral pain. Certain of the polypeptides described herein include the analgesic or anti-nociceptive tags such as the carboxy-terminal sequence AspPhe immediately following a Trp, Tyr or Phe (i.e., a chymotrypsin cleavage site) or following Lys or Arg (a trypsin cleavage site). Chymotrypsin in the intestinal tract will cleave such polypeptides immediately carboxy terminal to the Trp, Phe or Tyr residue, releasing the dipeptide, AspPhe. This dipeptide has been shown to have analgesic activity is animal models (Abdikkahi et al. 2001 Fundam Clin Pharmacol 15:117-23; Nikfar et al 1997, 29:583-6; Edmundson et al 1998 Clin Pharmacol Ther 63:580-93). In this manner such polypeptides can treat both pain and inflammation. Other analgesic polypeptides can be present at the carboxy terminus of the polypeptide (following a cleavage site) including: endomorphin-1, endomorphin-2, nocistatin, dalargin, lupron, ziconotide, and substance P. As described in greater detail below, various analgesic polypeptides and compounds can be covalently linked to or used in combination therapy with the therapeutic polypeptides described herein.

In the human body an inactive form of chymotrypsin, chymotrypsinogen is produced in the pancreas. When this inactive enzyme reaches the small intestine it is converted to active chymotrypsin by the excision of two di-peptides. Active chymotrypsin will cleave polypeptides at the polypeptide bond on the carboxy-terminal side of Trp, Tyr or Phe. The presence of active chymotrypsin in the intestinal tract will lead to cleavage of certain of the polypeptides described herein having an appropriately positioned chymotrypsin cleavage site. Certain of the polypeptides described herein include a Trp, Tyr or Phe immediately followed by a carboxy-terminal analgesic polypeptide. It is expected that chymotrypsin cleavage will release the analgesic polypeptide from polypeptide described herein having an appropriately positioned chymotrypsin cleavage site as the polypeptide passes through the intestinal tract.

Trypsinogen, like chymotrypsin, is a serine protease that is produced in the pancreas and is present in the digestive tract. The active form, trypsin, will cleave polypeptides having a Lys or Arg. The presence of active trypsin in the intestinal tract will lead to cleavage of certain of the polypeptides described herein having an appropriately positioned trypsin cleavage site. It is expected that chymotrypsin cleavage will release the analgesic polypeptide from polypeptide described herein having an appropriately positioned trypsin cleavage site as the polypeptide passes through the intestinal tract.

In some cases, the polypeptides described herein are produced as a prepro protein. The prepro protein can include any suitable prepro sequence, including but not limited to, for example, mnafllsalc llgawaalag gvtvqdgnfs fslesvkklk dlqepqepry gklrnfapip gepvvpilcs npnfpeelkp lckepnaqei lqrleeiaed (SEQ ID NO:), mgcraasgll pgvavvllll lqstqsvyiq yqgfrvqles mkklsdleaq wapsprlqaq sllpavchhp alpqdlqpvc asqeassifk tlrtia (SEQ ID NO:), lrtia (SEQ ID NO.), mnawllsvlc llgalavlve gvtvqdgdls fplesvkqlk hlrevqeptl mshkkfalrl pkpvapelcs qsafpealrp lcekpnaeei lqrleaiaqd (SEQ ID NO:), and msgsqlwaav llllvlqsaq gvyikyhgfq vqlesvkkln eleekqmsdp qqqksgllpd vcynpalpld lqpvcasqea astfkalrti a (SEQ ID NO:) or a bacterial leader sequence such as: mkksilfiflsvlsfspfaqdakpvesskekitleskkcniakksnksgpesmn. Where the polypeptide is produced by a bacterial cell, e.g., E. coli, the forgoing leader sequence will be cleaved and the mature polypeptide will be efficiently secreted from the bacterial cell. U.S. Pat. No. 5,395,490 describes vectors, expression systems and methods for the efficient production of certain mature polypeptides having disulfide bonds in bacterial cells and methods for achieving efficient secretion of such mature polypeptides. The vectors, expression systems and methods described in U.S. Pat. No. 5,395,490 can be used to produce the polypeptides of the present invention.

### Variant Polypeptides

The invention includes variant polypeptides that can include one, two, three, four, or five or more (e.g., 6, 7, 8, 9, 10, 11, 12, 13, 14, or 15) amino acid substitutions compared to any of the polypeptides described above. The substitution(s) can be conservative or non-conservative. The naturally-occurring amino acids can be substituted by D-isomers of any amino acid, non-natural amino acids, natural and non-natural amino acid analogs, and other groups. A conservative amino acid substitution results in the alteration of an amino acid for a similar acting amino acid, or amino acid of like charge, polarity, or hydrophobicity. At some positions, even conservative amino acid substitutions can reduce the activity of the polypeptide. A conservative substitution can substitute a naturally-occurring amino acid for a non-naturally-occurring amino acid. Among the naturally occurring amino acid substitutions generally considered conservative are:

| For Amino Acid | Code | Replace with any of |
|---|---|---|
| Alanine | Ala | Gly, Cys, Ser |
| Arginine | Arg | Lys, His |
| Asparagine | Asn | Asp, Glu, Gln, |
| Aspartic Acid | Asp | Asn, Glu, Gln |
| Cysteine | Cys | Met, Thr, Ser |
| Glutamine | Gln | Asn, Glu, Asp |
| Glutamic Acid | Glu | Asp, Asn, Gln |
| Glycine | Gly | Ala |
| Histidine | His | Lys, Arg |
| Isoleucine | Ile | Val, Leu, Met |
| Leucine | Leu | Val, Ile, Met |
| Lysine | Lys | Arg, His |
| Methionine | Met | Ile, Leu, Val |
| Phenylalanine | Phe | Tyr, His, Trp |
| Proline | Pro | |
| Serine | Ser | Thr, Cys, Ala |
| Threonine | Thr | Ser, Met, Val |
| Tryptophan | Trp | Phe, Tyr |
| Tyrosine | Tyr | Phe, His |
| Valine | Val | Leu, Ile, Met |

In some circumstances it can be desirable to treat patients with a variant polypeptide that binds to and activates intestinal GC-C receptor, but is less active or more active than the non-variant form of the polypeptide. Reduced activity can arise from reduced affinity for the receptor or a reduced ability to activate the receptor once bound or reduced stability of the polypeptide. Increased activity can arise from increased affinity for the receptor or an increased ability to activate the receptor once bound or increased stability of the polypeptide.

In some polypeptides one or both members of one or both pairs of Cys residues which normally form a disulfide bond can be replaced by homocysteine, penicillamine, 3-mercaptoproline (Kolodziej et al. 1996 Int J Pept Protein Res 48:274); β,β dimethylcysteine (Hunt et al. 1993 Int Pept Protein Res 42:249) or diaminopropionic acid (Smith et al. 1978 J Med Chem 21:117) to form alternative internal cross-links at the positions of the normal disulfide bonds.

### Production of Polypeptides

Useful polypeptides can be produced either in bacteria including, without limitation, E. coli, or in other existing systems for polypeptide or protein production (e.g., Bacillus subtilis, baculovirus expression systems using Drosophila Sf9 cells, yeast or filamentous fungal expression systems, mammalian cell expression systems), or they can be chemically synthesized.

If the polypeptide or variant polypeptide is to be produced in bacteria, e.g., E. coli, the nucleic acid molecule encoding the polypeptide may also encode a leader sequence that permits the secretion of the mature polypeptide from the cell. Thus, the sequence encoding the polypeptide can include the pre sequence and the pro sequence of, for example, a naturally-occurring bacterial ST polypeptide. The secreted, mature polypeptide can be purified from the culture medium.

The sequence encoding a polypeptide described herein is can be inserted into a vector capable of delivering and maintaining the nucleic acid molecule in a bacterial cell. The DNA molecule may be inserted into an autonomously replicating vector (suitable vectors include, for example, pGEM3Z and pcDNA3, and derivatives thereof). The vector nucleic acid may be a bacterial or bacteriophage DNA such as bacteriophage lambda or M13 and derivatives thereof. Construction of a vector containing a nucleic acid described herein can be followed by transformation of a host cell such as a bacterium. Suitable bacterial hosts include but are not limited to, E. coli, B subtilis, Pseudomonas, Salmonella. The genetic construct also includes, in addition to the encoding nucleic acid molecule, elements that allow expression, such as a promoter and regulatory sequences. The expression vectors may contain transcriptional control sequences that control transcriptional initiation, such as promoter, enhancer, operator, and repressor sequences. A variety of transcriptional control sequences are well known to those in the art. The expression vector can also include a translation regulatory sequence (e.g., an untranslated 5′ sequence, an untranslated 3′ sequence, or an internal ribosome entry site). The vector can be capable of autonomous replication or it can integrate into host DNA to ensure stability during polypeptide production.

The protein coding sequence that includes a polypeptide described herein can also be fused to a nucleic acid encoding a polypeptide affinity tag, e.g., glutathione S-transferase (GST), maltose E binding protein, protein A, FLAG tag, hexa-histidine, myc tag or the influenza HA tag, in order to facilitate purification. The affinity tag or reporter fusion joins the reading frame of the polypeptide of interest to the reading frame of the gene encoding the affinity tag such that a translational fusion is generated. Expression of the fusion gene results in translation of a single polypeptide that includes both the polypeptide of interest and the affinity tag. In some instances where affinity tags are utilized, DNA sequence encoding a protease recognition site will be fused between the reading frames for the affinity tag and the polypeptide of interest.

Genetic constructs and methods suitable for production of immature and mature forms of the polypeptides and variants described herein in protein expression systems other than bacteria, and well known to those skilled in the art, can also be used to produce polypeptides in a biological system.

Mature polypeptides and variants thereof can be synthesized by the solid-phase method using an automated polypeptide synthesizer. For example, the polypeptide can be synthesized on Cyc(4-CH₂ Bxl)-OCH₂-4-(oxymethyl)-phenylacetamidomethyl resin using a double coupling program. Protecting groups must be used appropriately to create the correct disulfide bond pattern. For example, the following protecting groups can be used: t-butyloxycarbonyl (alpha-amino groups); acetamidomethyl (thiol groups of Cys residues B and E); 4-methylbenzyl (thiol groups of Cys residues C and F); benzyl (y-carboxyl of glutamic acid and the hydroxyl group of threonine, if present); and bromobenzyl (phenolic group of tyrosine, if present). Coupling is effected with symmetrical anhydride of t-butoxylcarbonylamino acids or hydroxybenzotriazole ester (for asparagine or glutamine residues), and the polypeptide is deprotected and cleaved from the solid support in hydrogen fluoride, dimethyl sulfide, anisole, and p-thiocresol using 8/1/1/0.5 ratio (v/v/v/w) at 0° C. for 60 min. After removal of hydrogen fluoride and dimethyl sulfide by reduced pressure and anisole and p-thiocresol by extraction with ethyl ether and ethyl acetate sequentially, crude polypeptides are extracted with a mixture of 0.5M sodium phosphate buffer, pH 8.0 and N,N-dimethylformamide using 1/1 ratio, v/v. Disulfide bonds between Cys residues can be formed using dimethyl sulfoxide (Tam et al. (1991) J. Am. Chem. Soc. 113:6657-62). The resulting polypeptide is the purified by reverse-phase chromatography. In some cases it may be necessary to first dissolve the polypeptide in 50% acetic acid in water before disulfide bond formation. Saturated iodine solution in glacial acetic acid is added (1 ml iodine solution per 100 ml solution). After incubation at room temperature for 2 days in closed glass container, the solution is diluted five-fold with deionized water and extracted with ethyl ether four times for removal of unreacted iodine. After removal of the residual amount of ethyl ether by rotary evaporation the solution of crude product is lyophilized and purified by successive reverse-phase chromatography.

Peptides can also be synthesized by many other methods including solid phase synthesis using traditional FMOC protection (i.e., coupling with DCC-HOBt and deprotection with piperidine in DMF). Cys thiol groups can be trityl protected. Treatment with TFA can be used for final deprotection of the polypeptide and release of the polypeptide from the solid-state resin. In many cases air oxidation is sufficient to achieve proper disulfide bond formation.

### Intestinal GC-C Receptor Binding and Activity Assays

The ability of polypeptides, variant polypeptides and other compounds to bind to and activate the intestinal GC-C receptor can be tested using the T84 human colon carcinoma cell line (American Type Culture Collection (Bethesda, Md.).

Briefly, cells are grown to confluency in 24-well culture plates with a 1:1 mixture of Ham's F12 medium and Dulbecco's modified Eagle's medium (DMEM), supplemented with 5% fetal calf serum and are used at between passages 54 and 60.

Monolayers of T84 cells in 24-well plates are washed twice with 1 ml/well DMEM, then incubated at 37° C. for 10 min with 0.45 ml DMEM containing 1 mM isobutylmethylxanthine (IBMX), a cyclic nucleotide phosphodiesterase inhibitor. Test polypeptides (50 μl) are then added and incubated for 30 minutes at 37° C. The media is aspirated and the reaction is terminated by the addition of ice cold 0.5 ml of 0.1N HCl. The samples are held on ice for 20 minutes and then evaporated to dryness using a heat gun or vacuum centrifugation. The dried samples are resuspended in 0.5 ml of phosphate buffer provided in the Cayman Chemical Cyclic GMP EIA kit (Cayman Chemical, Ann Arbor, Mich.). Cyclic GMP is measured by EIA according to procedures outlined in the Cayman Chemical Cyclic GMP EIA kit.

For the binding assay, T84 cell monolayers in 24-well plates are washed twice with 1 ml of binding buffer (DMEM containing 0.05% bovine serum albumin and 25 mM HEPES, pH 7.2), then incubated for 30 min at 37° C. in the presence of mature radioactively labeled E. coli ST polypeptide and the test material at various concentrations. The cells are then washed 4 times with 1 ml of DMEM and solubilized with 0.5 ml/well 1N NaOH. The level of radioactivity in the solubilized material is then determined using standard methods.

### Murine Gastrointestinal Transit (GIT) Assay

In order to determine whether a test compound or a polypeptide, increases the rate of gastrointestinal transit, the test compound can be tested in the murine gastrointestinal transit (GIT) assay (Moon et al. Infection and Immunity 25:127, 1979). In this assay, charcoal, which can be readily visualized in the gastrointestinal tract is administered to mice after the administration of a test compound. The distance traveled by the charcoal is measured and expressed as a percentage of the total length of the colon.

Mice are fasted with free access to water for 12 to 16 hours before the treatment with polypeptide or control buffer. The polypeptides are orally administered at 1 μg/kg-1 mg/kg of polypeptide in buffer (20 mM Tris pH 7.5) seven minutes before being given an oral dose of 5% Activated Carbon (Aldrich 242276-250G). Control mice are administered buffer only before being given a dose of Activated Carbon. After 15 minutes, the mice are sacrificed and their intestines from the stomach to the cecum are dissected. The total length of the intestine as well as the distance traveled from the stomach to the charcoal front is measured for each animal and the results are expressed as the percent of the total length of the intestine traveled by the charcoal front. Results are reported as the average of 10 mice±standard deviation. A comparison of the distance traveled by the charcoal between the mice treated with polypeptide versus the mice treated with vehicle alone is performed using a Student's t test and a statistically significant difference is considered for P<0.05. Positive controls for this assay may include commercially available wild-type ST polypeptide (Sigma-Aldrich, St Louis, Mo.) and Zelnorm®, a drug approved for IBS that is an agonist for the serotonin receptor 5HT4.

Similar assays can be performed in other rodents, for example, rats. In addition, GIT assays can be performed and compared in wild-type versus rodents lacking the guanylate cyclase C receptor (GC-C KO), for example, using the GC-C KO mice described in Mann et al 1997 Biochem and Biophysical Research Communications 239:463.

### Suckling Mouse Model of Intestinal Secretion (SuMi Assay)

The polypeptides described herein can be tested for their ability to increase intestinal secretion using a suckling mouse model of intestinal secretion. In this model a test compound is administered to suckling mice that are between seven and nine days old. After the mice are sacrificed, the gastrointestinal tract from the stomach to the cecum is dissected (“guts”). The remains (“carcass”) as well as the guts are weighed and the ratio of guts to carcass weight is calculated. If the ratio is above 0.09, one can conclude that the test compound increases intestinal secretion. Controls for this assay may include wild-type ST polypeptide and Zelnorm®.

### Phenylbenzoquinone-Induced Writhing Model

The PBQ-induced writhing model can be used to assess pain control activity of the polypeptides and GC-C receptor agonists described herein. This model is described by Siegmund et al. (1957 Proc. Soc. Exp. Bio. Med. 95:729-731). Briefly, one hour after oral dosing with a test compound, e.g., a polypeptide, morphine or vehicle, 0.02% phenylbenzoquinone (PBQ) solution (12.5 mL/kg) is injected by intraperitoneal route into the mouse. The number of stretches and writhings are recorded from the 5^{th} to the 10^{th} minute after PBQ injection, and can also be counted between the 35^{th} and 40^{th} minute and between the 60^{th} and 65^{th} minute to provide a kinetic assessment. The results are expressed as the number of stretches and writhings (mean±SEM) and the percentage of variation of the nociceptive threshold calculated from the mean value of the vehicle-treated group. The statistical significance of any differences between the treated groups and the control group is determined by a Dunnett's test using the residual variance after a one-way analysis of variance (P<0.05) using SigmaStat Software.

### Colonic Hyperalgesia Animal Models

Hypersensitivity to colorectal distension is a common feature in patients with IBS and may be responsible for the major symptom of pain. Both inflammatory and non-inflammatory animal models of visceral hyperalgesia to distension have been developed to investigate the effect of compounds on visceral pain in IBS.

I. Trinitrobenzenesulphonic Acid (TNBS)-Induced Rectal Allodynia Model

Male Wistar rats (220-250 g) are premedicated with 0.5 mg/kg of acepromazine injected intraperitoneally (IP) and anesthetized by intramuscular administration of 100 mg/kg of ketamine. Pairs of nichrome wire electrodes (60 cm in length and 80 μm in diameter) are implanted in the striated muscle of the abdomen, 2 cm laterally from the white line. The free ends of electrodes are exteriorized on the back of the neck and protected by a plastic tube attached to the skin. Electromyographic (EMG) recordings are started 5 days after surgery. Electrical activity of abdominal striated muscle is recorded with an electroencephalograph machine (Mini VIII, Alvar, Paris, France) using a short time constant (0.03 sec.) to remove low-frequency signals (<3 Hz).

Ten days post surgical implantation, trinitrobenzenesulphonic acid (TNBS) is administered to induce rectal inflammation. TNBS (80 mg kg⁻¹ in 0.3 ml 50% ethanol) is administered intrarectally through a silicone rubber catheter introduced at 3 cm from the anus under light diethyl-ether anesthesia, as described (Morteau et al. 1994 Dig Dis Sci 39:1239). Following TNBS administration, rats are placed in plastic tunnels where they are severely limited in mobility for several days before colorectal distension (CRD). Experimental compound is administered one hour before CRD which is performed by insertion into the rectum, at 1 cm of the anus, a 4 cm long balloon made from a latex condom (Gue et al, 1997 Neurogastroenterol. Motil. 9:271). The balloon is fixed on a rigid catheter taken from an embolectomy probe (Fogarty). The catheter attached balloon is fixed at the base of the tail. The balloon, connected to a barostat is inflated progressively by step of 15 mmHg, from 0 to 60 mmHg, each step of inflation lasting 5 min. Evaluation of rectal sensitivity, as measured by EMG, is performed before (1-2 days) and 3 days following rectal instillation of TNBS.

The number of spike bursts that corresponds to abdominal contractions is determined per 5 min periods. Statistical analysis of the number of abdominal contractions and evaluation of the dose-effects relationships is performed by a one way analysis of variance (ANOVA) followed by a post-hoc (Student or Dunnett tests) and regression analysis for ED50 if appropriate.

II. Stress-Induced Hyperalgesia Model

Male Wistar Rats (200-250 g) are surgically implanted with nichrome wire electrodes as in the TNBS model. Ten days post surgical implantation, partial restraint stress (PRS), is performed as described by Williams et al. for two hours (Williams et al. 1988 Gastroenterology 64:611). Briefly, under light anaesthesia with ethyl-ether, the foreshoulders, upper forelimbs and thoracic trunk are wrapped in a confining harness of paper tape to restrict, but not prevent body movements. Control sham-stress animals are anaesthetized but not wrapped. Thirty minutes before the end of the PRS session, the animals are administered test-compound or vehicle. Thirty minutes to one hour after PRS completion, the CRD distension procedure is performed as described above for the TNBS model with barostat at pressures of 15, 30, 45 and 60 mm Hg. Statistical analysis on the number of bursts is determined and analyzed as in the TNBS model above.

III. Water Avoidance Stress-Induced Hyperalgesia Model

The effect of polypeptides/GC-C agonists described herein on basal visceral nociception in a model of water avoidance stress-induced visceral hyperalgesia in adult male Wistar rats can be tested. The stress involves confining rats to a platform surrounded by water for a period of 1 hour and then measuring their visceromotor response to colonic distension using electromyography (EMG).

At least 7 days prior to stress measurements, animals are deeply anesthetized with pentobarbital sodium (45 mg/kg) and equipped with electrodes implanted into the external oblique musculature, just superior to the inguinal ligament. Electrode leads are then tunneled subcutaneously and externalized laterally for future access. Following surgery, rats are housed in pairs and allowed to recover for at least 7 days. On the day of the experiment, animals are lightly anesthetized with halothane, and a lubricated latex balloon (6 cm) is inserted intra-anally into the descending colon. Animals are allowed to recover for 30 minutes, and colorectal distension (CRD) is initiated. The CRD procedure consists of graded intensities of phasic CRD (10, 20, 40, 60 mmHg; 20 s duration; 4 min inter-stimulus interval). Visceromotor response (VMR) to CRD is quantified by measuring EMG activity. To determine the effects of polypeptides/GC-C agonists described herein on basal visceral nociception, a baseline CRD is recorded. Animals are allowed 1 hour recovery and then the polypeptide/GC-C agonist described herein or vehicle is orally administered. At 1 hour following administration of polypeptide/GC-C agonist described herein or vehicle CRD is repeated.

To determine the effect of polypeptides/GC-C agonists described herein in a model of water avoidance stress-induced visceral hyperalgesia, a baseline CRD is recorded and then the animals were subjected to 1 hour of water avoidance stress. For water avoidance stress, the test apparatus consists of a Plexiglas tank with a block affixed to the center of the floor. The tank is filled with fresh room temperature water (25° C.) to within 1 cm of the top of the block. The animals are placed on the block for a period of 1 hour. The sham water avoidance stress consists in placing the rats on the same platform in a waterless container. A second CRD is performed at 24 hours post water avoidance stress. Following the second CRD, animals are allowed 1 hour recovery and then the polypeptide/GC-C agonist described herein or vehicle is orally administered. At 1 hour following administration of polypeptide/GC-C agonist described herein or vehicle CRD is repeated. Mean+/−SEM is be determined and compared in the presence and absence of water avoidance stress conditions.

Kd Determination and Binding Assays

To determine the affinity of polypeptides/GC-C agonists described herein for GC-C receptors found in rat intestinal mucosa, a competition binding assay is performed using rat intestinal epithelial cells. Epithelial cells from the small intestine of rats are obtained as described by Kessler et al. (J. Biol. Chem. 245: 5281-5288 (1970)). Briefly, animals are sacrificed and their abdominal cavities exposed. The small intestine is rinsed with 300 ml ice cold saline or PBS. 10 cm of the small intestine measured at 10 cm from the pylorus is removed and cut into 1 inch segments. Intestinal mucosa is extruded from the intestine by gentle pressure between a piece of parafilm and a P-1000 pipette tip. Intestinal epithelial cells are placed in 2 ml PBS and pipetted up and down with a 5 ml pipette to make a suspension of cells. Protein concentration in the suspension is measured using the Bradford method (Anal. Biochem. 72: 248-254 (1976)).

A competition binding assay is performed based on the method of Giannella et al. (Am. J. Physiol. 245: G492-G498) between [¹²⁵I] labeled control polypeptide (e.g. wild-type guanylin, uroguanylin or ST polypeptide) and a polypeptide/GC-C agonist described herein. The assay mixture contains: 0.5 ml of DME with 20 mM HEPES-KOH pH 7.0, 0.9 mg of the cell suspension listed above, 21.4 fmol [¹²⁵I]-labeled control polypeptide (42.8 pM), and different concentrations of competitor polypeptide/GC-C agonist described herein (0.01 to 1000 nM). The mixture is incubated at room temperature for 1 hour, and the reaction stopped by applying the mixture to GF/B glass-fiber filters (Whatman). The filters are washed with 5 ml ice-cold PBS and radioactivity is measured. Kd is determined. % B/Bo is the percentage of the ratio of radioactivity trapped in each sample (B) compared to the radioactivity retained in a control sample with no cold competitor (Bo).

Similar competition binding assays are performed in intestinal epithelial cells from wild-type and guanylate cyclase C knockout (GC-C KO; Mann et al. 1997 Biochem and Biophysical Research Communications 239:463) mice. Mouse intestinal epithelial cells are prepared identical to that above as for rat intestinal epithelial cells except the cells are homogenized with an Omni homogenizer for 20 seconds on the maximum setting to make a suspension of cells. A competition binding assay is performed identical to that described above between ¹²⁵I labeled polypeptide/GC-C agonist described herein and unlabeled polypeptide/GC-C agonist described herein (competitor).

### Pharmacokinetic Property Determination of Polypeptides/GC-C Agonists Described Herein

Serum samples are extracted from the whole blood of exposed (mice dosed orally or intravenously with polypeptide(s) described herein) and control mice, then injected directly (10 mL) onto an in-line solid phase extraction (SPE) column (Waters Oasis HLB 25 μm column, 2.0×15 mm direct connect) without further processing. The sample on the SPE column is washed with a 5% methanol, 95% dH₂O solution (2.1 mL/min, 1.0 minute), then loaded onto an analytical column using a valve switch that places the SPE column in an inverted flow path onto the analytical column (Waters Xterra MS C8 5 μm IS column, 2.1×20 mm). The sample is eluted from the analytical column with a reverse phase gradient (Mobile Phase A: 10 mM ammonium hydroxide in dH₂O, Mobile Phase B: 10 mM ammonium hydroxide in 80% acetonitrile and 20% methanol; 20% B for the first 3 minutes then ramping to 95% B over 4 min. and holding for 2 min., all at a flow rate of 0.4 mL/min.). At 9.1 minutes, the gradient returns to the initial conditions of 20% B for 1 min. polypeptide is eluted from the analytical column and is detected by triple-quadrapole mass spectrometry (MRM, 764 (+2 charge state)>182 (+1 charge state) Da; cone voltage=30V; collision=20 eV; parent resolution=2 Da at base peak; daughter resolution=2 Da at base peak). Instrument response is converted into concentration units by comparison with a standard curve using known amounts of chemically synthesized polypeptide(s) prepared and injected in mouse plasma using the same procedure.

Similarly, pharmacokinetic properties are determined in rats using LCMS methodology. Rat plasma samples containing the polypeptide are extracted using a Waters Oasis MAX 96 well solid phase extraction (SPE) plate. A 200 μL volume of rat plasma is mixed with 200 μL of ¹³C₉, ¹⁵N-labeled polypeptide in the well of a prepared SPE plate. The samples are drawn through the stationary phase with 15 mm Hg vacuum. All samples are rinsed with 200 μL of 2% ammonium hydroxide in water followed by 200 μL of 20% methanol in water. The samples are eluted with consecutive 100 μL volumes of 5/20/75 formic acid/water/methanol and 100 μL 5/15/80 formic acid/water/methanol. The samples are dried under nitrogen and resuspended in 100 μL of 20% methanol in water. Samples are analyzed by a Waters Quattro Micro mass spectrometer coupled to a Waters 1525 binary pump with a Waters 2777 autosampler. A 40 μL volume of each sample is injected onto a Thermo Hypersil GOLD C18 column (2.1×50 mm, 5 um). polypeptide is eluted by a gradient over 3 minutes with acetonitrile and water containing 0.05% trifluoroacetic acid. The Quattro Micro mass spectrometer is run in multiple reaction monitoring (MRM) mode using the mass transitions of, for example 764>182 or 682>136. Using this methodology, polypeptide is dosed orally and by IV to rats at 10 mg/kg. Pharmacokinetic properties including area under the curve and bioavailabilty are determined.

### In Vitro Proteolytic Stability

The stability of polypeptides/GC-C agonists described herein in the presence of several mammalian digestive enzymes is determined. Polypeptide/GC-C agonists described herein are exposed to a variety of in vitro conditions including digestive enzymes and low ph environments designed to simulate gastric fluid. Polypeptide/GC-C agonists described herein are incubated with chymotrypsin, trypsin, pepsin, aminopeptidase, carboxypeptidase A, and simulated gastric fluid (sgf) at ph 1.0. Samples are collected at 0, 3, and 24 h for all conditions except pepsin digestion and the SGF. For the latter two conditions, samples are obtained at 0, 1, and 3 h. Negative control samples are prepared for initial and final time points. A separate, positive activity control is run in parallel for each condition. All samples are analyzed by LC/MS.

### Effect on Bowel Habits

Peptide/GC-C agonists described herein can be administered to mammals (e.g. humans) to determine the effect on bowel habits (including Bristol Stool Form Scale score, stool frequency (number of stools per week), ease of passage and stool weight). polypeptide/GC-C agonist is administered in a single dose or multiple doses (for example, once daily over a consecutive 7 day period) and alterations in bowel habit are evaluated (for each collected bowel movement), for example, prior to dose, during dosage (for multiple dosing), and postdose.

### The Bristol Stool Form Scale is:

1: Separate hard lumps, like nuts
2: Sausage-shaped but lumpy
3: Like a sausage or snake but with cracks on its surface
4: Like a sausage or snake, smooth and soft
5: Soft blobs with clear-cut edges
6: Fluffy pieces with ragged edges, a mushy stool
7: Watery, no solid pieces
   The scale used to determine ease of passage is:
1. Manual disimpaction
2 Enema needed
3. Straining needed
4. Normal
5. Urgent without pain
6. Urgent with pain
7. Incontinent

### Rat Model of Postoperative Ileus.

Female CD rats are used to test the effect of polypeptides/GC-C agonists described herein on delayed transit induced by abdominal surgery and manual manipulation of the small intestine. Groups of at least nine rats undergo abdominal surgery under isoflurane anesthesia. Surgery consists of laparotomy and 5 minutes of gentle manual intestinal massage. Following recovery from anesthesia, rats are dosed orally with either polypeptide/GC-C agonist (for example, 10 μg/kg) described herein or vehicle (20 mM Tris) in a volume of 300 μl. 1 hour after dosing, intestinal transit rate is measured. Animals are again dosed with 300 μl of the test article followed immediately by 500 μl of a charcoal meal (10% charcoal, 10% gum arabic in water). To calculate the distance of the small intestine traveled by the charcoal front, after 20 minutes, the total length of the intestine as well as the distance traveled from the stomach to the charcoal front are measured for each animal.

Effect on cGMP Levels and Secretion in Ligated Loops Rodent Models

The effect of polypeptides/GC-C agonists described herein on cGMP levels and secretion are studied by injecting polypeptides/GC-C agonists described herein directly into an isolated loop in either wild-type or GC-C KO mice. This is done by surgically ligating a loop in the small intestine of the mouse. The methodology for ligated loop formation is similar to that described in London et al. 1997 Am J Physiol p. G93-105. The loop is roughly centered and is a length of 1-3 cm. The loops are injected with 100 μl of either SEQ ID NO:3 (5 μg) or vehicle (20 mM Tris, pH 7.5 or Krebs Ringer, 10 mM Glucose, HEPES buffer (KRGH)). Following a recovery time of 90 minutes the loops are excised. Weights are recorded for each loop before and after removal of the fluid contained therein. The length of each loop is also recorded. A weight to length ratio (W/L) for each loop is calculated to determine the effects of the polypeptide/GC-C agonist described herein on secretion.

To determine the effect of the polypeptide/GC-C agonist described herein on cGMP activity, fluid from the loop is collected in ice-cold trichloracetic acid (TCA) and stored at −80° C. for use in an assay to measure cGMP levels in the fluid. Intestinal fluid samples are TCA extracted, and cyclic GMP is measured by EIA according to procedures outlined in the Cayman Chemical Cyclic GMP EIA kit (Cayman Chemical, Ann Arbor, Mich.) to determine cyclic GMP levels in the intestinal fluid of the mouse in the presence of either polypeptide/GC-C agonist described herein or vehicle.

The effects of polypeptides/GC-C agonists described herein on cGMP levels and secretion in ligated loops in female CD rats can also be determined using protocols similar to those described above. In the case of the rat, however four loops of intestine are surgically ligated. The first three loops are distributed equally in the small intestine and the fourth loop is located in colon. Loops are 1 to 3 centimeters, and are injected with 200 μL of either polypeptide/agonist described herein (5 μg) or vehicle (Krebs Ringer, 10 mM glucose, HEPES buffer (KRGH)).

### Diuresis Related Experiments

### Effect on Diuresis and Natriuresis

The effect of polypeptides/GC-agonists described herein on diuresis and natriuresis can be determined using methodology similar to that described in WO06/001931 (examples 6 (p. 42) and 8 (p. 45)). Briefly, the polypeptide/agonist described herein (180-pmol) is infused for 60 min into a group of 5 anesthetized rats. Given an estimated rat plasma volume of 10 mL, the infusion rate is approximately 3 pmol/mL/min. Blood pressure, urine production, and sodium excretion are monitored for approximately 40 minutes prior to the infusion, during the infusion, and for approximately 50 minutes after the infusion to measure the effect of the polypeptide/GC-C agonist on diuresis and natriuresis. For comparison, a control group of five rats is infused with regular saline. Urine and sodium excretion can be assessed. Dose response can also be determined. polypeptide/GC-C agonist described herein is infused intravenously into rats over 60 minutes. Urine is collected at 30 minute intervals up to 180 minutes after termination of polypeptide/GC-C agonist infusion, and urine volume, sodium excretion, and potassium excretion are determined for each collection interval. Blood pressure is monitored continuously. For each dose a dose-response relationship for urine volume, sodium and potassium excretion can be determined. Plasma concentration of the polypeptide/GC-agonist is also determined before and after iv infusion.

### Rat Diuresis Experiment:

Female Sprague-Dawley rats (>170 g, 2-8 per group) are given 3.0 mL of iosotonic saline perorally, and then anesthetized with isoflurane/oxygen. Once an appropriate level of anesthesia has been achieved, a sterile polyurethane catheter (˜16 cm, 0.6 mm ID, 0.9 mm OD) is inserted 1.5-2.0 cm into the urethra and secured using 1-2 drops of veterinary bond adhesive applied to urethra/catheter junction. Rats are then dosed with either vehicle or test article via the intravenous or intraperitoneal route. Rats are then placed in appropriately sized rat restraint tubes, with the catheter protruding out of the restraint tube into a 10 mL graduated cylinder. Rats are allowed to regain consciousness, and the volume of urine excreted over a 1-5 hour duration is recorded periodically for each rat.

### Administration of Polypeptides and GC-C Receptor Agonists

For treatment of gastrointestinal disorders, the polypeptides and agonists described herein are preferably administered orally, e.g., as a tablet or cachet containing a predetermined amount of the active ingredient, pellet, gel, paste, syrup, bolus, electuary, slurry, sachet; capsule; powder; lyophilized powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The polypeptides and agonists can be co-administered with other agents used to treat gastrointestinal disorders including but not limited to the agents described herein. The polypeptides and agonists can also be administered by rectal suppository. For the treatment of disorders outside the gastrointestinal tract such as congestive heart failure and benign prostatic hypertrophy, polypeptides and agonists are preferably administered parenterally or orally.

The polypeptides described herein can be administered alone or in combination with other agents. For example, the polypeptides can be administered together with an analgesic polypeptide or compound. The analgesic polypeptide or compound can be covalently attached to a polypeptide described herein or it can be a separate agent that is administered together with or sequentially with a polypeptide described herein in a combination therapy.

Combination therapy can be achieved by administering two or more agents, e.g., a polypeptide described herein and an analgesic polypeptide or compound, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

Combination therapy can also include the administration of two or more agents via different routes or locations. For example, (a) one agent is administered orally and another agents is administered intravenously or (b) one agent is administered orally and another is administered locally. In each case, the agents can either simultaneously or sequentially. Approximated dosages for some of the combination therapy agents described herein are found in the “BNF Recommended Dose” column of tables on pages 11-17 of WO01/76632 (the data in the tables being attributed to the March 2000 British National Formulary) and can also be found in other standard formularies and other drug prescribing directories. For some drugs, the customary prescribed dose for an indication will vary somewhat from country to country.

The agents, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose (e.g. celphere, Celphere Beads®), diluents, lubricants, binders, disintegrating agents, and the like), etc. If desired, tablet dosages of the disclosed compositions may be coated by standard aqueous or nonaqueous techniques.

Compositions of the present invention may also optionally include other therapeutic ingredients, anti-caking agents, preservatives, sweetening agents, colorants, flavors, desiccants, plasticizers, dyes, glidants, anti-adherents, anti-static agents, surfactants (wetting agents), anti-oxidants, film-coating agents, and the like. Any such optional ingredient must be compatible with the compound described herein to insure the stability of the formulation.

The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and polypeptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as:

BINDERS: corn starch, potato starch, other starches, gelatin, natural and synthetic gums such as acacia, xanthan, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (e.g., povidone, crospovidone, copovidone, etc), methyl cellulose, Methocel, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and 105™, sold by FMC Corporation, Marcus Hook, Pa., USA), or mixtures thereof,

FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, dextrose, fructose, honey, lactose anhydrate, lactose monohydrate, lactose and aspartame, lactose and cellulose, lactose and microcrystalline cellulose, maltodextrin, maltose, mannitol, microcrystalline cellulose & guar gum, molasses, sucrose, or mixtures thereof,

DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums (like gellan), low-substituted hydroxypropyl cellulose, or mixtures thereof,

LUBRICANTS: calcium stearate, magnesium stearate, mineral oil, light mineral oil, glycerin, sorbitol, mannitol, polyethylene glycol, other glycols, stearic acid, sodium lauryl sulfate, sodium stearyl fumarate, vegetable based fatty acids lubricant, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Plano, Tex. USA), a pyrogenic silicon dioxide (CAB-O-SIL, Cabot Co., Boston, Mass. USA), or mixtures thereof,

ANTI-CAKING AGENTS: calcium silicate, magnesium silicate, silicon dioxide, colloidal silicon dioxide, talc, or mixtures thereof,

ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and

COATING AGENTS: sodium carboxymethyl cellulose, cellulose acetate phthalate, ethylcellulose, gelatin, pharmaceutical glaze, hydroxypropyl cellulose, hydroxypropyl methylcellulose (hypromellose), hydroxypropyl methyl cellulose phthalate, methylcellulose, polyethylene glycol, polyvinyl acetate phthalate, shellac, sucrose, titanium dioxide, carnauba wax, microcrystalline wax, gellan gum, maltodextrin, methacrylates, microcrystalline cellulose and carrageenan or mixtures thereof.

The formulation can also include other excipients and categories thereof including but not limited to L-histidine, Pluronic®, Poloxamers (such as Lutrol® and Poloxamer 188), ascorbic acid, glutathione, permeability enhancers (e.g. lipids, sodium cholate, acylcarnitine, salicylates, mixed bile salts, fatty acid micelles, chelators, fatty acid, surfactants, medium chain glycerides), protease inhibitors (e.g. soybean trypsin inhibitor, organic acids), pH lowering agents and absorption enhancers effective to promote bioavailability (including but not limited to those described in U.S. Pat. No. 6,086,918 and U.S. Pat. No. 5,912,014), creams and lotions (like maltodextrin and carrageenans); materials for chewable tablets (like dextrose, fructose, lactose monohydrate, lactose and aspartame, lactose and cellulose, maltodextrin, maltose, mannitol, microcrystalline cellulose and guar gum, sorbitol crystalline); parenterals (like mannitol and povidone); plasticizers (like dibutyl sebacate, plasticizers for coatings, polyvinylacetate phthalate); powder lubricants (like glyceryl behenate); soft gelatin capsules (like sorbitol special solution); spheres for coating (like sugar spheres); spheronization agents (like glyceryl behenate and microcrystalline cellulose); suspending/gelling agents (like carrageenan, gellan gum, mannitol, microcrystalline cellulose, povidone, sodium starch glycolate, xanthan gum); sweeteners (like aspartame, aspartame and lactose, dextrose, fructose, honey, maltodextrin, maltose, mannitol, molasses, sorbitol crystalline, sorbitol special solution, sucrose); wet granulation agents (like calcium carbonate, lactose anhydrous, lactose monohydrate, maltodextrin, mannitol, microcrystalline cellulose, povidone, starch), caramel, carboxymethylcellulose sodium, cherry cream flavor and cherry flavor, citric acid anhydrous, citric acid, confectioner's sugar, D&C Red No. 33, D&C Yellow #10 Aluminum Lake, disodium edetate, ethyl alcohol 15%, FD&C Yellow No. 6 aluminum lake, FD&C Blue #1 Aluminum Lake, FD&C Blue No. 1, FD&C blue no. 2 aluminum lake, FD&C Green No. 3, FD&C Red No. 40, FD&C Yellow No. 6 Aluminum Lake, FD&C Yellow No. 6, FD&C Yellow No. 10, glycerol palmitostearate, glyceryl monostearate, indigo carmine, lecithin, manitol, methyl and propyl parabens, mono ammonium glycyrrhizinate, natural and artificial orange flavor, pharmaceutical glaze, poloxamer 188, Polydextrose, polysorbate 20, polysorbate 80, polyvidone, pregelatinized corn starch, pregelatinized starch, red iron oxide, saccharin sodium, sodium carboxymethyl ether, sodium chloride, sodium citrate, sodium phosphate, strawberry flavor, synthetic black iron oxide, synthetic red iron oxide, titanium dioxide, and white wax.

Solid oral dosage forms may optionally be treated with coating systems (e.g. Opadry® fx film coating system, for example Opadry® blue (OY-LS-20921), Opadry® white (YS-2-7063), Opadry® white (YS-1-7040), and black ink (S-1-8106).

The agents either in their free form or as a salt can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(ε-caprolactone) and poly(alkylene oxide) (U.S. 20030068384) to create a sustained release formulation. Such formulations can be used to implants that release a polypeptide or another agent over a period of a few days, a few weeks or several months depending on the polymer, the particle size of the polymer, and the size of the implant (see, e.g., U.S. Pat. No. 6,620,422). Other sustained release formulations and polymers for use in are described in EP 0 467 389 A2, WO 93/24150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. 20030171296, U.S. 20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and. US20020019446. In such sustained release formulations microparticles (Delie and Blanco-Prieto 2005 Molecule 10:65-80) of polypeptide are combined with microparticles of polymer. One or more sustained release implants can be placed in the large intestine, the small intestine or both. U.S. Pat. No. 6,011,011 and WO 94/06452 describe a sustained release formulation providing either polyethylene glycols (i.e. PEG 300 and PEG 400) or triacetin. WO 03/053401 describes a formulation which may both enhance bioavailability and provide controlled release of the agent within the GI tract. Additional controlled release formulations are described in WO 02/38129, EP 326 151, U.S. Pat. No. 5,236,704, WO 02/30398, WO 98/13029; U.S. 20030064105, U.S. 20030138488A1, U.S. 20030216307A1, U.S. Pat. No. 6,667,060, WO 01/49249, WO 01/49311, WO 01/49249, WO 01/49311, and U.S. Pat. No. 5,877,224.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasally (including using a cannula), intraspinally, intrathecally, or by other routes. The agents can be administered orally, e.g., as a tablet or cachet containing a predetermined amount of the active ingredient, gel, pellet, paste, syrup, bolus, electuary, slurry, capsule, powder, lyophilized powder, granules, sachet, as a solution or a suspension in an aqueous liquid or a non-aqueous liquid, as an oil-in-water liquid emulsion or a water-in-oil liquid emulsion, via a micellar formulation (see, e.g. WO 97/11682) via a liposomal formulation (see, e.g., EP 736299, WO 99/59550 and WO 97/13500), via formulations described in WO 03/094886, via bilosome (bile-salt based vesicular system), via a dendrimer, or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115-124)). The agents can be administered using high-velocity transdermal particle injection techniques using the hydrogel particle formulation described in U.S. 20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranasally using the formulation described in U.S. Pat. No. 5,179,079. Formulations suitable for parenteral injection are described in WO 00/62759. The agents can be administered using the casein formulation described in U.S. 20030206939 and WO 00/06108. The agents can be administered using the particulate formulations described in U.S. 20020034536.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, i.e. HFA-134a and HFA-227, or a mixture thereof), dichlorodifluoromethane (or other chlorofluocarbon propellants such as a mixture of Propellants 11, 12, and/or 114), propane, nitrogen, and the like. Pulmonary formulations may include permeation enhancers such as fatty acids, saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. Pat. No. 6,524,557 and references therein. The surfactants described in U.S. Pat. No. 6,524,557, e.g., a C8-C16 fatty acid salt, a bile salt, a phospholipid, or alkyl saccaride are advantageous in that some of them also reportedly enhance absorption of the polypeptide in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, U.S. 2003019437, U.S. 20030165436, and WO 96/40089 (which includes vegetable oil). Sustained release formulations suitable for inhalation are described in U.S. 20010036481A1, 20030232019A1, and U.S. 20040018243A1 as well as in WO 01/13891, WO 02/067902, WO 03/072080, and WO 03/079885. Pulmonary formulations containing microparticles are described in WO 03/015750, U.S. 20030008013, and WO 00/00176.

Pulmonary formulations containing stable glassy state powder are described in U.S. 20020141945 and U.S. Pat. No. 6,309,671. Other aerosol formulations are described in EP 1338272A1 WO 90/09781, U.S. Pat. No. 5,348,730, U.S. Pat. No. 6,436,367, WO 91/04011, and U.S. Pat. No. 6,294,153 and U.S. Pat. No. 6,290,987 describes a liposomal based formulation that can be administered via aerosol or other means. Powder formulations for inhalation are described in U.S. 20030053960 and WO 01/60341. The agents can be administered intranasally as described in U.S. 20010038824. The agents can be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The agents described herein can be incorporated into pharmaceutically-acceptable nanoparticle, nanosphere, and nanocapsule formulations (Delie and Blanco-Prieto 2005 Molecule 10:65-80). Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, ultrafine particles (sized around 0.1 μm) can be designed using polymers able to be degraded in vivo (e.g. biodegradable polyalkyl-cyanoacrylate nanoparticles). Such particles are described in the prior art (Couvreur et al, 1980; 1988; zur Muhlen et al., 1998; Zambaux et al. 1998; Pinto-Alphandry et al., 1995 and U.S. Pat. No. 5,145,684).

The agents described herein can be formulated with pH sensitive materials which may include those described in WO04041195 (including the seal and enteric coating described therein) and pH-sensitive coatings that achieve delivery in the colon including those described in U.S. Pat. No. 4,910,021 and WO9001329. U.S. Pat. No. 4,910,021 describes using a pH-sensitive material to coat a capsule. WO9001329 describes using pH-sensitive coatings on beads containing acid, where the acid in the bead core prolongs dissolution of the pH-sensitive coating. U.S. Pat. No. 5,175,003 discloses a dual mechanism polymer mixture composed of pH-sensitive enteric materials and film-forming plasticizers capable of conferring permeability to the enteric material, for use in drug-delivery systems; a matrix pellet composed of a dual mechanism polymer mixture permeated with a drug and sometimes covering a pharmaceutically neutral nucleus; a membrane-coated pellet comprising a matrix pellet coated with a dual mechanism polymer mixture envelope of the same or different composition; and a pharmaceutical dosage form containing matrix pellets. The matrix pellet releases acid-soluble drugs by diffusion in acid pH and by disintegration at pH levels of nominally about 5.0 or higher. The agents described herein may be formulated in the pH triggered targeted control release systems described in WO04052339. The agents described herein may be formulated according to the methodology described in any of WO03105812 (extruded hyrdratable polymers); WO0243767 (enzyme cleavable membrane translocators); WO03007913 and WO03086297 (mucoadhesive systems); WO02072075 (bilayer laminated formulation comprising pH lowering agent and absorption enhancer); WO04064769 (amidated polypeptides); WO05063156 (solid lipid suspension with pseudotropic and/or thixotropic properties upon melting); WO03035029 and WO03035041 (erodible, gastric retentive dosage forms); U.S. Pat. No. 5,007,790 and U.S. Pat. No. 5,972,389 (sustained release dosage forms); WO04112711 (oral extended release compositions); WO05027878, WO02072033, and WO02072034 (delayed release compositions with natural or synthetic gum); WO05030182 (controlled release formulations with an ascending rate of release); WO05048998 (microencapsulation system); U.S. Pat. No. 5,952,314 (biopolymer); U.S. Pat. No. 5,108,758 (glassy amylose matrix delivery); U.S. Pat. No. 5,840,860 (modified starch based delivery). JP10324642 (delivery system comprising chitosan and gastric resistant material such as wheat gliadin or zein); U.S. Pat. No. 5,866,619 and U.S. Pat. No. 6,368,629 (saccharide containing polymer); U.S. Pat. No. 6,531,152 (describes a drug delivery system containing a water soluble core (Ca pectinate or other water-insoluble polymers) and outer coat which bursts (eg hydrophobic polymer-Eudragrit)); U.S. Pat. No. 6,234,464; U.S. Pat. No. 6,403,130 (coating with polymer containing casein and high methoxy pectin; WO0174175 (Maillard reaction product); WO05063206 (solubility increasing formulation); WO04019872 (transferring fusion proteins). The agents described herein may be formulated using gastrointestinal retention system technology (GIRES; Merrion Pharmaceuticals). GIRES comprises a controlled-release dosage form inside an inflatable pouch, which is placed in a drug capsule for oral administration. Upon dissolution of the capsule, a gas-generating system inflates the pouch in the stomach where it is retained for 16-24 hours, all the time releasing agents described herein.

The agents described herein can be formulated in an osmotic device including the ones disclosed in U.S. Pat. No. 4,503,030, U.S. Pat. No. 5,609,590 and U.S. Pat. No. 5,358,502. U.S. Pat. No. 4,503,030 discloses an osmotic device for dispensing a drug to certain pH regions of the gastrointestinal tract. More particularly, the invention relates to an osmotic device comprising a wall formed of a semi-permeable pH sensitive composition that surrounds a compartment containing a drug, with a passageway through the wall connecting the exterior of the device with the compartment. The device delivers the drug at a controlled rate in the region of the gastrointestinal tract having a pH of less than 3.5, and the device self-destructs and releases all its drug in the region of the gastrointestinal tract having a pH greater than 3.5, thereby providing total availability for drug absorption. U.S. Pat. Nos. 5,609,590 and 5,358,502 disclose an osmotic bursting device for dispensing a beneficial agent to an aqueous environment. The device comprises a beneficial agent and osmagent surrounded at least in part by a semi-permeable membrane. The beneficial agent may also function as the osmagent. The semi-permeable membrane is permeable to water and substantially impermeable to the beneficial agent and osmagent. A trigger means is attached to the semi-permeable membrane (e.g., joins two capsule halves). The trigger means is activated by a pH of from 3 to 9 and triggers the eventual, but sudden, delivery of the beneficial agent. These devices enable the pH-triggered release of the beneficial agent core as a bolus by osmotic bursting.

The agents described herein may be formulated based on the invention described in U.S. Pat. No. 5,316,774 which discloses a composition for the controlled release of an active substance comprising a polymeric particle matrix, where each particle defines a network of internal pores. The active substance is entrapped within the pore network together with a blocking agent having physical and chemical characteristics selected to modify the release rate of the active substance from the internal pore network. In one embodiment, drugs may be selectively delivered to the intestines using an enteric material as the blocking agent. The enteric material remains intact in the stomach but degrades under the pH conditions of the intestines. In another embodiment, the sustained release formulation employs a blocking agent, which remains stable under the expected conditions of the environment to which the active substance is to be released. The use of pH-sensitive materials alone to achieve site-specific delivery is difficult because of leaking of the beneficial agent prior to the release site or desired delivery time and it is difficult to achieve long time lags before release of the active ingredient after exposure to high pH (because of rapid dissolution or degradation of the pH-sensitive materials).

The agents may also be formulated in a hybrid system which combines pH-sensitive materials and osmotic delivery systems. These hybrid devices provide delayed initiation of sustained-release of the beneficial agent. In one device a pH-sensitive matrix or coating dissolves releasing osmotic devices that provide sustained release of the beneficial agent see U.S. Pat. Nos. 4,578,075, 4,681,583, and 4,851,231. A second device consists of a semipermeable coating made of a polymer blend of an insoluble and a pH-sensitive material. As the pH increases, the permeability of the coating increases, increasing the rate of release of beneficial agent see U.S. Pat. Nos. 4,096,238, 4,503,030, 4,522, 625, and 4,587,117.

The agents described herein may be formulated in terpolumers according to U.S. Pat. No. 5,484,610 which discloses terpolymers which are sensitive to pH and temperature which are useful carriers for conducting bioactive agents through the gastric juices of the stomach in a protected form. The terpolymers swell at the higher physiologic pH of the intestinal tract causing release of the bioactive agents into the intestine. The terpolymers are linear and are made up of 35 to 99 wt % of a temperature sensitive component, which imparts to the terpolymer LCST (lower critical solution temperature) properties below body temperatures, 1 to 30 wt % of a pH sensitive component having a pKa in the range of from 2 to 8 which functions through ionization or deionization of carboxylic acid groups to prevent the bioactive agent from being lost at low pH but allows bioactive agent release at physiological pH of about 7.4 and a hydrophobic component which stabilizes the LCST below body temperatures and compensates for bioactive agent effects on the terpolymers. The terpolymers provide for safe bioactive agent loading, a simple procedure for dosage form fabrication and the terpolymer functions as a protective carrier in the acidic environment of the stomach and also protects the bioactive agents from digestive enzymes until the bioactive agent is released in the intestinal tract.

The agents described herein may be formulated in pH sensitive polymers according to those described in U.S. Pat. No. 6,103,865. U.S. Pat. No. 6,103,865 discloses pH-sensitive polymers containing sulfonamide groups, which can be changed in physical properties, such as swellability and solubility, depending on pH and which can be applied for a drug-delivery system, bio-material, sensor, and the like, and a preparation method therefore. The pH-sensitive polymers are prepared by introduction of sulfonamide groups, various in pKa, to hydrophilic groups of polymers either through coupling to the hydrophilic groups of polymers, such as acrylamide, N,N-dimethylacrylamide, acrylic acid, N-isopropylacrylamide and the like or copolymerization with other polymerizable monomers. These pH-sensitive polymers may have a structure of linear polymer, grafted copolymer, hydrogel or interpenetrating network polymer.

The agents described herein may be formulated according U.S. Pat. No. 5,656,292 which discloses a composition for pH dependent or pH regulated controlled release of active ingredients especially drugs. The composition consists of a compactable mixture of the active ingredient and starch molecules substituted with acetate and dicarboxylate residues. The preferred dicarboxylate acid is succinate. The average substitution degree of the acetate residue is at least 1 and 0.2-1.2 for the dicarboxylate residue. The starch molecules can have the acetate and dicarboxylate residues attached to the same starch molecule backbone or attached to separate starch molecule backbones. The present invention also discloses methods for preparing said starch acetate dicarboxylates by transesterification or mixing of starch acetates and starch dicarboxylates respectively.

The agents described herein may be formulated according to the methods described in U.S. Pat. Nos. 5,554,147, 5,788,687, and 6,306,422 which disclose a method for the controlled release of a biologically active agent wherein the agent is released from a hydrophobic, pH-sensitive polymer matrix. The polymer matrix swells when the environment reaches pH 8.5, releasing the active agent. A polymer of hydrophobic and weakly acidic comonomers is disclosed for use in the controlled release system. Also disclosed is a specific embodiment in which the controlled release system may be used. The pH-sensitive polymer is coated onto a latex catheter used in ureteral catheterization. A ureteral catheter coated with a pH-sensitive polymer having an antibiotic or urease inhibitor trapped within its matrix will release the active agent when exposed to high pH urine.

The agents can be administered using COLAL® colonic drug delivery technology (U.S. Pat. No. 6,534,549) BTGInternational, Ltd.; Alizyme, plc; Cambridge, UK) in which small pellets containing the agents are coated with ethylcellulose and a specific form of amylose. This coating prevents drug release in the stomach and small intestine. When the pellets reach the colon the amylose in the coating is broken down by bacterial enzymes and the agent is released. The agents described herein may be formulated in/with bioadhesive polymers according to U.S. Pat. No. 6,365,187. Bioadhesive polymers in the form of, or as a coating on, microcapsules containing drugs or bioactive substances which may serve for therapeutic, or diagnostic purposes in diseases of the gastrointestinal tract, are described in U.S. Pat. No. 6,365,187. The polymeric microspheres all have a bioadhesive force of at least 11 mN/cm² (110 N/m2) Techniques for the fabrication of bioadhesive microspheres, as well as a method for measuring bioadhesive forces between microspheres and selected segments of the gastrointestinal tract in vitro are also described. This quantitative method provides a means to establish a correlation between the chemical nature, the surface morphology and the dimensions of drug-loaded microspheres on one hand and bioadhesive forces on the other, allowing the screening of the most promising materials from a relatively large group of natural and synthetic polymers which, from theoretical consideration, should be used for making bioadhesive microspheres. Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means. The agent can be fused to immunoglobulins or albumin, albumin variants or fragments thereof, or incorporated into a liposome to improve half-life. Thus the peptides described herein may be fused directly or via a peptide linker, water soluble polymer, or prodrug linker to albumin or an analog, fragment, or derivative thereof. Generally, the albumin proteins that are part of the fusion proteins of the present invention may be derived from albumin cloned from any species, including human. Human serum albumin (HSA) consists of a single non-glycosylated polypeptide chain of 585 amino acids with a formula molecular weight of 66,500. The amino acid sequence of human HSA is known [See Meloun, et al. (1975) FEBS Letters 58:136; Behrens, et al. (1975) Fed. Proc. 34:591; Lawn, et al. (1981) Nucleic Acids Research 9:6102-6114; Minghetti, et al. (1986) J. Biol. Chem. 261:6747, each of which are incorporated by reference herein]. A variety of polymorphic variants as well as analogs and fragments of albumin have been described. [See Weitkamp, et al., (1973) Ann. Hum. Genet. 37:219]. For example, in EP 322,094, various shorter forms of HSA. Some of these fragments of HSA are disclosed, including HSA (1-373), HSA (1-388), HSA (1-389), HSA (1-369), and HSA (1-419) and fragments between 1-369 and 1-419. EP 399,666 discloses albumin fragments that include HSA (1-177) and HSA (1-200) and fragments between HSA (1-177) and HSA (1-200). Methods related to albumin fusion proteins can be found in U.S. Pat. No. 7,056,701, U.S. Pat. No. 6,994,857, U.S. Pat. No. 6,946,134, U.S. Pat. No. 6,926,898, and U.S. Pat. No. 6,905,688 and the related priority documents and references cited therein. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature Reviews Drug Discovery 2: 214-221 and the references therein. polypeptides can also be modified with alkyl groups (e.g., C1-C20 straight or branched alkyl groups); fatty acid radicals; and combinations of PEG, alkyl groups and fatty acid radicals (see U.S. Pat. No. 6,309,633; Soltero et al., 2001 Innovations in Pharmaceutical Technology 106-110). The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in U.S. 20020055496, WO 00/47203, and U.S. Pat. No. 6,495,120. The agent can be delivered using nanoemulsion formulations described in WO 01/91728A2.

### Controlled Release Formulations

In general, one can provide for controlled release of the agents described herein through the use of a wide variety of polymeric carriers and controlled release systems including erodible and non-erodible matrices, osmotic control devices, various reservoir devices, enteric coatings and multiparticulate control devices.

Matrix devices are a common device for controlling the release of various agents. In such devices, the agents described herein are generally present as a dispersion within the polymer matrix, and are typically formed by the compression of a polymer/drug mixture or by dissolution or melting. The dosage release properties of these devices may be dependent upon the solubility of the agent in the polymer matrix or, in the case of porous matrices, the solubility in the sink solution within the pore network, and the tortuosity of the network. In one instance, when utilizing an erodible polymeric matrix, the matrix imbibes water and forms an aqueous-swollen gel that entraps the agent. The matrix then gradually erodes, swells, disintegrates or dissolves in the GI tract, thereby controlling release of one or more of the agents described herein. In non-erodible devices, the agent is released by diffusion through an inert matrix.

Agents described herein can be incorporated into an erodible or non-erodible polymeric matrix controlled release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or matrix that entraps the agent described herein. The aqueous-swollen matrix gradually erodes, swells, disintegrates or dissolves in the environment of use, thereby controlling the release of a compound described herein to the environment of use.

The erodible polymeric matrix into which an agent described herein can be incorporated may generally be described as a set of excipients that are mixed with the agent following its formation that, when contacted with the aqueous environment of use imbibes water and forms a water-swollen gel or matrix that entraps the drug form. Drug release may occur by a variety of mechanisms, for example, the matrix may disintegrate or dissolve from around particles or granules of the agent or the agent may dissolve in the imbibed aqueous solution and diffuse from the tablet, beads or granules of the device. One ingredient of this water-swollen matrix is the water-swellable, erodible, or soluble polymer, which may generally be described as an osmopolymer, hydrogel or water-swellable polymer. Such polymers may be linear, branched, or crosslinked. The polymers may be homopolymers or copolymers. In certain embodiments, they may be synthetic polymers derived from vinyl, acrylate, methacrylate, urethane, ester and oxide monomers. In other embodiments, they can be derivatives of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin), hydrophilic colloids (e.g. pectin), phosphatides (e.g. lecithin), alginates (e.g. ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent. In certain embodiments, the cellulosics for the erodible matrix comprises aqueous-soluble and aqueous-erodible cellulosics can include, for example, ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 daltons, for example, the Dow Methocel™ series E5, E15LV, E50LV and K100LY) and high viscosity (MW greater than 50,000 daltons, for example, E4MCR, E10MCR, K4M, K15M and K100M and the Methocel™ K series) HPMC. Other commercially available types of HPMC include the Shin Etsu Metolose 90SH series.

The choice of matrix material can have a large effect on the maximum drug concentration attained by the device as well as the maintenance of a high drug concentration. The matrix material can be a concentration-enhancing polymer, for example, as described in WO05/011634.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGITO, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride.

The erodible matrix polymer may contain a wide variety of the same types of additives and excipients known in the pharmaceutical arts, including osmopolymers, osmagens, solubility-enhancing or -retarding agents and excipients that promote stability or processing of the device.

Alternatively, the agents of the present invention may be administered by or incorporated into a non-erodible matrix device. In such devices, an agent described herein is distributed in an inert matrix. The agent is released by diffusion through the inert matrix. Examples of materials suitable for the inert matrix include insoluble plastics (e.g methyl acrylate-methyl methacrylate copolymers, polyvinyl chloride, polyethylene), hydrophilic polymers (e.g. ethyl cellulose, cellulose acetate, crosslinked polyvinylpyrrolidone (also known as crospovidone)), and fatty compounds (e.g. carnauba wax, microcrystalline wax, and triglycerides). Such devices are described further in Remington: The Science and Practice of Pharmacy, 20th edition (2000). Matrix controlled release devices may be prepared by blending an agent described herein and other excipients together, and then forming the blend into a tablet, caplet, pill, or other device formed by compressive forces. Such compressed devices may be formed using any of a wide variety of presses used in the fabrication of pharmaceutical devices. Examples include single-punch presses, rotary tablet presses, and multilayer rotary tablet presses, all well known in the art. See for example, Remington: The Science and Practice of Pharmacy, 20th Edition, 2000. The compressed device may be of any shape, including round, oval, oblong, cylindrical, or triangular. The upper and lower surfaces of the compressed device may be flat, round, concave, or convex.

In certain embodiments, when formed by compression, the device has a strength of at least 5 Kiloponds (Kp)/cm² (for example, at least 7 Kp/cm²). Strength is the fracture force, also known as the tablet hardness required to fracture a tablet formed from the materials, divided by the maximum cross-sectional area of the tablet normal to that force. The fracture force may be measured using a Schleuniger Tablet Hardness Tester, Model 6D. The compression force required to achieve this strength will depend on the size of the tablet, but generally will be greater than about 5 kP/cm². Friability is a well-know measure of a device's resistance to surface abrasion that measures weight loss in percentage after subjecting the device to a standardized agitation procedure. Friability values of from 0.8 to 1.0% are regarded as constituting the upper limit of acceptability. Devices having a strength of greater than 5 kP/cm² generally are very robust, having a friability of less than 0.5%. Other methods for forming matrix controlled-release devices are well known in the pharmaceutical arts. See for example, Remington: The Science and Practice of Pharmacy, 20th Edition, 2000.

As noted above, the agents described herein may also be incorporated into an osmotic control device. Such devices generally include a core containing one or more agents as described herein and a water permeable, non-dissolving and non-eroding coating surrounding the core which controls the influx of water into the core from an aqueous environment of use so as to cause drug release by extrusion of some or all of the core to the environment of use. In certain embodiments, the coating is polymeric, aqueous-permeable, and has at least one delivery port. The core of the osmotic device optionally includes an osmotic agent which acts to imbibe water from the surrounding environment via such a semi-permeable membrane. The osmotic agent contained in the core of this device may be an aqueous-swellable hydrophilic polymer or it may be an osmogen, also known as an osmagent. Pressure is generated within the device which forces the agent(s) out of the device via an orifice (of a size designed to minimize solute diffusion while preventing the build-up of a hydrostatic pressure head).

Osmotic agents create a driving force for transport of water from the environment of use into the core of the device. Osmotic agents include but are not limited to water-swellable hydrophilic polymers, and osmogens (or osmagens). Thus, the core may include water-swellable hydrophilic polymers, both ionic and nonionic, often referred to as osmopolymers and hydrogels. The amount of water-swellable hydrophilic polymers present in the core may range from about 5 to about 80 wt % (including for example, 10 to 50 wt %). Nonlimiting examples of core materials include hydrophilic vinyl and acrylic polymers, polysaccharides such as calcium alginate, polyethylene oxide (PEO), polyethylene glycol (PEG), polypropylene glycol (PPG), poly (2-hydroxyethyl methacrylate), poly (acrylic) acid, poly (methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolat. Other materials include hydrogels comprising interpenetrating networks of polymers that may be formed by addition or by condensation polymerization, the components of which may comprise hydrophilic and hydrophobic monomers such as those just mentioned. Water-swellable hydrophilic polymers include but are not limited to PEO, PEG, PVP, sodium croscarmellose, HPMC, sodium starch glycolate, polyacrylic acid and crosslinked versions or mixtures thereof.

The core may also include an osmogen (or osmagent). The amount of osmogen present in the core may range from about 2 to about 70 wt % (including, for example, from 10 to 50 wt %). Typical classes of suitable osmogens are water-soluble organic acids, salts and sugars that are capable of imbibing water to thereby effect an osmotic pressure gradient across the barrier of the surrounding coating. Typical useful osmogens include but are not limited to magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. In certain embodiments, the osmogen is glucose, lactose, sucrose, mannitol, xylitol, sodium chloride, including combinations thereof. The core may include a wide variety of additives and excipients that enhance the performance of the dosage form or that promote stability, tableting or processing. Such additives and excipients include tableting aids, surfactants, water-soluble polymers, pH modifiers, fillers, binders, pigments, disintegrants, antioxidants, lubricants and flavorants. Nonlimiting examples of additives and excipients include but are not limited to those described elsewhere herein as well as microcrystalline cellulose, metallic salts of acids (e.g. aluminum stearate, calcium stearate, magnesium stearate, sodium stearate, zinc stearate), pH control agents (e.g. buffers, organic acids, organic acid salts, organic and inorganic bases), fatty acids, hydrocarbons and fatty alcohols (e.g. stearic acid, palmitic acid, liquid paraffin, stearyl alcohol, and palmitol), fatty acid esters (e.g. glyceryl (mono- and di-) stearates, triglycerides, glyceryl (palmiticstearic) ester, sorbitan esters (e.g. sorbitan monostearate, saccharose monostearate, saccharose monopalmitate, sodium stearyl fumarate), polyoxyethylene sorbitan esters), surfactants (e.g. alkyl sulfates (e.g. sodium lauryl sulfate, magnesium lauryl sulfate), polymers (e.g. polyethylene glycols, polyoxyethylene glycols, polyoxyethylene, polyoxypropylene ethers, including copolymers thereof), polytetrafluoroethylene), and inorganic materials (e.g. talc, calcium phosphate), cyclodextrins, sugars (e.g. lactose, xylitol), sodium starch glycolate). Nonlimiting examples of disintegrants are sodium starch glycolate (e.g., Explotab™ CLV, (microcrystalline cellulose (e.g., Avicel™), microcrystalline silicified cellulose (e.g., ProSolv™), croscarmellose sodium (e.g., Ac-Di-Sol™). When the agent described herein is a solid amorphous dispersion formed by a solvent process, such additives may be added directly to the spray-drying solution when forming an agent described herein/concentration-enhancing polymer dispersion such that the additive is dissolved or suspended in the solution as a slurry, Alternatively, such additives may be added following the spray-drying process to aid in forming the final controlled release device.

A nonlimiting example of an osmotic device consists of one or more drug layers containing an agent described herein, such as a solid amorphous drug/polymer dispersion, and a sweller layer that comprises a water-swellable polymer, with a coating surrounding the drug layer and sweller layer. Each layer may contain other excipients such as tableting aids, osmagents, surfactants, water-soluble polymers and water-swellable polymers.

Such osmotic delivery devices may be fabricated in various geometries including bilayer (wherein the core comprises a drug layer and a sweller layer adjacent to each other), trilayer (wherein the core comprises a sweller layer sandwiched between two drug layers) and concentric (wherein the core comprises a central sweller agent surrounded by the drug layer). The coating of such a tablet comprises a membrane permeable to water but substantially impermeable to drug and excipients contained within. The coating contains one or more exit passageways or ports in communication with the drug-containing layer(s) for delivering the drug agent. The drug-containing layer(s) of the core contains the drug agent (including optional osmagents and hydrophilic water-soluble polymers), while the sweller layer consists of an expandable hydrogel, with or without additional osmotic agents.

When placed in an aqueous medium, the tablet imbibes water through the membrane, causing the agent to form a dispensable aqueous agent, and causing the hydrogel layer to expand and push against the drug-containing agent, forcing the agent out of the exit passageway. The agent can swell, aiding in forcing the drug out of the passageway. Drug can be delivered from this type of delivery system either dissolved or dispersed in the agent that is expelled from the exit passageway.

The rate of drug delivery is controlled by such factors as the permeability and thickness of the coating, the osmotic pressure of the drug-containing layer, the degree of hydrophilicity of the hydrogel layer, and the surface area of the device. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the device's surface area.

Other materials useful in forming the drug-containing agent, in addition to the agent described herein itself, include HPMC, PEO and PVP and other pharmaceutically acceptable carriers. In addition, osmagents such as sugars or salts, including but not limited to sucrose, lactose, xylitol, mannitol, or sodium chloride, may be added. Materials which are useful for forming the hydrogel layer include sodium CMC, PEO (e.g. polymers having an average molecular weight from about 5,000,000 to about 7,500,000 daltons), poly (acrylic acid), sodium (polyacrylate), sodium croscarmellose, sodium starch glycolat, PVP, crosslinked PVP, and other high molecular weight hydrophilic materials.

In the case of a bilayer geometry, the delivery port(s) or exit passageway(s) may be located on the side of the tablet containing the drug agent or may be on both sides of the tablet or even on the edge of the tablet so as to connect both the drug layer and the sweller layer with the exterior of the device. The exit passageway(s) may be produced by mechanical means or by laser drilling, or by creating a difficult-to-coat region on the tablet by use of special tooling during tablet compression or by other means.

The osmotic device can also be made with a homogeneous core surrounded by a semipermeable membrane coating, as in U.S. Pat. No. 3,845,770. The agent described herein can be incorporated into a tablet core and a semipermeable membrane coating can be applied via conventional tablet-coating techniques such as using a pan coater. A drug delivery passageway can then be formed in this coating by drilling a hole in the coating, either by use of a laser or mechanical means. Alternatively, the passageway may be formed by rupturing a portion of the coating or by creating a region on the tablet that is difficult to coat, as described above. In one embodiment, an osmotic device comprises: (a) a single-layer compressed core comprising: (i) an agent described herein, (ii) a hydroxyethylcellulose, and (iii) an osmagent, wherein the hydroxyethylcellulose is present in the core from about 2.0% to about 35% by weight and the osmagent is present from about 15% to about 70% by weight; (b) a water-permeable layer surrounding the core; and (c) at least one passageway within the water-permeable layer (b) for delivering the drug to a fluid environment surrounding the tablet. In certain embodiments, the device is shaped such that the surface area to volume ratio (of a water-swollen tablet) is greater than 0.6 mm⁻¹ (including, for example, greater than 1.0 mm⁻¹). The passageway connecting the core with the fluid environment can be situated along the tablet band area. In certain embodiments, the shape is an oblong shape where the ratio of the tablet tooling axes, i.e., the major and minor axes which define the shape of the tablet, are between 1.3 and 3 (including, for example, between 1.5 and 2.5). In one embodiment, the combination of the agent described herein and the osmagent have an average ductility from about 100 to about 200 Mpa, an average tensile strength from about 0.8 to about 2.0 Mpa, and an average brittle fracture index less than about 0.2. The single-layer core may optionally include a disintegrant, a bioavailability enhancing additive, and/or a pharmaceutically acceptable excipient, carrier or diluent.

In certain embodiments, entrainment of particles of agents described herein in the extruding fluid during operation of such osmotic device is desirable. For the particles to be well entrained, the agent drug form is dispersed in the fluid before the particles have an opportunity to settle in the tablet core. One means of accomplishing this is by adding a disintegrant that serves to break up the compressed core into its particulate components. Nonlimiting examples of standard disintegrants include materials such as sodium starch glycolate (e.g., Explotab™ CLV), microcrystalline cellulose (e.g., Avicel™), microcrystalline silicified cellulose (e.g., ProSoIv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the device. Non-gelling, non-swelling disintegrants provide a more rapid dispersion of the drug particles within the core as water enters the core. In certain embodiments, non-gelling, non-swelling disintegrants are resins, for example, ion-exchange resins. In one embodiment, the resin is Amberlite™ IRP 88 (available from Rohm and Haas, Philadelphia, Pa.). When used, the disintegrant is present in amounts ranging from about 50-74% of the core agent.

Water-soluble polymers are added to keep particles of the agent suspended inside the device before they can be delivered through the passageway(s) (e.g., an orifice). High viscosity polymers are useful in preventing settling. However, the polymer in combination with the agent is extruded through the passageway(s) under relatively low pressures. At a given extrusion pressure, the extrusion rate typically slows with increased viscosity. Certain polymers in combination with particles of the agent described herein form high viscosity solutions with water but are still capable of being extruded from the tablets with a relatively low force. In contrast, polymers having a low weight-average, molecular weight (<about 300,000) do not form sufficiently viscous solutions inside the tablet core to allow complete delivery due to particle settling. Settling of the particles is a problem when such devices are prepared with no polymer added, which leads to poor drug delivery unless the tablet is constantly agitated to keep the particles from settling inside the core. Settling is also problematic when the particles are large and/or of high density such that the rate of settling increases.

In certain embodiments, the water-soluble polymers for such osmotic devices do not interact with the drug. In certain embodiments the water-soluble polymer is a non-ionic polymer. A nonlimiting example of a non-ionic polymer forming solutions having a high viscosity yet still extrudable at low pressures is Natrosol™ 250H (high molecular weight hydroxyethylcellulose, available from Hercules Incorporated, Aqualon Division, Wilmington, Del.; MW equal to about 1 million daltons and a degree of polymerization equal to about 3,700). Natrosol 250H™ provides effective drug delivery at concentrations as low as about 3% by weight of the core when combined with an osmagent. Natrosol 250H™ NF is a high-viscosity grade nonionic cellulose ether that is soluble in hot or cold water. The viscosity of a 1% solution of Natrosol 250H using a Brookfield LVT (30 rpm) at 25° C. is between about 1, 500 and about 2,500 cps.

In certain embodiments, hydroxyethylcellulose polymers for use in these monolayer osmotic tablets have a weight-average, molecular weight from about 300,000 to about 1.5 million. The hydroxyethylcellulose polymer is typically present in the core in an amount from about 2.0% to about 35% by weight.

Another example of an osmotic device is an osmotic capsule. The capsule shell or portion of the capsule shell can be semipermeable. The capsule can be filled either by a powder or liquid consisting of an agent described herein, excipients that imbibe water to provide osmotic potential, and/or a water-swellable polymer, or optionally solubilizing excipients. The capsule core can also be made such that it has a bilayer or multilayer agent analogous to the bilayer, trilayer or concentric geometries described above.

Another class of osmotic device useful in this invention comprises coated swellable tablets, for example, as described in EP378404. Coated swellable tablets comprise a tablet core comprising an agent described herein and a swelling material, preferably a hydrophilic polymer, coated with a membrane, which contains holes, or pores through which, in the aqueous use environment, the hydrophilic polymer can extrude and carry out the agent. Alternatively, the membrane may contain polymeric or low molecular weight water-soluble porosigens. Porosigens dissolve in the aqueous use environment, providing pores through which the hydrophilic polymer and agent may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser or other mechanical means. In this class of osmotic devices, the membrane material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release devices may also be multilayered, as described, for example, in EP378404.

When an agent described herein is a liquid or oil, such as a lipid vehicle formulation, for example as described in WO05/011634, the osmotic controlled-release device may comprise a soft-gel or gelatin capsule formed with a composite wall and comprising the liquid formulation where the wall comprises a barrier layer formed over the external surface of the capsule, an expandable layer formed over the barrier layer, and a semipermeable layer formed over the expandable layer. A delivery port connects the liquid formulation with the aqueous use environment. Such devices are described, for example, in U.S. Pat. No. 6,419,952, U.S. Pat. No. 6,342,249, U.S. Pat. No. 5,324,280, U.S. Pat. No. 4,672,850, U.S. Pat. No. 4,627,850, U.S. Pat. No. 4,203,440, and U.S. Pat. No. 3,995,631.

The osmotic controlled release devices of the present invention can also comprise a coating. In certain embodiments, the osmotic controlled release device coating exhibits one or more of the following features: is water-permeable, has at least one port for the delivery of drug, and is non-dissolving and non-eroding during release of the drug formulation, such that drug is substantially entirely delivered through the delivery port(s) or pores as opposed to delivery primarily via permeation through the coating material itself. Delivery ports include any passageway, opening or pore whether made mechanically, by laser drilling, by pore formation either during the coating process or in situ during use or by rupture during use. In certain embodiments, the coating is present in an amount ranging from about 5 to 30 wt % (including, for example, 10 to 20 wt %) relative to the core weight.

One form of coating is a semipermeable polymeric membrane that has the port(s) formed therein either prior to or during use. Thickness of such a polymeric membrane may vary between about 20 and 800 μm (including, for example, between about 100 to 500 μm). The diameter of the delivery port (s) may generally range in size from 0.1 to 3000 μm or greater (including, for example, from about 50 to 3000 μm in diameter). Such port(s) may be formed post-coating by mechanical or laser drilling or may be formed in situ by rupture of the coatings; such rupture may be controlled by intentionally incorporating a relatively small weak portion into the coating. Delivery ports may also be formed in situ by erosion of a plug of water-soluble material or by rupture of a thinner portion of the coating over an indentation in the core. In addition, delivery ports may be formed during coating, as in the case of asymmetric membrane coatings of the type disclosed in U.S. Pat. No. 5,612,059 and U.S. Pat. No. 5,698,220. The delivery port may be formed in situ by rupture of the coating, for example, when a collection of beads that may be of essentially identical or of a variable agent are used. Drug is primarily released from such beads following rupture of the coating and, following rupture, such release may be gradual or relatively sudden. When the collection of beads has a variable agent, the agent may be chosen such that the beads rupture at various times following administration, resulting in the overall release of drug being sustained for a desired duration.

Coatings may be dense, microporous or asymmetric, having a denser region supported by a thick porous region such as those disclosed in U.S. Pat. No. 5,612,059 and U.S. Pat. No. 5,698,220. When the coating is dense the coating can be composed of a water-permeable material. When the coating is porous, it may be composed of either a water-permeable or a water-impermeable material. When the coating is composed of a porous water-impermeable material, water permeates through the pores of the coating as either a liquid or a vapor. Nonlimiting examples of osmotic devices that utilize dense coatings include U.S. Pat. No. 3,995,631 and U.S. Pat. No. 3,845,770. Such dense coatings are permeable to the external fluid such as water and may be composed of any of the materials mentioned in these patents as well as other water-permeable polymers known in the art.

The membranes may also be porous as disclosed, for example, in U.S. Pat. No. 5,654,005 and U.S. Pat. No. 5,458,887 or even be formed from water-resistant polymers. U.S. Pat. No. 5,120,548 describes another suitable process for forming coatings from a mixture of a water-insoluble polymer and a leachable water-soluble additive. The porous membranes may also be formed by the addition of pore-formers as disclosed in U.S. Pat. No. 4,612,008. In addition, vapor-permeable coatings may even be formed from extremely hydrophobic materials such as polyethylene or polyvinylidene difluorid that, when dense, are essentially water-impermeable, as long as such coatings are porous. Materials useful in forming the coating include but are not limited to various grades of acrylic, vinyls, ethers, polyamides, polyesters and cellulosic derivatives that are water-permeable and water-insoluble at physiologically relevant pHs, or are susceptible to being rendered water-insoluble by chemical alteration such as by crosslinking Nonlimiting examples of suitable polymers (or crosslinked versions) useful in forming the coating include plasticized, unplasticized and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxiated ethylene-vinylacetate, EC, PEG, PPG, PEG/PPG copolymers, PVP, HEC, HPC, CMC, CMEC, HPMC, HPMCP, HPMCAS, HPMCAT, poly (acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes and synthetic waxes. In various embodiments, the coating agent comprises a cellulosic polymer, in particular cellulose ethers, cellulose esters and cellulose ester-ethers, i.e., cellulosic derivatives having a mixture of ester and ether substituents, the coating materials are made or derived from poly (acrylic) acids and esters, poly (methacrylic) acids and esters, and copolymers thereof, the coating agent comprises cellulose acetate, the coating comprises a cellulosic polymer and PEG, the coating comprises cellulose acetate and PEG.

Coating is conducted in conventional fashion, typically by dissolving or suspending the coating material in a solvent and then coating by dipping, spray coating or by pan-coating. In certain embodiments, the coating solution contains 5 to 15 wt % polymer. Typical solvents useful with the cellulosic polymers mentioned above include but are not limited to acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, water, and mixtures thereof. Pore-formers and non-solvents (such as water, glycerol and ethanol) or plasticizers (such as diethyl phthalate) may also be added in any amount as long as the polymer remains soluble at the spray temperature. Pore-formers and their use in fabricating coatings are described, for example, in U.S. Pat. No. 5,612,059. Coatings may also be hydrophobic microporous layers wherein the pores are substantially filled with a gas and are not wetted by the aqueous medium but are permeable to water vapor, as disclosed, for example, in U.S. Pat. No. 5,798,119. Such hydrophobic but water-vapor permeable coatings are typically composed of hydrophobic polymers such as polyalkenes, polyacrylic acid derivatives, polyethers, polysulfones, polyethersulfones, polystyrenes, polyvinyl halides, polyvinyl esters and ethers, natural waxes and synthetic waxes. Hydrophobic microporous coating materials include but are not limited to polystyrene, polysulfones, polyethersulfones, polyethylene, polypropylene, polyvinyl chloride, polyvinylidene fluoride and polytetrafluoroethylene. Such hydrophobic coatings can be made by known phase inversion methods using any of vapor-quench, liquid quench, thermal processes, leaching soluble material from the coating or by sintering coating particles. In thermal processes, a solution of polymer in a latent solvent is brought to liquid-liquid phase separation in a cooling step. When evaporation of the solvent is not prevented, the resulting membrane will typically be porous. Such coating processes may be conducted by the processes disclosed, for example, in U.S. Pat. No. 4,247,498, U.S. Pat. No. 4,490,431 and U.S. Pat. No. 4,744,906. Osmotic controlled-release devices may be prepared using procedures known in the pharmaceutical arts. See for example, Remington: The Science and Practice of Pharmacy, 20th Edition, 2000.

As further noted above, the agents described herein may be provided in the form of microparticulates, generally ranging in size from about 10 μm to about 2 mm (including, for example, from about 100 μm to 1 mm in diameter). Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes known in the art. Such multiparticulates may be made by any known process, such as wet- and dry-granulation processes, extrusion/spheronization, roller-compaction, melt-congealing, or by spray-coating seed cores. For example, in wet- and dry-granulation processes, the agent described herein and optional excipients may be granulated to form multiparticulates of the desired size. Other excipients, such as a binder (e.g., microcrystalline cellulose), may be blended with the agent to aid in processing and forming the multiparticulates. In the case of wet granulation, a binder such as microcrystalline cellulose may be included in the granulation fluid to aid in forming a suitable multiparticulate. See, for example, Remington: The Science and Practice of Pharmacy, 20″ Edition, 2000. In any case, the resulting particles may themselves constitute the therapeutic composition or they may be coated by various film-forming materials such as enteric polymers or water-swellable or water-soluble polymers, or they may be combined with other excipients or vehicles to aid in dosing to patients.

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences (18th Edition, Mack Publishing Company, 1995).

### Kits

The agents described herein and combination therapy agents can be packaged as a kit that includes single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation.

Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. gastrointestinal motility disorders, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, irritable bowel syndrome, post-operative ileus, ulcerative colitis, chronic constipation, and disorders and conditions associated with constipation (e.g. constipation associated with use of opiate pain killers, post-surgical constipation, and constipation associated with neuropathic disorders as well as other conditions and disorders described herein). The kit may optionally comprise a second pharmaceutical composition comprising one or more additional agents including but not limited to those including analgesic polypeptides and compounds, a phosphodiesterase inhibitor, an agent used to treat gastrointestinal and other disorders (including those described herein), an agent used to treat constipation, an antidiarrheal agent, an insulin or related compound (including those described herein), an anti-hypertensive agent, an agent useful in the treatment of respiratory and other disorders, an anti-obesity agent, an anti-diabetic agents, an agent that activates soluble guanylate cyclase and a pharmaceutically acceptable carrier, vehicle or diluent. The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Methods to increase chemical and/or physical stability of the agents the described herein are found in U.S. Pat. No. 6,541,606, U.S. Pat. No. 6,068,850, U.S. Pat. No. 6,124,261, U.S. Pat. No. 5,904,935, and WO 00/15224, U.S. 20030069182 (via the addition of nicotinamide), U.S. 20030175230A1, U.S. 20030175230A1, U.S. 20030175239A1, U.S. 20020045582, U.S. 20010031726, WO 02/26248, WO 03/014304, WO 98/00152A1, WO 98/00157A1, WO 90/12029, WO 00/04880, and WO 91/04743, WO 97/04796 and the references cited therein.

Methods to increase bioavailability of the agents described herein are found in U.S. Pat. No. 6,008,187, U.S. Pat. No. 5,424,289, U.S. 20030198619, WO 90/01329, WO 01/49268, WO 00/32172, and WO 02/064166. Glycyrrhizinate can also be used as an absorption enhancer (see, e.g., EP397447). WO 03/004062 discusses Ulex europaeus I (UEAl) and UEAI mimetics which may be used to target the agents described herein to the GI tract. The bioavailability of the agents described herein can also be increased by addition of oral bioavailability-enhancing agents such as those described in U.S. Pat. No. 6,818,615 including but not limited to: cyclosporins (including cyclosporins A through Z as defined in Table 1 of U.S. Pat. No. 6,818,615), for example, cyclosporin A (cyclosporin), cyclosporin F, cyclosporin D, dihydro cyclosporin A, dihydro cyclosporin C, acetyl cyclosporin A, PSC-833, (Me-Ile-4)-cyclosporin (SDZ-NIM 811) (both from Sandoz Pharmaceutical Corp.), and related oligopeptides produced by species in the genus Topycladium); antifungals including but not limited to ketoconazole; cardiovascular drug including but not limited to MS-209 (BASF), amiodarone, nifedipine, reserpine, quinidine, nicardipine, ethacrynic acid, propafenone, reserpine, amiloride; anti-migraine natural products including but not limited to ergot alkaloids; antibiotics including but not limited to cefoperazone, tetracycline, chloroquine, fosfomycin; antiparasitics including but not limited to ivermectin; multi-drug resistance reversers including but not limited to VX-710 and VX-853 (Vertex Pharmaceutical Incorporated); tyrosine kinase inhibitors including but not limited to genistein and related isoflavonoids, quercetin; protein kinase C inhibitors including but not limited to calphostin; apoptosis inducers including but not limited to ceramides; and agents active against endorphin receptors including but not limited to morphine, morphine congeners, other opioids and opioid. antagonists including (but not limited to) naloxone, naltrexone and nalmefene).

The agents described herein can be fused to a modified version of the blood serum protein transferrin. U.S. 20030221201, U.S. 20040023334, U.S. 20030226155, WO 04/020454, and WO 04/019872 discuss the manufacture and use of transferrin fusion proteins. Transferrin fusion proteins may improve circulatory half life and efficacy, decrease undesirable side effects and allow reduced dosage.

The polypeptides and agonists described herein can be recombinantly expressed in bacteria. Bacteria expressing the polypeptide or agonists can be administered orally, rectally, mucosally or in via some other mode of administration including but not limited to those described herein. Bacterial hosts suitable for such administration include but are not limited to certain Lactobacteria (e.g. Lactococcus lactis, Lactobacillus plantarum, Lact. rhamnosus and Lact. paracasei ssp. Paracasie and other species found in normal human flora (Ahrne et al. Journal of Applied Microbiology 1998 85:88)), certain Streptococcus sp. (e.g. S. gordonii), and certain B. subtilis strains (including pSM539 described in Porzio et al. BMC Biotechnology 2004 4:27). The polypeptides and agonists described herein can be administered using the Heliobacter based preparation methods described in WO06/015445.

### Dosage

The dose range for adult humans is generally from 0.005 mg to 10 g/day orally. Tablets or other forms of presentation provided in discrete units may conveniently contain an amount of compound described herein which is effective at such dosage or as a multiple of the same, for instance, units containing 5 mg to 500 mg, usually around 10 mg to 200 mg. The precise amount of compound administered to a patient will be the responsibility of the attendant physician. However, the dose employed will depend on a number of factors, including the age and sex of the patient, the precise disorder being treated, and its severity.

A dosage unit (e.g. an oral dosage unit) can include from, for example, 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or GC-C agonist described herein. In certain embodiments the dosage unit and daily dose are equivalent. In various embodiments, the dosage unit is administered with food at anytime of the day, without food at anytime of the day, with food after an overnight fast (e.g. with breakfast), at bedtime after a low fat snack. In various embodiments, the dosage unit is administered once a day, twice a day, three times a day, four times a day, five times a day, six times a day. The dosage unit can optionally comprise other agents.

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 50 mg to 650 mg (e.g. 50 mg, 100 mg, 150 mg, 200 mg, 250 mg, 300 mg, 350 mg, 400 mg, 450 mg, 500 mg, 550 mg, 600 mg) of Modulon® (trimebutine maleate).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 1 mg to 80 mg (e.g. 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg) of Propulsid® (cisapride).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 10 mg to 600 mg (e.g. 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 200 mg, 250 mg, 300 mg, 350 mg, 400 mg, 450 mg, 500 mg, 550 mg, 600 mg) of Bentyl®/Bentylol® (diciclomine).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 1 mg to 25 mg (e.g. 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg) of Questran® (cholestyramine).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 100 mg to 3000 mg (e.g. 100 mg, 200 mg, 300 mg, 400 mg, 500 mg, 600 mg, 625 mg, 700 mg, 800 mg, 900 mg, 1000 mg, 1250 mg, 1300 mg, 1400 mg, 1500 mg, 1600 mg, 1700 mg, 1800 mg, 1875 mg, 1900 mg, 2000 mg, 2100 mg, 2200 mg, 2300 mg, 2400 mg, 2500 mg,) of Equalactin®/Fibercon® (Calcium Polycarbophil).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 1 mg to 20 mg (e.g. 1 mg, 2 mg, 2.5 mg, 3 mg, 4 mg, 5 mg, 6 mg, 7 mg, 7.5 mg, 8 mg, 9 mg, 10 mg, 11 mg, 12 mg, 12.5 mg, 13 mg, 14 mg, 15 mg, 16 mg, 17.5 mg, 18 mg, 19 mg, 20 mg) of darifenacin (Enablex®).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 1 mg to 250 mg (e.g. 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg) of Ondansetron HCl (Zofran®).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 1 mg to 3000 mg (e.g. 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 250 mg, 300 mg, 350 mg, 400 mg, 450 mg, 500 mg, 750 mg, 1000 mg, 1250 mg, 1500 mg, 1750 mg, 2000 mg, 2250 mg, 2500 mg, 2750 mg, 3000 mg) of Cimetropium (Alginor®).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 1 mg to 1000 mg (e.g. 1 mg, 5 mg, 10 mg, 15 mg, 20 mg, 25 mg, 30 mg, 35 mg, 40 mg, 45 mg, 50 mg, 75 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg) of Dolasetron (Anzemet®).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 1 mg to 180 mg (e.g. 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg) of Zelnorm® (tegaserod).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 1 μg to 500 μg (e.g. 1 μg, 5 μg, 10 μg, 50 μg, 75 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg) of Levsin® (hyoscyamine sulfate).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 50 mg to 500 mg (e.g. 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 500 mg) of Dicetel® (pinaverium bromide).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 50 mg to 500 mg (e.g. 50 mg, 75 mg, 100 mg, 125 mg, 135 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg) of mebeverine (DUSPATAL®, DUSPATALIN®, COLOFAC MR®, COLOTAL®).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 1 mg to 120 mg (e.g. 1 mg, 2.5 mg, 5 mg, 7.5 mg, 10 mg, 12.5 mg, 15 mg, 20 mg, 25 mg, 30 mg, 40 mg, 50 mg, 60 mg, 70 mg, 80 mg, 90 mg, 100 mg, 110 mg, 120 mg) of Propanthiline bromide (Pro-Banthine®).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 100 μg to 5000 μg (e.g. 100 μg, 200 μg, 300 μg, 400 μg, 500 μg, 600 μg, 700 μg, 800 μg, 900 μg, 1000 μg, 1250 μg, 1500 μg, 1750 μg, 2000 μg, 2250 μg, 2500 μg, 2750 μg, 3000 μg, 3500 μg, 4000 μg, 4500 μg, 5000 μg) of Granisetron (Kytril®).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 50 μg to 3000 μg (e.g. 50 μg, 100 μg, 200 μg, 300 μg, 400 μg, 500 μg, 600 μg, 700 μg, 800 μg, 900 μg, 1000 μg, 1250 μg, 1500 μg, 1750 μg, 2000 μg, 2250 μg, 2500 μg, 2750 μg, 3000 μg) of Lotronex® (alosetron hydrochloride).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 10 mg to 600 mg (e.g. 10 mg, 20 mg, 30 mg, 40 mg, 50 mg, 60 mg, 70 mg, 80 mg, 90 mg, 100 mg, 125 mg, 150 mg, 175 mg, 200 mg, 250 mg, 300 mg, 350 mg, 400 mg, 450 mg, 500 mg, 550 mg, 600 mg) of Xifaxan® (rifaximin).

A dosage unit (e.g. an oral dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 10 mg to 600 mg (e.g. 10 mg, 20 mg, 30 mg, 40 mg, 50 mg, 60 mg, 70 mg, 80 mg, 90 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, 400 mg, 420 mg, 440 mg, 460 mg, 480 mg, 500 mg, 520 mg, 540 mg, 560 mg, 580 mg, 600 mg) of furosemide (Lasix).

A dosage unit (e.g. an oral, intravenous or intramuscular dosage unit) can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide or agonist described herein and from 0.2 mg to 10 mg (e.g. 0.2 mg, 0.4 mg, 0.5 mg, 0.75 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg) of bumetanide (Bumex®).

The precise amount of each of the two or more active ingredients in a dosage unit will depend on the desired dosage of each component. Thus, it can be useful to create a dosage unit that will, when administered according to a particular dosage schedule (e.g., a dosage schedule specifying a certain number of units and a particular timing for administration), deliver the same dosage of each component as would be administered if the patient was being treated with only a single component. In other circumstances, it might be desirable to create a dosage unit that will deliver a dosage of one or more components that is less than that which would be administered if the patient was being treated only with a single component. Finally, it might be desirable to create a dosage unit that will deliver a dosage of one or more components that is greater than that which would be administered if the patient was being treated only with a single component. The pharmaceutical composition can include additional ingredients including but not limited to the excipients described herein. In certain embodiments, one or more therapeutic agents of the dosage unit may exist in an extended or control release formulation and additional therapeutic agents may not exist in extended release formulation. For example, a polypeptide or agonist described herein may exist in a controlled release formulation or extended release formulation in the same dosage unit with another agent that may or may not be in either a controlled release or extended release formulation. Thus, in certain embodiments, it may be desirable to provide for the immediate release of one or more of the agents described herein, and the controlled release of one or more other agents.

In certain embodiments the dosage unit and daily dose are equivalent. In certain embodiments the dosage unit and the daily dose are not equivalent. In various embodiments, the dosage unit is administered twenty minutes prior to food consumption, twenty minutes after food consumption, with food at anytime of the day, without food at anytime of the day, with food after an overnight fast (e.g. with breakfast), at bedtime after a low fat snack. In various embodiments, the dosage unit is administered once a day, twice a day, three times a day, four times a day, five times a day, six times a day.

When two or more active ingredients are combined in single dosage form, chemical interactions between the active ingredients may occur. For example, acidic and basic active ingredients can react with each other and acidic active ingredients can facilitate the degradation of acid labile substances. Thus, in certain dosage forms, acidic and basic substances can be physically separated as two distinct or isolated layers in a compressed tablet, or in the core and shell of a press-coated tablet. Additional agents that are compatible with acidic as well as basic substances, have the flexibility of being placed in either layer. In certain multiple layer compositions at least one active ingredient can be enteric-coated. In certain embodiments thereof at least one active ingredient can be presented in a controlled release form. In certain embodiments where a combination of three or more active substances are used, they can be presented as physically isolated segments of a compressed mutlilayer tablet, which can be optionally film coated.

The therapeutic combinations described herein can be formulated as a tablet or capsule comprising a plurality of beads, granules, or pellets. All active ingredients including the vitamins of the combination are formulated into granules or beads or pellets that are further coated with a protective coat, an enteric coat, or a film coat to avoid the possible chemical interactions. Granulation and coating of granules or beads is done using techniques well known to a person skilled in the art. At least one active ingredient can present in a controlled release form. Finally these coated granules or beads are filled into hard gelatin capsules or compressed to form tablets.

The therapeutic combinations described herein can be formulated as a capsule comprising microtablets or minitablets of all active ingredients. Microtablets of the individual agents can be prepared using well known pharmaceutical procedures of tablet making like direct compression, dry granulation or wet granulation. Individual microtablets can be filled into hard gelatin capsules. A final dosage form may comprise one or more microtablets of each individual component. The microtablets may be film coated or enteric coated.

The therapeutic combinations described herein can be formulated as a capsule comprising one or more microtablets and powder, or one or more microtablets and granules or beads. In order to avoid interactions between drugs, some active ingredients of a said combination can be formulated as microtablets and the others filled into capsules as a powder, granules, or beads. The microtablets may be film coated or enteric coated. At least one active ingredient can be presented in controlled release form.

The therapeutic combinations described herein can be formulated wherein the active ingredients are distributed in the inner and outer phase of tablets. In an attempt to divide chemically incompatible components of proposed combination, few interacting components are converted in granules or beads using well known pharmaceutical procedures in prior art. The prepared granules or beads (inner phase) are then mixed with outer phase comprising the remaining active ingredients and at least one pharmaceutically acceptable excipient. The mixture thus comprising inner and outer phase is compressed into tablets or molded into tablets. The granules or beads can be controlled release or immediate release beads or granules, and can further be coated using an enteric polymer in an aqueous or non-aqueous system, using methods and materials that are known in the art.

The therapeutic combinations described herein can be formulated as single dosage unit comprising suitable buffering agent. All powdered ingredients of said combination are mixed and a suitable quantity of one or more buffering agents is added to the blend to minimize possible interactions.

The agents described herein, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

### Analgesic Agents in Combitherapy

The polypeptides and agonists described herein can be used in combination therapy with an analgesic agent, e.g., an analgesic compound or an analgesic polypeptide. These polypeptides and compounds can be administered with the polypeptides described herein (simultaneously or sequentially). They can also be optionally covalently linked or attached to an agent described herein to create therapeutic conjugates. Among the useful analgesic agents are: Ca channel blockers, 5HT receptor antagonists (for example 5HT3, 5HT4 and 5HT1 receptor antagonists), opioid receptor agonists (loperamide, fedotozine, and fentanyl), NK1 receptor antagonists, CCK receptor agonists (e.g., loxiglumide), NK1 receptor antagonists, NK3 receptor antagonists, norepinephrine-serotonin reuptake inhibitors (NSRI), vanilloid and cannabanoid receptor agonists, and sialorphin. Analgesics agents in the various classes are described in the literature.

Among the useful analgesic polypeptides are sialorphin-related polypeptides, including those comprising the amino acid sequence QHNPR (SEQ ID NO:), including: VQHNPR (SEQ ID NO:); VRQHNPR (SEQ ID NO:); VRGQHNPR (SEQ ID NO:); VRGPQHNPR (SEQ ID NO:); VRGPRQHNPR (SEQ ID NO:); VRGPRRQHNPR (SEQ ID NO:); and RQHNPR (SEQ ID NO:). Sialorphin-related polypeptides bind to neprilysin and inhibit neprilysin-mediated breakdown of substance P and Met-enkephalin. Thus, compounds or polypeptides that are inhibitors of neprilysin are useful analgesic agents which can be administered with the polypeptides described herein in a co-therapy or linked to the polypeptides described herein, e.g., by a covalent bond. Sialophin and related polypeptides are described in U.S. Pat. No. 6,589,750; U.S. 20030078200 A1; and WO 02/051435 A2.

Opioid receptor antagonists and agonists can be administered with the polypeptides described herein in co-therapy or linked to the agent described herein, e.g., by a covalent bond. For example, opioid receptor antagonists such as naloxone, naltrexone, methyl nalozone, nalmefene, cypridime, beta funaltrexamine, naloxonazine, naltrindole, and nor-binaltorphimine are thought to be useful in the treatment of IBS. It can be useful to formulate opioid antagonists of this type is a delayed and sustained release formulation such that initial release of the antagonist is in the mid to distal small intestine and/or ascending colon. Such antagonists are described in WO 01/32180 A2. Enkephalin pentapeptide (HOE825; Tyr-D-Lys-Gly-Phe-L-homoserine) is an agonist of the mu and delta opioid receptors and is thought to be useful for increasing intestinal motility (Eur. J. Pharm. 219:445, 1992), and this polypeptide can be used in conjunction with the polypeptides described herein. Also useful is trimebutine which is thought to bind to mu/delta/kappa opioid receptors and activate release of motilin and modulate the release of gastrin, vasoactive intestinal polypeptide, gastrin and glucagons. Kappa opioid receptor agonists such as fedotozine, asimadoline, and ketocyclazocine, and compounds described in WO03/097051 and WO05/007626 can be used with or linked to the polypeptides described herein. In addition, mu opioid receptor agonists such as morphine, diphenyloxylate, frakefamide (H-Tyr-D-Ala-Phe(F)-Phe-NH₂; WO 01/019849 A1) and loperamide can be used.

Tyr-Arg (kyotorphin) is a dipeptide that acts by stimulating the release of met-enkephalins to elicit an analgesic effect (J. Biol. Chem. 262:8165, 1987). Kyotorphin can be used with or linked to the polypeptides described herein.

Chromogranin-derived polypeptide (CgA 47-66; see, e.g., Ghia et al. 2004 Regulatory polypeptides 119:199) can be used with or linked to the polypeptides described herein.

CCK receptor agonists such as caerulein from amphibians and other species are useful analgesic agents that can be used with or linked to the polypeptides described herein.

Conotoxin polypeptides represent a large class of analgesic polypeptides that act at voltage gated Ca channels, NMDA receptors or nicotinic receptors. These polypeptides can be used with or linked to the polypeptides described herein.

Peptide analogs of thymulin (FR Application 2830451) can have analgesic activity and can be used with or linked to the polypeptides described herein.

CCK (CCKa or CCKb) receptor antagonists, including loxiglumide and dexloxiglumide (the R-isomer of loxiglumide) (WO 88/05774) can have analgesic activity and can be used with or linked to the polypeptides described herein.

Other useful analgesic agents include 5-HT4 agonists such as tegaserod (Zelnorm®), mosapride, metoclopramide, zacopride, cisapride, renzapride, benzimidazolone derivatives such as BIMU 1 and BIMU 8, and lirexapride. Such agonists are described in: EP1321142 A1, WO 03/053432A1, EP 505322 A1, EP 505322 B1, U.S. Pat. No. 5,510,353, EP 507672 A1, EP 507672 B1, and U.S. Pat. No. 5,273,983.

Calcium channel blockers such as ziconotide and related compounds described in, for example, EP625162B1, U.S. Pat. No. 5,364,842, U.S. Pat. No. 5,587,454, U.S. Pat. No. 5,824,645, U.S. Pat. No. 5,859,186, U.S. Pat. No. 5,994,305, U.S. Pat. No. 6,087,091, U.S. Pat. No. 6,136,786, WO 93/13128 A1, EP 1336409 A1, EP 835126 A1, EP 835126 B1, U.S. Pat. No. 5,795,864, U.S. Pat. No. 5,891,849, U.S. Pat. No. 6,054,429, WO 97/01351 A1, can be used with or linked to the polypeptides described herein.

Various antagonists of the NK-1, NK-2, and NK-3 receptors (for a review see Giardina et al. 2003 Drugs 6:758) can be can be used with or linked to the polypeptides described herein.

NK1 receptor antagonists such as: aprepitant (Merck & Co Inc), vofopitant, ezlopitant (Pfizer, Inc.), R-673 (Hoffmann-La Roche Ltd), SR-48968 (Sanofi Synthelabo), CP-122,721 (Pfizer, Inc.), GW679769 (Glaxo Smith Kline), TAK-637 (Takeda/Abbot), SR-14033, and related compounds described in, for example, EP 873753 A1, US 20010006972 A1, US 20030109417 A1, WO 01/52844 A1, can be used with or linked to the polypeptides described herein.

NK-2 receptor antagonists such as nepadutant (Menarini Ricerche SpA), saredutant (Sanofi-Synthelabo), GW597599 (Glaxo Smith Kline), SR-144190 (Sanofi-Synthelabo) and UK-290795 (Pfizer Inc) can be used with or linked to the polypeptides described herein.

NK3 receptor antagonists such as osanetant (SR-142801; Sanofi-Synthelabo), SSR-241586, talnetant and related compounds described in, for example, WO 02/094187 A2, EP 876347 A1, WO 97/21680 A1, U.S. Pat. No. 6,277,862, WO 98/11090, WO 95/28418, WO 97/19927, and Boden et al. (J Med Chem. 39:1664-75, 1996) can be used with or linked to the polypeptides described herein.

Norepinephrine-serotonin reuptake inhibitors (NSRI) such as milnacipran and related compounds described in WO 03/077897 A1 can be used with or linked to the polypeptides described herein.

Vanilloid receptor antagonists such as arvanil and related compounds described in WO 01/64212 A1 can be used with or linked to the polypeptides described herein.

The analgesic polypeptides and compounds can be administered with the polypeptides and agonists described herein (simultaneously or sequentially). The analgesic agents can also be covalently linked to the polypeptides and agonists described herein to create therapeutic conjugates. Where the analgesic is a polypeptide and is covalently linked to an agent described herein the resulting polypeptide may also include at least one trypsin cleavage site. When present within the polypeptide, the analgesic polypeptide may be preceded by (if it is at the carboxy terminus) or followed by (if it is at the amino terminus) a trypsin cleavage site that allows release of the analgesic polypeptide.

In addition to sialorphin-related polypeptides, analgesic polypeptides include: AspPhe, endomorphin-1, endomorphin-2, nocistatin, dalargin, lupron, ziconotide, and substance P.

### Diabetes, Obesity and Other Disorders

Pharmaceutical compositions comprising at least two of: 1) an agent that stimulates the production of cAMP (e.g., glucagon-like polypeptide 1 (GLP-1)); 2) an agent that inhibits the degradation of a cyclic nucleotide (e.g., a phosphodiesterase inhibitor); and 3) a polypeptide or agonist described herein useful for treating diabetes and obesity. Such compositions may also be useful for treating secondary hyperglycemias in connection with pancreatic diseases (chronic pancreatitis, pancreasectomy, hemochromatosis) or endocrine diseases (acromegaly, Cushing's syndrome, pheochromocytoma or hyperthyreosis), drug-induced hyperglycemias (benzothiadiazine saluretics, diazoxide or glucocorticoids), pathologic glucose tolerance, hyperglycemias, dyslipoproteinemias, adiposity, hyperlipoproteinemias and/or hypotensions. The phosphodiesterase inhibitor can be specific for a particular phosphodiesterase (e.g., Group III or Group IV) or a non-specific phosphodiesterase inhibitor, such as papaverine, theophylline, enprofyllines and/or IBMX. Specific phosphodiesterase inhibitors which inhibit group III phosphodiesterases (cGMP-inhibited phosphodiesterases), including indolidane (LY195115), cilostamide (OPC 3689), lixazinone (RS 82856), Y-590, imazodane (CI914), SKF 94120, quazinone, ICI 153,110, cilostazole, bemorandane (RWJ 22867), siguazodane (SK&F 94-836), adibendane (BM 14,478), milrinone (WIN 47203), enoximone (MDL 17043), pimobendane (UD-CG 115), MCI-154, saterinone (BDF 8634), sulmazole (ARL 115), UD-CG 212, motapizone, piroximone, and ICI 118233 can be useful. In addition, phosphodiesterase inhibitors which inhibit group IV phosphodiesterases (cAMP-specific phosphodiesterases), such as rolipram ZK 62711; pyrrolidone), imidazolidinone (RO 20-1724), etazolate (SQ 65442), denbufylline (BRL 30892), ICI63197, and RP73401 can be used.

### Other Agents for Use in Combitherapy

Also within the invention are pharmaceutical compositions comprising a polypeptide or agonists described herein and a second therapeutic agent. The second therapeutic agent can be administered to treat any condition for which it is useful, including conditions that are not considered to be the primary indication for treatment with the second therapeutic agent. The second therapeutic agent can be administered simultaneously or sequentially. The second therapeutic agent can be covalently linked to the polypeptides and agonists described herein to create a therapeutic conjugate. When the second therapeutic agent is another polypeptide, a linker including those described herein may be used between the polypeptide described herein and the second therapeutic polypeptide.

Examples of additional therapeutic agents to treat gastrointestinal and other disorders include

agents to treat constipation (e.g., a chloride channel activator such as the bicylic fatty acid, Lubiprostone (formerly known as SPI-0211; Sucampo Pharmaceuticals, Inc.; Bethesda, Md.), a laxative (eg. a bulk-forming laxative (e.g. nonstarch polysaccharides, Colonel Tablet (polycarbophil calcium), Plantago Ovata®, Equalactin® (Calcium Polycarbophil)), fiber (e.g. FIBERCON® (Calcium Polycarbophil), an osmotic laxative, a stimulant laxative (such as diphenylmethanes (e.g. bisacodyl), anthraquinones (e.g. cascara, senna), and surfactant laxatives (e.g. castor oil, docusates), an emollient/lubricating agent (such as mineral oil, glycerine, and docusates), MiraLax (Braintree Laboratories, Braintree Mass.), dexloxiglumide (Forest Laboratories, also known as CR 2017 Rottapharm (Rotta Research Laboratorium SpA)), saline laxatives, enemas, suppositories, and CR 3700 (Rottapharm (Rotta Research Laboratorium SpA);
acid reducing agents such as proton pump inhibitors (e.g., omeprazole (Prilosec®), esomeprazole (Nexium®), lansoprazole (Prevacid®), pantoprazole (Protonix®) and rabeprazole (Aciphex®)) and Histamine H2-receptor antagonist (also known as H2 receptor blockers including cimetidine, ranitidine, famotidine and nizatidine);
prokinetic agents including itopride, octreotide, bethanechol, metoclopramide (Reglan®), domperidone (Motilium®), erythromycin (and derivatives thereof) or cisapride (Propulsid®);
Prokineticin polypeptides homologs, variants and chimeras thereof including those described in U.S. Pat. No. 7,052,674 which can be used with or linked to the polypeptides described herein;
pro-motility agents such as the vasostatin-derived polypeptide, chromogranin A (4-16) (see, e.g., Ghia et al. 2004 Regulatory polypeptides 121:31) or motilin agonists (e.g., GM-611 or mitemcinal fumarate) or nociceptin/Orphanin FQ receptor modulators (US20050169917);
other peptides which can bind to and/or activate GC-C including those described in US20050287067;
complete or partial 5HT (e.g. 5HT1, 5HT2, 5HT3, 5HT4) receptor agonists or antagonists (including 5HT1A antagonists (e.g. AGI-001 (AGI therapeutics), 5HT2B antagonists (e.g. PGN1091 and PGN1164 (Pharmagene Laboratories Limited), and 5HT4 receptor agonists (such as tegaserod (ZELNORM®), prucalopride, mosapride, metoclopramide, zacopride, cisapride, renzapride, benzimidazolone derivatives such as BIMU 1 and BIMU 8, and lirexapride). Such agonists/modulatos are described in: EP1321142 A1, WO 03/053432A1, EP 505322 A1, EP 505322 B1, U.S. Pat. No. 5,510,353, EP 507672 A1, EP 507672 B1, U.S. Pat. No. 5,273,983, and U.S. Pat. No. 6,951,867); 5HT3 receptor agonists such as MKC-733; and 5HT3 receptor antagonists such as DDP-225 (MCI-225; Dynogen Pharmaceuticals, Inc.), cilansetron (Calmactin®), alosetron (Lotronex®), Ondansetron HCl (Zofran®), Dolasetron (ANZEMET®), palonosetron (Aloxi®), Granisetron (Kytril®), YM060 (ramosetron; Astellas Pharma Inc.; ramosetron may be given as a daily dose of 0.002 to 0.02 mg as described in EP01588707) and ATI-7000 (Aryx Therapeutics, Santa Clara Calif.);
muscarinic receptor agonists;
anti-inflammatory agents;
antispasmodics including but not limited to anticholinergic drugs (like dicyclomine (e.g. Colimex®, Formulex®, Lomine®, Protylol®, Viscerol®, Spasmoban®, Bentyl®, Bentylol®), hyoscyamine (e.g. IB-Stat®, Nulev®, Levsin®, Levbid®, Levsinex Timecaps®, Levsin/SL®, Anaspaz®, A-Spas S/L®, Cystospaz®, Cystospaz-M®, Donnamar®, Colidrops Liquid Pediatric®, Gastrosed®, Hyco Elixir®, Hyosol®, Hyospaz®, Hyosyne®, Losamine®, Medispaz®, Neosol®, Spacol®, Spasdel®, Symax®, Symax SL®), Donnatal (e.g. Donnatal Extentabs®), clidinium (e.g. Quarzan, in combination with Librium=Librax), methantheline (e.g. Banthine), Mepenzolate (e.g. Cantil), homatropine (e.g. hycodan, Homapin), Propantheline bromide (e.g. Pro-Banthine), Glycopyrrolate (e.g. Robinul®, Robinul Forte®), scopolamine (e.g. Transderm-Scop®, Transderm-V®), hyosine-N-butylbromide (e.g. Buscopan®), Pirenzepine (e.g. Gastrozepin®) Propantheline Bromide (e.g. Propanthel®), dicycloverine (e.g. Merbentyl®), glycopyrronium bromide (e.g. Glycopyrrolate®), hyoscine hydrobromide, hyoscine methobromide, methanthelinium, and octatropine); peppermint oil; and direct smooth muscle relaxants like cimetropium bromide, mebeverine (DUSPATAL®, DUSPATALIN®, COLOFAC MR®, COLOTAL®), otilonium bromide (octilonium), pinaverium (e.g. Dicetel® (pinaverium bromide; Solvay S.A.)), Spasfon® (hydrated phloroglucinol and trimethylphloroglucinol) and trimebutine (including trimebutine maleate (Modulon®);
antidepressants, including but not limited to those listed herein, as well as tricyclic antidepressants like amitriptyline (Elavil®), desipramine (Norpramin®), imipramine (Tofranil®), amoxapine (Asendin®), nortriptyline; the selective serotonin reuptake inhibitors (SSRI's) like paroxetine (Paxil®), fluoxetine (Prozac®), sertraline (Zoloft®), and citralopram (Celexa®); and others like doxepin (Sinequan®) and trazodone (Desyrel®);
centrally-acting analgesic agents such as opioid receptor agonists, opioid receptor antagonists (e.g., naltrexone);
agents for the treatment of Inflammatory bowel disease;
agents for the treatment of Crohn's disease and/or ulcerative colitis (e.g., alequel (Enzo Biochem, Inc.; Farmingsale, N.Y.), the anti-inflammatory polypeptide RDP58 (Genzyme, Inc.; Cambridge, Mass.), and TRAFICET-EN™ (ChemoCentryx, Inc.; San Carlos, Calif.);
agents that treat gastrointestinal or visceral pain;
agents that increase cGMP levels (as described in US20040121994) like adrenergic receptor antagonists, dopamine receptor agonists and PDE (phosphodiesterase) inhibitors including but not limited to those disclosed herein;
purgatives that draw fluids to the intestine (e.g., VISICOL®, a combination of sodium phosphate monobasic monohydrate and sodium phosphate dibasic anhydrate);
Corticotropin Releasing Factor (CRF) receptor antagonists (including NBI-34041 (Neurocrine Biosciences, San Diego, Calif.), CRH9-41, astressin, R121919 (Janssen Pharmaceutica), CP154,526, NBI-27914, Antalarmin, DMP696 (Bristol-Myers Squibb) CP-316,311 (Pfizer, Inc.), SB723620 (GSK), GW876008 (Neurocrine/Glaxo Smith Kline), ONO-2333Ms (Ono Pharmaceuticals), TS-041 (Janssen), AAG561 (Novartis) and those disclosed in U.S. Pat. No. 5,063,245, U.S. Pat. No. 5,861,398, US20040224964, US20040198726, US20040176400, US20040171607, US20040110815, US20040006066, and US20050209253);
glucagon-like polypeptides (glp-1) and analogues thereof (including exendin-4 and GTP-010 (Gastrotech Pharma A)) and inhibitors of DPP-IV (DPP-IV mediates the inactivation of glp-1);
tofisopam, enantiomerically-pure R-tofisopam, and pharmaceutically-acceptable salts thereof (US 20040229867);
tricyclic anti-depressants of the dibenzothiazepine type including but not limited to Dextofisopam® (Vela Pharmaceuticals), tianeptine (Stablon®) and other agents described in U.S. Pat. No. 6,683,072;
(E)-4 (1,3bis(cyclohexylmethyl)-1,2,34,-tetrahydro-2,6-diono-9H-purin-8-yl)cinnamic acid nonaethylene glycol methyl ether ester and related compounds described in WO 02/067942;
the probiotic PROBACTRIX® (The BioBalance Corporation; New York, N.Y.) which contains microorganisms useful in the treatment of gastrointestinal disorders;
antidiarrheal drugs including but not limited to loperamide (Imodium, Pepto Diarrhea), diphenoxylate with atropine (Lomotil, Lomocot), cholestyramine (Questran, Cholybar), atropine (Co-Phenotrope, Diarsed, Diphenoxylate, Lofene, Logen, Lonox, Vi-Atro, atropine sulfate injection) and Xifaxan® (rifaximin; Salix Pharmaceuticals Ltd), TZP-201 (Tranzyme Pharma Inc.), the neuronal acetylcholine receptor (nAChR) blocker AGI-004 (AGI therapeutics), and bismuth subsalicylate (Pepto-bismol);
anxiolytic drugs including but not limited to Ativan (lorazepam), alprazolam (Xanax®), chlordiazepoxide/clidinium (Librium®, Librax®), clonazepam (Klonopin®), clorazepate (Tranxene®), diazepam (Valium®), estazolam (ProSom®), flurazepam (Dalmane®), oxazepam (Serax®), prazepam (Centrax®), temazepam (Restoril®), triazolam (Halcion®;
Bedelix® (Montmorillonite beidellitic; Ipsen Ltd), Solvay SLV332 (ArQule Inc), YKP (SK Pharma), Asimadoline (Tioga Pharmaceuticals/Merck), AGI-003 (AGI Therapeutics);
neurokinin antagonists including those described in US20060040950;
potassium channel modulators including those described in US7,002,015;
the serotonin modulator AZD7371 (AstraZeneca Plc);
M3 muscarinic receptor antagonists such as darifenacin (Enablex; Novartis AG and zamifenacin (Pfizer);
herbal and natural therapies including but not limited to acidophilus, chamomile tea, evening primrose oil, fennel seeds, wormwood, comfrey, and compounds of Bao-Ji-Wan (magnolol, honokiol, imperatorin, and isoimperatorin) as in U.S. Pat. No. 6,923,992; and
compositions comprising lysine and an anti-stress agent for the treatment of irritable bowel syndrome as described in EP01550443.

The polypeptides and agonists described herein can be used in combination therapy with insulin and related compounds including primate, rodent, or rabbit insulin including biologically active variants thereof including allelic variants, more preferably human insulin available in recombinant form. Sources of human insulin include pharmaceutically acceptable and sterile formulations such as those available from Eli Lilly (Indianapolis, Ind. 46285) as Humulin™ (human insulin rDNA origin). See the THE PHYSICIAN'S DESK REFERENCE, 55.sup.th Ed. (2001) Medical Economics, Thomson Healthcare (disclosing other suitable human insulins). The polypeptides and agonists described herein can also be used in combination therapy with agents that can boost insulin effects or levels of a subject upon administration, e.g. glipizide and/or rosiglitazone. The polypeptides and agonists described herein can be used in combitherapy with SYMLIN® (pramlintide acetate) and Exenatide® (synthetic exendin-4; a 39 aa polypeptide).

The polypeptides and agonists described herein can also be used in combination therapy with agents (e.g., Entereg™ (alvimopan; formerly called adolor/ADL 8-2698), conivaptan and related agents describe in U.S. Pat. No. 6,645,959) used for the treatment of postoperative ileus and other disorders.

The polypeptides and agonists described herein can be used in combination therapy with an anti-hypertensive agent including but not limited to:

(1) diuretics, such as thiazides, including chlorthalidone, chlorthiazide, dichlorophenamide, hydroflumethiazide, indapamide, polythiazide, and hydrochlorothiazide; loop diuretics, such as bumetanide, ethacrynic acid, furosemide, and torsemide; potassium sparing agents, such as amiloride, and triamterene; carbonic anhydrase inhibitors, osmotics (such as glycerin) and aldosterone antagonists, such as spironolactone, epirenone, and the like;
(2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like;
(3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like;
(4) angiotensin converting enzyme (ACE) inhibitors such as benazepril; captopril; ceranapril; cilazapril; delapril; enalapril; enalopril; fosinopril; imidapril; lisinopril; losinopril; moexipril; quinapril; quinaprilat; ramipril; perindopril; perindropril; quanipril; spirapril; tenocapril; trandolapril, and zofenopril, and the like;
(5) neutral endopeptidase inhibitors such as omapatrilat, cadoxatril and ecadotril, fosidotril, sampatrilat, AVE7688, ER4030, and the like;
(6) endothelin antagonists such as tezosentan, A308165, and YM62899, and the like;
(7) vasodilators such as hydralazine, clonidine, minoxidil, and nicotinyl alcohol, and the like;
(8) angiotensin II receptor antagonists such as aprosartan, candesartan, eprosartan, irbesartan, losartan, olmesartan, pratosartan, tasosartan, telmisartan, valsartan, and EXP-3137, FI6828K, and RNH6270, and the like;
(9) α/β adrenergic blockers such as nipradilol, arotinolol and amosulalol, and the like;
(10) alpha 1 blockers, such as terazosin, urapidil, prazosin, tamsulosin, bunazosin, trimazosin, doxazosin, naftopidil, indoramin, WHP 164, and XEN010, and the like;
(11) alpha 2 agonists such as lofexidine, tiamenidine, moxonidine, rilmenidine and guanobenz, and the like;
(12) aldosterone inhibitors, and the like; and
(13) angiopoietin-2-binding agents such as those disclosed in WO03/030833.

Specific anti-hypertensive agents that can be used in combination with polypeptides and agonists described herein include, but are not limited to:

diuretics, such as thiazides (e.g., chlorthalidone, cyclothiazide (CAS RN 2259-96-3), chlorothiazide (CAS RN 72956-09-3, which may be prepared as disclosed in US2809194), dichlorophenamide, hydroflumethiazide, indapamide, polythiazide, bendroflumethazide, methyclothazide, polythiazide, trichlormethazide, chlorthalidone, indapamide, metolazone, quinethazone, althiazide (CAS RN 5588-16-9, which may be prepared as disclosed in British Patent No. 902,658), benzthiazide (CAS RN 91-33-8, which may be prepared as disclosed in U.S. Pat. No. 3,108,097), buthiazide (which may be prepared as disclosed in British Patent Nos. 861,367), and hydrochlorothiazide), loop diuretics (e.g. bumetanide, ethacrynic acid, furosemide, and torasemide), potassium sparing agents (e.g. amiloride, and triamterene (CAS Number 396-01-0)), and aldosterone antagonists (e.g. spironolactone (CAS Number 52-01-7), epirenone, and the like); β-adrenergic blockers such as Amiodarone (Cordarone, Pacerone), bunolol hydrochloride (CAS RN 31969-05-8, Parke-Davis), acebutolol (±N-[3-Acetyl-4-[2-hydroxy-3-[(1 methylethyl)amino]propoxy]phenyl]-butanamide, or (±)-3′-Acetyl-4′-[2-hydroxy-3-(isopropylamino) propoxy]butyranilide), acebutolol hydrochloride (e.g. Sectral®, Wyeth-Ayerst), alprenolol hydrochloride (CAS RN 13707-88-5 see Netherlands Patent Application No. 6,605,692), atenolol (e.g. Tenormin®, AstraZeneca), carteolol hydrochloride (e.g. Cartrol® Filmtab®, Abbott), Celiprolol hydrochloride (CAS RN 57470-78-7, also see in U.S. Pat. No. 4,034,009), cetamolol hydrochloride (CAS RN 77590-95-5, see also U.S. Pat. No. 4,059,622), labetalol hydrochloride (e.g. Normodyne®, Schering), esmolol hydrochloride (e.g. Brevibloc®, Baxter), levobetaxolol hydrochloride (e.g. Betaxon™ Ophthalmic Suspension, Alcon), levobunolol hydrochloride (e.g. Betagan® Liquifilm® with C CAP® Compliance Cap, Allergan), nadolol (e.g. Nadolol, Mylan), practolol (CAS RN 6673-35-4, see also U.S. Pat. No. 3,408,387), propranolol hydrochloride (CAS RN 318-98-9), sotalol hydrochloride (e.g. Betapace AF™, Berlex), timolol (2-Propanol, 1-[(1,1-dimethylethyl)amino]-3-[[4-4(4-morpholinyl)-1,2,5-thiadiazol-3-yl]oxy]-, hemihydrate, (S)-, CAS RN 91524-16-2), timolol maleate (S)-1-[(1,1-dimethylethyl)amino]-3-[[4-(4-morpholinyl)-1,2,5-thiadiazol-3-yl]oxy]-2-propanol (Z)-2-butenedioate (1:1) salt, CAS RN 26921-17-5), bisoprolol (2-Propanol, 1-[4-[[2-(1-methylethoxy)ethoxy]-methyl]phenoxyl]-3-[(1-meth-ylethyl)amino]-, (±), CAS RN 66722-44-9), bisoprolol fumarate (such as (±)-1-[4-[[2-(1-Methylethoxy)ethoxy]methyl]phenoxy]-3-[(1-methylethyl)amino]-2-propanol(E)-2-butenedioate (2:1) (salt), e.g., Zebeta™, Lederle Consumer), nebivalol (2H-1-Benzopyran-2-methanol, αα′-[iminobis(methylene)]bis[6-fluoro-3,4-dihydro-, CAS RN 99200-09-6 see also U.S. Pat. No. 4,654,362), cicloprolol hydrochloride, such 2-Propanol, 1-[4-[2-(cyclopropylmethoxy)ethoxy]phenoxy]-3-[1-methylethyl)amino]-, hydrochloride, A.A.S. RN 63686-79-3), dexpropranolol hydrochloride (2-Propano-1,1-[1-methylethy)-amino]-3-(1-naphthalenyloxy)-hydrochloride (CAS RN 13071-11-9), diacetolol hydrochloride (Acetamide, N-[3-acetyl-4-[2-hydroxy-3-[(1-methyl-ethyl)amino]propoxy][phenyl]-, monohydrochloride CAS RN 69796-04-9), dilevalol hydrochloride (Benzamide, 2-hydroxy-5-[1-hydroxy-2-[1-methyl-3-phenylpropyl)amino]ethyl]-, monohydrochloride, CAS RN 75659-08-4), exaprolol hydrochloride (2-Propanol, 1-(2-cyclohexylphenoxy)-3-[(1-methylethyl)amino]-, hydrochloride CAS RN 59333-90-3), flestolol sulfate (Benzoic acid, 2-fluoro-,3-[[2-[aminocarbonyl)amino]-dimethylethyl]amino]-2-hydroxypropyl ester, (±)-sulfate (1:1) (salt), CAS RN 88844-73-9; metalol hydrochloride (Methanesulfonamide, N-[4-[1-hydroxy-2-(methylamino)propyl]phenyl]-, monohydrochloride CAS RN 7701-65-7), metoprolol 2-Propanol, 1-[4-(2-methoxyethyl)phenoxy]-3-[1-methylethyl)amino]-; CAS RN 37350-58-6), metoprolol tartrate (such as 2-Propanol, 1-[4-(2-methoxyethyl)phenoxy]-3-[(1-methylethyl)amino]-, e.g., Lopressor®, Novartis), pamatolol sulfate (Carbamic acid, [2-[4-[2-hydroxy-3-[(1-methylethyl)amino]propoxyl]phenyl]-ethyl]-, methyl ester, (±) sulfate (salt) (2:1), CAS RN 59954-01-7), penbutolol sulfate (2-Propanol, 1-(2-cyclopentylphenoxy)-3-[1,1-dimethylethyl)amino]1, (S)—, sulfate (2:1) (salt), CAS RN 38363-32-5), practolol (Acetamide, N-[4-[2-hydroxy-3-[(1-methylethyl)amino]-propoxy]phenyl]-, CAS RN 6673-35-4;) tiprenolol hydrochloride (Propanol, 1-[(1-methylethyl)amino]-3-[2-(methylthio)-phenoxy]-, hydrochloride, (±), CAS RN 39832-43-4), tolamolol (Benzamide, 4-[2-[[2-hydroxy-3-(2-methylphenoxy)-propyl]amino]ethoxyl]-, CAS RN 38103-61-6), bopindolol, indenolol, pindolol, propanolol, tertatolol, and tilisolol, and the like; calcium channel blockers such as besylate salt of amlodipine (such as 3-ethyl-5-methyl-2-(2-aminoethoxymethyl)-4-(2-chlorophenyl)-1,4-dihydro-6-methyl-3,5-pyridinedicarboxylate benzenesulphonate, e.g., Norvasc®, Pfizer), clentiazem maleate (1,5-Benzothiazepin-4(5H)-one, 3-(acetyloxy)-8-chloro-5-[2-(dimethylamino)ethyl]-2,3-dihydro-2-(4-methoxyphenyl)-(2S-cis)-, (Z)-2-butenedioate (1:1), see also U.S. Pat. No. 4,567,195), isradipine (3,5-Pyridinedicarboxylic acid, 4-(4-benzofurazanyl)-1,4-dihydro-2,6-dimethyl-, methyl 1-methylethyl ester, (±)-4(4-benzofurazanyl)-1,4-dihydro-2,6-dimethyl-3,5-pyridinedicarboxylate, see also U.S. Pat. No. 4,466,972); nimodipine (such as is isopropyl (2-methoxyethyl) 1,4-dihydro-2,6-dimethyl-4-(3-nitrophenyl)-3,5-pyridine-dicarboxylate, e.g. Nimotop®, Bayer), felodipine (such as ethyl methyl 4-(2,3-dichlorophenyl)-1,4-dihydro-2,6-dimethyl-3,5-pyridinedicarboxylate-, e.g. Plendil® Extended-Release, AstraZeneca LP), nilvadipine (3,5-Pyridinedicarboxylic acid, 2-cyano-1,4-dihydro-6-methyl-4-(3-nitrophenyl)-,3-methyl 5-(1-methylethyl) ester, also see U.S. Pat. No. 3,799,934), nifedipine (such as 3,5-pyridinedicarboxylic acid, 1,4-dihydro-2,6-dimethyl-4-(2-nitrophenyl)-, dimethyl ester, e.g., Procardia XL® Extended Release Tablets, Pfizer), diltiazem hydrochloride (such as 1,5-Benzothiazepin-4(5H)-one,3-(acetyloxy)-5[2-(dimethylamino)ethyl]-2,-3-dihydro-2(4-methoxyphenyl)-, monohydrochloride, (+)-cis., e.g., Tiazac®, Forest), verapamil hydrochloride (such as benzeneacetronitrile, (alpha)-[[3-[[2-(3,4-dimethoxyphenyl)ethyl]methylamino]propyl]-3,4-dimethoxy-(alpha)-(1-methylethyl) hydrochloride, e.g., Isoptin® SR, Knoll Labs), teludipine hydrochloride (3,5-Pyridinedicarboxylic acid, 2-[(dimethylamino)methyl]4-[2-[(1E)-3-(1,1-dimethylethoxy)-3-oxo-1-propenyl]phenyl]-1,4-dihydro-6-methyl-, diethyl ester, monohydrochloride) CAS RN 108700-03-4), belfosdil (Phosphonic acid, [2-(2-phenoxyethyl)-1,3-propane-diyl]bis-, tetrabutyl ester CAS RN 103486-79-9), fostedil (Phosphonic acid, [[4-(2-benzothiazolyl)phenyl]methyl]-, diethyl ester CAS RN 75889-62-2), aranidipine, azelnidipine, barnidipine, benidipine, bepridil, cinaldipine, clevidipine, efonidipine, gallopamil, lacidipine, lemildipine, lercanidipine, monatepil maleate (1-piperazinebutanamide, N-(6,11-dihydrodibenzo(b,e)thiepin-11-yl)₄-(4-fluorophenyl)-, (±)-, (Z)-2-butene dioate (1:1) (±)—N-(6,11-Dihydrodibenzo(b,e)thiep-in-11-yl)-4-(p-fluorophenyl)-1-piperazinebutyramide maleate (1:1) CAS RN 132046-06-1), nicardipine, nisoldipine, nitrendipine, manidipine, pranidipine, and the like; T-channel calcium antagonists such as mibefradil; angiotensin converting enzyme (ACE) inhibitors such as benazepril, benazepril hydrochloride (such as 3-[[1-(ethoxycarbonyl)-3-phenyl-(1S)-propyl]amino]-2,3,4,5-tetrahydro-2-oxo-1H-1-(3S)-benzazepine-1-acetic acid monohydrochloride, e.g., Lotrel®, Novartis), captopril (such as 1-[(2S)-3-mercapto-2-methylpropionyl]-L-proline, e.g., Captopril, Mylan, CAS RN 62571-86-2 and others disclosed in U.S. Pat. No. 4,046,889), ceranapril (and others disclosed in U.S. Pat. No. 4,452,790), cetapril (alacepril, Dainippon disclosed in Eur. Therap. Res. 39:671 (1986); 40:543 (1986)), cilazapril (Hoffman-LaRoche) disclosed in J. Cardiovasc. Pharmacol. 9:39 (1987), indalapril (delapril hydrochloride (2H-1,2,4-Benzothiadiazine-7-sulfonamide, 3-bicyclo[2.2.1]hept-5-en-2-yl-6-chloro-3,4-dihydro-, 1,1-dioxide CAS RN 2259-96-3); disclosed in U.S. Pat. No. 4,385,051), enalapril (and others disclosed in U.S. Pat. No. 4,374,829), enalopril, enaloprilat, fosinopril, ((such as L-proline, 4-cyclohexyl-1-[[[2-methyl-1-(1-oxopropoxy)propoxy](4-phenylbutyl)phosphinyl]acetyl]-, sodium salt, trans—, e.g., Monopril, Bristol-Myers Squibb and others disclosed in U.S. Pat. No. 4,168,267), fosinopril sodium (L-Proline, 4-cyclohexyl-1-[[(R)-[(1S)-2-methyl-1-(1-ox-opropoxy)propox), imidapril, indolapril (Schering, disclosed in J. Cardiovasc. Pharmacol. 5:643, 655 (1983)), lisinopril (Merck), losinopril, moexipril, moexipril hydrochloride (3-Isoquinolinecarboxylic acid, 2-[(2S)-2-[[(1S)-1-(ethoxycarbonyl)-3-phenylpropyl]amino]-1-oxopropyl]-1,-2,3,4-tetrahydro-6,7-dimethoxy-, monohydrochloride, (3S)-CAS RN 82586-52-5), quinapril, quinaprilat, ramipril (Hoechsst) disclosed in EP 79022 and Curr. Ther. Res. 40:74 (1986), perindopril erbumine (such as 2S,3aS,7aS-1-[(S)—N—[(S)-1-Carboxybutyl]alanyl]hexahydro-2-indolinecarboxylic acid, 1-ethyl ester, compound with tert-butylamine (1:1), e.g., Aceon®, Solvay), perindopril (Servier, disclosed in Eur. J. din. Pharmacol. 31:519 (1987)), quanipril (disclosed in U.S. Pat. No. 4,344,949), spirapril (Schering, disclosed in Acta. Pharmacol. Toxicol. 59 (Supp. 5):173 (1986)), tenocapril, trandolapril, zofenopril (and others disclosed in U.S. Pat. No. 4,316,906), rentiapril (fentiapril, disclosed in Clin. Exp. Pharmacol. Physiol. 10:131 (1983)), pivopril, YS980, teprotide (Bradykinin potentiator BPP9a CAS RN 35115-60-7), BRL 36,378 (Smith Kline Beecham, see EP80822 and EP60668), MC-838 (Chugai, see C.A. 102:72588v and Jap. J. Pharmacol. 40:373 (1986), CGS 14824 (Ciba-Geigy, 3-([1-ethoxycarbonyl-3-phenyl-(15)-propyl]amino)-2,3,4,5-tetrahydro-2-ox-o-1-(3S)-benzazepine-1 acetic acid HCl, see U.K. Patent No. 2103614), CGS 16,617 (Ciba-Geigy, 3(S)-[[(1S)-5-amino-1-carboxypentyl]amino]-2,3,4,-5-tetrahydro-2-oxo-1H-1-benzazepine-1-ethanoic acid, see U.S. Pat. No. 4,473,575), Ru 44570 (Hoechst, see Arzneimittelforschung 34:1254 (1985)), R 31-2201 (Hoffman-LaRoche see FEBS Lett. 165:201 (1984)), C1925 (Pharmacologist 26:243, 266 (1984)), WY-44221 (Wyeth, see J. Med. Chem. 26:394 (1983)), and those disclosed in US2003006922 (paragraph 28), U.S. Pat. No. 4,337,201, U.S. Pat. No. 4,432,971 (phosphonamidates); neutral endopeptidase inhibitors such as omapatrilat (Vanlev®), CGS 30440, cadoxatril and ecadotril, fasidotril (also known as aladotril or alatriopril), sampatrilat, mixanpril, and gemopatrilat, AVE7688, ER4030, and those disclosed in U.S. Pat. No. 5,362,727, U.S. Pat. No. 5,366,973, U.S. Pat. No. 5,225,401, U.S. Pat. No. 4,722,810, U.S. Pat. No. 5,223,516, U.S. Pat. No. 4,749,688, U.S. Pat. No. 5,552,397, U.S. Pat. No. 5,504,080, U.S. Pat. No. 5,612,359, U.S. Pat. No. 5,525,723, EP0599444, EP0481522, EP0599444, EP0595610, EP0534363, EP534396, EP534492, EP0629627;
endothelin antagonists such as tezosentan, A308165, and YM62899, and the like; vasodilators such as hydralazine (apresoline), clonidine (clonidine hydrochloride (1H-Imidazol-2-amine, N-(2,6-dichlorophenyl)4,5-dihydro-, monohydrochloride CAS RN 4205-91-8), catapres, minoxidil (loniten), nicotinyl alcohol (roniacol), diltiazem hydrochloride (such as 1,5-Benzothiazepin-4(5H)-one,3-(acetyloxy)-5[2-(dimethylamino)ethyl]-2,-3-dihydro-2(4-methoxyphenyl)-, monohydrochloride, (+)-cis, e.g., Tiazac®, Forest), isosorbide dinitrate (such as 1,4:3,6-dianhydro-D-glucitol 2,5-dinitrate e.g., Isordil® Titradose®, Wyeth-Ayerst), sosorbide mononitrate (such as 1,4:3,6-dianhydro-D-glucito-1,5-nitrate, an organic nitrate, e.g., Ismo®, Wyeth-Ayerst), nitroglycerin (such as 2,3 propanetriol trinitrate, e.g., Nitrostat® Parke-Davis), verapamil hydrochloride (such as benzeneacetonitrile, (±)-(alpha)[3-[[2-(3,4dimethoxyphenyl)ethyl]methylamino]propyl]-3,4-dimethoxy-(alpha)-(1-methylethyl) hydrochloride, e.g., Covera HS® Extended-Release, Searle), chromonar (which may be prepared as disclosed in U.S. Pat. No. 3,282,938), clonitate (Annalen 1870 155), droprenilamine (which may be prepared as disclosed in DE2521113), lidoflazine (which may be prepared as disclosed in U.S. Pat. No. 3,267,104); prenylamine (which may be prepared as disclosed in U.S. Pat. No. 3,152,173), propatyl nitrate (which may be prepared as disclosed in French Patent No. 1,103,113), mioflazine hydrochloride (1-piperazineacetamide, 3-(aminocarbonyl)₄-[4,4-bis(4-fluorophenyl)butyl]-N-(2,6-dichlorophenyl)-, dihydrochloride CAS RN 83898-67-3), mixidine (Benzeneethanamine, 3,4-dimethoxy-N-(1-methyl-2-pyrrolidinylidene)-Pyrrolidine, 2-[(3,4-dimethoxyphenethypimino]-1-methyl-1-Methyl-2-[(3,4-dimethoxyphenethyl)imino]pyrrolidine CAS RN 27737-38-8), molsidomine (1,2,3-Oxadiazolium, 5-[(ethoxycarbonyl)amino]-3-(4-morpholinyl)-, inner salt CAS RN 25717-80-0), isosorbide mononitrate (D-Glucitol, 1,4:3,6-dianhydro-, 5-nitrate CAS RN 16051-77-7), erythrityl tetranitrate (1,2,3,4-Butanetetrol, tetranitrate, (2R,3S)-rel-CAS RN 7297-25-8), clonitrate(1,2-Propanediol, 3-chloro-, dinitrate (7CI, 8CI, 9CI) CAS RN 2612-33-1), dipyridamole Ethanol, 2,2′,″, 2′-[(4,8-di-1-piperidinylpyrimido[5,4-d]pyrimidine-2,6-diyl)dinitrilo]tetrakis-CAS RN 58-32-2), nicorandil (CAS RN 65141-46-0 3-), pyridinecarboxamide(N-[2-(nitrooxy)ethyl]-Nisoldipine-3,5-Pyridinedicarboxylic acid, 1,4-dihydro-2,6-dimethyl-4-(2-nitrophenyl)-, methyl 2-methylpropyl ester CAS RN 63675-72-9), nifedipine-3,5-Pyridinedicarboxylic acid, 1,4-dihydro-2,6-dimethyl-4-(2-nitrophenyl)-, dimethyl ester CAS RN 21829-25-4), perhexyline maleate (Piperidine, 2-(2,2-dicyclohexylethyl)-, (2Z)-2-butenedioate (1:1) CAS RN 6724-53-4), oxprenolol hydrochloride (2-Propanol, 1-[(1-methylethyl)amino]-3-[2-(2-propenyloxy)phenoxy]-, hydrochloride CAS RN 6452-73-9), pentrinitrol(1,3-Propanediol, 2,2-bis[(nitrooxy)methyl]-, mononitrate (ester) CAS RN 1607-17-6), verapamil (Benzeneacetonitrile, α-[3-[[2-(3,4-dimethoxyphenyl)ethyl]-methylamino]propyl]-3,4-dimethoxy-α-(1-methylethyl)-CAS RN 52-53-9) and the like; angiotensin II receptor antagonists such as, aprosartan, zolasartan, olmesartan, pratosartan, FI6828K, RNH6270, candesartan (1H-Benzimidazole-7-carboxylic acid, 2-ethoxy-1-[[2′-(1H-tetrazol-5-yl)[1,1′-biphenyl]4-yl]methyl]-CAS RN 139481-59-7), candesartan cilexetil ((+/−)-1-(cyclohexylcarbonyloxy)ethyl-2-ethoxy-1-[[2′-(1H-tetrazol-5-yl)biphenyl-4-yl]-1H-benzimidazole carboxylate, CAS RN 145040-37-5, U.S. Pat. No. 5,703,110 and U.S. Pat. No. 5,196,444), eprosartan (3-[1-4-carboxyphenylmethyl)-2-n-butyl-imidazol-5-yl]-(2-thienylmethyl) propenoic acid, U.S. Pat. No. 5,185,351 and U.S. Pat. No. 5,650,650), irbesartan (2-n-butyl-3-[[2′-(1h-tetrazol-5-yl)biphenyl-4-yl]methyl]1,3-diazazspiro[4,4]non-1-en-4-one, U.S. Pat. No. 5,270,317 and U.S. Pat. No. 5,352,788), losartan (2-N-butyl-4-chloro-5-hydroxymethyl-1-[(2′-(1H-tetrazol-5-yl)biphenyl-4-yl)-methyl]imidazole, potassium salt, U.S. Pat. No. 5,138,069, U.S. Pat. No. 5,153,197 and U.S. Pat. No. 5,128,355), tasosartan (5,8-dihydro-2,4-dimethyl-8-[(2′-(1H-tetrazol-5-yl)[1,1′-biphenyl]4-yl)methyl]-pyrido[2,3-d]pyrimidin-7(6H)-one, U.S. Pat. No. 5,149,699), telmisartan (4′-[(1,4-dimethyl-2′-propyl-(2,6′-bi-1H-benzimidazol)-1′-yl)]-[1,1′-biphenyl]-2-carboxylic acid, CAS RN 144701-48-4, U.S. Pat. No. 5,591,762), milfasartan, abitesartan, valsartan (Diovan® (Novartis), (S)—N-valeryl-N-[[2′-(1H-tetrazol-5-yl)biphenyl-4-yl)methyl]valine, U.S. Pat. No. 5,399,578), EXP-3137 (2-N-butyl-4-chloro-1-[(2′-(1H-tetrazol-5-yl)biphenyl-4-yl)-methyl]imidazole-5-carboxylic acid, U.S. Pat. No. 5,138,069, U.S. Pat. No. 5,153,197 and U.S. Pat. No. 5,128,355), 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-)IH-tetrazol-5-yl)biphenyl-4-ylmethyl]quinazolin-4(3H)-one, 3-[2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl)[1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5[[2′-(1H-tetrazol-5-yl)[1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo[1,5-c]pyrimidin-2(3H)-one, 2,7-diethyl-5-[[2′-(5-tetrazoly)biphenyl-4-yl]methyl]-5H-pyrazolo[1,5-b][1,2,4]triazole potassium salt, 2-[2-butyl-4,5-dihydro-4-oxo-3-[2′-(1H-tetrazol-5-yl)-4-biphenylmethyl]-3H-imidazol[4,5-c]pyridine-5-ylmethyl]benzoic acid, ethyl ester, potassium salt, 3-methoxy-2,6-dimethyl-4-[[2′(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinylisodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidzole-5-carboxylic acid 1-ethoxycarbonyloxyethyl ester, those disclosed in patent publications EP475206, EP497150, EP539086, EP539713, EP535463, EP535465, EP542059, EP497121, EP535420, EP407342, EP415886, EP424317, EP435827, EP433983, EP475898, EP490820, EP528762, EP324377, EP323841, EP420237, EP500297, EP426021, EP480204, EP429257, EP430709, EP434249, EP446062, EP505954, EP524217, EP514197, EP514198, EP514193, EP514192, EP450566, EP468372, EP485929, EP503162, EP533058, EP467207 EP399731, EP399732, EP412848, EP453210, EP456442, EP470794, EP470795, EP495626, EP495627, EP499414, EP499416, EP499415, EP511791, EP516392, EP520723, EP520724, EP539066, EP438869, EP505893, EP530702, EP400835, EP400974, EP401030, EP407102, EP411766, EP409332, EP412594, EP419048, EP480659, EP481614, EP490587, EP467715, EP479479, EP502725, EP503838, EP505098, EP505111 EP513,979 EP507594, EP510812, EP511767, EP512675, EP512676, EP512870, EP517357, EP537937, EP534706, EP527534, EP540356, EP461040, EP540039, EP465368, EP498723, EP498722, EP498721, EP515265, EP503785, EP501892, EP519831, EP532410, EP498361, EP432737, EP504888, EP508393, EP508445, EP403159, EP403158, EP425211, EP427463, EP437103, EP481448, EP488532, EP501269, EP500409, EP540400, EP005528, EP028834, EP028833, EP411507, EP425921, EP430300, EP434038, EP442473, EP443568, EP445811, EP459136, EP483683, EP518033, EP520423, EP531876, EP531874, EP392317, EP468470, EP470543, EP502314, EP529253, EP543263, EP540209, EP449699, EP465323, EP521768, EP415594, WO92/14468, WO93/08171, WO93/08169, WO91/00277, WO91/00281, WO91/14367, WO92/00067, WO92/00977, WO92/20342, WO93/04045, WO93/04046, WO91/15206, WO92/14714, WO92/09600, WO92/16552, WO93/05025, WO93/03018, WO91/07404, WO92/02508, WO92/13853, WO91/19697, WO91/11909, WO91/12001, WO91/11999, WO91/15209, WO91/15479, WO92/20687, WO92/20662, WO92/20661, WO93/01177, WO91/14679, WO91/13063, WO92/13564, WO91/17148, WO91/18888, WO91/19715, WO92/02257, WO92/04335, WO92/05161, WO92/07852, WO92/15577, WO93/03033, WO91/16313, WO92/00068, WO92/02510, WO92/09278, WO9210179, WO92/10180, WO92/10186, WO92/10181, WO92/10097, WO92/10183, WO92/10182, WO92/10187, WO92/10184, WO92/10188, WO92/10180, WO92/10185, WO92/20651, WO93/03722, WO93/06828, WO93/03040, WO92/19211, WO92/22533, WO92/06081, WO92/05784, WO93/00341, WO92/04343, WO92/04059, U.S. Pat. No. 5,104,877, U.S. Pat. No. 5,187,168, U.S. Pat. No. 5,149,699, U.S. Pat. No. 5,185,340, U.S. Pat. No. 4,880,804, U.S. Pat. No. 5,138,069, U.S. Pat. No. 4,916,129, U.S. Pat. No. 5,153,197, U.S. Pat. No. 5,173,494, U.S. Pat. No. 5,137,906, U.S. Pat. No. 5,155,126, U.S. Pat. No. 5,140,037, U.S. Pat. No. 5,137,902, U.S. Pat. No. 5,157,026, U.S. Pat. No. 5,053,329, U.S. Pat. No. 5,132,216, U.S. Pat. No. 5,057,522, U.S. Pat. No. 5,066,586, U.S. Pat. No. 5,089,626, U.S. Pat. No. 5,049,565, U.S. Pat. No. 5,087,702, U.S. Pat. No. 5,124,335, U.S. Pat. No. 5,102,880, U.S. Pat. No. 5,128,327, U.S. Pat. No. 5,151,435, U.S. Pat. No. 5,202,322, U.S. Pat. No. 5,187,159, U.S. Pat. No. 5,198,438, U.S. Pat. No. 5,182,288, U.S. Pat. No. 5,036,048, U.S. Pat. No. 5,140,036, U.S. Pat. No. 5,087,634, U.S. Pat. No. 5,196,537, U.S. Pat. No. 5,153,347, U.S. Pat. No. 5,191,086, U.S. Pat. No. 5,190,942, U.S. Pat. No. 5,177,097, U.S. Pat. No. 5,212,177, U.S. Pat. No. 5,208,234, U.S. Pat. No. 5,208,235, U.S. Pat. No. 5,212,195, U.S. Pat. No. 5,130,439, U.S. Pat. No. 5,045,540, U.S. Pat. No. 5,041,152, and U.S. Pat. No. 5,210,204, and pharmaceutically acceptable salts and esters thereof; α/β adrenergic blockers such as nipradilol, arotinolol, amosulalol, bretylium tosylate (CAS RN: 61-75-6), dihydroergtamine mesylate (such as ergotaman-3′,6′,18-trione,9,-10-dihydro-12′-hydroxy-2′-methyl-5′-(phenylmethyl)-,(5′(α))-, monomethanesulfonate, e.g., DHE 45® Injection, Novartis), carvedilol (such as (±)-1-(Carbazol-4-yloxy)-3-[[2-(o-methoxyphenoxy)ethyl]amino]-2-propanol, e.g., Coreg®, SmithKline Beecham), labetalol (such as 5-[1-hydroxy-2-[(1-methyl-3-phenylpropyl)amino]ethyl]salicylamide monohydrochloride, e.g., Normodyne®, Schering), bretylium tosylate (Benzenemethanaminium, 2-bromo-N-ethyl-N,N-dimethyl-, salt with 4-methylbenzenesulfonic acid (1:1) CAS RN 61-75-6), phentolamine mesylate (Phenol, 3-[[(4,5-dihydro-1H-imidazol-2-yl)methyl](4-methylphenyl)amino]-, monomethanesulfonate (salt) CAS RN 65-28-1), solypertine tartrate (5H-1,3-Dioxolo[4,5-f]indole, 7-[2-[4-(2-methoxyphenyl)-1-piperazinyl]ethyl]-, (2R,3R)-2,3-dihydroxybutanedioate (1:1) CAS RN 5591-43-5), zolertine hydrochloride (piperazine, 1-phenyl-4-[2-(1H-tetrazol-5-yl)ethyl]-, monohydrochloride (8Cl, 9Cl) CAS RN 7241-94-3) and the like;
α adrenergic receptor blockers, such as alfuzosin (CAS RN: 81403-68-1), terazosin, urapidil, prazosin (Minipress®), tamsulosin, bunazosin, trimazosin, doxazosin, naftopidil, indoramin, WHP 164, XEN010, fenspiride hydrochloride (which may be prepared as disclosed in U.S. Pat. No. 3,399,192), proroxan (CAS RN 33743-96-3), and labetalol hydrochloride and combinations thereof; α 2 agonists such as methyldopa, methyldopa HCL, lofexidine, tiamenidine, moxonidine, rilmenidine, guanobenz, and the like;
aldosterone inhibitors, and the like; renin inhibitors including Aliskiren (SPP 100; Novartis/Speedel); angiopoietin-2-binding agents such as those disclosed in WO03/030833; anti-angina agents such as ranolazine (hydrochloridel-piperazineacetamide, N-(2,6-dimethylphenyl)-4-[2-hydroxy-3-(2-methoxyphenoxy)propyl]-, dihydrochloride CAS RN 95635-56-6), betaxolol hydrochloride (2-Propanol, 1-[4-[2 (cyclopropylmethoxy)ethyl]phenoxy]-3-[(1-methylethyl)amino]-, hydrochloride CAS RN 63659-19-8), butoprozine hydrochloride (Methanone, [4-[3(dibutylamino)propoxy]phenyl](2-ethyl-3-indolizinyl)-, monohydrochloride CAS RN 62134-34-3), cinepazet maleatel-piperazineacetic acid, 4-[1-oxo-3-(3,4,5-trimethoxyphenyl)-2-propenyl]-, ethyl ester, (2Z)-2-butenedioate (1:1) CAS RN 50679-07-7), tosifen (Benzenesulfonamide, 4-methyl-N—[[[(1S)-1-methyl-2-phenylethyl]amino]carbonyl]-CAS RN 32295-184), verapamilhydrochloride (Benzeneacetonitrile, α-[3-[[2-(3,4-dimethoxyphenyl)ethyl]methylamino]propyl]-3,4-dimethoxy-α-(1-methylethyl)-, monohydrochloride CAS RN 152-114), molsidomine (1,2,3-Oxadiazolium, 5-[(ethoxycarbonyl)amino]-3-(4-morpholinyl)-, inner salt CAS RN 25717-80-0), and ranolazine hydrochloride (1-piperazineacetamide, N-(2,6-dimethylphenyl)₄-[2-hydroxy-3-(2-meth-oxyphenoxy)propyl]-, dihydrochloride CAS RN 95635-56-6); tosifen (Benzenesulfonamide, 4-methyl-N-[[[(1S)-1-methyl-2-phenylethyl]amino]carbonyl]-CAS RN 32295-184); adrenergic stimulants such as guanfacine hydrochloride (such as N-amidino-2-(2,6-dichlorophenyl) acetamide hydrochloride, e.g., Tenex® Tablets available from Robins); methyldopa-hydrochlorothiazide (such as levo-3-(3,4-dihydroxyphenyl)-2-methylalanine) combined with Hydrochlorothiazide (such as 6-chloro-3,4-dihydro-2H-1,2,4-benzothiadiazine-7-sulfonamide 1,1-dioxide, e.g., the combination as, e.g., Aldoril® Tablets available from Merck), methyldopa-chlorothiazide (such as 6-chloro-2H-1,2,4-benzothiadiazine-7-sulfonamide 1,1-dioxide and methyldopa as described above, e.g., Aldoclor®, Merck), clonidine hydrochloride (such as 2-(2,6-dichlorophenylamino)-2-imidazoline hydrochloride and chlorthalidone (such as 2-chloro-5-(1-hydroxy-3-oxo-1-isoindolinyl) benzenesulfonamide), e.g., Combipres®, Boehringer Ingelheim), clonidine hydrochloride (such as 2-(2,6-dichlorophenylamino)-2-imidazoline hydrochloride, e.g., Catapres®, Boehringer Ingelheim), clonidine (1H-Imidazol-2-amine, N-(2,6-dichlorophenyl)4,5-dihydro-CAS RN 4205-90-7), Hyzaar (Merck; a combination of losartan and hydrochlorothiazide), Co-Diovan (Novartis; a combination of valsartan and hydrochlorothiazide, Lotrel (Novartis; a combination of benazepril and amlodipine) and Caduet (Pfizer; a combination of amlodipine and atorvastatin), and those agents disclosed in US20030069221.

The polypeptides and agonists described herein can be used in combination therapy with one or more of the following agents useful in the treatment of respiratory and other disorders including but not limited to:

(1) β-agonists including but not limited to: albuterol (PROVENTIL®, SALBUTAMOl®, VENTOLIN®), bambuterol, bitoterol, clenbuterol, fenoterol, formoterol, isoetharine (BRONKOSOL®, BRONKOMETER®), metaproterenol (ALUPENT®, METAPREL®), pirbuterol (MAXAIR®), reproterol, rimiterol, salmeterol, terbutaline (BRETHAIRE®, BRETHINE®, BRICANYL®), adrenalin, isoproterenol (ISUPREL®), epinephrine bitartrate (PRIMATENE®), ephedrine, orciprenline, fenoterol and isoetharine;
(2) steroids, including but not limited to beclomethasone, beclomethasone dipropionate, betamethasone, budesonide, bunedoside, butixocort, dexamethasone, flunisolide, fluocortin, fluticasone, hydrocortisone, methyl prednisone, mometasone, predonisolone, predonisone, tipredane, tixocortal, triamcinolone, and triamcinolone acetonide;
(3) β2-agonist-corticosteroid combinations [e.g., salmeterol-fluticasone (ADVAIR®), formoterol-budesonid (SYMBICORT®)];
(4) leukotriene D4 receptor antagonists/leukotriene antagonists/LTD4 antagonists (i.e., any compound that is capable of blocking, inhibiting, reducing or otherwise interrupting the interaction between leukotrienes and the Cys LTI receptor) including but not limited to: zafirlukast, montelukast, montelukast sodium (SINGULAIR®), pranlukast, iralukast, pobilukast, SKB-106,203 and compounds described as having LTD4 antagonizing activity described in U.S. Pat. No. 5,565,473;
(5) 5-lipoxygenase inhibitors and/or leukotriene biosynthesis inhibitors [e.g., zileuton and BAY1005 (CA registry 128253-31-6)];
(6) histamine H1 receptor antagonists/antihistamines (i.e., any compound that is capable of blocking, inhibiting, reducing or otherwise interrupting the interaction between histamine and its receptor) including but not limited to: astemizole, acrivastine, antazoline, azatadine, azelastine, astamizole, bromopheniramine, bromopheniramine maleate, carbinoxamine, carebastine, cetirizine, chlorpheniramine, chloropheniramine maleate, cimetidine, clemastine, cyclizine, cyproheptadine, descarboethoxyloratadine, dexchlorpheniramine, dimethindene, diphenhydramine, diphenylpyraline, doxylamine succinate, doxylamine, ebastine, efletirizine, epinastine, farnotidine, fexofenadine, hydroxyzine, hydroxyzine, ketotifen, levocabastine, levocetirizine, levocetirizine, loratadine, meclizine, mepyramine, mequitazine, methdilazine, mianserin, mizolastine, noberastine, norasternizole, noraztemizole, phenindamine, pheniramine, picumast, promethazine, pynlamine, pyrilamine, ranitidine, temelastine, terfenadine, trimeprazine, tripelenamine, and triprolidine;
(7) an anticholinergic including but not limited to: atropine, benztropine, biperiden, flutropium, hyoscyamine (e.g. Levsin®; Levbid®; Levsin/SL®, Anaspaz®, Levsinex Timecaps®, NuLev®), ilutropium, ipratropium, ipratropium bromide, methscopolamine, oxybutinin, rispenzepine, scopolamine, and tiotropium;
(8) an anti-tussive including but not limited to: dextromethorphan, codeine, and hydromorphone;
(9) a decongestant including but not limited to: pseudoephedrine and phenylpropanolamine;
(10) an expectorant including but not limited to: guafenesin, guaicolsulfate, terpin, ammonium chloride, glycerol guaicolate, and iodinated glycerol;
(11) a bronchodilator including but not limited to: theophylline and aminophylline;
(12) an anti-inflammatory including but not limited to: fluribiprofen, diclophenac, indomethacin, ketoprofen, S-ketroprophen, tenoxicam;
(13) a PDE (phosphodiesterase) inhibitor including but not limited to those disclosed herein;
(14) a recombinant humanized monoclonal antibody [e.g. xolair (also called omalizumab), rhuMab, and talizumab];
(15) a humanized lung surfactant including recombinant forms of surfactant proteins SP-B, SP-C or SP-D [e.g. SURFAXIN®, formerly known as dsc-104 (Discovery Laboratories)],
(16) agents that inhibit epithelial sodium channels (ENaC) such as amiloride and related compounds;
(17) antimicrobial agents used to treat pulmonary infections such as acyclovir, amikacin, amoxicillin, doxycycline, trimethoprin sulfamethoxazole, amphotericin B, azithromycin, clarithromycin, roxithromycin, clarithromycin, cephalosporins (ceffoxitin, cefmetazole etc), ciprofloxacin, ethambutol, gentimycin, ganciclovir, imipenem, isoniazid, itraconazole, penicillin, ribavirin, rifampin, rifabutin, amantadine, rimantidine, streptomycin, tobramycin, and vancomycin;
(18) agents that activate chloride secretion through Ca++ dependent chloride channels (such as purinergic receptor (P2Y(2) agonists);
(19) agents that decrease sputum viscosity, such as human recombinant DNase 1, (Pulmozyme®);
(20) nonsteroidal anti-inflammatory agents (acemetacin, acetaminophen, acetyl salicylic acid, alclofenac, alminoprofen, apazone, aspirin, benoxaprofen, bezpiperylon, bucloxic acid, carprofen, clidanac, diclofenac, diclofenac, diflunisal, diflusinal, etodolac, fenbufen, fenbufen, fenclofenac, fenclozic acid, fenoprofen, fentiazac, feprazone, flufenamic acid, flufenisal, flufenisal, fluprofen, flurbiprofen, flurbiprofen, furofenac, ibufenac, ibuprofen, indomethacin, indomethacin, indoprofen, isoxepac, isoxicam, ketoprofen, ketoprofen, ketorolac, meclofenamic acid, meclofenamic acid, mefenamic acid, mefenamic acid, miroprofen, mofebutazone, nabumetone oxaprozin, naproxen, naproxen, niflumic acid, oxaprozin, oxpinac, oxyphenbutazone, phenacetin, phenylbutazone, phenylbutazone, piroxicam, piroxicam, pirprofen, pranoprofen, sudoxicam, tenoxican, sulfasalazine, sulindac, sulindac, suprofen, tiaprofenic acid, tiopinac, tioxaprofen, tolfenamic acid, tolmetin, tolmetin, zidometacin, zomepirac, and zomepirac); and
(21) aerosolized antioxidant therapeutics such as S-Nitrosoglutathione.

The polypeptides and agonists described herein can be used in combination therapy with an anti-obesity agent. Suitable such agents include, but are not limited to:

11β HSD-1 (11-beta hydroxy steroid dehydrogenase type 1) inhibitors, such as BVT 3498, BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][11]annulene, and those compounds disclosed in WO01/90091, WO01/90090, WO01/90092 and WO02/072084;
5HT antagonists such as those in WO03/037871, WO03/037887, and the like;
5HT1a modulators such as carbidopa, benserazide and those disclosed in U.S. Pat. No. 6,207,699, WO03/031439, and the like;
5HT2c (serotonin receptor 2c) agonists, such as BVT933, DPCA37215, 1K264, PNU 22394, WAY161503, R-1065, SB 243213 (Glaxo Smith Kline) and YM 348 and those disclosed in U.S. Pat. No. 3,914,250, WO00/77010, WO02/36596, WO02/48124, WO02/10169, WO01/66548, WO02/44152, WO02/51844, WO02/40456, and WO02/40457;
5HT6 receptor modulators, such as those in WO03/030901, WO03/035061, WO03/039547, and the like;
acyl-estrogens, such as oleoyl-estrone, disclosed in del Mar-Grasa, M. et al., Obesity Research, 9:202-9 (2001) and Japanese Patent Application No. JP 2000256190;
anorectic bicyclic compounds such as 1426 (Aventis) and 1954 (Aventis), and the compounds disclosed in WO00/18749, WO01/32638, WO01/62746, WO01/62747, and WO03/015769;
CB 1 (cannabinoid-1 receptor) antagonist/inverse agonists such as rimonabant (Acomplia; Sanofi), SR-147778 (Sanofi), SR-141716 (Sanofi), BAY 65-2520 (Bayer), and SLV 319 (Solvay), and those disclosed in patent publications U.S. Pat. No. 4,973,587, U.S. Pat. No. 5,013,837, U.S. Pat. No. 5,081,122, U.S. Pat. No. 5,112,820, U.S. Pat. No. 5,292,736, U.S. Pat. No. 5,532,237, U.S. Pat. No. 5,624,941, U.S. Pat. No. 6,028,084, U.S. Pat. No. 6,509,367, U.S. Pat. No. 6,509,367, WO96/33159, WO97/29079, WO98/31227, WO98/33765, WO98/37061, WO98/41519, WO98/43635, WO98/43636, WO99/02499, WO00/10967, WO00/10968, WO01/09120, WO01/58869, WO01/64632, WO01/64633, WO01/64634, WO01/70700, WO01/96330, WO02/076949, WO03/006007, WO03/007887, WO03/020217, WO03/026647, WO03/026648, WO03/027069, WO03/027076, WO03/027114, WO03/037332, WO03/040107, WO03/086940, WO03/084943 and EP658546;
CCK-A (cholecystokinin-A) agonists, such as AR-R 15849, GI 181771 (GSK), JMV-180, A-71378, A-71623 and SR146131 (Sanofi), and those described in U.S. Pat. No. 5,739,106;
CNTF (Ciliary neurotrophic factors), such as GI-181771 (Glaxo-SmithKline), SR146131 (Sanofi Synthelabo), butabindide, PD 170,292, and PD 149164 (Pfizer);
CNTF derivatives, such as Axokine® (Regeneron), and those disclosed in WO94/09134, WO98/22128, and WO99/43813;
dipeptidyl peptidase IV (DP-IV) inhibitors, such as isoleucine thiazolidide, valine pyrrolidide, NVP-DPP728, LAF237, P93/01, P 3298, TSL 225 (tryptophyl-1,2,3,4-tetrahydroisoquinoline-3-carboxylic acid; disclosed by Yamada et al, Bioorg. & Med. Chem. Lett. 8 (1998) 1537-1540), TMC-2A/2B/2C, CD26 inhibtors, FE 999011, P9310/K364, VIP 0177, SDZ 274-444, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996) and the compounds disclosed patent publications. WO99/38501, WO99/46272, WO99/67279 (Probiodrug), WO99/67278 (Probiodrug), WO99/61431 (Probiodrug), WO02/083128, WO02/062764, WO03/000180, WO03/000181, WO03/000250, WO03/002530, WO03/002531, WO03/002553, WO03/002593, WO03/004498, WO03/004496, WO03/017936, WO03/024942, WO03/024965, WO03/033524, WO03/037327 and EP1258476;
growth hormone secretagogue receptor agonists/antagonists, such as NN703, hexarelin, MK-0677 (Merck), SM-130686, CP-424391 (Pfizer), LY 444,711 (Eli Lilly), L-692,429 and L-163,255, and such as those disclosed in U.S. Ser. No. 09/662,448, U.S. provisional application 60/203,335, U.S. Pat. No. 6,358,951, US2002049196, US2002/022637, WO01/56592 and WO02/32888;
H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO02/15905, WO03/024928 and WO03/024929;
leptin derivatives, such as those disclosed in U.S. Pat. No. 5,552,524, U.S. Pat. No. 5,552,523, U.S. Pat. No. 5,552,522, U.S. Pat. No. 5,521,283, WO96/23513, WO96/23514, WO96/23515, WO96/23516, WO96/23517, WO96/23518, WO96/23519, and WO96/23520;
leptin, including recombinant human leptin (PEG-OB, Hoffman La Roche) and recombinant methionyl human leptin (Amgen);

lipase inhibitors, such as tetrahydrolipstatin (orlistat/Xenical®), Triton WR1339, RHC80267, lipstatin, teasaponin, diethylumbelliferyl phosphate, FL-386, WAY-121898, Bay-N-3176, valilactone, esteracin, ebelactone A, ebelactone B, and RHC 80267, and those disclosed in patent publications WO01/77094, U.S. Pat. No. 4,598,089, U.S. Pat. No. 4,452,813, U.S. Pat. No. 5,512,565, U.S. Pat. No. 5,391,571, U.S. Pat. No. 5,602,151, U.S. Pat. No. 4,405,644, U.S. Pat. No. 4,189,438, and U.S. Pat. No. 4,242,453;

lipid metabolism modulators such as maslinic acid, erythrodiol, ursolic acid uvaol, betulinic acid, betulin, and the like and compounds disclosed in WO03/011267;
Mc4r (melanocortin 4 receptor) agonists, such as CHIR86036 (Chiron), ME-10142, ME-10145, and HS-131 (Melacure), and those disclosed in PCT publication Nos. WO99/64002, WO00/74679, WO01/991752, WO01/25192, WO01/52880, WO01/74844, WO01/70708, WO01/70337, WO01/91752, WO02/059095, WO02/059107, WO02/059108, WO02/059117, WO02/06276, WO02/12166, WO02/11715, WO02/12178, WO02/15909, WO02/38544, WO02/068387, WO02/068388, WO02/067869, WO02/081430, WO03/06604, WO03/007949, WO03/009847, WO03/009850, WO03/013509, and WO03/031410;
Mc5r (melanocortin 5 receptor) modulators, such as those disclosed in WO97/19952, WO00/15826, WO00/15790, US20030092041;
melanin-concentrating hormone 1 receptor (MCHR) antagonists, such as T-226296 (Takeda), SB 568849, SNP-7941 (Synaptic), and those disclosed in patent publications WO01/21169, WO01/82925, WO01/87834, WO02/051809, WO02/06245, WO02/076929, WO02/076947, WO02/04433, WO02/51809, WO02/083134, WO02/094799, WO03/004027, WO03/13574, WO03/15769, WO03/028641, WO03/035624, WO03/033476, WO03/033480, JP13226269, and JP1437059;
mGluR5 modulators such as those disclosed in WO03/029210, WO03/047581, WO03/048137, WO03/051315, WO03/051833, WO03/053922, WO03/059904, and the like;
serotoninergic agents, such as fenfluramine (such as Pondimin® (Benzeneethanamine, N-ethyl-alpha-methyl-3-(trifluoromethyl)-, hydrochloride), Robbins), dexfenfluramine (such as Redux® (Benzeneethanamine, N-ethyl-alpha-methyl-3-(trifluoromethyl)-, hydrochloride), Interneuron) and sibutramine ((Meridia®, Knoll/Reductil™) including racemic mixtures, as optically pure isomers (+) and (−), and pharmaceutically acceptable salts, solvents, hydrates, clathrates and prodrugs thereof including sibutramine hydrochloride monohydrate salts thereof, and those compounds disclosed in U.S. Pat. No. 4,746,680, U.S. Pat. No. 4,806,570, and U.S. Pat. No. 5,436,272, US20020006964, WO01/27068, and WO01/62341;
NE (norepinephrine) transport inhibitors, such as GW 320659, despiramine, talsupram, and nomifensine;
NPY 1 antagonists, such as BIBP3226, J-115814, BIBO 3304, LY-357897, CP-671906, GI-264879A, and those disclosed in U.S. Pat. No. 6,001,836, WO96/14307, WO01/23387, WO99/51600, WO01/85690, WO01/85098, WO01/85173, and WO01/89528;
NPY5 (neuropeptide Y Y5) antagonists, such as 152,804, GW-569180A, GW-594884A, GW-587081X, GW-548118X, FR235208, FR226928, FR240662, FR252384, 1229U91, GI-264879A, CGP71683A, LY-377897, LY-366377, PD-160170, SR-120562A, SR-120819A, JCF-104, and H409/22 and those compounds disclosed in patent publications U.S. Pat. No. 6,140,354, U.S. Pat. No. 6,191,160, U.S. Pat. No. 6,218,408, U.S. Pat. No. 6,258,837, U.S. Pat. No. 6,313,298, U.S. Pat. No. 6,326,375, U.S. Pat. No. 6,329,395, U.S. Pat. No. 6,335,345, U.S. Pat. No. 6,337,332, U.S. Pat. No. 6,329,395, U.S. Pat. No. 6,340,683, EP01010691, EP-01044970, WO97/19682, WO97/20820, WO97/20821, WO97/20822, WO97/20823, WO98/27063, WO00/107409, WO00/185714, WO00/185730, WO00/64880, WO00/68197, WO00/69849, WO/0113917, WO01/09120, WO01/14376, WO01/85714, WO01/85730, WO01/07409, WO01/02379, WO01/23388, WO01/23389, WO01/44201, WO01/62737, WO01/62738, WO01/09120, WO02/20488, WO02/22592, WO02/48152, WO02/49648, WO02/051806, WO02/094789, WO03/009845, WO03/014083, WO03/022849, WO03/028726 and Norman et al., J. Med. Chem. 43:4288-4312 (2000);
opioid antagonists, such as nalmefene (REVEX®), 3-methoxynaltrexone, methylnaltrexone, naloxone, and naltrexone (e.g. PT901; Pain Therapeutics, Inc.) and those disclosed in US20050004155 and WO00/21509;
orexin antagonists, such as SB-334867-A and those disclosed in patent publications WO01/96302, WO01/68609, WO02/44172, WO02/51232, WO02/51838, WO02/089800, WO02/090355, WO03/023561, WO03/032991, and WO03/037847;
PDE inhibitors (e.g. compounds which slow the degradation of cyclic AMP (cAMP) and/or cyclic GMP (cGMP) by inhibition of the phosphodiesterases, which can lead to a relative increase in the intracellular concentration of cAMP and cGMP; possible PDE inhibitors are primarily those substances which are to be numbered among the class consisting of the PDE3 inhibitors, the class consisting of the PDE4 inhibitors and/or the class consisting of the PDE5 inhibitors, in particular those substances which can be designated as mixed types of PDE3/4 inhibitors or as mixed types of PDE3/4/5 inhibitors) such as those disclosed in patent publications DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EP0112987, EP0116948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. No. 4,963,561, U.S. Pat. No. 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DE1116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577), WO9307124, EP0163965, EP0393500, EP0510562, EP0553174, WO9501338 and WO9603399, as well as PDE5 inhibitors (such as RX-RA-69, SCH-51866, KT-734, vesnarinone, zaprinast, SKF-96231, ER-21355, BF/GP-385, NM-702 and sildenafil (Viagra™)), PDE4 inhibitors (such as etazolate, ICI63197, RP73401, imazolidinone (RO-20-1724), MEM 1414 (R1533/R1500; Pharmacia Roche), denbufylline, rolipram, oxagrelate, nitraquazone, Y-590, DH-6471, SKF-94120, motapizone, lixazinone, indolidan, olprinone, atizoram, KS-506-G, dipamfylline, BMY-43351, atizoram, arofylline, filaminast, PDB-093, UCB-29646, CDP-840, SKF-107806, piclamilast, RS-17597, RS-25344-000, SB-207499, TIBENELAST, SB-210667, SB-211572, SB-211600, SB-212066, SB-212179, GW-3600, CDP-840, mopidamol, anagrelide, ibudilast, aminone, pimobendan, cilostazol, quazinone and N-(3,5-dichloropyrid-4-yl)-3-cyclopropylmethoxy-4-difluoromethoxybenzamide, PDE3 inhibitors (such as ICI153, 100, bemorandane (RWJ 22867), MCI-154, UD-CG 212, sulmazole, ampizone, cilostamide, carbazeran, piroximone, imazodan, CI-930, siguazodan, adibendan, saterinone, SKF-95654, SDZ-MKS-492, 349-U-85, emoradan, EMD-53998, EMD-57033, NSP-306, NSP-307, revizinone, NM-702, WIN-62582 and WIN-63291, enoximone and milrinone, PDE3/4 inhibitors (such as benafentrine, trequinsin, ORG-30029, zardaverine, L-686398, SDZ-ISQ-844, ORG-20241, EMD-54622, and tolafentrine) and other PDE inhibitors (such as vinpocetin, papaverine, enprofylline, cilomilast, fenoximone, pentoxifylline, roflumilast, tadalafil (Clalis®), theophylline, and vardenafil (Levitra®);
Neuropeptide Y2 (NPY2) agonists include but are not limited to: polypeptide YY and fragments and variants thereof (e.g. YY3-36 (PYY3-36) (N. Engl. J. Med. 349:941, 2003; IKPEAPGE DASPEELNRY YASLRHYLNL VTRQRY (SEQ ID NO:XXX)) and PYY agonists such as those disclosed in WO02/47712, WO03/026591, WO03/057235, and WO03/027637;
serotonin reuptake inhibitors, such as, paroxetine, fluoxetine (Prozac™), fluvoxamine, sertraline, citalopram, and imipramine, and those disclosed in U.S. Pat. No. 6,162,805, U.S. Pat. No. 6,365,633, WO03/00663, WO01/27060, and WO01/162341;
thyroid hormone β agonists, such as KB-2611 (KaroBioBMS), and those disclosed in WO02/15845, WO97/21993, WO99/00353, GB98/284425, U.S. Provisional Application No. 60/183,223, and Japanese Patent Application No. JP 2000256190;
UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid, and those disclosed in WO99/00123;
β3 (beta adrenergic receptor 3) agonists, such as AJ9677/TAK677 (Dainippon/Takeda), L750355 (Merck), CP331648 (Pfizer), CL-316,243, SB 418790, BRL-37344, L-796568, BMS-196085, BRL-35135A, CGP12177A, BTA-243, GW 427353, Trecadrine, Zeneca D7114, N-5984 (Nisshin Kyorin), LY-377604 (Lilly), SR 59119A, and those disclosed in U.S. Pat. No. 5,541,204, U.S. Pat. No. 5,770,615, U.S. Pat. No. 5,491,134, U.S. Pat. No. 5,776,983, US488064, U.S. Pat. No. 5,705,515, U.S. Pat. No. 5,451,677, WO94/18161, WO95/29159, WO97/46556, WO98/04526 and WO98/32753, WO01/74782, WO02/32897, WO03/014113, WO03/016276, WO03/016307, WO03/024948, WO03/024953 and WO03/037881;
noradrenergic agents including, but not limited to, diethylpropion (such as Tenuate® (1-propanone, 2-(diethylamino)-1-phenyl-, hydrochloride), Merrell), dextroamphetamine (also known as dextroamphetamine sulfate, dexamphetamine, dexedrine, Dexampex, Ferndex, Oxydess II, Robese, Spancap #1), mazindol ((or 5-(p-chlorophenyl)-2,5-dihydro-3H-imidazo[2,1-a]isoindol-5-ol) such as Sanorex®, Novartis or Mazanor®, Wyeth Ayerst), phenylpropanolamine (or Benzenemethanol, alpha-(1-aminoethyl)-, hydrochloride), phentermine ((or Phenol, 3-[[4,5-duhydro-1H-imidazol-2-yl)ethyl](4-methylpheny-1)amino], monohydrochloride) such as Adipex-P®, Lemmon, FASTIN®, Smith-Kline Beecham and Ionamin®, Medeva), phendimetrazine ((or (2S,3S)-3,4-Dimethyl-2-phenylmorpholine L-(+)-tartrate (1:1)) such as Metra® (Forest), Plegine® (Wyeth-Ayerst), Prelu-2® (Boehringer Ingelheim), and Statobex® (Lemmon), phendamine tartrate (such as Thephorin® (2,3,4,9-Tetrahydro-2-methyl-9-phenyl-1H-indenol[2,1-c]pyridine L-(+)-tartrate (1:1)), Hoffmann-LaRoche), methamphetamine (such as Desoxyn®, Abbot ((S)—N, (alpha)-dimethylbenzeneethanamine hydrochloride)), and phendimetrazine tartrate (such as Bontril® Slow-Release Capsules, Amarin (−3,4-Dimethyl-2-phenylmorpholine Tartrate);
fatty acid oxidation upregulator/inducers such as Famoxin® (Genset);
monamine oxidase inhibitors including but not limited to befloxatone, moclobemide, brofaromine, phenoxathine, esuprone, befol, toloxatone, pirlindol, amiflamine, sercloremine, bazinaprine, lazabemide, milacemide, caroxazone and other certain compounds as disclosed by WO01/12176; and
other anti-obesity agents such as 5HT-2 agonists, ACC (acetyl-CoA carboxylase) inhibitors such as those described in WO03/072197, alpha-lipoic acid (alpha-LA), AOD9604, appetite suppressants such as those in WO03/40107, ATL-962 (Alizyme PLC), benzocaine, benzphetamine hydrochloride (Didrex), bladderwrack (focus vesiculosus), BRS3 (bombesin receptor subtype 3) agonists, bupropion, caffeine, CCK agonists, chitosan, chromium, conjugated linoleic acid, corticotropin-releasing hormone agonists, dehydroepiandrosterone, DGAT1 (diacylglycerol acyltransferase 1) inhibitors, DGAT2 (diacylglycerol acyltransferase 2) inhibitors, dicarboxylate transporter inhibitors, ephedra, exendin-4 (an inhibitor of glp-1) FAS (fatty acid synthase) inhibitors (such as Cerulenin and C75), fat resorption inhibitors (such as those in WO03/053451, and the like), fatty acid transporter inhibitors, natural water soluble fibers (such as psyllium, plantago, guar, oat, pectin), galanin antagonists, galega (Goat's Rue, French Lilac), garcinia cambogia, germander (teucrium chamaedrys), ghrelin antibodies and ghrelin antagonists (such as those disclosed in WO01/87335, and WO02/08250), polypeptide hormones and variants thereof which affect the islet cell secretion, such as the hormones of the secretin/gastric inhibitory polypeptide (GIP)/vasoactive intestinal polypeptide (VIP)/pituitary adenylate cyclase activating polypeptide (PACAP)/glucagon-like polypeptide II (GLP-II)/glicentin/glucagon gene family and/or those of the adrenomedullin/amylin/calcitonin gene related polypeptide (CGRP) gene family including GLP-1 (glucagon-like polypeptide 1) agonists (e.g. (1) exendin-4, (2) those GLP-1 molecules described in US20050130891 including GLP-1(7-34), GLP-1 (7-35), GLP-1 (7-36) or GLP-1 (7-37) in its C-terminally carboxylated or amidated form or as modified GLP-1 polypeptides and modifications thereof including those described in paragraphs 17-44 of US20050130891, and derivatives derived from GLP-1-(7-34)COOH and the corresponding acid amide are employed which have the following general formula:

R-NH-HAEGTFTSDVSYLEGQAAKEFIAWLVK-CONH₂

wherein R═H or an organic compound having from 1 to 10 carbon atoms. Preferably, R is the residue of a carboxylic acid. Particularly preferred are the following carboxylic acid residues: formyl, acetyl, propionyl, isopropionyl, methyl, ethyl, propyl, isopropyl, n-butyl, sec-butyl, tert-butyl.) and glp-1 (glucagon-like polypeptide-1), glucocorticoid antagonists, glucose transporter inhibitors, growth hormone secretagogues (such as those disclosed and specifically described in U.S. Pat. No. 5,536,716), interleukin-6 (IL-6) and modulators thereof (as in WO03/057237, and the like), L-carnitine, Mc3r (melanocortin 3 receptor) agonists, MCH2R (melanin concentrating hormone 2R) agonist/antagonists, melanin concentrating hormone antagonists, melanocortin agonists (such as Melanotan II or those described in WO 99/64002 and WO 00/74679), nomame herba, phosphate transporter inhibitors, phytopharm compound 57 (CP 644,673), pyruvate, SCD-1 (stearoyl-CoA desaturase-1) inhibitors, T71 (Tularik, Inc., Boulder Colo.), Topiramate (Topimax®, indicated as an anti-convulsant which has been shown to increase weight loss), transcription factor modulators (such as those disclosed in WO03/026576), β-hydroxy steroid dehydrogenase-1 inhibitors (β-HSD-1), β-hydroxy-β-methylbutyrate, p57 (Pfizer), Zonisamide (Zonegran™, indicated as an anti-epileptic which has been shown to lead to weight loss), and the agents disclosed in US20030119428 paragraphs 20-26.

The polypeptides and agonists described herein can be used in therapeutic combination with one or more anti-diabetic agents, including but not limited to:

PPARγ agonists such as glitazones (e.g., WAY-120,744, AD 5075, balaglitazone, ciglitazone, darglitazone (CP-86325, Pfizer), englitazone (CP-68722, Pfizer), isaglitazone (MIT/J&J), MCC-555 (Mitsibishi disclosed in U.S. Pat. No. 5,594,016), pioglitazone (such as such as Actos™ pioglitazone; Takeda), rosiglitazone (Avandia™; Smith Kline Beecham), rosiglitazone maleate, troglitazone (Rezulin®, disclosed in U.S. Pat. No. 4,572,912), rivoglitazone (CS-011, Sankyo), GL-262570 (Glaxo Welcome), BRL49653 (disclosed in WO98/05331), CLX-0921, 5-BTZD, GW-0207, LG-100641, JJT-501 (JPNT/P&U), L-895645 (Merck), R-119702 (Sankyo/Pfizer), NN-2344 (Dr. Reddy/NN), YM-440 (Yamanouchi), LY-300512, LY-519818, R483 (Roche), T131 (Tularik), and the like and compounds disclosed in U.S. Pat. No. 4,687,777, U.S. Pat. No. 5,002,953, U.S. Pat. No. 5,741,803, U.S. Pat. No. 5,965,584, U.S. Pat. No. 6,150,383, U.S. Pat. No. 6,150,384, U.S. Pat. No. 6,166,042, U.S. Pat. No. 6,166,043, U.S. Pat. No. 6,172,090, U.S. Pat. No. 6,211,205, U.S. Pat. No. 6,271,243, U.S. Pat. No. 6,288,095, U.S. Pat. No. 6,303,640, U.S. Pat. No. 6,329,404, U.S. Pat. No. 5,994,554, WO97/10813, WO97/27857, WO97/28115, WO97/28137, WO97/27847, WO00/76488, WO03/000685, WO03/027112, WO03/035602, WO03/048130, WO03/055867, and pharmaceutically acceptable salts thereof;
biguanides such as metformin hydrochloride (N,N-dimethylimidodicarbonimidic diamide hydrochloride, such as Glucophage™, Bristol-Myers Squibb); metformin hydrochloride with glyburide, such as Glucovance™, Bristol-Myers Squibb); buformin (Imidodicarbonimidic diamide, N-butyl-); etoformine (1-Butyl-2-ethylbiguanide, Schering A. G.); other metformin salt forms (including where the salt is chosen from the group of, acetate, benzoate, citrate, ftimarate, embonate, chlorophenoxyacetate, glycolate, palmoate, aspartate, methanesulphonate, maleate, parachlorophenoxyisobutyrate, formate, lactate, succinate, sulphate, tartrate, cyclohexanecarboxylate, hexanoate, octanoate, decanoate, hexadecanoate, octodecanoate, benzenesulphonate, trimethoxybenzoate, paratoluenesulphonate, adamantanecarboxylate, glycoxylate, glutamate, pyrrolidonecarboxylate, naphthalenesulphonate, 1-glucosephosphate, nitrate, sulphite, dithionate and phosphate), and phenformin;
protein tyrosine phosphatase-1B (PTP-1B) inhibitors, such as A-401,674, KR 61639, OC-060062, OC-83839, OC-297962, MC52445, MC52453, ISIS113715, and those disclosed in WO99/585521, WO99/58518, WO99/58522, WO99/61435, WO03/032916, WO03/032982, WO03/041729, WO03/055883, WO02/26707, WO02/26743, JP2002114768, and pharmaceutically acceptable salts and esters thereof;
sulfonylureas such as acetohexamide (e.g. Dymelor, Eli Lilly), carbutamide, chlorpropamide (e.g. Diabinese®, Pfizer), gliamilide (Pfizer), gliclazide (e.g. Diamcron, Servier Canada Inc), glimepiride (e.g. disclosed in U.S. Pat. No. 4,379,785, such as Amaryl™, Aventis), glipentide, glipizide (e.g. Glucotrol or Glucotrol XL Extended Release, Pfizer), gliquidone, glisolamide, glyburide/glibenclamide (e.g. Micronase or Glynase Prestab, Pharmacia & Upjohn and Diabeta, Aventis), tolazamide (e.g. Tolinase), and tolbutamide (e.g. Orinase), and pharmaceutically acceptable salts and esters thereof;
meglitinides such as repaglinide (e.g. Pranidin®, Novo Nordisk), KAD1229 (PF/Kissei), and nateglinide (e.g. Starlix®, Novartis), and pharmaceutically acceptable salts and esters thereof; a glucoside hydrolase inhibitors (or glucoside inhibitors) such as acarbose (e.g. Precose™, Bayer disclosed in U.S. Pat. No. 4,904,769), miglitol (such as GLYSET™, Pharmacia & Upjohn disclosed in U.S. Pat. No. 4,639,436), camiglibose (Methyl 6-deoxy-6-[(2R,3R,4R,5S)-3,4,5-trihydroxy-2-(hydroxymethyl)piperidino]-alpha-D-glucopyranoside, Marion Merrell Dow), voglibose (Takeda), adiposine, emiglitate, pradimicin-Q, salbostatin, CKD-711, MDL-25,637, MDL-73,945, and MOR 14, and the compounds disclosed in U.S. Pat. No. 4,062,950, U.S. Pat. No. 4,174,439, U.S. Pat. No. 4,254,256, U.S. Pat. No. 4,701,559, U.S. Pat. No. 4,639,436, U.S. Pat. No. 5,192,772, U.S. Pat. No. 4,634,765, U.S. Pat. No. 5,157,116, U.S. Pat. No. 5,504,078, U.S. Pat. No. 5,091,418, U.S. Pat. No. 5,217,877, US51091 and WO01/47528 (polyamines);
α-amylase inhibitors such as tendamistat, trestatin, and A1-3688, and the compounds disclosed in U.S. Pat. No. 4,451,455, U.S. Pat. No. 4,623,714, and U.S. Pat. No. 4,273,765;
SGLT2 inhibtors including those disclosed in U.S. Pat. No. 6,414,126 and U.S. Pat. No. 6,515,117;
an aP2 inhibitor such as disclosed in U.S. Pat. No. 6,548,529;
insulin secreatagogues such as linogliride, A-4166, forskilin, dibutyrl cAMP, isobutylmethylxanthine (IBMX), and pharmaceutically acceptable salts and esters thereof;
fatty acid oxidation inhibitors, such as clomoxir, and etomoxir, and pharmaceutically acceptable salts and esters thereof;
A2 antagonists, such as midaglizole, isaglidole, deriglidole, idazoxan, earoxan, and fluparoxan, and pharmaceutically acceptable salts and esters thereof;
insulin and related compounds (e.g. insulin mimetics) such as biota, LP-100, novarapid, insulin detemir, insulin lispro, insulin glargine, insulin zinc suspension (lente and ultralente), Lys-Pro insulin, GLP-1 (1-36) amide, GLP-1 (73-7) (insulintropin, disclosed in U.S. Pat. No. 5,614,492), LY-315902 (Lilly), GLP-1 (7-36)-NH2), AL-401 (AutoImmune), certain compositions as disclosed in U.S. Pat. No. 4,579,730, U.S. Pat. No. 4,849,405, U.S. Pat. No. 4,963,526, U.S. Pat. No. 5,642,868, U.S. Pat. No. 5,763,396, U.S. Pat. No. 5,824,638, U.S. Pat. No. 5,843,866, U.S. Pat. No. 6,153,632, U.S. Pat. No. 6,191,105, and WO 85/05029, and primate, rodent, or rabbit insulin including biologically active variants thereof including allelic variants, more preferably human insulin available in recombinant form (sources of human insulin include pharmaceutically acceptable and sterile formulations such as those available from Eli Lilly (Indianapolis, Ind. 46285) as Humulin™ (human insulin rDNA origin), also see the THE PHYSICIAN'S DESK REFERENCE, 55.sup.th Ed. (2001) Medical Economics, Thomson Healthcare (disclosing other suitable human insulins);
non-thiazolidinediones such as JT-501 and farglitazar (GW-2570/GI-262579), and pharmaceutically acceptable salts and esters thereof;
PPARα/γ dual agonists such as AR-H039242 (Aztrazeneca), GW-409544 (Glaxo-Wellcome), BVT-142, CLX-0940, GW-1536, GW-1929, GW-2433, KRP-297 (Kyorin Merck; 5-[(2,4-Dioxo thiazolidinyl)methyl]methoxy-N-[[4-(trifluoromethyl)phenyl]methyl]benzamide), L-796449, LR-90, MK-0767 (Merck/Kyorin/Banyu), SB 219994, muraglitazar (BMS), tesaglitzar (Astrazeneca), reglitazar (JTT-501) and those disclosed in WO99/16758, WO99/19313, WO99/20614, WO99/38850, WO00/23415, WO00/23417, WO00/23445, WO00/50414, WO01/00579, WO01/79150, WO02/062799, WO03/004458, WO03/016265, WO03/018010, WO03/033481, WO03/033450, WO03/033453, WO03/043985, WO 031053976, U.S. application Ser. No. 09/664,598, filed Sep. 18, 2000, Murakami et al. Diabetes 47, 1841-1847 (1998), and pharmaceutically acceptable salts and esters thereof;
other insulin sensitizing drugs;
VPAC2 receptor agonists;
GLK modulators, such as those disclosed in WO03/015774;
retinoid modulators such as those disclosed in WO03/000249;
GSK 3β/GSK 3 inhibitors such as 4-[2-(2-bromophenyl)-4-(4-fluorophenyl-1H-imidazol-5-yl]pyridine and those compounds disclosed in WO03/024447, WO03/037869, WO03/037877, WO03/037891, WO03/068773, EP1295884, EP1295885, and the like;
glycogen phosphorylase (HGLPa) inhibitors such as CP-368,296, CP-316,819, BAYR3401, and compounds disclosed in WO01/94300, WO02/20530, WO03/037864, and pharmaceutically acceptable salts or esters thereof;
ATP consumption promotors such as those disclosed in WO03/007990;
TRB3 inhibitors;
vanilloid receptor ligands such as those disclosed in WO03/049702;
hypoglycemic agents such as those disclosed in WO03/015781 and WO03/040114;
glycogen synthase kinase 3 inhibitors such as those disclosed in WO03/035663
agents such as those disclosed in WO99/51225, US20030134890, WO01/24786, and WO03/059870;
insulin-responsive DNA binding protein-1 (IRDBP-1) as disclosed in WO03/057827, and the like;
adenosine A2 antagonists such as those disclosed in WO03/035639, WO03/035640, and the like;
PPARδ agonists such as GW 501516, GW 590735, and compounds disclosed in JP10237049 and WO02/14291;
dipeptidyl peptidase IV (DP-IV) inhibitors, such as isoleucine thiazolidide, NVP-DPP728A (1-[[[2-[(5-cyanopyridin-2-yl)amino]ethyl]amino]acetyl]-2-cyano-(S)-pyrrolidine, disclosed by Hughes et al, Biochemistry, 38(36), 11597-11603, 1999), P32/98, NVP-LAF-237, P3298, TSL225 (tryptophyl-1,2,3,4-tetrahydro-isoquinoline-3-carboxylic acid, disclosed by Yamada et al, Bioorg. & Med. Chem. Lett. 8 (1998) 1537-1540), valine pyrrolidide, TMC-2A/2B/2C, CD-26 inhibitors, FE999011, P9310/K364, VIP 0177, DPP4, SDZ 274-444, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996), and the compounds disclosed in U.S. Pat. No. 6,395,767, U.S. Pat. No. 6,573,287, U.S. Pat. No. 6,395,767 (compounds disclosed include BMS-477118, BMS-471211 and BMS 538,305), WO99/38501, WO99/46272, WO99/67279, WO99/67278, WO99/61431 WO03/004498, WO03/004496, EP1258476, WO02/083128, WO02/062764, WO03/000250, WO03/002530, WO03/002531, WO03/002553, WO03/002593, WO03/000180, and WO03/000181;
GLP-1 agonists such as exendin-3 and exendin-4 (including the 39 aa polypeptide synthetic exendin-4 called Exenatide®), and compounds disclosed in US2003087821 and NZ 504256, and pharmaceutically acceptable salts and esters thereof;
peptides including amlintide and Symlin® (pramlintide acetate); and
glycokinase activators such as those disclosed in US2002103199 (fused heteroaromatic compounds) and WO02/48106 (isoindolin-1-one-substituted propionamide compounds).
The polypeptides and agonists described herein useful in the treatment of obesity can be administered as a cotherapy with electrostimulation (US20040015201).
The polypeptides and agonists described herein can be used in combination therapy with agents that activate soluble guanylate cyclase, for example those described in US20040192680.

The polypeptides and agonists described herein can be used in combination therapy with a phosphodiesterase inhibitor. PDE inhibitors are those compounds which slow the degradation of cyclic AMP (cAMP) and/or cyclic GMP (cGMP) by inhibition of the phosphodiesterases, which can lead to a relative increase in the intracellular concentration of cAMP and/or cGMP. Possible PDE inhibitors are primarily those substances which are to be numbered among the class consisting of the PDE3 inhibitors, the class consisting of the PDE4 inhibitors and/or the class consisting of the PDE5 inhibitors, in particular those substances which can be designated as mixed types of PDE3/4 inhibitors or as mixed types of PDE3/4/5 inhibitors. By way of example, those PDE inhibitors may be mentioned such as are described and/or claimed in the following patent applications and patents: DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EP0112987, EP0116948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. Nos. 4,963,561, 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DE1116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577) and WO9307124, EP0163965, EP0393500, EP0510562, EP0553174, WO9501338 and WO9603399. PDE5 inhibitors which may be mentioned by way of example are RX-RA-69, SCH-51866, KT-734, vesnarinone, zaprinast, SKF-96231, ER-21355, BF/GP-385, NM-702 and sildenafil (Viagra®). PDE4 inhibitors which may be mentioned by way of example are RO-20-1724, MEM 1414 (R1533/R1500; Pharmacia Roche), DENBUFYLLINE, ROLIPRAM, OXAGRELATE, NITRAQUAZONE, Y-590, DH-6471, SKF-94120, MOTAPIZONE, LIXAZINONE, INDOLIDAN, OLPRINONE, ATIZORAM, KS-506-G, DIPAMFYLLINE, BMY-43351, ATIZORAM, AROFYLLINE, FILAMINAST, PDB-093, UCB-29646, CDP-840, SKF-107806, PICLAMILAST, RS-17597, RS-25344-000, SB-207499, TIBENELAST, SB-210667, SB-211572, SB-211600, SB-212066, SB-212179, GW-3600, CDP-840, MOPIDAMOL, ANAGRELIDE, IBUDILAST, AMRINONE, PIMOBENDAN, CILOSTAZOL, QUAZINONE and N-(3,5-dichloropyrid-4-yl)-3-cyclopropylmethoxy-4-difluoromethoxybenzamide. PDE3 inhibitors which may be mentioned by way of example are SULMAZOLE, AMPIZONE, CILOSTAMIDE, CARBAZERAN, PIROXIMONE, IMAZODAN, CI-930, SIGUAZODAN, ADIBENDAN, SATERINONE, SKF-95654, SDZ-MKS-492, 349-U-85, EMORADAN, EMD-53998, EMD-57033, NSP-306, NSP-307, REVIZINONE, NM-702, WIN-62582 and WIN-63291, ENOXIMONE and MILRINONE. PDE3/4 inhibitors which may be mentioned by way of example are BENAFENTRINE, TREQUINSIN, ORG-30029, ZARDAVERINE, L-686398, SDZ-ISQ-844, ORG-20241, EMD-54622, and TOLAFENTRINE. Other PDE inhibitors include: cilomilast, pentoxifylline, roflumilast, tadalafil (Clalis®), theophylline, and vardenafil (Levitra®), zaprinast (PDE5 specific).

The polypeptides and agonists described herein can be used in combination therapy (for example, in order to decrease or inhibit uterine contractions) with a tocolytic agent including but not limited to beta-adrenergic agents, magnesium sulfate, prostaglandin inhibitors, and calcium channel blockers.

The polypeptides and agonists described herein can be used in combination therapy with an anti-neoplastic agents including but not limited to alkylating agents, epipodophyllotoxins, nitrosoureas, antimetabolites, vinca alkaloids, anthracycline antibiotics, nitrogen mustard agents, and the like. Particular anti-neoplastic agents may include tamoxifen, taxol, etoposide and 5-fluorouracil. The polypeptides and agonists described herein can be used in combination therapy (for example as in a chemotherapeutic composition) with an antiviral and monoclonal antibody therapies.

The polypeptides and agonists described herein can be used in combination therapy (for example, in prevention/treatment of congestive heart failure or another method described herein) with the partial agonist of the nociceptin receptor ORL1 described by Dooley et al. (The Journal of Pharmacology and Experimental Therapeutics, 283 (2): 735-741, 1997). The agonist is a hexapeptide having the amino acid sequence Ac-RYY (RK) (WI) (RK)-NH2 (“the Dooley polypeptide”), where the brackets show allowable variation of amino acid residue. Thus Dooley polypeptide can include but are not limited to KYYRWR, RYYRWR, KWRYYR, RYYRWK, RYYRWK (all-D amin acids), RYYRIK, RYYRIR, RYYKIK, RYYKIR, RYYKWR, RYYKWK, RYYRWR, RYYRWK, RYYRIK, RYYKWR, RYYKWK, RYYRWK and KYYRWK, wherein the amino acid residues are in the L-form unless otherwise specified. The polypeptides and agonists described herein can also be used in combination therapy with polypeptide conjugate modifications of the Dooley polypeptide described in WO0198324.

### Methods of Treatment

A number of disorders might be treated with GC-C receptor agonists and agents that increase cGMP levels including the polypeptides and agonists described herein.

The polypeptides and agonists described herein can be used alone or in combination therapy for the treatment or prevention of congestive heart failure. Such agents can be used in combination with natriuretic polypeptides (e.g., atrial natriuretic polypeptide, brain natriuretic polypeptide or C-type natriuretic polypeptide), a diuretic, or an inhibitor of angiotensin converting enzyme.

The polypeptides and agonists described herein can be used alone or in combination therapy for the treatment or prevention of benign prostatic hyperplasia (BPH). Such agents can be used in combination with one or more agents for treatment of BPH, for example, a 5-alpha reductase inhibitor (e.g., finasteride) or an alpha adrenergic inhibitor (e.g., doxazosine).

The polypeptides and agonists described herein can be used alone or in combination therapy for the treatment, prevention or reduction of visceral pain associated with a gastrointestinal disorder or pain associated with another disorder.

The polypeptides and agonists described herein can be used alone or in combination therapy for the treatment or prevention of obesity-related disorders (e.g. disorders that are associated with, caused by, or result from obesity). Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrhythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia. The agents described herein may be used to reduce or control body weight (or fat) or to prevent and/or treat obesity or other appetite related disorders related to the excess consumption of food, ethanol and other appetizing substances. The agents may be used to modulate lipid metabolism, reduce body fat (e.g. via increasing fat utilization) or reduce (or suppress) appetite (e.g. via inducing satiety). Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastroesophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The agents of the present invention are also useful for reducing the risk of secondary outcomes of obesity, such as reducing the risk of left ventricular hypertrophy.

The polypeptides and agonists described herein can be used alone or in combination therapy for the treatment or prevention of gastrointestinal related disorders including: chronic intestinal pseudo-obstruction (Ogilvie's syndrome), colonic pseudoobstruction, Crohn's disease, dyspepsia (including functional dyspepsia or nonulcer dyspepsia), duodenogastric reflux, functional bowel disorder, functional gastrointestinal disorders, functional heartburn, gastroesophageal reflux disease (GERD), gastrointestinal motility disorders, gastroparesis (e.g. idopathic gastroparesis), hypertrophic pyloric stenosis, Inflammatory bowel disease, irritable bowel syndrome (IBS), post-operative ileus, and ulcerative colitis. The polypeptides and agonists described herein can be used alone or in combination therapy to patient suffering from or susceptible to GI disorders relating to damage to the GI tract stemming from impact or surgical intervention. The polypeptides and agonists described herein can be used alone or in combination therapy to patients at risk for or having particular diseases associated with hypomotility (e.g. colonic inertia) or stasis in the GI tract. For example, diabetic neuropathy, anorexia nervosa, and achlorhydria are frequently accompanied by gastric hypomotility. Damage to the GI tract following surgical intervention, for instance, can result in substantial gastric stasis. The polypeptides and agonists described herein can be administered alone or in combination therapy to patients susceptible to or having a GI disorder associated with diabetes (e.g. diabetic gastropathy). The polypeptides and agonists described herein can be used alone or in combination therapy to prevent and/or treat GI disorders characterized by at least one of nausea, vomiting, heartburn, postprandial discomfort, diarrhea, constipation, indigestion or related symptoms. The polypeptides and agonists described herein can be used alone or in combination therapy to prevent and/or treat GI disorders associated with at least one of diabetes, anorexia nervosa, bulimia, achlorhydria, achalasia, anal fissure, haemorrhoids, irritable bowel syndrome, intestinal pseudoobstruction, scleroderma and gastrointestinal damage.

The polypeptides and agonists described herein can be used to prevent and/or treat constipation. Constipation can be used to describe bowel patterns which include one or more of hard, small, infrequent stools; the sensation of difficulty in passing stool, specifically excessive or ineffectual straining; the sensation of incomplete evacuation. Constipation has also been described as the passage of stool less than a certain number (e.g. 3) of times per week. A number of conditions can be associated with constipation. Constipation can be associated with numerous disorders and conditions. For example, constipation can be (1) associated with the use of a therapeutic agent (e.g. antihypertensives, anticonvulsants, antispasmodics, analgesics, anticholinergics, antidepressants, antipsychotics, cation-containing agents, anticonvulsants, ganglion blockers, vinca alkaloids); (2) associated with a muscular, neuropathic, metabolic or endocrine disorder (including but not limited to myotonic dystrophy, dermamyositis, systemic sclerosis, sclerodoma, amyloidosis (neurologic or muscular), ischemia, tumor of the central nervous system, autonomic neuropathy, Chagas disease, cystic fibrosis, diabetes mellitus, Hirschsprung disease, hyperthyroidism, hypocalcaemia, hypothyroidism, Multiple Sclerosis, neurofibromatosis, Parkinson's disease, and spinal cord lesions (for example, related to sacral nerve damage related to trauma or a tumor or the enteric nervous system)); (3) post-surgical constipation (postoperative ileus); (4) associated with a structural colon alteration (for example that associated with Neoplasm, stricture, volvulus, anorectal, inflammation, prolapse, rectocele, or fissure); (5) associated with the a gastrointestinal disorder; (6) associated with a systemic illness or disorder (for example, electrolyte abnormalities, thyroid disease, diabetes mellitus, panhypopituitarism, Addison's disease, pheochromocytoma, uremia, porphyria); (7) chronic constipation; (8) associated with the use of analgesic drugs (e.g. opioid induced constipation); (9) associated with megacolon; and (10) idiopathic constipation (functional constipation). Functional constipation can be associated with normal transit, slow transit (e.g. one or fewer bowel movements per week) and pelvic floor dyssynergia. Pelvic floor dyssynergia is considered a disorder of the rectum and anus although these patients also have abnormal contractions throughout the colon. Patients with pelvic floor dyssynergia have abnormal colonic pressure waves prior to defecation and present with symptoms that may include a sensation of incomplete evacuation, excessive straining, a need for digital disimpaction, perianal heaviness, and tenesmus. Constipation can be associated with bloating and abdominal pain. The polypeptides and agonists described herein can be used to prevent and/or treat low stool frequency or poor stool consistency.

The polypeptides and agonists described herein can be used to treat decreased intestinal motility, slow digestion or slow stomach emptying. The polypeptides and agonists can be used to relieve one or more symptoms of IBS (bloating, pain, constipation), GERD (acid reflux into the esophagus), duodenogastric reflux, functional dyspepsia, or gastroparesis (nausea, vomiting, bloating, delayed gastric emptying) and other disorders described herein. The polypeptides and agonists described herein can be used to treat flatulence.

The polypeptides and agonists described herein can be used to increase intestinal motility, slow colonic transit, and to prevent and/or treat gastrointestinal immotility and other conditions calling for laxative or stool softener therapy. Gastrointestinal immotility can include constipation, and also includes delayed oral cecal transit time, irregular Taxation, and other related gastrointestinal motility disfunction including impaction. Impaction is a condition where a large mass of dry, hard stool develops in the rectum, often due to chronic constipation. This mass may be so hard that it cannot be excreted. The subjects affected by constipation or gastrointestinal immotility can be refractory to laxative therapy and/or stool softener therapy.

The polypeptides and agonists described herein can be used for the treatment or prevention of cancer, pre-cancerous growths, or metastatic growths. For example, they can be used for the prevention or treatment of: colorectal/local metastasized colorectal cancer, intestinal polyps, gastrointestinal tract cancer, lung cancer, cancer or pre-cancerous growths or metastatic growths of epithelial cells, polyps, breast, colorectal, lung, ovarian, pancreatic, prostatic, renal, stomach, bladder, liver, esophageal and testicular carcinoma, carcinoma (e.g., basal cell, basosquamous, Brown-Pearce, ductal carcinoma, Ehrlich tumor, Krebs, Merkel cell, small or non-small cell lung, oat cell, papillary, bronchiolar, squamous cell, transitional cell, (Walker), leukemia (e.g., B-cell, T-cell, HTLV, acute or chronic lymphocytic, mast cell, myeloid), histiocytonia, histiocytosis, Hodgkin's disease, non-Hodgkin's lymphoma, plasmacytoma, reticuloendotheliosis, adenoma, adeno-carcinoma, adenofibroma, adenolymphoma, ameloblastoma, angiokeratoma, angiolymphoid hyperplasia with eosinophilia, sclerosing angioma, angiomatosis, apudoma, branchionia, malignant carcinoid syndrome, carcinoid heart disease, carcinosarcoma, cementoma, cholangioma, cholesteatoma, chondrosarcoma, chondroblastoma, chondrosarcoma, chordoma, choristoma, craniopharyngioma, chrondrorna, cylindroma, cystadenocarcinoma, cystadenoma, cystosarconia phyllodes, dysgenninoma, ependymoma, Ewing sarcoma, fibroma, fibrosarcoma, giant cell tumor, ganglioneuroma, glioblastoma, glomangioma, granulosa cell tumor, gynandroblastoma, hamartoma, hemangioendothelioma, hemangioma, hemangio-pericytoma, hemangiosarcoma, hepatoma, islet cell tumor, Kaposi sarcoma, leiomyoma, leiomyosarcoma, leukosarcoma, Leydig cell tumor, lipoma, liposarcoma, lymphaugioma, lymphangiomyoma, lymphangiosarcoma, medulloblastoma, meningioma, mesenchymoma, mesonephroma, mesothelioma, myoblastoma, myoma, myosarcoma, myxoma, myxosarcoma, neurilemmoma, neuroma, neuroblastoma, neuroepithelioma, neurofibroma, neurofibromatosis, odontoma, osteoma, osteosarcoma, papilloma, paraganglioma, paraganglionia. nonchromaffin, pinealoma, rhabdomyoma, rhabdomyosarcoma, Sertoli cell tumor, teratoma, theca cell tumor, and other diseases in which cells have become dysplastic, immortalized, or transformed.

The polypeptides and agonists described herein can be used for the treatment or prevention of: Familial Adenomatous Polyposis (FAP) (autosomal dominant syndrome) that precedes colon cancer, hereditary nonpolyposis colorectal cancer (HNPCC), and inherited autosomal dominant syndrome.

For treatment or prevention of cancer, pre-cancerous growths and metastatic growths, the polypeptides and agonists described herein can be used in combination therapy with radiation or chemotherapeutic agents, an inhibitor of a cGMP-dependent phosphodiesterase or a selective cyclooxygenase-2 inhibitor. A number of selective cyclooxygenase-2 inhibitors are described in US20010024664, U.S. Pat. No. 5,380,738, U.S. Pat. No. 5,344,991, U.S. Pat. No. 5,393,790, U.S. Pat. No. 5,434,178, U.S. Pat. No. 5,474,995, U.S. Pat. No. 5,510,368, WO02/062369, WO 96/06840, WO 96/03388, WO 96/03387, WO 96/19469, WO 96/25405, WO 95/15316, WO 94/15932, WO 94/27980, WO 95/00501, WO 94/13635, WO 94/20480, and WO 94/26731, the disclosures of which are herein incorporated by reference. [Pyrazol-1-yl]benzenesulfonamides have also been described as inhibitors of cyclooxygenase-2.

The polypeptides and agonists described herein can be used in the treatment or prevention of inflammation. Thus, they can be used alone or in combination with an inhibitor of cGMP-dependent phosphodiesterase or a selective cyclooxygenase-2 inhibitor for treatment of: organ inflammation, IBD (e.g, Crohn's disease, ulcerative colitis), asthma, nephritis, hepatitis, pancreatitis, bronchitis, cystic fibrosis, ischemic bowel diseases, intestinal inflammations/allergies, coeliac disease, proctitis, eosinophilic gastroenteritis, mastocytosis, and other inflammatory disorders. The polypeptides and agonists described herein can be used alone or in combination therapy in the treatment or prevention of gastrointestinal tract inflammation (e.g. inflammation associated with a gastrointestinal disorder, gastrointestinal tract infection, or another disorder). They can be used alone or in combination therapy with phenoxyalkycarboxylic acid derivatives for the treatment of interstitial cystitis, irritable bowel syndrome, ulcerative colitis, and other inflammatory conditions, as mentioned in US20050239902A1.

The polypeptides and agonists described herein can also be used to treat or prevent insulin-related disorders, for example: II diabetes mellitus, hyperglycemia, obesity, disorders associated with disturbances in glucose or electrolyte transport and insulin secretion in cells, or endocrine disorders. They can be also used in insulin resistance treatment and post-surgical and non-post surgery decrease in insulin responsiveness.

The polypeptides and agonists described herein can be used to prevent and/or treat pulmonary and respiratory related disorders, including, inhalation, ventilation and mucus secretion disorders, pulmonary hypertension, chronic obstruction of vessels and airways, acute respiratory failure, and irreversible obstructions of vessels and bronchi. One may administer an agent described herein for treating bronchospasm, for inducing bronchodilation, for treating chronic obstructive pulmonary disease (including chronic bronchitis with normal airflow), for treating asthma (including bronchial asthma, intrinsic asthma, extrinsic asthma, acute asthma, chronic or inveterate asthma (e.g. late asthma and airways hyper-responsiveness), dust-induced asthma, allergen-induced asthma, viral-induced asthma, cold-induced asthma, pollution-induced asthma and exercise-induced asthma) and for treating rhinitis (including acute-, allergic, hatrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis). The polypeptides described herein may also be useful in the treatment of dry eye disease and chronic sinusitis. The polypeptides described herein may also be used to prevent and/or treat disorders characterized by acute pulmonary vasoconstriction such as may result from pneumonia, traumatic injury, aspiration or inhalation injury, fat embolism in the lung, acidosis inflammation of the lung, adult respiratory distress syndrome, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, herapin-protamine reactions, sepsis, status asthmaticus or hypoxia (including iatrogenic hypoxia) and other forms of reversible pulmonary vasoconstriction. Such pulmonary disorders also are also characterized by inflammation of the lung including those associated with the migration into the lung of nonresident cell types including the various leucocyte subclasses. Also included in the respiratory disorders contemplated are: bullous disease, cough, chronic cough associated with inflammation or iatrogenic induced, airway constriction, pigeon fancier's disease, eosinophilic bronchitis, asthmatic bronchitis, chronic bronchitis with airway obstruction (chronic obstructive bronchitis), eosinophilic lung disease, emphysema, farmer's lung, allergic eye diseases (including allergic conjunctivitis, vernal conjunctivitis, vernal keratoconjunctivitis, and giant papillary conjunctivitis), idiopathic pulmonary fibrosis, cystic fibrosis, diffuse pan bronchiolitis and other diseases which are characterized by inflammation of the lung and/or excess mucosal secretion. Other physiological events which are contemplated to be prevented, treated or controlled include platelet activation in the lung, chronic inflammatory diseases of the lung which result in interstitial fibrosis, such as interstitial lung diseases (ILD) (e.g., idiopathic pulmonary fibrosis, or ILD associated with rheumatoid arthritis, or other autoimmune conditions), chronic obstructive pulmonary disease (COPD) (such as irreversible COPD), chronic sinusitis, fibroid lung, hypersensitivity lung diseases, hypersensitivity pneumonitis, idiopathic interstitial pneumonia, nasal congestion, nasal polyposis, and otitis media.

The polypeptides and agonists described herein can be used alone or in combitherapy to prevent or treat: retinopathy, nephropathy, diabetic angiopathy, and edema formation

The polypeptides and agonists described herein can be used alone or in combitherapy to prevent or treat neurological disorders, for example, headache, tension-type headache, migraines, anxiety, stress, cognitive disorders, cerebral ischemia, brain trauma, movement disorders, aggression, psychosis, seizures, panic attacks, hysteria, sleep disorders, depression, schizoaffective disorders, sleep apnea, attention deficit syndromes, memory loss, dementia, memory and learning disorders as discussed in Moncada and Higgs 1995 FASEB J. 9:1319-1330; Severina 1998 Biochemistry 63:794; Lee et al. 2000 PNAS 97: 10763-10768; Hobbs 1997 TIPS 18:484-491; Murad 1994 Adv. Pharmacol. 26:1-335; and Denninger et al. 1999 Biochim. Biophys. Acta 1411:334-350 and narcolepsy. They may also be used as a sedative.

The polypeptides and detectably polypeptides and agonists described herein can be used as markers to identify, detect, stage, or diagnosis diseases and conditions of small intestine, including, without limitation: Crohn's disease, colitis, inflammatory bowel disease, tumors, benign tumors, such as benign stromal tumors, adenoma, angioma, adenomatous (pedunculated and sessile) polyps, malignant, carcinoid tumors, endocrine cell tumors, lymphoma, adenocarcinoma, foregut, midgut, and hindgut carcinoma, gastroinstestinal stromal tumor (GIST), such as leiomyoma, cellular leiomyoma, leiomyoblastoma, and leiomyosarcoma, gastrointestinal autonomic nerve tumor, malabsorption syndromes, celiac diseases, diverticulosis, Meckel's diverticulum, colonic diverticula, megacolon, Hirschsprung's disease, irritable bowel syndrome, mesenteric ischemia, ischemic colitis, colorectal cancer, colonic polyposis, polyp syndrome, intestinal adenocarcinoma, Liddle syndrome, Brody myopathy, infantile convulsions, and choreoathetosis

The polypeptides and agonists described herein can be conjugated to another molecule (e.g., a diagnostic or therapeutic molecule) to target cells bearing the GC-C receptor, e.g., cystic fibrosis lesions and specific cells lining the intestinal tract. Thus, they can be used to target radioactive moieties or therapeutic moieties (active moieties like a radionuclide, an enzyme, a fluorescent label, a metal chelating group, a chemiluminescent label, a bioluminescent label, a chemotherapeutic, a toxin, an inactive prodrug, a radiosensitizing agent, a photodynamic agent) to the intestine to aid in imaging and diagnosing or treating colorectal/metastasized or local colorectal cancer. In addition, they can be used to deliver antisense molecules or nucleic acid molecules (like normal copies of the p53 tumor suppressor gene) to the intestinal tract. The polypeptides and agonists described herein can also be used to increase the number of GC-C molecules on the surface of a cell. In some embodiments the cell is a metastasized colorectal cancer cell. In one embodiment the polypeptide or agonist described herein is therapeutically conjugated to a second agent. In certain embodiments, the second agent can be radioactive or radiostable. In certain embodiments the second agent can be selected from the group consisting of a compound that causes cell death, a compound that inhibits cell division, a compound that induces cell differentiation, a chemotherapeutic, a toxin and a radiosensitizing agent. In certain embodiments the second agent can be selected from the group consisting of: methotrexate, doxorubicin, daunorubicin, cytosinarabinoside, etoposide, 5-4 fluorouracil, melphalan, chlorambucil, cis-platin, vindesine, mitomycin, bleomycin, purothionin, macromomycin, 1,4-benzoquinone derivatives, trenimon, ricin, ricin A chain, Pseudomonas exotoxin, diphtheria toxin, Clostridium perfringens phospholipase C, bovine pancreatic ribonuclease, pokeweed antiviral protein, abrin, abrin A chain, cobra venom factor, gelonin, saporin, modeccin, viscumin, volkensin, nitroimidazole, metronidazole and misonidazole. In certain embodiments the second agent can be a cytoxic agent selected from the group consisting of cemadotin, a derivative of cemadotin, a derivative of hemiasterlin, esperamicin C, neocarzinostatin, maytansinoid DM1, 7-chloromethyl-10,11 methylenedioxy-camptothecin, rhizoxin, and the halichondrin B analog, ER-086526.

The polypeptides and agonists described herein can be used alone or in combination therapy to prevent and/or treat inner ear disorders, e.g., to prevent and/or treat Meniere's disease (including symptoms thereof such as vertigo, hearing loss, tinnitus, sensation of fullness in the ear), Mal de debarquement syndrome, otitis externa, otitis media, otorrhea, acute mastoiditis, otosclerosis, otic pain, otic bleeding, otic inflammation, Lermoyez's syndrome, vestibular neuronitis, benign paroxysmal positional vertigo (BPPV), herpes zoster oticus, Ramsay Hunt's syndrome, herpes, labyrinthitis, purulent labyrinthitis, perilymph fistulas, presbycusis, ototoxicity (including drug-induced ototoxicity), neuromias (including acoustic neuromas), aerotitis media, infectious myringitis, bullous myringitis, squamous cell carcinoma, basal cell carcinoma, pre-cancerous otic conditions, nonchromaffin paragangliomas, chemodectomas, glomus jugulare tumors, glomus tympanicum tumors, perichondritis, aural eczematoid dermatitis, malignant external otitis, subperichondrial hematoma, ceruminomas, impacted cerumen, sebaceous cysts, osteomas, keloids, otalgia, tinnitus, tympanic membrane infection, tympanitis, otic furuncles, petrositis, conductive and sensorineural hearing loss, epidural abscess, lateral sinus thrombosis, subdural empyema, otitic hydrocephalus, Dandy's syndrome, bullous myringitis, diffuse external otitis, foreign bodies, keratosis obturans, otic neoplasm, otomycosis, trauma, acute barotitis media, acute eustachian tube obstruction, postsurgical otalgia, cholesteatoma, infections related to an otic surgical procedure, and complications associated with any of said disorders. The polypeptides and agonists described herein can be used alone or in combination therapy to maintain fluid homeostasis in the inner ear, neuronitis (including viral neuronitis), ganglionitis, geniculate.

The polypeptides and agonists described herein can be used alone or in combination therapy to prevent and/or treat disorders associated with fluid and sodium retention, e.g., diseases of the electrolyte-water/electrolyte transport system within the kidney, gut and urogenital system, congestive heart failure, hypertension, hypotension, salt dependent forms of high blood pressure, hepatic edema, and liver cirrhosis. In addition they can be used to facilitate diuresis or control intestinal fluid. The polypeptides and agonists described herein can also be used to treat disorders where there is abnormal proliferation of epithelial cells within the kidney (e.g. as in the case of renal cancer). In some cases, the methods entail treatment of an individual that is salt sensitive, e.g., an individual that exhibits greater than average changes in blood pressure in response to changes in salt intake. Some salt sensitive individuals may have a greater than 5-mm Hg decrease in mean blood pressure when daily salt intake is reduced from 260 mmol to 20 mmol. There are various tests for assessing salt sensitivity (see, for example, de la Sierra et al. 2002 Journal of Human Hypertension 16:256).

The polypeptides and agonists described herein can be used alone or in combination therapy to prevent and/or treat kidney disease. “Kidney disease” includes renal failure (including acute renal failure), renal insufficiency, nephrotic edema, glomerulonephritis, pyelonephritis, kidney failure, chronic renal failure, nephritis, nephrosis, azotemia, uremia, immune renal disease, acute nephritic syndrome, rapidly progressive nephritic syndrome, nephrotic syndrome, Berger's Disease, chronic nephritic/proteinuric syndrome, tubulointerstital disease, nephrotoxic disorders, renal infarction, atheroembolic renal disease, renal cortical necrosis, malignant nephroangiosclerosis, renal vein thrombosis, renal tubular acidosis, renal glucosuria, nephrogenic diabetes insipidus, Bartter's Syndrome, Liddle's Syndrome, polycystic kidney disease, medullary cystic disease, medullary sponge kidney, hereditary nephritis, and nail-patella syndrome, along with any disease or disorder that relates to the renal system and related disorders, as well as symptoms indicative of, or related to, renal or kidney disease and related disorders.

The polypeptides and agonists described herein can be used alone or in combination therapy to prevent or treat polycystic kidney disease. Polycystic kidney disease” “PKD” (also called “polycystic renal disease”) refers to a group of disorders characterized by a large number of cysts distributed throughout dramatically enlarged kidneys. The resultant cyst development leads to impairment of kidney function and can eventually cause kidney failure. “PKD” specifically includes autosomal dominant polycystic kidney disease (ADPKD) and recessive autosomal recessive polycystic kidney disease (ADPKD), in all stages of development, regardless of the underlying cause.

The polypeptides and agonists described herein can be used alone or in combination therapy to prevent and/or treat disorders associated with bicarbonate secretion, e.g., Cystic Fibrosis.

The polypeptides and agonists described herein can be used alone or in combination therapy to prevent and/or treat disorders associated with bile secretion. In addition, they can be used to facilitate or control chloride and bile fluid secretion in the gall bladder.

The polypeptides and agonists described herein can be used alone or in combination therapy to prevent and/or treat disorders associated with liver cell regeneration. This may include administration of the polypeptides and agonists to liver transplant recipients and to patients with drug or alcohol induced-liver damage. Furthermore, the polypeptides and agonists may be useful to treat liver damage as in the case of viral mediated hepatitis. The polypeptides and agonists described herein may be used alone or in combination to prevent and/or treat liver abscess, liver cancer (either primary or metastatic), cirrhosis (such as cirrhosis caused by the alcohol consumption or primary biliary cirrhosis), amebic liver abscess, autoimmune hepatitis, biliary atresia, coccidioidomycosis disseminated, δ agent (hepatitis δ), hemochromatosis, hepatitis a, hepatitis b, hepatitis c, or any other acute, subacute, fulminant or chronic hepatitis of viral, metabolic or toxic etiology, hepatocellular carcinoma, pyogenic liver abscess, Reye's syndrome, sclerosing cholangitis, Wilson's disease, drug induced hepatotoxicity, or fulminant or acute liver failure. The polypeptides and agonists may be used in stimulating hepatic regeneration after surgical hepatectomy.

The polypeptides and agonists described herein can be used alone or in combination therapy to prevent and/or treat myocardial infraction, coronary artery disease, nitrate-induced tolerance, nitrate tolerance, diastolic dysfunction, angina pectoris, stable, unstable and variant (Prinzmetal) angina, atherosclerosis, thrombosis, endothelial dysfunction, cardiac edema, stroke, conditions of reduced blood vessel patency, e.g., postpercutaneous transluminal coronary angioplasty (post-PTCA), and peripheral vascular disease.

The polypeptides and agonists described herein can be used alone or in combination therapy to prevent and/or treat glaucoma.

The polypeptides and agonists described herein can be used alone or in combination therapy to prevent and/or treat immunodeficiency.

The polypeptides and agonists described herein can be used alone or in combination therapy to prevent and/or treat bladder outlet obstruction and incontinence.

The polypeptides and agonists described herein can be used alone or in combination therapy to prevent and/or treat male (e.g. erectile dysfunction) or female sexual dysfunction, dysmenorrhea, endometriosis, polycystic ovary syndrome, vaginal dryness, uterine pain, or pelvic pain. These polypeptides and agonists described herein can be utilized as tocolytic agents that decrease or arrest uterine contractions. The polypeptides and agonists described herein can be used to prevent/treat premature/preterm labor. Premature or preterm labor can be associated with, for example, an illness/disorder/condition of the mother (such as pre-eclampsia, high blood pressure or diabetes, abnormal shape or size of the uterus, weak or short cervix, hormone imbalance, vaginal infection that spreads to the uterus, abnormalities of the placenta, such as placenta previa, and excessive amniotic fluid), premature rupture of the amniotic membranes (“water breaks”), large fetus, and more than one fetus. The polypeptides or agonists described herein can be used to prevent uterine rupture. The polypeptides or agonists described herein can be used treat rapid uterine contractions (for example, associated with placental abruption wherein the placental abruption is associated with hypertension, diabetes, a multiply pregnancy, an unusually large amount of amniotic fluid, numerous previous deliveries, or advanced maternal age (e.g. >40 years old). In certain embodiments they can be used in combination with a phosphodiesterase inhibitor. The polypeptides and agonists described herein can be used alone or in combination therapy to prevent and/or treat infertility, for example, male infertility due to poor sperm quality, decreased sperm motility or low sperm count.

The polypeptides and agonists described herein can be used alone or in combination therapy to prevent and/or treat osteopenia disorders (bone loss disorders). “Bone loss disorders” include conditions and diseases wherein the inhibition of bone loss and/or the promotion of bone formation is desirable. Among such conditions and diseases are osteoporosis, osteomyelitis, Paget's disease (osteitis deformans), periodontitis, hypercalcemia, osteonecrosis, osteosarcoma, osteolyic metastases, familial expansile osteolysis, prosthetic loosening, periprostetic osteolysis, bone loss attendant rheumatoid arthritis, and cleiodocranial dysplasia (CCD). Osteoporosis includes primary osteoporosis, endocrine osteoporosis (hyperthyroidism, hyperparathyroidism, Cushing's syndrome, and acromegaly), hereditary and congenital forms of osteoporosis (osteogenesis imperfecta, homocystinuria, Menkes' syndrome, and Rile-Day syndrome) and osteoporosis due to immobilization of extremitiesosteomyelitis, or an infectious lesion in bone leading to bone loss. The polypeptides and agonists can be used alone or in combination therapy to stimulating bone regeneration. The bone regeneration may be following reconstruction of bone defects in cranio-maxillofacial surgery, or following an implant into bone, for example a dental implant, bone supporting implant, or prosthesis. The bone regeneration may also be following a bone fracture.

The polypeptides and agonists described herein may be used alone or in combination therapy (for example, with other agents that increase cGMP) to prevent or treat disorders related to an alteration in cGMP including, but not limited to Alzheimer's disease, psoriasis, skin necrosis, scarring, fibrosis, baldness, Kawasaki's Disease, nutcracker esophagus (US20050245544), septic shock, NSAID-induced gastric disease or disorder, ischemic renal disease or disorder, peptic ulcer, sickle cell anemia, epilepsy, and a neuroinflammatory disease or disorder (for example as described in WO05105765).

### Treatment of the Side-Effects of Opioid Administration

The polypeptides described herein (e.g. the GCC agonist polypeptides described herein) may be useful in the treatment of one or more side effects of opioid administration, e.g., opioid induced constipation, nausea and/or vomiting. In the case of constipation, the polypeptide can be administered at a dosage to induce laxation within a desired time (e.g., within 15 minutes, 30 minutes, 1 hour, 2 hours, 3 hours, 4 hours, 5 hours, 6 hours, 7 hours, 8 hours, 9 hours, 10 hours, 12 hours, 18 hours or 24 hours).

The polypeptide can be administered to maintain regular bowel movements in a patient who is a chronic opioid user (e.g., a terminally-ill patient). The administration can be via any convenient route (e.g., sublingual, parenteral, intravenous, subcutaneous).

Thus, the polypeptides described herein can be administered to a patient that is taking one or more of the following opioids: Acetorphine, Acetyldihydrocodeine, Acetylmorphone, Alfentanil, Allylprodine, Anileridine, Bemidone, Benzylmorphine, Bezitramide, Buprenorphine, Butorphanol, Carfentanil/Carfentanyl, Clonitazene, Codeine, Codeine-N-Oxide, Codeinone, Cyclazocine, Cyclorphan, Desomorphine, Dextromoramide, Dextropropoxyphene, Dezocine, Diacetyldihydromorphine, Diamorphine/Diacetylmorphine (Heroin), Diethylthiambutene, Difenoxin, Dihydrocodeine, Dihydrocodeinone Enol Acetate, Dihydroetorphine, Dihydroisocodeine, Dihydromorphine, Dimethylthiambutene, Diphenoxylate, Dipropanoylmorphine, Drobetabol, Ethylketocyclazocine, Ethylmorphine, Etonitazene, Etorphine, Fentanyl, Hydrocodone, Hydromorphone, Isomethadone, Ketobemidone, Laudanum, Lefetamine, Levallorphan, Levo-Alphacetylmethadol (LAAM), Levomethorphan, Levorphanol, Loperamide, Meptazinol, Metazocine, Methadone, Monoacetylmorphine, Morphine, Morphine-6-Glucuronide, Morphine-N-Oxide, Morphinone, MPPP (1-Methyl 4-Phenyl 4-Propionoxypiperidine), Myorphine, Nalbuphine/Nalbufine, Nicocodeine, Nicodicodeine, Nicomorphine, Norcodeine, Ohmefentanyl, Oxycodone, Oxymorphone, Pentazocine, PEPAP (1-Phenethyl-4-Phenyl-4-Piperidinol Acetate (Ester)), Pethidine (Meperidine), Phenadoxone, Phenazocine, Phenoperidine, Pholcodeine, Piminodine, Piritramide, Prodine, Propiram, Propoxyphene, Racemethorphan, Remifentanil, Sufentanil, Thebaine, Thiofentanil/Thiofentanyl, Tilidine, and Tramadol. The peptide can be co-administered with or co-formulated with any of the preceeding peptides.

Where the polypeptide described herein is co-formulated with an opioid the composition may further include one or more other active ingredients that may be conventionally employed in analgesic and/or cough-cold-antitussive combination products. Such conventional ingredients include, for example, aspirin, acetaminophen, phenylpropanolamine, phenylephrine, chlorpheniramine, caffeine, and/or guaifenesin. Typical or conventional ingredients that may be included in the opioid component are described, for example, in the Physicians' Desk Reference, 1999, the disclosures of which are hereby incorporated herein by reference, in their entirety.

In addition, the composition may further include one or more compounds that may be designed to enhance the analgesic potency of the opioid and/or to reduce analgesic tolerance development. Such compounds include, for example, dextromethorphan or other NMDA antagonists (Mao, M. J. et al., Pain 1996, 67, 361), L-364,718 and other CCK antagonists (Dourish, C. T. et al., Eur J Pharmacol 1988, 147, 469), NOS inhibitors (Bhargava, H. N. et al., Neuropeptides 1996, 30, 219), PKC inhibitors (Bilsky, E. J. et al., J Pharmacol Exp Ther 1996, 277, 484), and dynorphin antagonists or antisera (Nichols, M. L. et al., Pain 1997, 69, 317). The disclosures of each of the foregoing documents are hereby incorporated herein by reference, in their entireties.

The combination products, such as pharmaceutical compositions comprising opioids in combination with a polypeptide described herein may be in any dosage form, such as those described herein, and can also be administered in various ways, as described herein. In a preferred embodiment, the combination products of the disclosure are formulated together, in a single dosage form (that is, combined together in one capsule, tablet, powder, or liquid, etc.). When the combination products are not formulated together in a single dosage form, the opioid compounds and the polypeptides described herein may be administered at the same time (that is, together), or in any order. When not administered at the same time, preferably the administration of an opioid and a polypeptide described herein occurs less than about one hour apart, less than about 30 minutes apart, less than about 15 minutes apart, and less than about 5 minutes apart. Administration of the combination of an opioid and a polypeptide described herein can be, for example, oral, although other routes of administration, as described above, are contemplated to be within the scope of the present disclosure. Although it is the opioids and polypeptides described herein may both be administered in the same fashion (that is, for example, both orally), if desired, they may each be administered in different fashions (that is, for example, one component of the combination product may be administered orally, and another component may be administered intravenously). The dosage of the combination products of the disclosure may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired.

Although the proper dosage of the combination products of this disclosure will be readily ascertainable by one skilled in the art, by way of general guidance, where an opioid compounds is combined with a polypeptide described herein, for example, typically a daily dosage may range from about 0.01 to about 100 milligrams, 0.1 to about 10 milligrams of the opioid, 15 to about 200 milligrams, 1, 2, 3, 4, 5, 6, 7, 8, 9, 10 milligrams of opioid per kilogram of patient body weight. The opioid-GCC agonist combination product can include, for example, from 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 10 μg, 20 μg, 30 μg, 40 μg, 50 μg, 60 μg, 70 μg, 75 μg, 80 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg of a polypeptide described herein.

When provided as a single dosage form, the potential exists for a chemical interaction between the combined active ingredients (for example, an opioid and a polypeptide described herein). For this reason, the preferred dosage forms of the combination products of this disclosure are formulated such that although the active ingredients are combined in a single dosage form, the physical contact between the active ingredients is minimized (that is, reduced).

In order to minimize contact, one embodiment of this disclosure where the product is orally administered provides for a combination product wherein one active ingredient is enteric coated. By enteric coating one or more of the active ingredients, it is possible not only to minimize the contact between the combined active ingredients, but also, it is possible to control the release of one of these components in the gastrointestinal tract such that one of these components is not released in the stomach but rather is released in the intestines. Another embodiment of this disclosure where oral administration is desired provides for a combination product wherein one of the active ingredients is coated with a sustained-release material which effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

Dosage forms of the combination products include those wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present disclosure can be in the form of capsules wherein one active ingredient is compressed into a tablet or in the form of a plurality of microtablets, particles, granules or non-perils, which are then enteric coated. These enteric coated microtablets, particles, granules or non-perils are then placed into a capsule or compressed into a capsule along with a granulation of the other active ingredient.

These as well as other ways of minimizing contact between the components of combination products of the present disclosure, whether administered in a single dosage form or administered in separate forms but at the same time by the same manner, will be readily apparent to those skilled in the art in light of the present disclosure.

### Peptides as Immunogens

The polypeptides described herein can be used as immunogens to create antibodies for immunoassays. The polypeptides described herein can be used as immunogens to treat and/or prevent one or more disease symptoms associated with traveler's diarrhea and for vaccination against pathogens, including but not limited to enterotoxigenic E. coli (ETEC). They may also be used in vaccines which also comprise interleukin 18 and either saponin adjuvant or CpG adjuvant for example as described in WO05039634 and WO05039630. The methods described in US20040146534, U.S. Pat. No. 4,220,584, U.S. Pat. No. 4,285,391, U.S. Pat. No. 5,182,109, U.S. Pat. No. 4,603,049, U.S. Pat. No. 4,545,931, U.S. Pat. No. 4,886,663, U.S. Pat. No. 4,758,655, WO08402700, FR2525592, and FR2532850 can be similarly used to create immunogens comprising the polypeptides described herein. U.S. Pat. No. 6,043,057, U.S. Pat. No. 5,834,246, U.S. Pat. No. 5,268,276, and EP368819, specifically describe an expression system containing CTB (cholera toxin Beta subunit) fused to an ST-like polypeptide under a foreign promoter for use as a vaccine. The nucleic acids that encode the polypeptides described herein may be use as genetic vaccines as described in US20050260605 and WO0148018. The nucleic acid molecules may also be used for the manufacture of a functional ribonucleic acid, wherein the functional ribonucleic acid is selected from the group comprising ribozymes, antisense nucleic acids and siRNA (as described in WO05103073).

1. A purified polypeptide comprising at least 10 contiguous amino acids of SEQ ID NO:X
TVQDGNFSFSLESVKKLKDLQEPQEPRVGKLRNFAPIPGEPVVPILCSNPNFPEE LKPLCKEPNAQEILQRLEEIAEDPGTCEICAYAACTGC (SEQ ID NO:X; human proguanylin).

2. The purified polypeptide of claim 1 wherein the polypeptide is selected from:
a) a polypeptide comprising amino acids 1-16 of SEQ ID NO:X;
b) a polypeptide comprising amino acids 1-47 of SEQ ID NO:X;
c) a polypeptide comprising amino acids 1-61 of SEQ ID NO:X;
d) a polypeptide comprising amino acids 1-72 of SEQ ID NO:X;
e) a polypeptide comprising amino acids 17-47 of SEQ ID NO:X;
f) a polypeptide comprising amino acids 17-61 of SEQ ID NO:X;
g) a polypeptide comprising amino acids 17-72 of SEQ ID NO:X;
h) a polypeptide comprising amino acids 17-94 of SEQ ID NO:X;
i) a polypeptide comprising amino acids 48-61 of SEQ ID NO:X;
j) a polypeptide comprising amino acids 48-72 of SEQ ID NO:X;
k) a polypeptide comprising amino acids 48-94 of SEQ ID NO:X;
l) a polypeptide comprising amino acids 62-72 of SEQ ID NO:X;
m) a polypeptide comprising amino acids 62-94 of SEQ ID NO:X;
n) a polypeptide comprising amino acids 73-94 of SEQ ID NO:X;
o) a polypeptide comprising amino acids 1-23 of SEQ ID NO:X;
p) a polypeptide comprising amino acids 48-66 of SEQ ID NO:X;
q) a polypeptide comprising amino acids 48-71 of SEQ ID NO:X;
r) a polypeptide comprising amino acids 48-76 of SEQ ID NO:X;
s) a polypeptide comprising amino acids 43-61 of SEQ ID NO:X;
t) a polypeptide comprising amino acids 38-61 of SEQ ID NO:X;
u) a polypeptide comprising amino acids 33-61 of SEQ ID NO:X;
v) a polypeptide comprising amino acids 43-66 of SEQ ID NO:X;
w) a polypeptide comprising amino acids 38-66 of SEQ ID NO:X;
x) a polypeptide comprising amino acids 33-66 of SEQ ID NO:X;
y) a polypeptide comprising amino acids 43-71 of SEQ ID NO:X;
z) a polypeptide comprising amino acids 38-71 of SEQ ID NO:X;
aa) a polypeptide comprising amino acids 33-71 of SEQ ID NO:X;
ab) a polypeptide comprising amino acids 43-76 of SEQ ID NO:X;
ac) a polypeptide comprising amino acids 38-76 of SEQ ID NO:X; and
ad) a polypeptide comprising amino acids 33-76 of SEQ ID NO:X.

3. The purified polypeptide of claim 1 wherein the polypeptide is selected from:
a) a polypeptide consisting of amino acids 1-16 of SEQ ID NO:X;
b) a polypeptide consisting of amino acids 1-47 of SEQ ID NO:X;
c) a polypeptide consisting of amino acids 1-61 of SEQ ID NO:X;
d) a polypeptide consisting of amino acids 1-72 of SEQ ID NO:X;
e) a polypeptide consisting of amino acids 17-47 of SEQ ID NO:X;
f) a polypeptide consisting of amino acids 17-61 of SEQ ID NO:X;
g) a polypeptide consisting of amino acids 17-72 of SEQ ID NO:X;
h) a polypeptide consisting of amino acids 17-94 of SEQ ID NO:X;
i) a polypeptide consisting of amino acids 48-61 of SEQ ID NO:X;
j) a polypeptide consisting of amino acids 48-72 of SEQ ID NO:X;
k) a polypeptide consisting of amino acids 48-94 of SEQ ID NO:X;
l) a polypeptide consisting of amino acids 62-72 of SEQ ID NO:X;
m) a polypeptide consisting of amino acids 62-94 of SEQ ID NO:X;
n) a polypeptide consisting of amino acids 73-94 of SEQ ID NO:X;
o) a polypeptide consisting of amino acids 1-23 of SEQ ID NO:X;
p) a polypeptide consisting of amino acids 48-66 of SEQ ID NO:X;
q) a polypeptide consisting of amino acids 48-71 of SEQ ID NO:X;
r) a polypeptide consisting of amino acids 48-76 of SEQ ID NO:X;
s) a polypeptide consisting of amino acids 43-61 of SEQ ID NO:X;
t) a polypeptide consisting of amino acids 38-61 of SEQ ID NO:X;
u) a polypeptide consisting of amino acids 33-61 of SEQ ID NO:X;
v) a polypeptide consisting of amino acids 43-66 of SEQ ID NO:X;
w) a polypeptide consisting of amino acids 38-66 of SEQ ID NO:X;
x) a polypeptide consisting of amino acids 33-66 of SEQ ID NO:X;
y) a polypeptide consisting of amino acids 43-71 of SEQ ID NO:X;
z) a polypeptide consisting of amino acids 38-71 of SEQ ID NO:X;
aa) a polypeptide consisting of amino acids 33-71 of SEQ ID NO:X;
ab) a polypeptide consisting of amino acids 43-76 of SEQ ID NO:X;
ac) a polypeptide consisting of amino acids 38-76 of SEQ ID NO:X; and
ad) a polypeptide consisting of amino acids 33-76 of SEQ ID NO:X.

4. The purified polypeptide of claim 1 wherein the polypeptide is selected from:
a) a polypeptide consisting essentially of amino acids 1-16 of SEQ ID NO:X;
b) a polypeptide consisting essentially of amino acids 1-47 of SEQ ID NO:X;
c) a polypeptide consisting essentially of amino acids 1-61 of SEQ ID NO:X;
d) a polypeptide consisting essentially of amino acids 1-72 of SEQ ID NO:X;
e) a polypeptide consisting essentially of amino acids 17-47 of SEQ ID NO:X;
f) a polypeptide consisting essentially of amino acids 17-61 of SEQ ID NO:X;
g) a polypeptide consisting essentially of amino acids 17-72 of SEQ ID NO:X;
h) a polypeptide consisting essentially of amino acids 17-94 of SEQ ID NO:X;
i) a polypeptide consisting essentially of amino acids 48-61 of SEQ ID NO:X;
j) a polypeptide consisting essentially of amino acids 48-72 of SEQ ID NO:X;
k) a polypeptide consisting essentially of amino acids 48-94 of SEQ ID NO:X;
l) a polypeptide consisting essentially of amino acids 62-72 of SEQ ID NO:X;
m) a polypeptide consisting essentially of amino acids 62-94 of SEQ ID NO:X;
n) a polypeptide consisting essentially of amino acids 73-94 of SEQ ID NO:X;
o) a polypeptide consisting essentially of amino acids 1-23 of SEQ ID NO:X;
p) a polypeptide consisting essentially of amino acids 48-66 of SEQ ID NO:X;
q) a polypeptide consisting essentially of amino acids 48-71 of SEQ ID NO:X;
r) a polypeptide consisting essentially of amino acids 48-76 of SEQ ID NO:X;
s) a polypeptide consisting essentially of amino acids 43-61 of SEQ ID NO:X;
t) a polypeptide consisting essentially of amino acids 38-61 of SEQ ID NO:X;
u) a polypeptide consisting essentially of amino acids 33-61 of SEQ ID NO:X;
v) a polypeptide consisting essentially of amino acids 43-66 of SEQ ID NO:X;
w) a polypeptide consisting essentially of amino acids 38-66 of SEQ ID NO:X;
x) a polypeptide consisting essentially of amino acids 33-66 of SEQ ID NO:X;
y) a polypeptide consisting essentially of amino acids 43-71 of SEQ ID NO:X;
z) a polypeptide consisting essentially of amino acids 38-71 of SEQ ID NO:X;
aa) a polypeptide consisting essentially of amino acids 33-71 of SEQ ID NO:X;
ab) a polypeptide consisting essentially of amino acids 43-76 of SEQ ID NO:X;
ac) a polypeptide consisting essentially of amino acids 38-76 of SEQ ID NO:X; and
ad) a polypeptide consisting essentially of amino acids 33-76 of SEQ ID NO:X.

5. A purified polypeptide comprising at least 0.10 contiguous amino acids of SEQ ID NO:X1
X₁ X₂ X₃ X₄ X₅ X₆ X₇ X₈ X₉ X₁₀ X₁₁ L E X₁₄ V K X₁₇ L, X₁₉ X₂₀ L X₂₂ X₂₃ X₂₄ X₂₅ X₂₆ X₂₇ X₂₈ X₂₉ X₃₀ X₃₁ X₃₂ X₃₃ X₃₄ X₃₅ X₃₆ X₃₇ X₃₈ X₃₉ X₄₀ X₄₁ X₄₂ X₄₃ X₄₄ X₄₅ X₄₆ X₄₇ X₄₈ X₄₉ X₅₀ C X₅₂ X₅₃ X₅₄ X₅₅ X₅₆ X₅₇ P X₅₉ X₆₀ X₆₁ X₆₂ P X₆₄ C X₆₆ X₆₇ X₆₈ X₆₉ X₇₀ X₇₁ X₇₂ X₇₃ X₇₄ X₇₅ R L X₇₈ X₇₉ X₈₀ X₈₁ X₈₂ X₈₃ P G T C E I C A Y A A C T G C X₉₉ (SEQ ID NO:X1-guanylin consensus 1) wherein:
X₁ is V or S;
X₂ is T, L, I, Y or E;
X₃ is V or F;
X₄ is Q or K;
X₅ is D or E;
X₆ is G or N;
X₇ is D, N, E or G;
X₈ is F or L;
X₉ is S, T or K;
X₁₀ is F or Y;
X₁₁ is S or P;
X₁₄ is S or A;
X₁₇ is K, Q or R;
X₁₉ is K or H;
X₂₀ is D, E, A, H or G;
X₂₂ is Q, R, G, M, A;
X₂₃ is E, Q or D;
X₂₄ is A, S, E, V, L or P;
X₂₅ is Q, N, P, G or S;
X₂₆ is E, K, M or V;
X₂₇ is G, L or is missing;
X₂₈ is Q, S, R, A or is missing;
X₂₉ is E, K, S, A or is missing;
X₃₀ is P, V, M or A;
X₃₁ is R, Q, T, I, A or is missing;
X₃₂ is L, V, I, G, N or S;
X₃₃ is P, G, R, V, M, A, P;
X₃₄ is S, R or K;
X₃₅ is H, L, I, N or K;
X₃₆ is R, K or is missing;
X₃₇ is N, K or is missing;
X₃₈ is F or is missing;
X₃₉ is A or is missing;
X₄₀ is P, L or is missing;
X₄₁ is I, R or is missing;
X₄₂ is L, P, F, V, R or is missing;
X₄₃ is G, V, D, P, L, A or is missing;
X₄₄ is G, E, K, A, Q, R or S;
X₄₅ is P, S, H or K;
X₄₆ is V, I, P, A or Q;
X₄₇ is A, V, I, A, L, G or T;
X₄₈ is P, A, S, Y or is missing;
X₄₉ is I, Q, V, N, G, E, H, S or F;
X₅₀ is L, A or P;
X₅₂ is S, N, A, Q or G;
X₅₃ is S or missing;
X₅₄ is H, N, D, S, L, F, or Q;
X₅₅ is P, S, L or K;
X₅₆ is A, K, N, T, G, or Q;
X₅₇ is F or L;
X₅₉ is E, K or Q;
X₆₀ is E, A or D;
X₆₁ is L or F;
X₆₂ is K, R, Q or L;
X₆₄ is L, I or V;
X₆₆ is K, E, Q, T or R;
X₆₇ is E, K, R or Q;
X₆₈ is P, S, E or R;
X₆₉ is N, D or G;
X₇₀ is A or S;
X₇₁ is E, Q, P, A or S;
X₇₂ is E, D, Q, M or A;
X₇₃ is I, A, or S;
X₇₄ is L, F or V;
X₇₅ is Q, E, D, N, G or A;
X₇₈ is E, A, G or C;
X₇₉ is E, A, V, S, L or M;
X₈₀ is I or V;
X₈₁ is A or P;
X₈₂ is E, Q, A or S;
X₈₃ is D or E; and
X₉₉ is F or is missing.

6. The purified polypeptide of claim 5 wherein:
X₁ is V;
X₂ is T or L;
X₃ is V or F;
X₄ is Q or K;
X₅ is D or E;
X₆ is G or N;
X₇ is D or N;
X₈ is F or L;
X₉ is S;
X₁₀ is F or Y;
X₁₁ is S or P;
X₁₄ is S;
X₁₇ is K;
X₁₉ is K;
X₂₀ is D or E;
X₂₂ is Q or R;
X₂₃ is E;
X₂₄ is V, L or P;
X₂₅ is Q or P;
X₂₆ is E or K;
X₂₇ is missing;
X₂₈ is missing;
X₂₉ is missing;
X₃₀ is P or V;
X₃₁ is R
X₃₂ is L or V;
X₃₃ is P or G;
X₃₄ is S, R or K;
X₃₅ is H or L;
X₃₆ is R, K or is missing;
X₃₇ is N, K or is missing;
X₃₈ is F or is missing;
X₃₉ is A or is missing;
X₄₀ is P or is missing;
X₄₁ is I, R or is missing;
X₄₂ is L or P;
X₄₃ is G or L;
X₄₄ is G, E or K,
X₄₅ is P or S;
X₄₆ is V or A;
X₄₇ is A or V;
X₄₈ is P or is missing;
X₄₉ is I or Q;
X₅₀ is L;
X₅₂ is S;
X₅₃ is missing;
X₅₄ is H, N, or D;
X₅₅ is P or S;
X₅₆ is A, K or N;
X₅₇ is F or L;
X₅₉ is E;
X₆₀ is E or A;
X₆₁ is L;
X₆₂ is K or R;
X₆₄ is L, I or V;
X₆₆ is K, E, Q, T or R;
X₆₇ is E or K;
X₆₈ is P;
X₆₉ is N;
X₇₀ is A;
X₇₁ is E or Q;
X₇₂ is E;
X₇₃ is I;
X₇₄ is L;
X₇₅ is Q or E;
X₇₈ is E or A;
X₇₉ is E or A;
X₈₀ is I;
X₈₁ is A;
X₈₂ is E or Q;
X₈₃ is D; and
X₉₉ is missing.

7. A purified polypeptide comprising at least 10 contiguous amino acids of SEQ ID NO:X2
X₁ X₂ X₃ X₄ X₅ X₆ X₇ X₈ X₉ X₁₀ X₁₁ L E X₁₄ V K X₁₇ L X₁₉ X₂₀ L X₂₂ X₂₃ X₂₄ X₂₅ X₂₆ X₂₇ X₂₈ X₂₉ X₃₀ X₃₁ X₃₂ X₃₃ X₃₄ X₃₅ X₃₆ X₃₇ X₃₈ X₃₉ X₄₀ X₄₁ X₄₂ X₄₃ X₄₄ X₄₅ X₄₆ X₄₇ X₄₈ X₄₉ X₅₀ C X₅₂ X₅₃ X₅₄ X₅₅ X₅₆ X₅₇ P X₅₉ X₆₀ X_{6I} X₆₂ P X₆₄ C X₆₆ X₆₇ X₆₈ X₆₉ X₇₀ X₇₁ X₇₂ X₇₃ X₇₄ X₇₅ R L X₇₈ X₇₉ X₈₀ X₈₁ X₈₂ X₈₃ P X₈₅ X₈₆ C E I C A X₉₂ A A C X₉₆ G C X₉₉ (SEQ ID NO:X2-guanylin consensus 2), wherein:
X₁ is. V or S;
X₂ is T, L, I, Y or E;
X₃ is V or F;
X₄ is Q or K;
X₅ is D or E;
X₆ is G or N;
X₇ is D, N, E or G;
X₈ is F or L;
X₉ is S, T or K;
X₁₀ is F or Y;
X₁₁ is S or P;
X₁₄ is S or A;
X₁₇ is K, Q or R;
X₁₉ is K or H;
X₂₀ is D, E, A, H or G;
X₂₂ is Q, R, G, M, A;
X₂₃ is E, Q or D;
X₂₄ is A, S, E, V, L or P;
X₂₅ is Q, N, P, G or S;
X₂₆ is E, K, M or V;
X₂₇ is G, L or is missing;
X₂₈ is Q, S, R, A or is missing;
X₂₉ is E, K, S, A or is missing;
X₃₀ is P, V, M or A;
X₃₁ is R, Q, T, I, A or is missing;
X₃₂ is L, V, I, G, N or S;
X₃₃ is P, G, R, V, M, A, P;
X₃₄ is S, R or K;
X₃₅ is H, L, I, N or K;
X₃₆ is R, K or is missing;
X₃₇ is N, K or is missing;
X₃₈ is F or is missing;
X₃₉ is A or is missing;
X₄₀ is P, L or is missing;
X₄₁ is I, R or is missing;
X₄₂ is L, P, F, V, R or is missing;
X₄₃ is G, V, D, P, L, A or is missing;
X₄₄ is G, E, K, A, Q, R or S;
X₄₅ is P, S, H or K;
X₄₆ is V, I, P, A or Q;
X₄₇ is A, V, I, A, L, G or T;
X₄₈ is P, A, S, Y or is missing;
X₄₉ is I, Q, V, N, G, E, H, S or F;
X₅₀ is L, A or P;
X₅₂ is S, N, A, Q or G;
X₅₃ is S or missing;
X₅₄ is H, N, D, S, L, F, or Q;
X₅₅ is P, S, L or K;
X₅₆ is A, K, N, T, G, or Q;
X₅₇ is F or L;
X₅₉ is E, K or Q;
X₆₀ is E, A or D;
X₆₁ is L or F;
X₆₂ is K, R, Q or L;
X₆₄ is L, I or V;
X₆₆ is K, E, Q, T or R;
X₆₇ is E, K, R or Q;
X₆₈ is P, S, E or R;
X₆₉ is N, D or G;
X₇₀ is A or S;
X₇₁ is E, Q, P, A or S;
X₇₂ is E, D, Q, M or A;
X₇₃ is I, A, or S;
X₇₄ is L, F or V;
X₇₅ is Q, E, D, N, G or A;
X₇₈ is E, A, G or C;
X₇₉ is E, A, V, S, L or M;
X₈₀ is I or V;
X₈₁ is A or P;
X₈₂ is E, Q, A or S;
X₈₃ is D or E;
X₈₅ is G, S, R, or N;
X₈₆ is S or T;
X₉₂ is Y or F;
X₉₆ is T or A; and
X₉₉ is F or is missing.

8. The purified polypeptide of claim 7 wherein:
X₁ is V;
X₂ is T or L;
X₃ is V or F;
X₄ is Q or K;
X₅ is D or E;
X₆ is G or N;
X₇ is D or N;
X₈ is F or L;
X₉ is S;
X₁₀ is F or Y;
X₁₁ is S or P;
X₁₄ is S;
X₁₇ is K;
X₁₉ is K;
X₂₀ is D or E;
X₂₂ is Q or R;
X₂₃ is E;
X₂₄ is V, L or P;
X₂₅ is Q or P;
X₂₆ is E or K;
X₂₇ is missing;
X₂₈ is missing;
X₂₉ is missing;
X₃₀ is P or V;
X₃₁ is R
X₃₂ is L or V;
X₃₃ is P or G;
X₃₄ is S, R or K;
X₃₅ is H or L;
X₃₆ is R, K or is missing;
X₃₇ is N, K or is missing;
X₃₈ is F or is missing;
X₃₉ is A or is missing;
X₄₀ is P or is missing;
X₄₁ is I, R or is missing;
X₄₂ is L or P;
X₄₃ is G or L;
X₄₄ is G, E or K,
X₄₅ is P or S;
X₄₆ is V or A;
X₄₇ is A or V;
X₄₈ is P or is missing;
X₄₉ is I or Q;
X₅₀ is L;
X₅₂ is S;
X₅₃ is missing;
X₅₄ is H, N, or D;
X₅₅ is P or S;
X₅₆ is A, K or N;
X₅₇ is F or L;
X₅₉ is E;
X₆₀ is E or A;
X₆₁ is L;
X₆₂ is K or R;
X₆₄ is L, I or V;
X₆₆ is K, E, Q, T or R;
X₆₇ is E or K;
X₆₈ is P;
X₆₉ is N;
X₇₀ is A;
X₇₁ is E or Q;
X₇₂ is E;
X₇₃ is I;
X₇₄ is L;
X₇₅ is Q or E;
X₇₈ is E or A;
X₇₉ is E or A;
X₈₀ is I;
X₈₁ is A;
X₈₂ is E or Q;
X₈₃ is D;
X₈₅ is G, or S;
X₈₆ is T;
X₉₂ is Y;
X₉₆ is T; and
X₉₉ is missing.

9-11. (canceled)

12. A purified polypeptide consisting of a polypeptide fragment of SEQ ID NO:X comprising at least 10 contiguous amino acids of SEQ ID NO:X VTVQDGNFSFSLESVKKLKDLQEPQEPRVGKLRNFAPIPGEPVVPILCSNPNFPEELKPLC KEPNAQEILQRLEEIAEDPGTCEICAYAACTGC (SEQ ID NO:X; human proguanylin).

13. The purified polypeptide of claim 12 wherein the polypeptide fragment of SEQ ID NO:X is selected from:
a) a polypeptide comprising amino acids 1-16 of SEQ ID NO:X;
b) a polypeptide comprising amino acids 1-47 of SEQ ID NO:X;
c) a polypeptide comprising amino acids 1-61 of SEQ ID NO:X;
d) a polypeptide comprising amino acids 1-72 of SEQ ID NO:X;
e) a polypeptide comprising amino acids 17-47 of SEQ ID NO:X;
f) a polypeptide comprising amino acids 17-61 of SEQ ID NO:X;
g) a polypeptide comprising amino acids 17-72 of SEQ ID NO:X;
h) a polypeptide comprising amino acids 17-94 of SEQ ID NO:X;
i) a polypeptide comprising amino acids 48-61 of SEQ ID NO:X;
j) a polypeptide comprising amino acids 48-72 of SEQ ID NO:X;
k) a polypeptide comprising amino acids 48-94 of SEQ ID NO:X;
l) a polypeptide comprising amino acids 62-72 of SEQ ID NO:X;
m) a polypeptide comprising amino acids 62-94 of SEQ ID NO:X;
n) a polypeptide comprising amino acids 73-94 of SEQ ID NO:X;
o) a polypeptide comprising amino acids 1-23 of SEQ ID NO:X;
p) a polypeptide comprising amino acids 48-66 of SEQ ID NO:X;
q) a polypeptide comprising amino acids 48-71 of SEQ ID NO:X;
r) a polypeptide comprising amino acids 48-76 of SEQ ID NO:X;
s) a polypeptide comprising amino acids 43-61 of SEQ ID NO:X;
t) a polypeptide comprising amino acids 38-61 of SEQ ID NO:X;
u) a polypeptide comprising amino acids 33-61 of SEQ ID NO:X;
v) a polypeptide comprising amino acids 43-66 of SEQ ID NO:X;
w) a polypeptide comprising amino acids 38-66 of SEQ ID NO:X;
x) a polypeptide comprising amino acids 33-66 of SEQ ID NO:X;
y) a polypeptide comprising amino acids 43-71 of SEQ ID NO:X;
z) a polypeptide comprising amino acids 38-71 of SEQ ID NO:X;
aa) a polypeptide comprising amino acids 33-71 of SEQ ID NO:X;
ab) a polypeptide comprising amino acids 43-76 of SEQ ID NO:X;
ac) a polypeptide comprising amino acids 38-76 of SEQ ID NO:X; and
ad) a polypeptide comprising amino acids 33-76 of SEQ ID NO:X.

14. The purified polypeptide of claim 12 wherein the polypeptide fragment of SEQ ID NO:X is selected from:
a) a polypeptide consisting of amino acids 1-16 of SEQ ID NO:X;
b) a polypeptide consisting of amino acids 1-47 of SEQ ID NO:X;
c) a polypeptide consisting of amino acids 1-61 of SEQ ID NO:X;
d) a polypeptide consisting of amino acids 1-72 of SEQ ID NO:X;
e) a polypeptide consisting of amino acids 17-47 of SEQ ID NO:X;
f) a polypeptide consisting of amino acids 17-61 of SEQ ID NO:X;
g) a polypeptide consisting of amino acids 17-72 of SEQ ID NO:X;
h) a polypeptide consisting of amino acids 17-94 of SEQ ID NO:X;
i) a polypeptide consisting of amino acids 48-61 of SEQ ID NO:X;
j) a polypeptide consisting of amino acids 48-72 of SEQ ID NO:X;
k) a polypeptide consisting of amino acids 48-94 of SEQ ID NO:X;
l) a polypeptide consisting of amino acids 62-72 of SEQ ID NO:X;
m) a polypeptide consisting of amino acids 62-94 of SEQ ID NO:X;
n) a polypeptide consisting of amino acids 73-94 of SEQ ID NO:X;
o) a polypeptide consisting of amino acids 1-23 of SEQ ID NO:X;
p) a polypeptide consisting of amino acids 48-66 of SEQ ID NO:X;
q) a polypeptide consisting of amino acids 48-71 of SEQ ID NO:X;
r) a polypeptide consisting of amino acids 48-76 of SEQ ID NO:X;
s) a polypeptide consisting of amino acids 43-61 of SEQ ID NO:X;
t) a polypeptide consisting of amino acids 38-61 of SEQ ID NO:X;
u) a polypeptide consisting of amino acids 33-61 of SEQ ID NO:X;
v) a polypeptide consisting of amino acids 43-66 of SEQ ID NO:X;
w) a polypeptide consisting of amino acids 38-66 of SEQ ID NO:X;
x) a polypeptide consisting of amino acids 33-66 of SEQ ID NO:X;
y) a polypeptide consisting of amino acids 43-71 of SEQ ID NO:X;
z) a polypeptide consisting of amino acids 38-71 of SEQ ID NO:X;
aa) a polypeptide consisting of amino acids 33-71 of SEQ ID NO:X;
ab) a polypeptide consisting of amino acids 43-76 of SEQ ID NO:X;
ac) a polypeptide consisting of amino acids 38-76 of SEQ ID NO:X; and
ad) a polypeptide consisting of amino acids 33-76 of SEQ ID NO:X.

15. The purified polypeptide of claim 12 wherein the polypeptide fragment of SEQ ID NO:X is selected from:
a) a polypeptide consisting essentially of amino acids 1-16 of SEQ ID NO:X;
b) a polypeptide consisting essentially of amino acids 1-47 of SEQ ID NO:X;
c) a polypeptide consisting essentially of amino acids 1-61 of SEQ ID NO:X;
d) a polypeptide consisting essentially of amino acids 1-72 of SEQ ID NO:X;
e) a polypeptide consisting essentially of amino acids 17-47 of SEQ ID NO:X;
f) a polypeptide consisting essentially of amino acids 17-61 of SEQ ID NO:X;
g) a polypeptide consisting essentially of amino acids 17-72 of SEQ ID NO:X;
h) a polypeptide consisting essentially of amino acids 17-94 of SEQ ID NO:X;
i) a polypeptide consisting essentially of amino acids 48-61 of SEQ ID NO:X;
j) a polypeptide consisting essentially of amino acids 48-72 of SEQ ID NO:X;
k) a polypeptide consisting essentially of amino acids 48-94 of SEQ ID NO:X;
l) a polypeptide consisting essentially of amino acids 62-72 of SEQ ID NO:X;
m) a polypeptide consisting essentially of amino acids 62-94 of SEQ ID NO:X;
n) a polypeptide consisting essentially of amino acids 73-94 of SEQ ID NO:X;
o) a polypeptide consisting essentially of amino acids 1-23 of SEQ ID NO:X;
p) a polypeptide consisting essentially of amino acids 48-66 of SEQ ID NO:X;
q) a polypeptide consisting essentially of amino acids 48-71 of SEQ ID NO:X;
r) a polypeptide consisting essentially of amino acids 48-76 of SEQ ID NO:X;
s) a polypeptide consisting essentially of amino acids 43-61 of SEQ ID NO:X;
t) a polypeptide consisting essentially of amino acids 38-61 of SEQ ID NO:X;
u) a polypeptide consisting essentially of amino acids 33-61 of SEQ ID NO:X;
v) a polypeptide consisting essentially of amino acids 43-66 of SEQ ID NO:X;
w) a polypeptide consisting essentially of amino acids 38-66 of SEQ ID NO:X;
x) a polypeptide consisting essentially of amino acids 33-66 of SEQ ID NO:X;
y) a polypeptide consisting essentially of amino acids 43-71 of SEQ ID NO:X;
z) a polypeptide consisting essentially of amino acids 38-71 of SEQ ID NO:X;
aa) a polypeptide consisting essentially of amino acids 33-71 of SEQ ID NO:X;
ab) a polypeptide consisting essentially of amino acids 43-76 of SEQ ID NO:X;
ac) a polypeptide consisting essentially of amino acids 38-76 of SEQ ID NO:X; and
ad) a polypeptide consisting essentially of amino acids 33-76 of SEQ ID NO:X.

16-148. (canceled)
